(19) United States
(12) Patent Application Publication
(10) Pub. No.: US 2024/0117202 A1
(43) Pub. Date: Apr. 11, 2024

(54) FORMULATIONS AND APPLICATIONS THEREOF IN OPTOELECTRONIC FIELD

(71) Applicant: Zhejiang Brilliant Optoelectronic Technology Co.,Ltd., Taizhou (CN)

(72) Inventors: Junyou PAN, Taizhou (CN); Xiang CHEN, Taizhou (CN); Jiahui TAN, Taizhou (CN)

(73) Assignee: Zhejiang Brilliant Optoelectronic Technology Co.,Ltd., Taizhou (CN)

(21) Appl. No.: 18/483,364

(22) Filed: Oct. 9, 2023

Related U.S. Application Data
(63) Continuation of application No. PCT/CN2022/085357, filed on Apr. 6, 2022.

(30) Foreign Application Priority Data
Apr. 7, 2021 (CN) 202110370887.3

(51) Int. Cl. C09D 5/22; C09D 7/20; C09D 7/63; C09D 133/12; C09K 11/06; H10K 50/844

(52) U.S. Cl. CPC C09D 5/22; C09D 7/20; C09D 7/63; C09D 133/12; C09K 11/06; H10K 50/844; C08K 5/55

(57) ABSTRACT

Disclosed are formulations including an organic compound H, an emitter E, and an organic resin. Also provided are organic functional films containing the formulations. Further provided are organic light-emitting devices containing the formulations.

### CROSS-REFERENCE TO RELATED APPLICATIONS

The present application is a continuation of International Application No. PCT/CN2022/085357, filed on Apr. 6, 2022, which claims priority to Chinese Patent Application No. 202110370887.3, filed on Apr. 7, 2021. All of the aforementioned applications are incorporated herein by reference in their entireties.

### TECHNICAL FIELD

The present disclosure relates to the field of organic optoelectronic material and device technology, and in particular to a formulation, an organic functional film, an organic light-emitting device, and the applications thereof in the optoelectronic field.

### BACKGROUND

According to the principles of colorimetry, the narrower the full width at half maximum (FWHM) of the lights perceived by the human eyes is, the higher the color purity, and thus the more vivid the color display would be. Display devices with narrow-FWHM red, green and blue primary light are able to show vivid views with high color gamut and high visual quality.

The current mainstream full-color displays are achieved mainly in two ways. The first method is to actively emit red, green and blue lights, typically such as RGB-OLED display. The current mature technology is to fabricate light-emitting devices with three colors by vacuum evaporation with fine metal masks, which is complex, at high cost and difficult to achieve high-resolution display over 600 ppi. The second method is using color converters to convert the single-color light from the light-emitting devices into different colors, thereby achieving a full-color display. For example, Samsung combines blue OLEDs with red and green quantum dots (QD) films as the color converters. In this case, the fabrication of the light emitting devices is much simpler, and thus higher yield. Furthermore, the manufacture of the color converters can be achieved by different technologies, such as vacuum evaporation, ink-jet printing, transfer printing, photolithography, etc., appliable to a variety of display products with very different resolution requirements from low resolution large-size TV (around only 50 ppi) to high resolution silicon-based micro-display (over 3000 ppi).

Currently, there are mainly two types of color conversion materials used in mainstream color converters. The first one is an inorganic nanocrystal, commonly known as a quantum dot, which is a nanoparticle (especially is a quantum dot) of an inorganic semiconductor material (InP, CdSe, CdS, ZnSe, etc.) with a diameter of 2 nm to 8 nm. Limited by the current synthesis and separation technology of quantum dots, the FWHMs of CD-containing quantum dots typically range from 25 nm to 40 nm, which meet the display requirements of NTSC for color purity. Meanwhile, Cd-free quantum dots generally come with larger FWHMs of 35 nm to 75 nm. In addition, the extinction coefficient is generally low, requiring thicker films, the typical 10 μm or more is needed to achieve complete absorption of blue light, which is a great challenge for mass production processes, especially for Samsung's technology of combing blue OLED with red-green quantum dots. The second one is an organic dye, comprising various organic conjugated small molecules with chromophores. This organic dye generally has high extinction coefficient, but the intra-molecular thermal relaxation and the large vibration energy are always non-negligible, leading to the large FWHM (typically over 60 nm) of its emission spectrum.

The present inventors have disclosed small molecules and polymer materials with narrow FWHM in two previous applications. Although their extinction coefficients are greatly improved compared with quantum dots, further optimization is still needed. Moreover, due to the complex synthesis of this organic dye, which is used to make thick film at a high cost, the material as a color converter still needs to be improved.

### SUMMARY

In one aspect, the present disclosure provides a formulation comprising an organic compound H, an emitter E, and an organic resin, where 1) the emission spectrum of the organic compound H is on the short wavelength side of the absorption spectrum of the emitter E, and at least partially overlaps with the absorption spectrum of the emitter E; 2) the FWHM of the emission spectrum of the emitter E≤55 nm.

In addition or alternatively, the emitter E comprises a structural unit of formula (1) or (2):

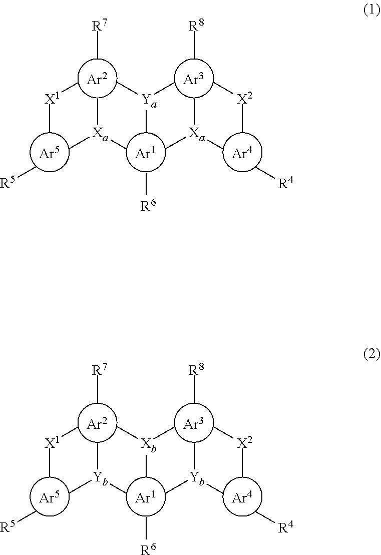

Where each of Ar¹ to Ar³ is independently selected from an aromatic group or a heteroaromatic group containing 5 to 24 ring atoms;

each of Ar⁴ and Ar⁵ is independently selected from null, an aromatic group or a heteroaromatic group containing 5 to 24 ring atoms;

when neither Ar⁴ nor Ar⁵ is null, each of Xₐ and X_{b} at each occurrence is independently selected from N, C(R⁹), or Si(R⁹); each of Yₐ and Y_{b} at each occurrence is independently selected from B, P═O, C(R⁹), or Si(R⁹);

when Ar⁴ and/or Ar⁵ is null, each X_{b} is selected from N, C(R⁹), or Si(R⁹); each Yₐ is selected from B, P═O, C(R⁹), or Si(R⁹); each of Xₐ and Y_{b} at each occurrence is independently selected from N(R⁹), C(R⁹ R¹⁰), Si(R⁹ R¹⁰), C═O, O, C═N(R⁹), C═C(R⁹ R¹⁰), P(R⁹), P(═O)R⁹, S, S═O, or SO₂;

each of X¹ and X² is independently selected from null or a bridging group;

each of R⁴ to R¹⁰ at each occurrence is independently selected from the group consisting of —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —NO₂, —CF₃, —Cl, —Br, —F , —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, and a disubstituted unit in any position of the above substituents or a combination thereof, where one or more R⁴-R¹⁰ may form a monocyclic or polycyclic aliphatic or aromatic ring system with each other and/or with the rings bonded thereto.

In addition or alternatively, the formulation further comprises at least one solvent.

In another aspect, the present disclosure also provides an organic functional film comprising a formulation as described herein.

In yet another aspect, the present disclosure further provides an optoelectronic device comprising a formulation or an organic functional film as described herein.

In yet another aspect, the present disclosure further provides an organic light-emitting device comprising a substrate, a first electrode, an organic light-emitting layer, a second electrode, a color conversion layer, and an encapsulation layer in sequence from bottom to top, the second electrode is at least partially transparent, where 1) the color conversion layer comprises a formulation as described herein; 2) the color conversion layer absorbs 90% or more of the light emitted by the organic light-emitting layer through the second electrode; 3) the emission spectrum of the organic compound H is on the short wavelength side of the absorption spectrum of the emitter E, and at least partially overlaps with the absorption spectrum of the emitter E; 4) the FWHM of the emission spectrum of the emitter E≤55 nm.

Beneficial effects: in the formulation as described herein, the organic compound H has high extinction coefficient, the emitter E has high luminescence efficiency and narrow emission FWHM. Moreover, and the energy transfer efficiency between the organic compound H and the emitter E is high, thereby optimizing separately the absorption and luminescence functions, and facilitating the preparation of high-efficiency color converters with a thin thickness, meeting the requirements of high color gamut displays. Furthermore, the organic compound H can be selected from the compounds easy to synthesize. Due to the high proportion of the organic compound H in the formulation, the cost could be greatly reduced.

### BRIEF DESCRIPTION OF THE DRAWINGS

FIG. 1 is a schematic view of a red, green and blue (RGB) three-color display device.

FIG. 2 is the emission spectrum of compound H4 in toluene, and the absorption and emission spectrum of compound E1 in toluene.

FIG. 3 is the emission spectrum of compound H11 in toluene, and the absorption and emission spectrum of compound E1 in toluene.

FIG. 4 is the emission spectrum of compound H12 in toluene, and the absorption and emission spectrum of compound E1 in toluene.

FIG. 5 is the emission spectrum of compound H13 in toluene, and the absorption and emission spectrum of compound E1 in toluene.

FIG. 6 is the emission spectrum of compound H14 in toluene, and the absorption and emission spectrum of compound E1 in toluene.

FIG. 7 is the absorption and emission spectrum of compound E3 in toluene.

FIG. 8 is the absorption and emission spectrum of compound E4 in toluene.

FIG. 9 is the absorption and emission spectrum of compound E6 in toluene.

### DETAILED DESCRIPTION OF THE EMBODIMENTS

The present disclosure provides a formulation, an organic functional film, an organic light-emitting device, and the applications thereof in the optoelectronic field.

In order to facilitate understanding of the present disclosure, the present disclosure will be described in detail below with reference to the accompanying drawings, in which the preferred embodiments of the present disclosure are shown. The present disclosure may be embodied in many different forms and is not limited to the embodiments described herein. Rather, these embodiments are provided so that the understanding of the disclosure of the present disclosure will be more thorough.

Unless otherwise defined, all technical and scientific terms used herein have the same meaning as commonly understood by one of ordinary skill in the art belonging to the present disclosure. The terms used herein in the description of the present disclosure are used only for the purpose of describing specific embodiments and are not intended to be limiting of the present disclosure. As used herein, the term “and/or” includes any and all combinations of one or more of the relevant listed items.

As used herein, the terms “host material”, “matrix material” have the same meaning, and they are interchangeable with each other.

As used herein, the terms “formulation”, “printing ink”, and “ink” have the same meaning, and they are interchangeable with each other.

In one aspect, the present disclosure provides a formulation comprising an organic compound H, an emitter E, and an organic resin, where 1) the emission spectrum of the organic compound H is on the short wavelength side of the absorption spectrum of the emitter E, and at least partially overlaps with the absorption spectrum of the emitter E; 2) the FWHM of the emission spectrum of the emitter E≤55 nm.

In some embodiments, the FWHM of the emission spectrum of the emitter E≤50 nm, preferably ≤40 nm, more preferably ≤35 nm, and most preferably ≤30 nm.

In some embodiments, the photoluminescence quantum yield (PLQY) of the emitter E≥60%, preferably≥65%, more preferably ≥70%, and most preferably ≥80%.

In some embodiments, the emitter E comprises a structural unit of formula (1) or (2):

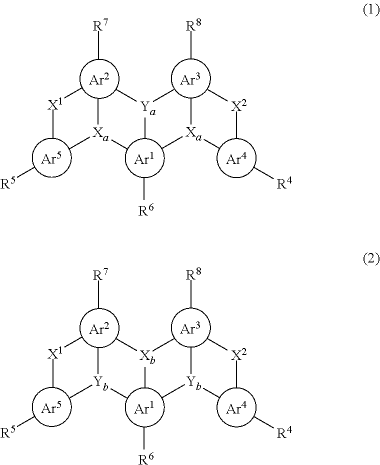

Where each of Ar¹ to Ar³ is independently selected from an aromatic group or a heteroaromatic group containing 5 to 24 ring atoms; each of Ar⁴ and Ar⁵ is independently selected from null, an aromatic group or a heteroaromatic group containing 5 to 24 ring atoms; when neither Ar⁴ nor Ar⁵ is null, each of Xₐ and X_{b} at each occurrence is independently selected from N, C(R⁹), or Si(R⁹); each of Yₐ and Y_{b} at each occurrence is independently selected from B, P═O, C(R⁹), or Si(R⁹); when Ar⁴ and/or Ar⁵ is null, each X_{b} is selected from N, C(R⁹), or Si(R⁹); each Yₐ is selected from B, P═O, C(R⁹), or Si(R⁹); each of Xₐ and Y_{b} at each occurrence is independently selected from N(R⁹), C(R⁹R¹⁰), Si(R⁹R¹⁰), C═O, O, C═N(R⁹), C═C(R⁹R¹⁰), P(R⁹), P(═O)R⁹, S, S═O, or SO₂; each of X¹ and X² is independently selected from null or a bridging group;

each of R⁴ to R¹⁰ at each occurrence is independently selected from the group consisting of —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, and a disubstituted unit in any position of the above substituents or a combination thereof, where one or more R⁴-R¹⁰ may form a monocyclic or polycyclic aliphatic or aromatic ring system with each other and/or with the rings bonded thereto.

In some embodiments, each of R⁴ to R¹⁰ at each occurrence is independently selected from the group consisting of —H, -D, a C₁-C₁₀ linear alkyl group, a C₁-C₁₀ linear haloalkyl group, a C₁-C₁₀ linear alkoxy group, a C₁-C₁₀ linear thioalkoxy group, a C₃-C₁₀ branched/cyclic alkyl group, a C₃-C₁₀ branched/cyclic haloalkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, and a disubstituted unit in any position of the above substituents or a combination thereof, where one or more R⁴-R¹⁰ may form a monocyclic or polycyclic aliphatic or aromatic ring system with each other and/or with the rings bonded thereto.

In some embodiments, the emitter E comprises a structural unit of formula (1a) or (2a):

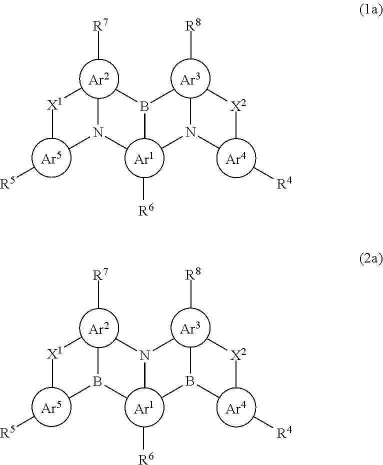

Where Ar¹ to Ar⁵, X¹, X², R⁴ to R⁸ are identically defined as described above.

In some embodiments, each of X¹ and X² is O or S; in some embodiments, each of X¹ and X² is O.

In some embodiments, at least one of X¹ or X² is null; particularly preferably, both are null, in which case the emitter E comprises a structural unit of formula (1b) or (2b):

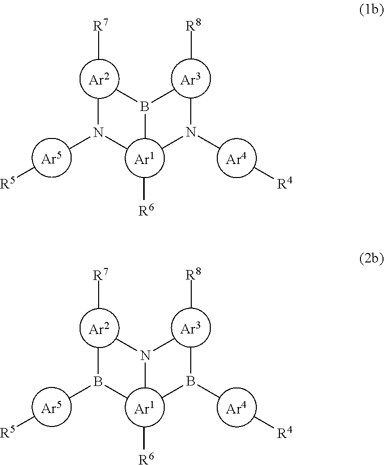

Where Ar¹ to Ar⁵, R⁴ to R⁸ are identically defined as described above.

In some embodiments, at least one of X¹ or X² is a single bond; particularly preferably, both are single bonds, in which case the emitter E comprises a structural unit of formula (1c) or (2c):

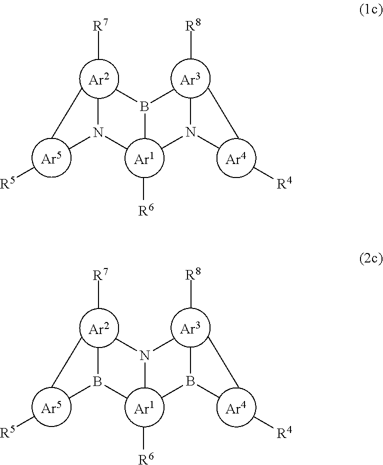

Where Ar¹ to Ar⁵, R⁴ to R⁸ are identically defined as described above.

In some embodiments, X¹ and X² at each occurrence are the same or different di-bridging groups; the preferred di-bridging groups are selected form the following formulas:

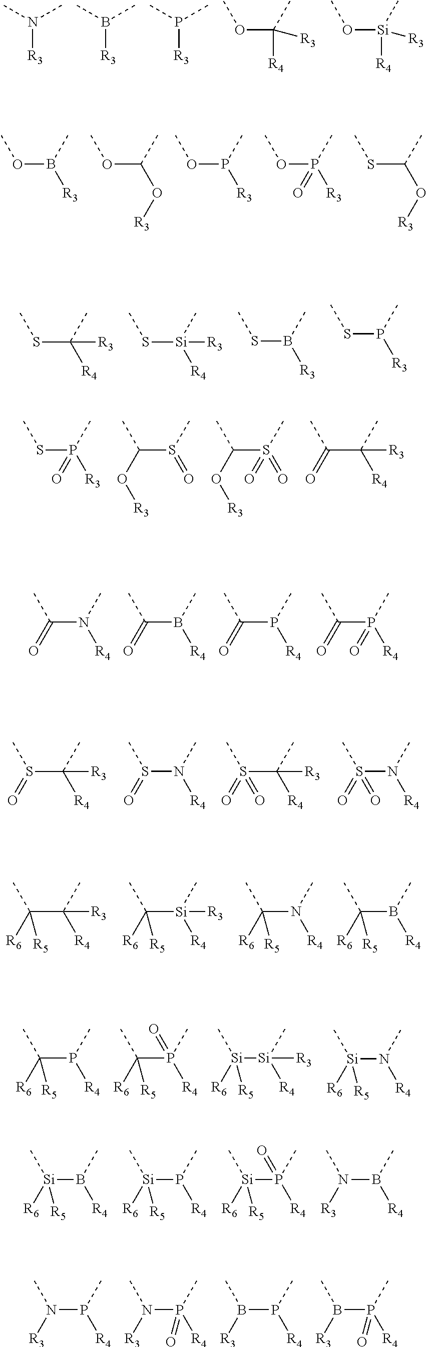

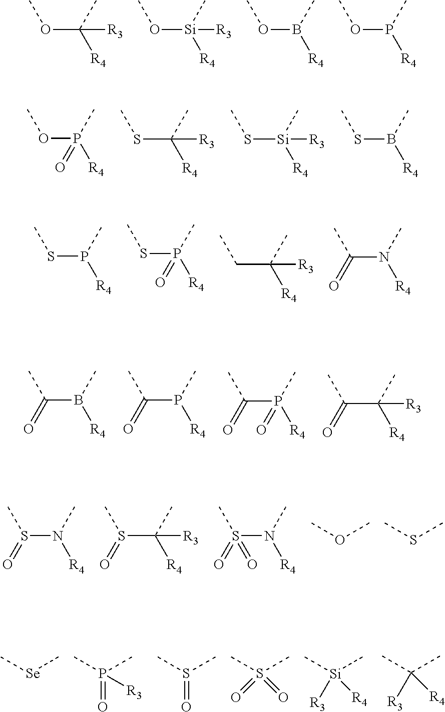

Where R³, R⁴, R⁵, and R⁶ are identically defined as the above-mentioned R⁴, and the dashed bonds refer to the covalent bonds connecting to the adjacent structural units.

For the purposes of the present disclosure, the aromatic ring system contains 5 to 10 carbon atoms in the ring system, the heteroaromatic ring system contains 1 to 10 carbon atoms, together with at least one heteroatom in the ring system, while the total number of carbon atoms and heteroatoms is at least 4. The heteroatoms are preferably selected from Si, N, P, O, S and/or Ge, particularly preferably selected from Si, N, P, O and/or S. For the purposes of the present disclosure, the aromatic or heteroaromatic ring systems contain not only aromatic or heteroaromatic groups, but also have a plurality of aryl or heteroaryl groups spaced by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like are also considered to be aromatic ring systems for the purposes of this disclosure.

For the purposes of the present disclosure, the any H atom on the organic compound H or the emitter E may be optionally substituted with R⁴. R⁴ is defined as above, which may be preferably selected from: (1) a C₁-C₁₀ alkyl group, particularly preferably selected from the following groups: methyl, ethyl, n-propyl, isopropyl, cyclopropyl, n-butyl, isobutyl, sec-butyl, t-butyl, cyclobutyl, 2-methylbutyl, n-pentyl, n-hexyl, cyclohexyl, n-heptyl, cycloheptyl, n-octyl, cyclooctyl, 2-methylheptyl, trifluoromethyl, pentafluoroethyl, 2,2,2-trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, or octenyl; (2) a C₁-C₁₀ alkoxy group, particularly preferably methoxy, ethoxy, n-propoxy, isopropoxy, n-butoxy, isobutoxy, sec-butoxy, tert-butoxy, or 2-methylbutoxy; (3) a C₂-C₁₀ aryl or heteroaryl group, which may be monovalent or divalent depending on the application, and in each case can also be optionally substituted with the group R⁴ mentioned above and may be attached to an aromatic or heteroaromatic ring at any desired position, particularly preferably selected from the following: benzene, naphthalene, anthracene, dihydropyrene, chrysene, pyrene, fluoranthene, naphthacene, pentacene, benzopyrene, furan, benzofuran, isobenzofuran, dibenzofuran, thiophene, benzothiophene, isobenzothiophene, thiofluorene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenimidazole, pyridimidazole, pyrazine-imidazole, quinoxaline-imidazole, oxazole, benzoxazole, naphthoxazole, anthracenazole, phenoxazole, isoxazole, 1,2-thiazole, 1,3-thiazole, benzothiazole, pyridazine, benzopyridazine, pyrimidine, benzopyrimidine, quinoxaline, pyrazine, phenazine, 1,5-naphthyridine, azocarbazole, benzocholine, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thinadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, or benzothiadiazole. For the purposes of the present disclosure, aromatic and heteroaromatic ring systems are particularly considered to be, in addition to the above-mentioned aryl and heteroaryl groups, also refer to biphenylene, terphenylene, fluorene, spirobifluorene, dihydrophenanthrene, tetrahydropyrene, cis-indenofluorene, or trans-indenofluorene.

In some embodiments, Ar¹ to Ar⁵ of the emitter E at each occurrence are the same or different and are independently selected from the group consisting of aromatic or heteroaromatic groups with 5 to 20 ring atoms; preferably 5 to 18 ring atoms, more preferably 5 to 15 ring atoms; and most preferably 5 to 10 ring atoms; they may be unsubstituted or substituted with one or two R⁴. Preferred aromatic or heteraromatic groups include benzene, naphthalene, anthracene, phenanthrene, pyridine, pyrene, or thiophene.

In some embodiments, each of Ar¹ to Ar⁵ is independently selected from the following structural formulas:

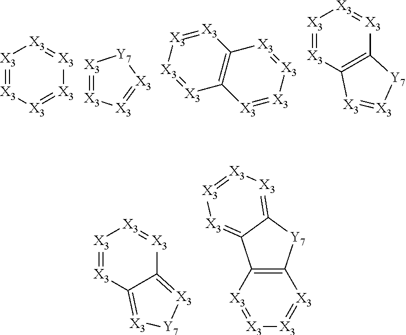

Where each X₃ is CR¹¹ or N; each Y₇ is selected from NR¹¹, CR¹²R¹³, SiR¹⁴R¹⁵, C(═O), S, or O; R¹¹, R¹², R¹³, R¹⁴, R¹⁵ are identically defined as the above-mentioned R⁴;

Further, each of Ar¹, Ar², Ar³, Ar⁴, and Ar⁵ is independently selected from one of the following structural formulas or any combination thereof, which may be further substituted arbitrarily:

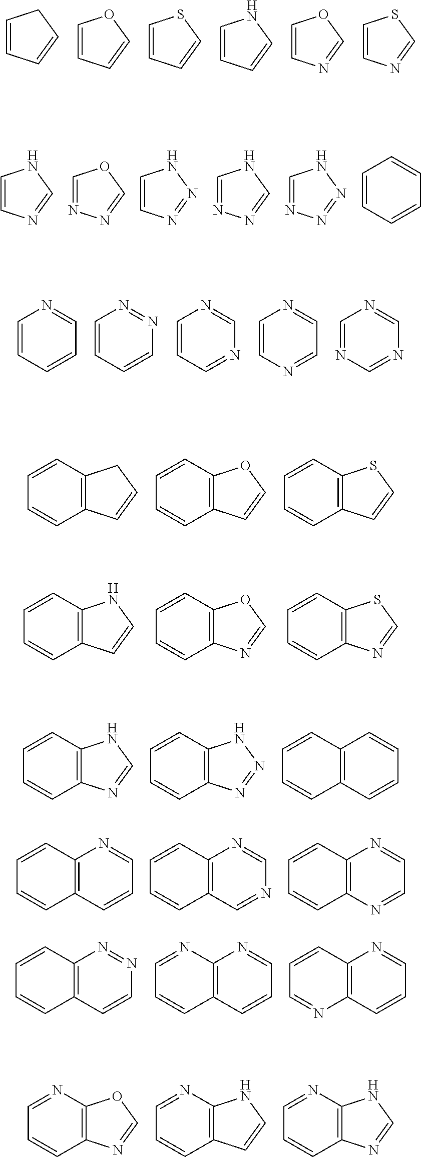

In some embodiments, each of Ar¹ to Ar⁵ is phenyl group.

In some embodiments, at least one of Ar⁴ or Ar⁵ is null; particularly preferably, both are null, in which case the emitter E comprises a structural unit of formula (1b), or (2b), or (1e), or (2e):

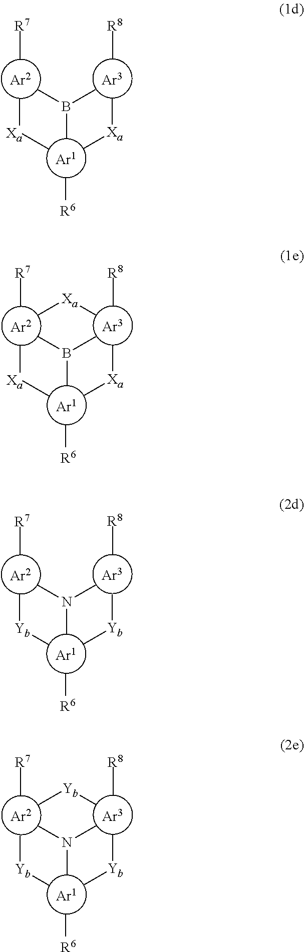

Where Ar¹ to Ar³, Xₐ, Y_{b}, and R⁶ to R⁸ are identically defined as described above.

Preferably, each Xₐ in the formulas (1d) and (1e) are independently selected from N(R⁹), C(R⁹R¹⁰), Si(R⁹R¹⁰), O, or S.

Preferably, each Y_{b} in the formulas (2d) and (2e) are independently selected from C═O, O, S, P(═O)R⁹, S═O, or SO₂; and particularly preferably from C═O.

In some embodiments, the emitter E comprises a structural unit of formulas (1f)-(1i):

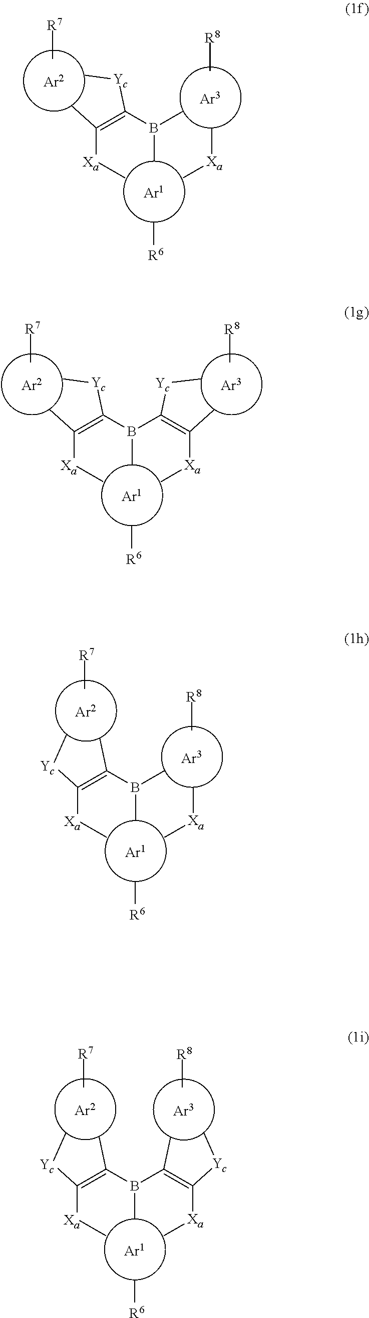

Where each Y_{c} is O or S; Ar¹ to Ar³, Xₐ, R⁶ to R⁸ are identically defined as described above.

In some embodiments, the above-mentioned Ar², Ar³ are preferably selected from the following structural units, which may be further substituted arbitrarily:

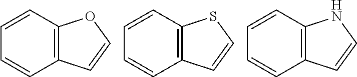

In some embodiments, in the structural units of formulas (1)-(1i), (2)-(2e), each of R⁴ to R⁸ may be same or different in multiple occurrences, comprising the following structural units or any combination thereof:

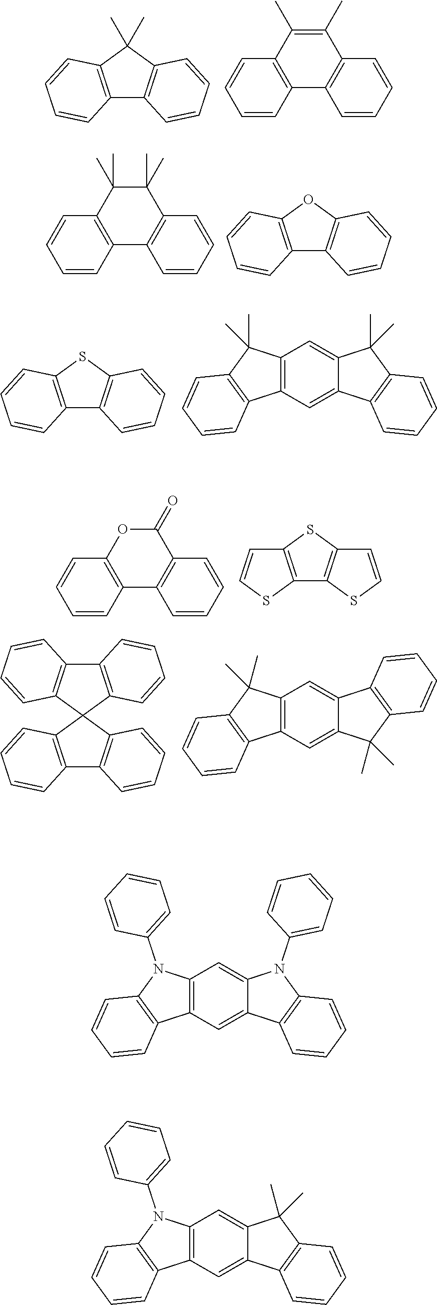

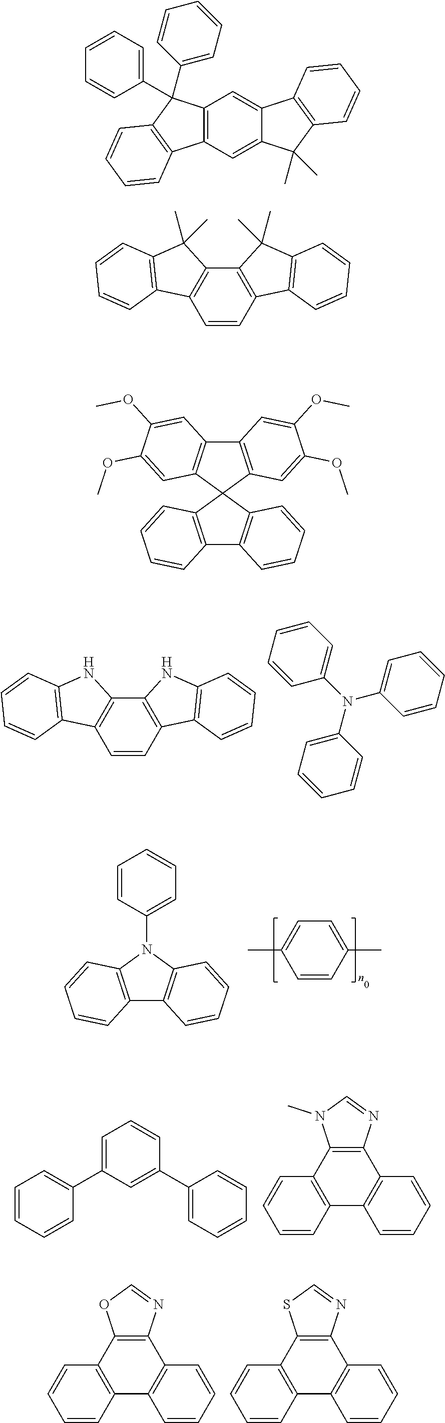

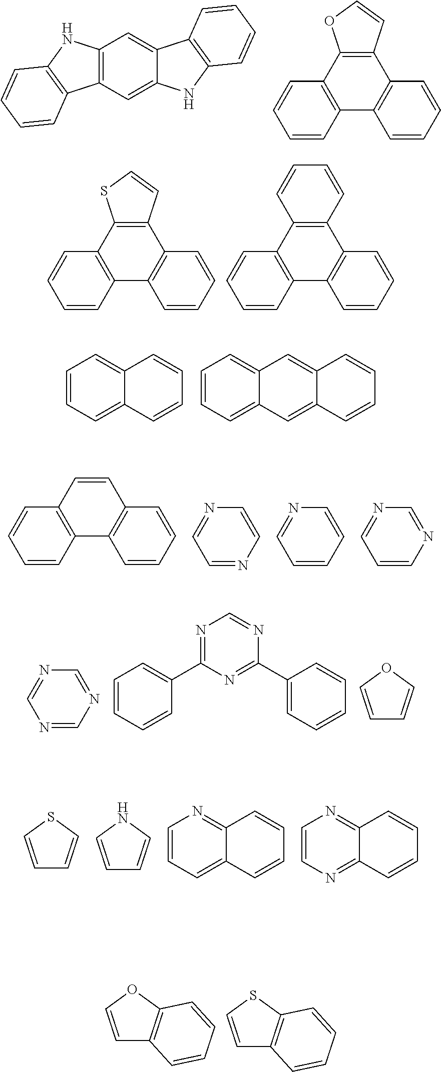

Where n₀ is 1, or 2, or 3, or 4.

In some embodiments, the structure of the emitter E is shown below:

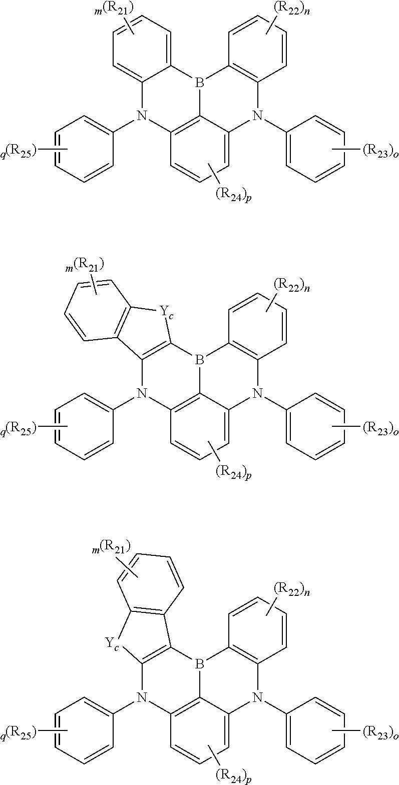

Where each Y_{c} is defined as described above; each of R₂₁ to R₂₅ is independently selected from the group consisting of —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O(—H), an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₂₁-R₂₅ may form a monocyclic or polycyclic aliphatic or aromatic ring system with each other and/or with the rings bonded thereto; and at least one of R₂₁ to R₂₅ comprises an alcohol-soluble or a water-soluble group; m, n are integers from 0 to 4; o, q are integer from 0 to 5; p is an integer from 0 to 3.

Preferably, each of R₂₁ to R₂₅ is independently selected from the group consisting of —H, -D, a C₁-C₁₀ linear alkyl group, a C₁-C₁₀ alkoxy group, a C₁-C₁₀ thioalkoxy group, a C₃-C₁₀ branched/cyclic alkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O(—H), an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to20 ring atoms, an aryloxy or heteroaryloxy group containing 5 to 20 ring atoms, and any combination thereof, where one or more R₂₁-R₂₅ may form a monocyclic or polycyclic aliphatic or aromatic ring system with each other and/or with the rings bonded thereto.

In the embodiments of the present disclosure, the energy level structure of the organic material, triplet energy level (T1), singlet energy level (S1), highest occupied molecular orbital (HOMO), lowest unoccupied molecular orbital (LUMO), and oscillator strength f play key roles. The determination of these energy levels is introduced as follows.

HOMO and LUMO energy levels can be measured by optoelectronic effect, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT) are becoming effective method for calculating the molecular orbital energy levels.

The triplet energy level T1 of an organic material can be measured by low-temperature time-resolved spectroscopy, or calculated by quantum simulation (for example, by Time-dependent DFT), for instance with the commercial software Gaussian 03W (Gaussian Inc.), the specific simulation method is described below. The singlet energy level S1 of the organic material can be determined by the absorption spectrum or the emission spectrum, and can also be calculated by quantum simulation (such as Time-dependent DFT); the oscillator strength f can also be calculated by quantum simulation (such as Time-dependent DFT).

It should be noted that the absolute values of HOMO, LUMO, T1 and S1 may vary depending on the measurement method or calculation method used. Even for the same method, different ways of evaluation, for example, using either the onset or peak value of a CV curve as reference, may result in different (HOMO/LUMO) values. Therefore, reasonable and meaningful comparison should be carried out by using the same measurement and evaluation methods. In the embodiments of the present disclosure, the values of HOMO, LUMO, T1 and S1 are based on the Time-dependent DFT simulation, which however should not exclude the applications of other measurement or calculation methods.

In some embodiments, the S1-T1 of the emitter E≤0.30 eV, preferably ≤0.25 eV, more preferably ≤0.20 eV, further preferably ≤0.15 eV, and most preferably ≤0.10 eV.

In some embodiments, the emitter E of the formulation is a small molecule or a polymer.

In some embodiments, the emitter E has good solubility in the resin or resin prepolymer.

In some embodiments, the emitter E comprises at least one alcoholic-soluble or water-soluble group, as disclosed in a contemporaneous patent with the application No. CN 202110370884.X. The patent document above is specially incorporated herein by reference in their entirety.

In some embodiments, the emitter E comprises at least two alcohol-soluble or water-soluble groups.

In some embodiments, the emitter E comprises at least three alcohol-soluble or water-soluble groups.

In some embodiments, the alcohol-soluble or water-soluble group of the emitter E is selected from: alcohols, aldehydes, acids, crown ethers, polyethers, or primary amines.

Preferably, the alcohol-soluble or water-soluble group is selected from the following structures:

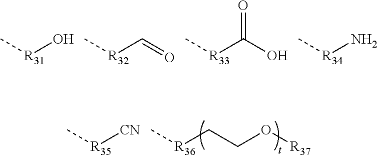

Where R₃₁-R₃₇ are independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)13 X where X represents a halogen atom), a formyl group (—C(═O(—H), an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 40 ring atoms, an aryloxy or heteroaryloxy group containing 5 to 40 ring atoms, and any combination thereof, where one or more R₃₁-R₃₇ may form a monocyclic or polycyclic aliphatic or aromatic ring system with each other and/or with the rings bonded thereto; t is an integer >0.

Furthermore, the individual H atoms or CH₂ groups of the present disclosure may be substituted with the above-mentioned groups or R. R is selected from C₁-C₄₀ alkyl groups, preferably selected from the group consisting of methyl, ethyl, n-propyl, isopropyl, cyclopropyl, n-butyl, isobutyl, sec-butyl, t-butyl, cyclobutyl, methylbutyl, n-pentyl, sec-pentyl, cyclopentyl, n-hexyl, cyclohexyl, n-heptyl, cycloheptyl, n-octyl, cyclooctyl, ethylhexyl, trifluoromethyl, pentafluoroethyl, trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, and octynyl; C₁-C₄₀ alkoxy groups, such as methoxy, trifluoromethoxy, ethoxy, n-propoxy, isopropoxy, n-butoxy, isobutoxy, sec-butoxy, tert-butoxy, or methylbutoxy.

Examples of emitters E are listed below, but not limited to:

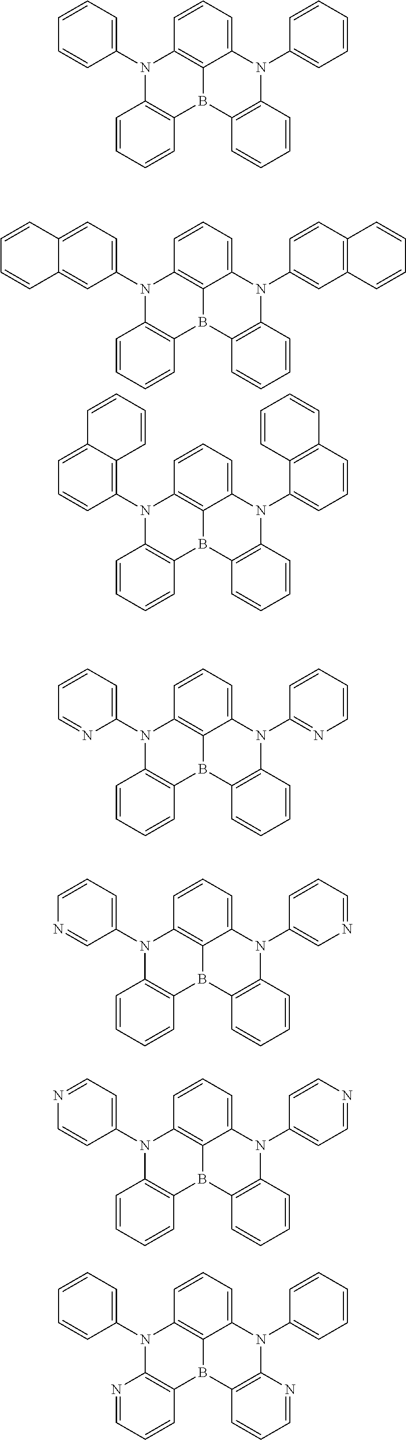

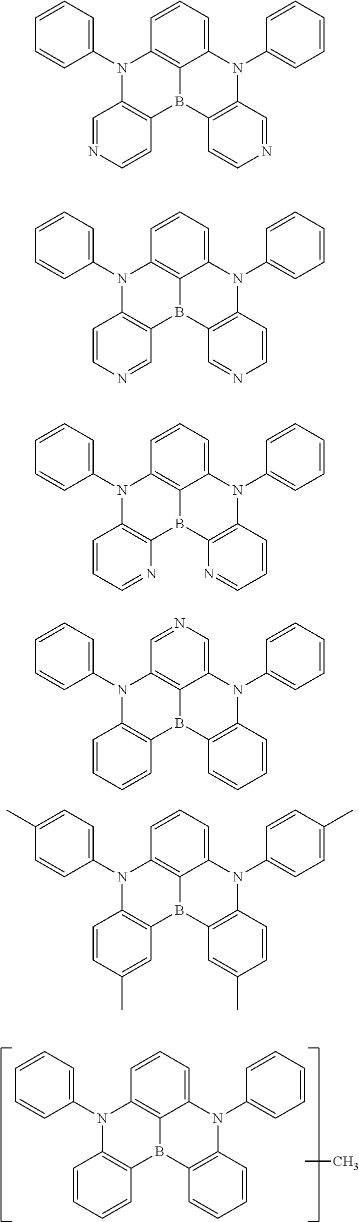

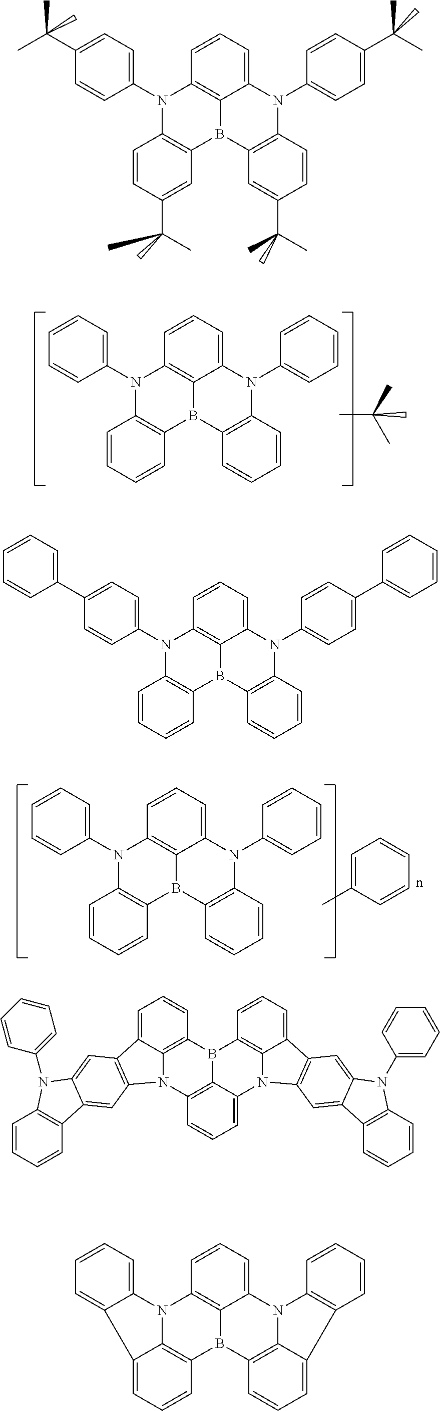

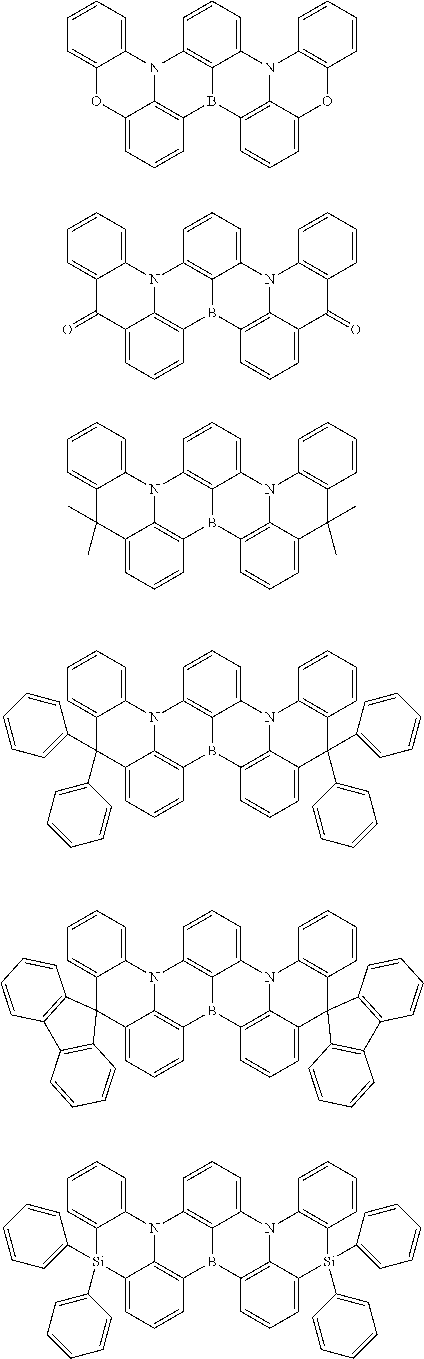 [SMILES: c1ccc2c(c1)Oc1cccc3c1N2c1cccc2c1B3c1cccc3c1N2c1ccccc1O3 ; O=c1c2ccccc2n2c3c(cccc13)B1c3c-2cccc3-n2c3ccccc3c(=O)c3cccc1c32 ; CC1(C)c2ccccc2N2c3cccc4c3B(c3cccc1c32)c1cccc2c1N4c1ccccc1C2(C)C ; c1ccc(C2(c3ccccc3)c3ccccc3N3c4cccc5c4B(c4cccc2c43)c2cccc3c2N5c2ccccc2C3(c2ccccc2)c2ccccc2)cc1 ; c1ccc2c(c1)-c1ccccc1C21c2ccccc2N2c3cccc4c3B(c3cccc1c32)c1cccc2c1N4c1ccccc1C21c2ccccc2-c2ccccc21 ; c1ccc([Si]2(c3ccccc3)c3ccccc3N3c4cccc5c4B(c4cccc2c43)c2cccc3c2N5c2ccccc2[Si]3(c2ccccc2)c2ccccc2)cc1]

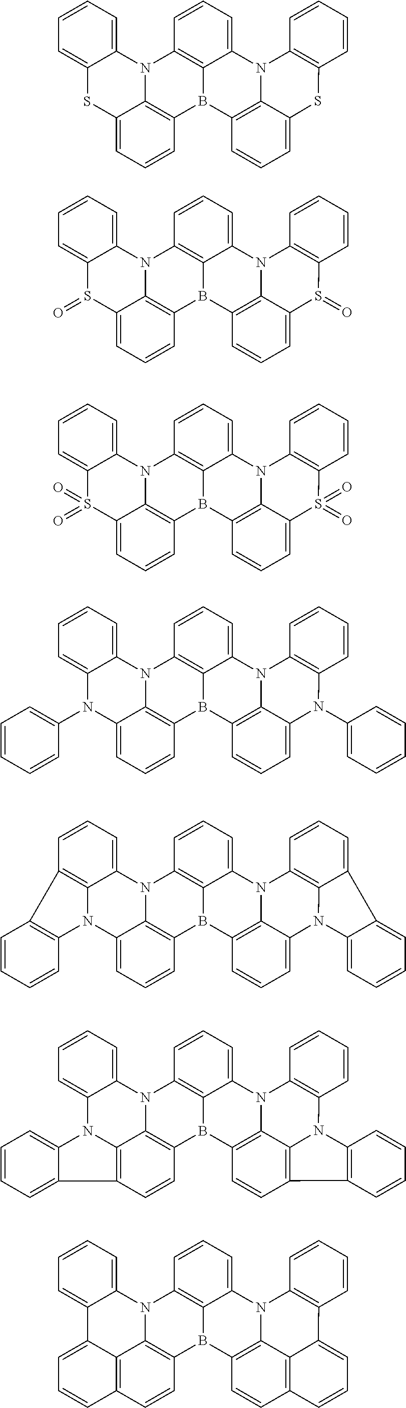

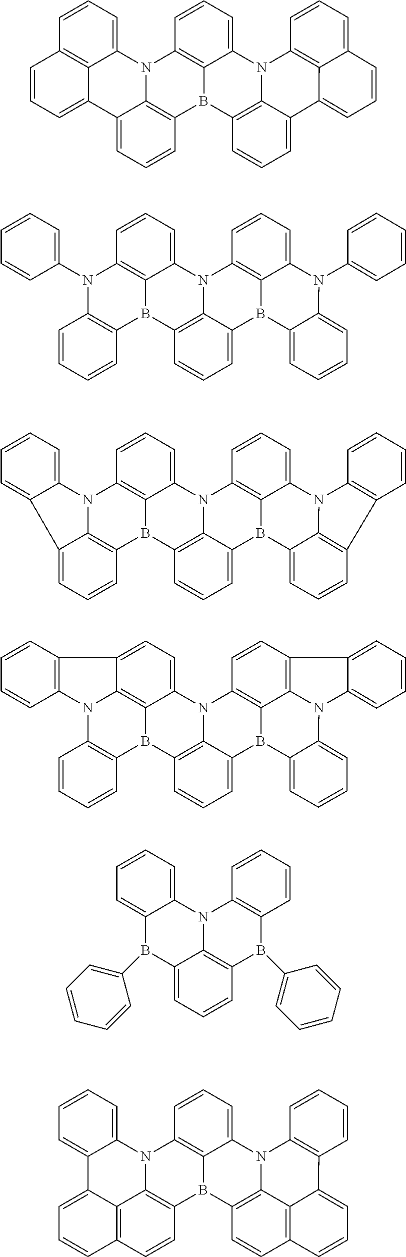

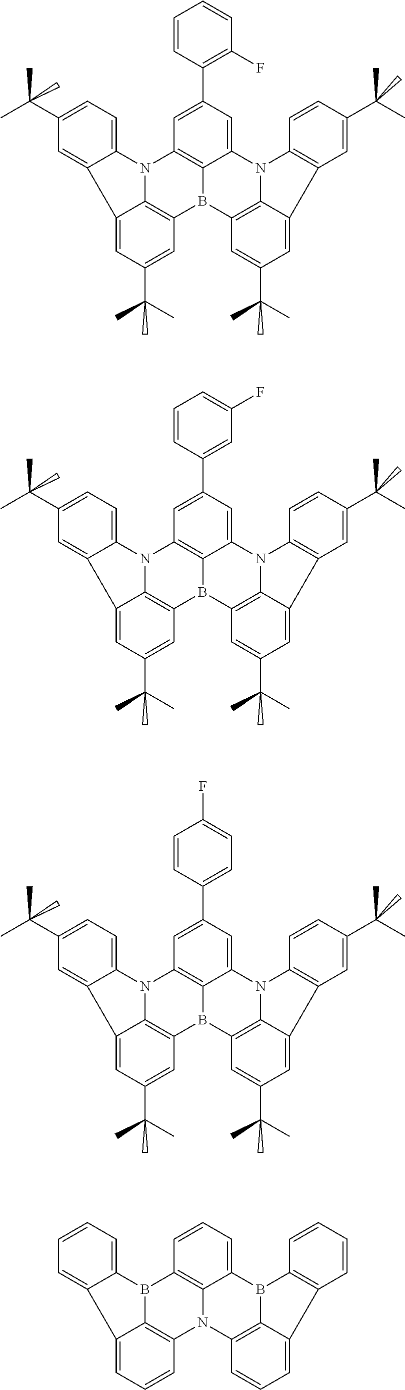

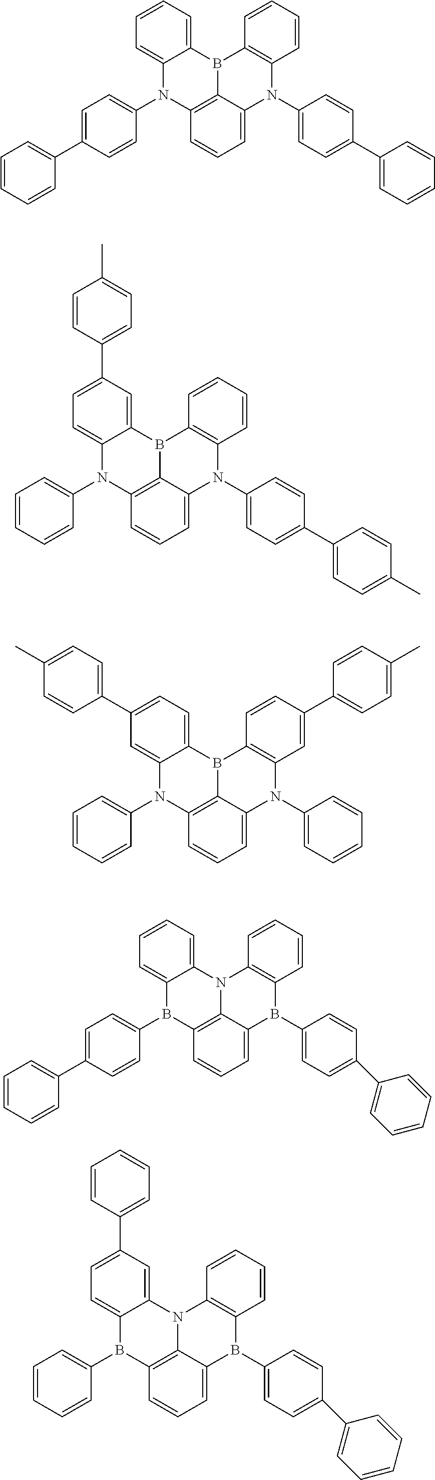

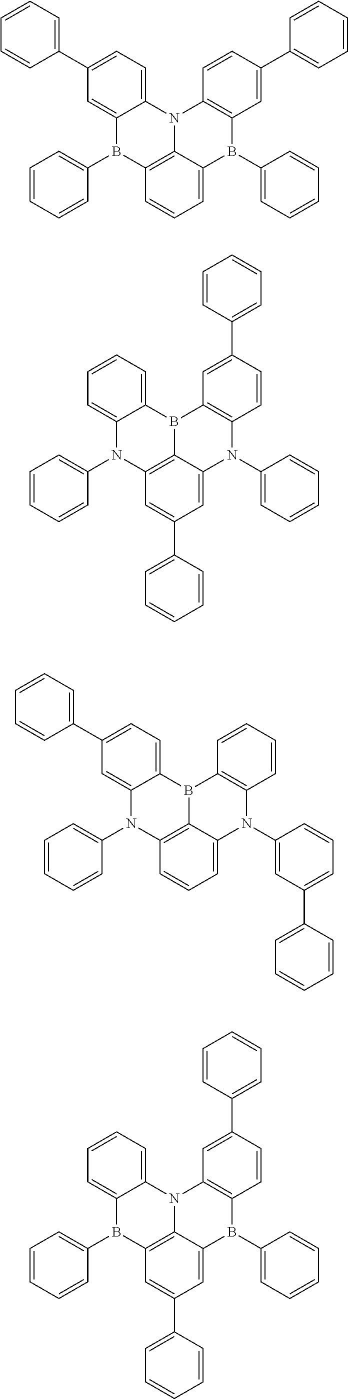 [SMILES: c1ccc(B2c3cc(-c4ccccc4)ccc3N3c4ccc(-c5ccccc5)cc4B(c4ccccc4)c4cccc2c43)cc1 ; c1ccc(-c2ccc3c(c2)B2c4ccccc4N(c4ccccc4)c4cc(-c5ccccc5)cc(c42)N3c2ccccc2)cc1 ; c1ccc(-c2cccc(N3c4ccccc4B4c5ccc(-c6ccccc6)cc5N(c5ccccc5)c5cccc3c54)c2)cc1 ; c1ccc(B2c3ccccc3N3c4cc(-c5ccccc5)ccc4B(c4ccccc4)c4cc(-c5ccccc5)cc2c43)cc1]

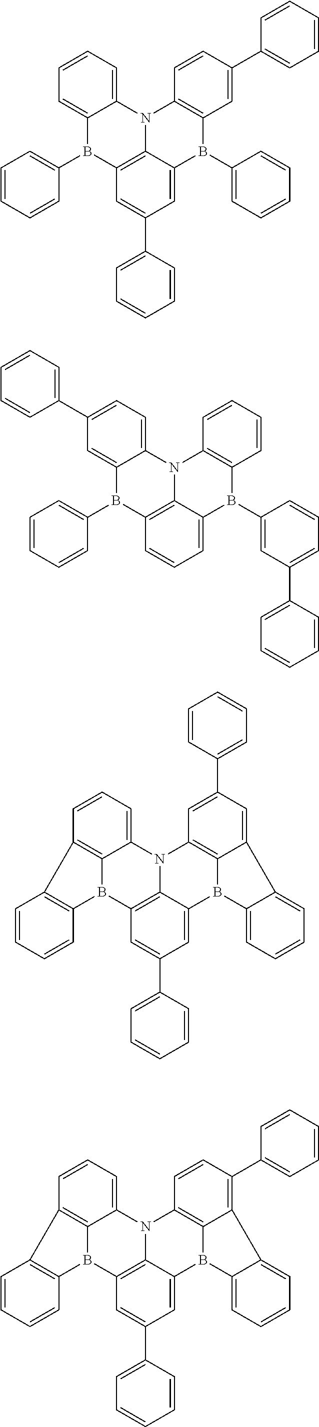 [SMILES: c1ccc(B2c3ccccc3N3c4ccc(-c5ccccc5)cc4B(c4ccccc4)c4cc(-c5ccccc5)cc2c43)cc1 ; c1ccc(B2c3cc(-c4ccccc4)ccc3N3c4ccccc4B(c4cccc(-c5ccccc5)c4)c4cccc2c43)cc1 ; c1ccc(-c2cc3c4c(c2)B2c5ccccc5-c5cc(-c6ccccc6)cc(c52)N4c2cccc4c2B3c2ccccc2-4)cc1 ; c1ccc(-c2cc3c4c(c2)B2c5ccccc5-c5c(-c6ccccc6)ccc(c52)N4c2cccc4c2B3c2ccccc2-4)cc1]

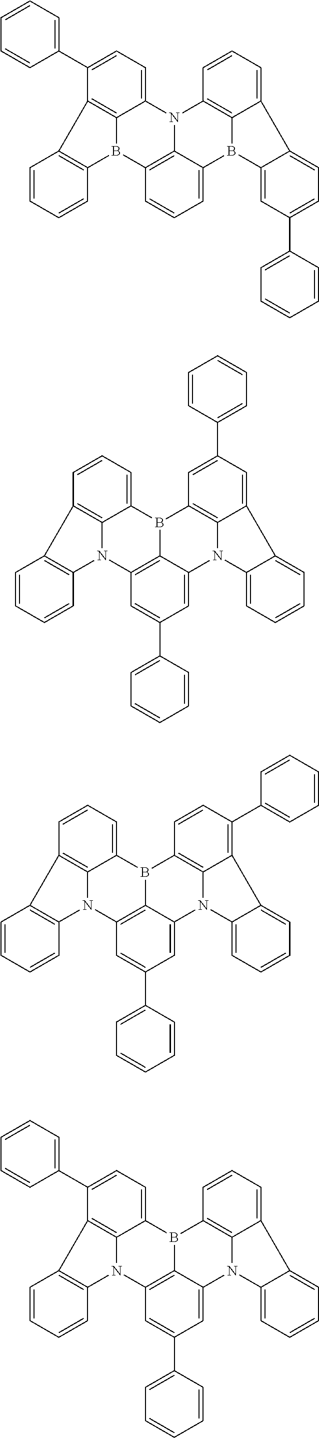 [SMILES: c1ccc(-c2ccc3c(c2)B2c4cccc5c4N(c4cccc-3c42)c2ccc(-c3ccccc3)c3c2B5c2ccccc2-3)cc1 ; c1ccc(-c2cc3c4c(c2)-n2c5ccccc5c5cc(-c6ccccc6)cc(c52)B4c2cccc4c5ccccc5n-3c24)cc1 ; c1ccc(-c2cc3c4c(c2)-n2c5ccccc5c5c(-c6ccccc6)ccc(c52)B4c2cccc4c5ccccc5n-3c24)cc1 ; c1ccc(-c2cc3c4c(c2)-n2c5ccccc5c5c(-c6ccccc6)ccc(c52)B4c2cccc4c5ccccc5n-3c24)cc1]

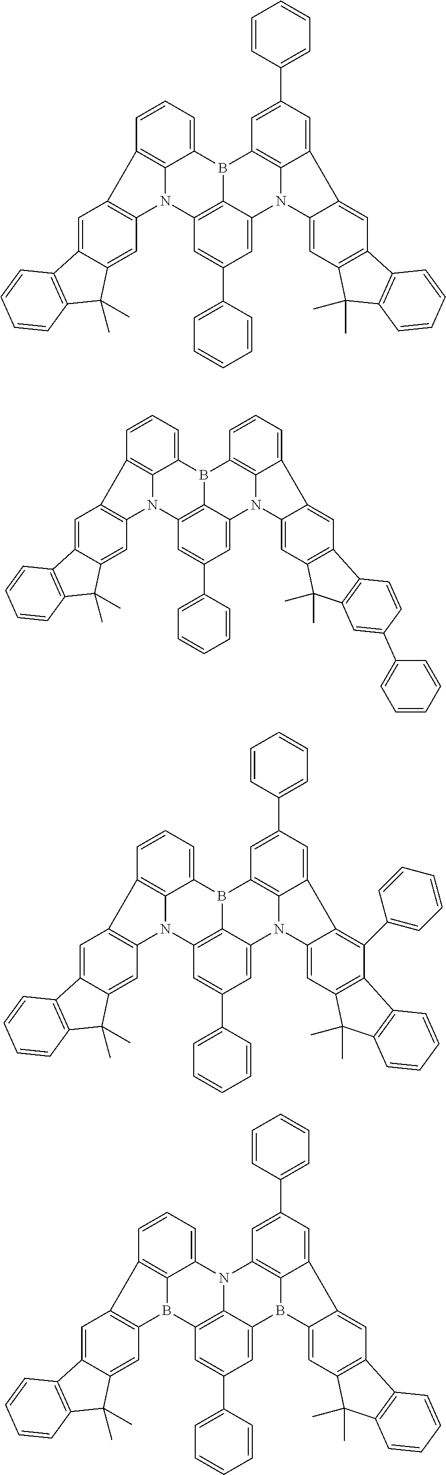 [SMILES: CC1(C)c2ccccc2-c2cc3c4cccc5c4n(c3cc21)-c1cc(-c2ccccc2)cc2c1B5c1cc(-c3ccccc3)cc3c4cc5c(cc4n-2c13)C(C)(C)c1ccccc1-5 ; CC1(C)c2ccccc2-c2cc3c4cccc5c4n(c3cc21)-c1cc(-c2ccccc2)cc2c1B5c1cccc3c4cc5c(cc4n-2c13)C(C)(C)c1cc(-c2ccccc2)ccc1-5 ; CC1(C)c2ccccc2-c2cc3c4cccc5c4n(c3cc21)-c1cc(-c2ccccc2)cc2c1B5c1cc(-c3ccccc3)cc3c4c(-c5ccccc5)c5c(cc4n-2c13)C(C)(C)c1ccccc1-5 ; CC1(C)c2ccccc2-c2cc3c(cc21)B1c2cc(-c4ccccc4)cc4c2N(c2cccc-3c21)c1cc(-c2ccccc2)cc2c1B4c1cc3c(cc1-2)-c1ccccc1C3(C)C]

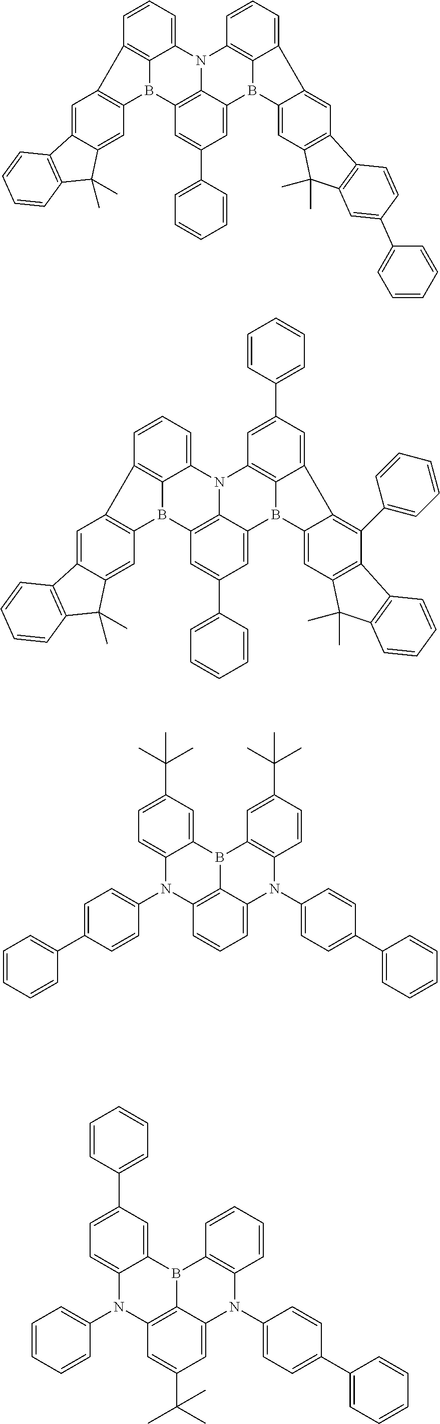 [SMILES: CC1(C)c2ccccc2-c2cc3c(cc21)B1c2cc(-c4ccccc4)cc4c2N(c2cccc-3c21)c1cccc2c1B4c1cc3c(cc1-2)-c1ccc(-c2ccccc2)cc1C3(C)C ; CC1(C)c2ccccc2-c2cc3c(cc21)B1c2cc(-c4ccccc4)cc4c2N(c2cccc-3c21)c1cc(-c2ccccc2)cc2c1B4c1cc3c(c(-c4ccccc4)c1-2)-c1ccccc1C3(C)C ; CC(C)(C)c1ccc2c(c1)B1c3cc(C(C)(C)C)ccc3N(c3ccc(-c4ccccc4)cc3)c3cccc(c31)N2c1ccc(-c2ccccc2)cc1 ; CC(C)(C)c1cc2c3c(c1)N(c1ccccc1)c1ccc(-c4ccccc4)cc1B3c1ccccc1N2c1ccc(-c2ccccc2)cc1]

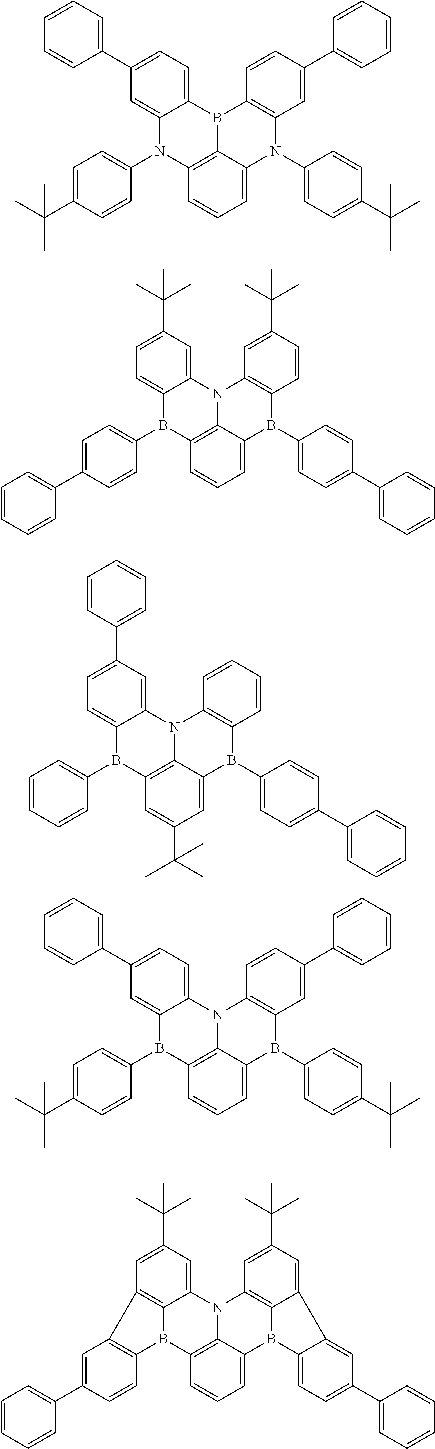

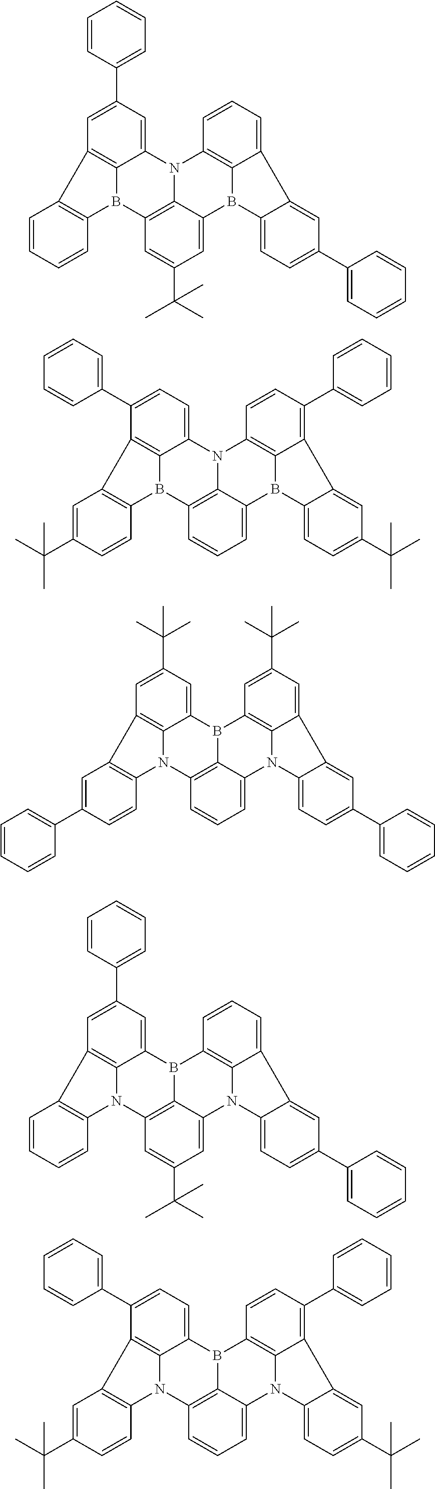

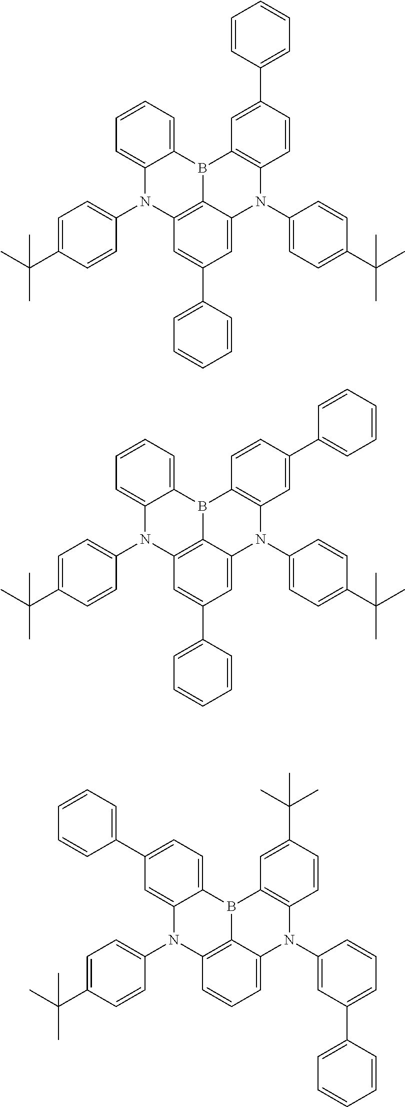

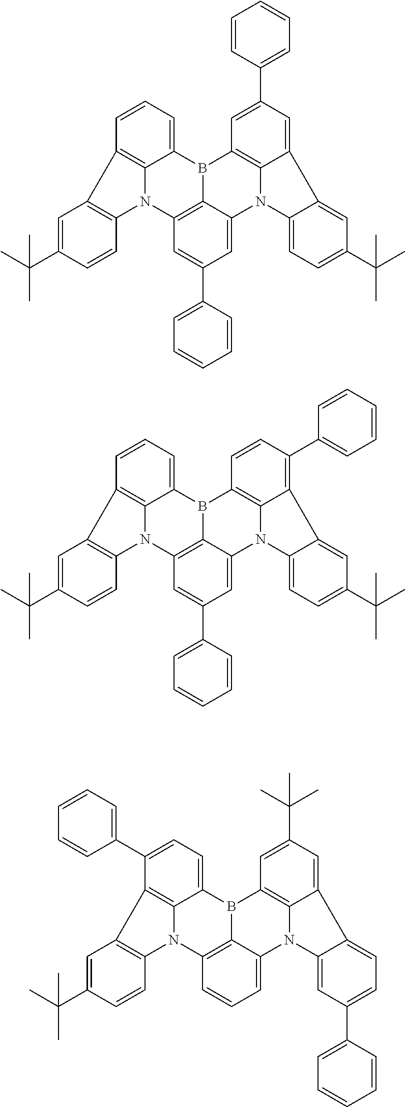

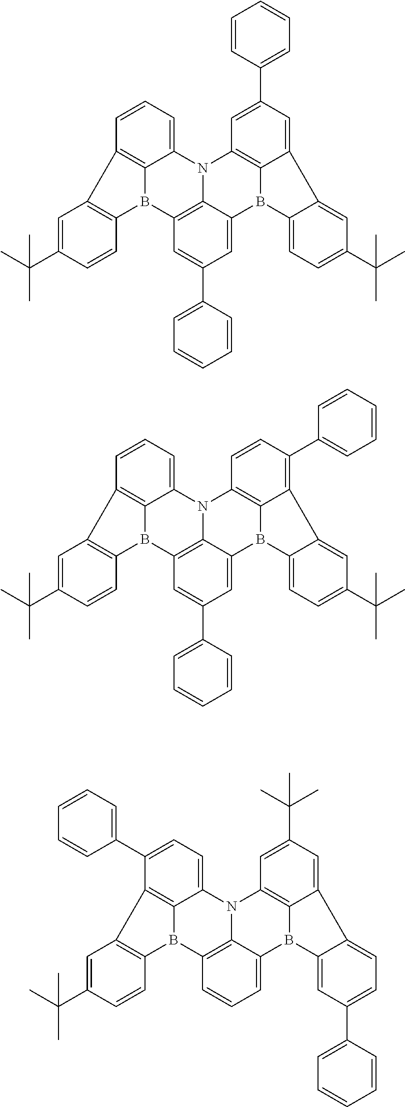

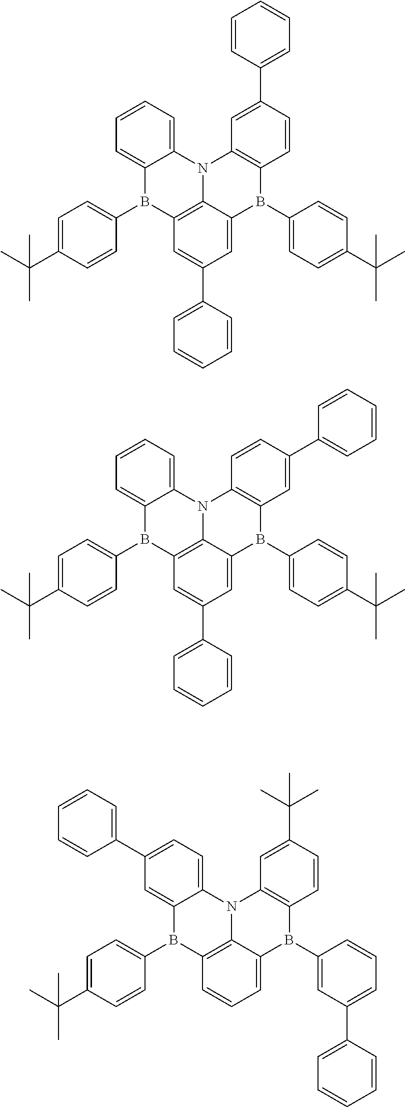

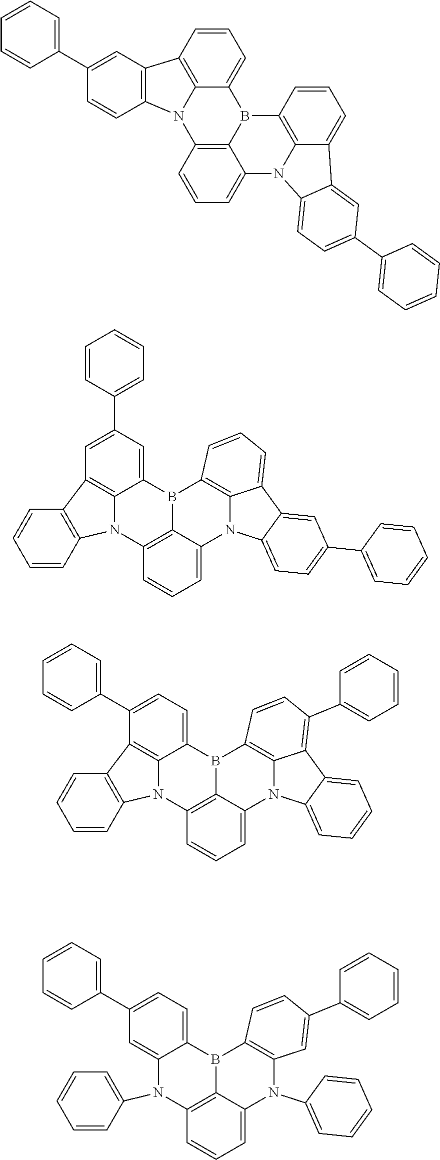 [SMILES: c1ccc(-c2ccc3c(c2)c2cccc4c2n3-c2cccc3c2B4c2cccc4c5cc(-c6ccccc6)ccc5n-3c24)cc1 ; c1ccc(-c2ccc3c(c2)c2cccc4c2n3-c2cccc3c2B4c2cc(-c4ccccc4)cc4c5ccccc5n-3c24)cc1 ; c1ccc(-c2ccc3c4c2c2ccccc2n4-c2cccc4c2B3c2ccc(-c3ccccc3)c3c5ccccc5n-4c23)cc1 ; c1ccc(-c2ccc3c(c2)N(c2ccccc2)c2cccc4c2B3c2ccc(-c3ccccc3)cc2N4c2ccccc2)cc1]

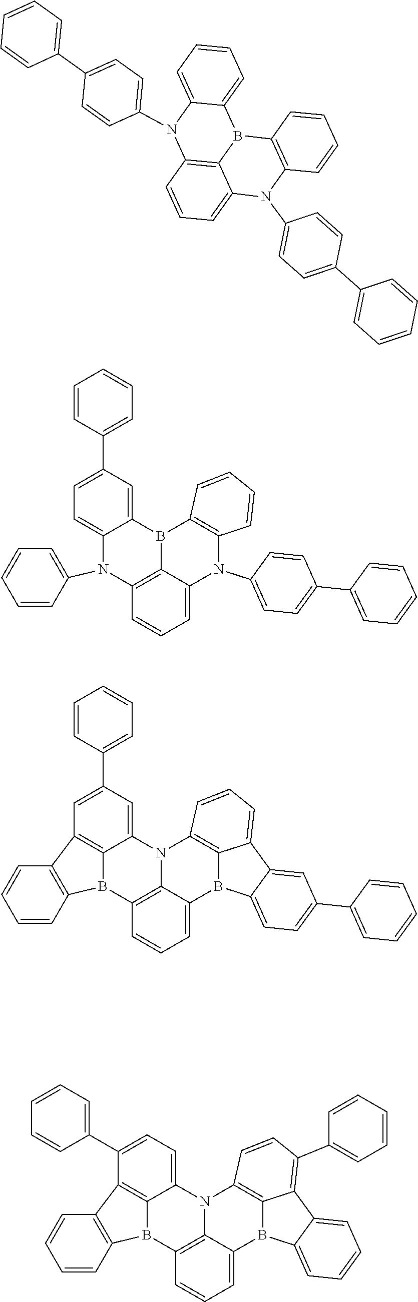 [SMILES: c1ccc(-c2ccc(N3c4ccccc4B4c5ccccc5N(c5ccc(-c6ccccc6)cc5)c5cccc3c54)cc2)cc1 ; c1ccc(-c2ccc(N3c4ccccc4B4c5cc(-c6ccccc6)ccc5N(c5ccccc5)c5cccc3c54)cc2)cc1 ; c1ccc(-c2ccc3c(c2)-c2cccc4c2B3c2cccc3c2N4c2cc(-c4ccccc4)cc4c2B3c2ccccc2-4)cc1 ; c1ccc(-c2ccc3c4c2-c2ccccc2B4c2cccc4c2N3c2ccc(-c3ccccc3)c3c2B4c2ccccc2-3)cc1]

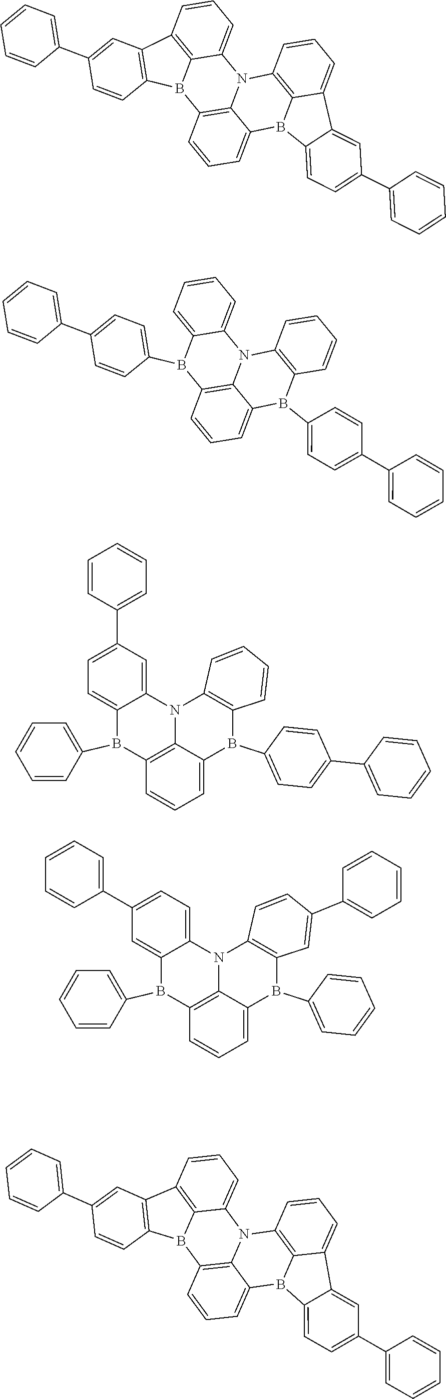 [SMILES: c1ccc(-c2ccc3c(c2)-c2cccc4c2B3c2cccc3c2N4c2cccc4c2B3c2ccc(-c3ccccc3)cc2-4)cc1 ; c1ccc(-c2ccc(B3c4ccccc4N4c5ccccc5B(c5ccc(-c6ccccc6)cc5)c5cccc3c54)cc2)cc1 ; c1ccc(B2c3ccc(-c4ccccc4)cc3N3c4ccccc4B(c4ccc(-c5ccccc5)cc4)c4cccc2c43)cc1 ; c1ccc(B2c3cc(-c4ccccc4)ccc3N3c4ccc(-c5ccccc5)cc4B(c4ccccc4)c4cccc2c43)cc1 ; c1ccc(-c2ccc3c(c2)-c2cccc4c2B3c2cccc3c2N4c2cccc4c2B3c2ccc(-c3ccccc3)cc2-4)cc1]

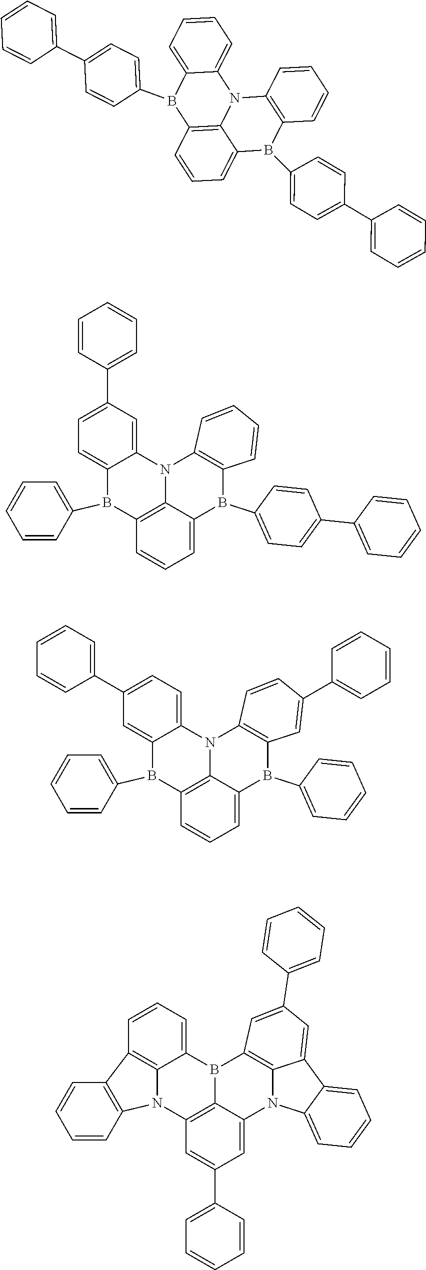 [SMILES: c1ccc(-c2ccc(B3c4ccccc4N4c5ccccc5B(c5ccc(-c6ccccc6)cc5)c5cccc3c54)cc2)cc1 ; c1ccc(B2c3ccc(-c4ccccc4)cc3N3c4ccccc4B(c4ccc(-c5ccccc5)cc4)c4cccc2c43)cc1 ; c1ccc(B2c3cc(-c4ccccc4)ccc3N3c4ccc(-c5ccccc5)cc4B(c4ccccc4)c4cccc2c43)cc1 ; c1ccc(-c2cc3c4c(c2)-n2c5ccccc5c5cc(-c6ccccc6)cc(c52)B4c2cccc4c5ccccc5n-3c24)cc1]

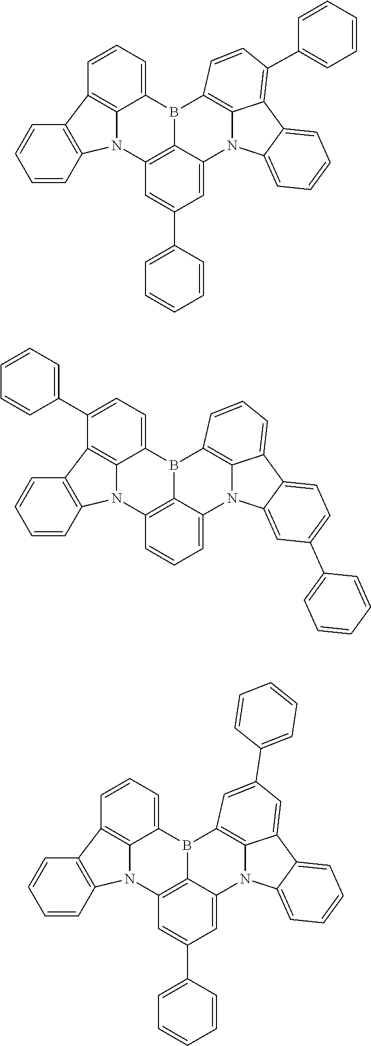 [SMILES: c1ccc(-c2cc3c4c(c2)-n2c5ccccc5c5c(-c6ccccc6)ccc(c52)B4c2cccc4c5ccccc5n-3c24)cc1 ; c1ccc(-c2ccc3c4cccc5c4n(c3c2)-c2cccc3c2B5c2ccc(-c4ccccc4)c4c5ccccc5n-3c24)cc1 ; c1ccc(-c2cc3c4c(c2)-n2c5ccccc5c5cc(-c6ccccc6)cc(c52)B4c2cccc4c5ccccc5n-3c24)cc1]

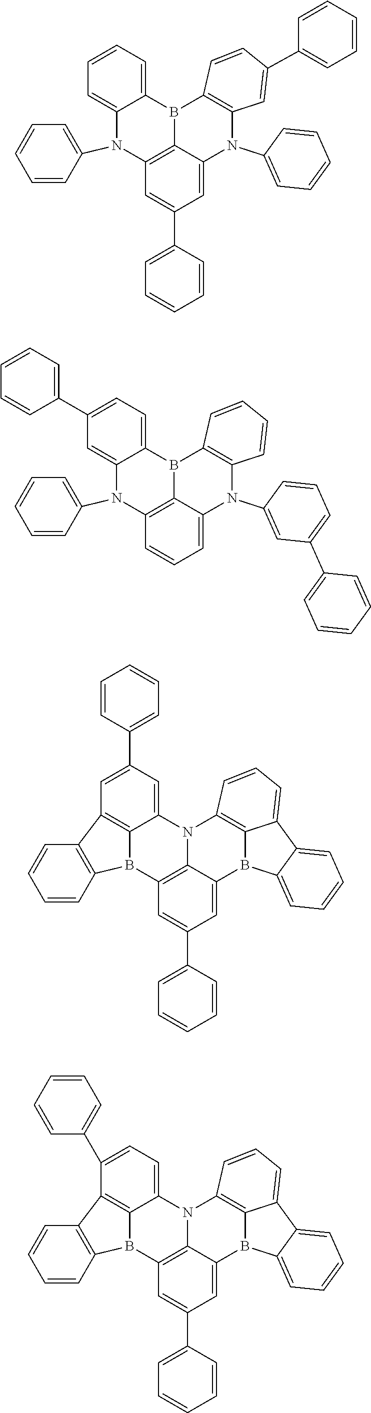 [SMILES: c1ccc(-c2ccc3c(c2)N(c2ccccc2)c2cc(-c4ccccc4)cc4c2B3c2ccccc2N4c2ccccc2)cc1 ; c1ccc(-c2cccc(N3c4ccccc4B4c5ccc(-c6ccccc6)cc5N(c5ccccc5)c5cccc3c54)c2)cc1 ; c1ccc(-c2cc3c4c(c2)B2c5ccccc5-c5cc(-c6ccccc6)cc(c52)N4c2cccc4c2B3c2ccccc2-4)cc1 ; c1ccc(-c2cc3c4c(c2)B2c5ccccc5-c5c(-c6ccccc6)ccc(c52)N4c2cccc4c2B3c2ccccc2-4)cc1]

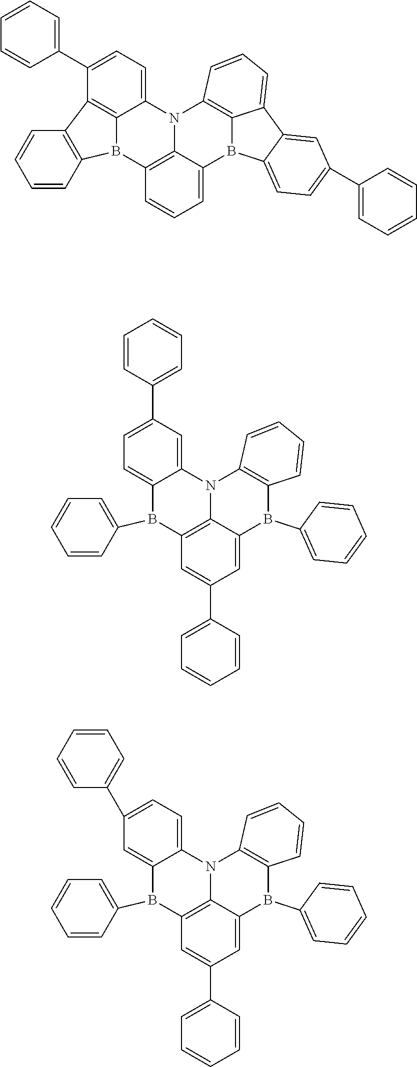 [SMILES: c1ccc(-c2ccc3c(c2)-c2cccc4c2B3c2cccc3c2N4c2ccc(-c4ccccc4)c4c2B3c2ccccc2-4)cc1 ; c1ccc(B2c3ccccc3N3c4cc(-c5ccccc5)ccc4B(c4ccccc4)c4cc(-c5ccccc5)cc2c43)cc1 ; c1ccc(B2c3ccccc3N3c4ccc(-c5ccccc5)cc4B(c4ccccc4)c4cc(-c5ccccc5)cc2c43)cc1]

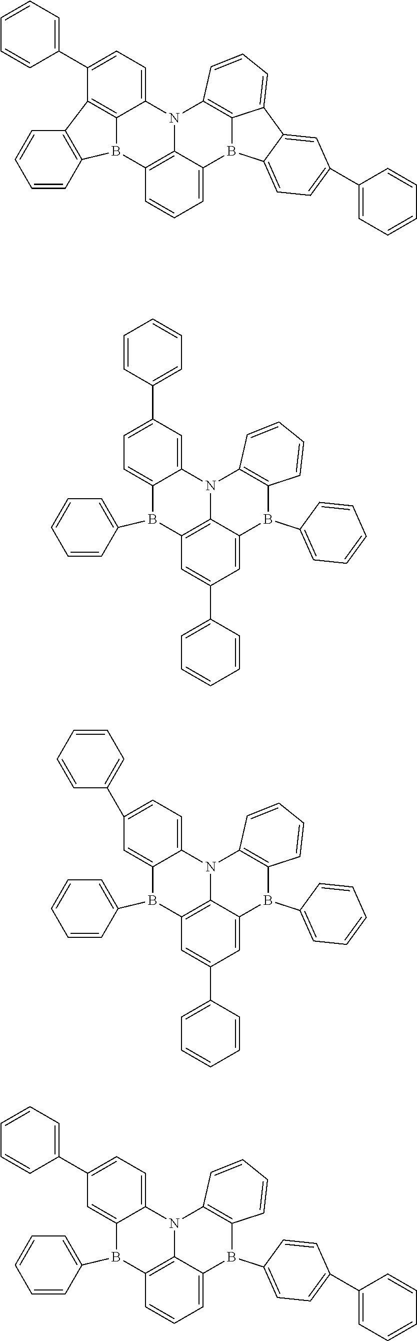 [SMILES: c1ccc(-c2ccc3c(c2)-c2cccc4c2B3c2cccc3c2N4c2ccc(-c4ccccc4)c4c2B3c2ccccc2-4)cc1 ; c1ccc(B2c3ccccc3N3c4cc(-c5ccccc5)ccc4B(c4ccccc4)c4cc(-c5ccccc5)cc2c43)cc1 ; c1ccc(B2c3ccccc3N3c4ccc(-c5ccccc5)cc4B(c4ccccc4)c4cc(-c5ccccc5)cc2c43)cc1 ; c1ccc(B2c3cc(-c4ccccc4)ccc3N3c4ccccc4B(c4ccc(-c5ccccc5)cc4)c4cccc2c43)cc1]

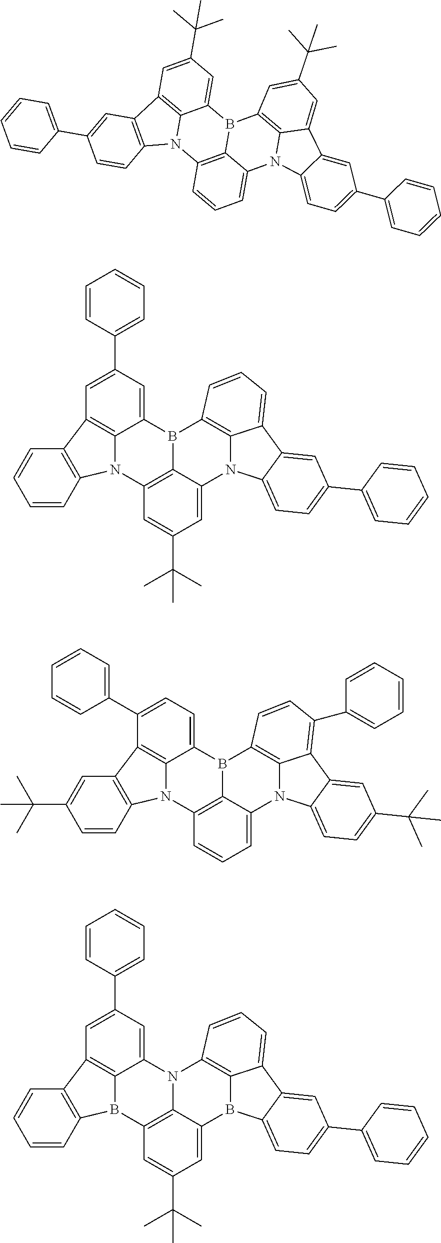 [SMILES: CC(C)(C)c1cc2c3c(c1)c1cc(-c4ccccc4)ccc1n3-c1cccc3c1B2c1cc(C(C)(C)C)cc2c4cc(-c5ccccc5)ccc4n-3c12 ; CC(C)(C)c1cc2c3c(c1)-n1c4ccccc4c4cc(-c5ccccc5)cc(c41)B3c1cccc3c4cc(-c5ccccc5)ccc4n-2c13 ; CC(C)(C)c1ccc2c(c1)c1c(-c3ccccc3)ccc3c1n2-c1cccc2c1B3c1ccc(-c3ccccc3)c3c4cc(C(C)(C)C)ccc4n-2c13 ; CC(C)(C)c1cc2c3c(c1)B1c4ccccc4-c4cc(-c5ccccc5)cc(c41)N3c1cccc3c1B2c1ccc(-c2ccccc2)cc1-3]

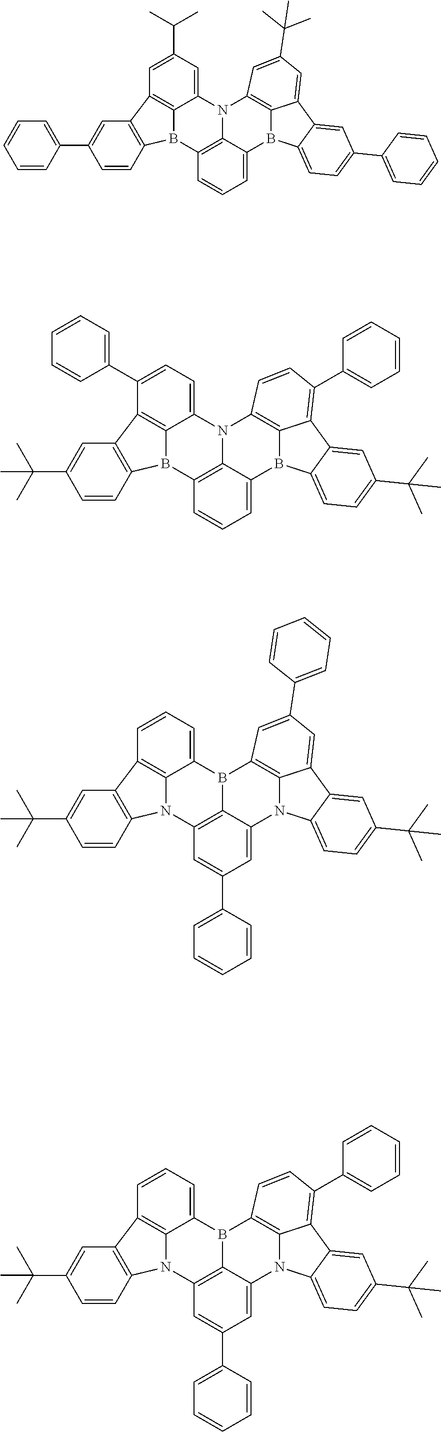

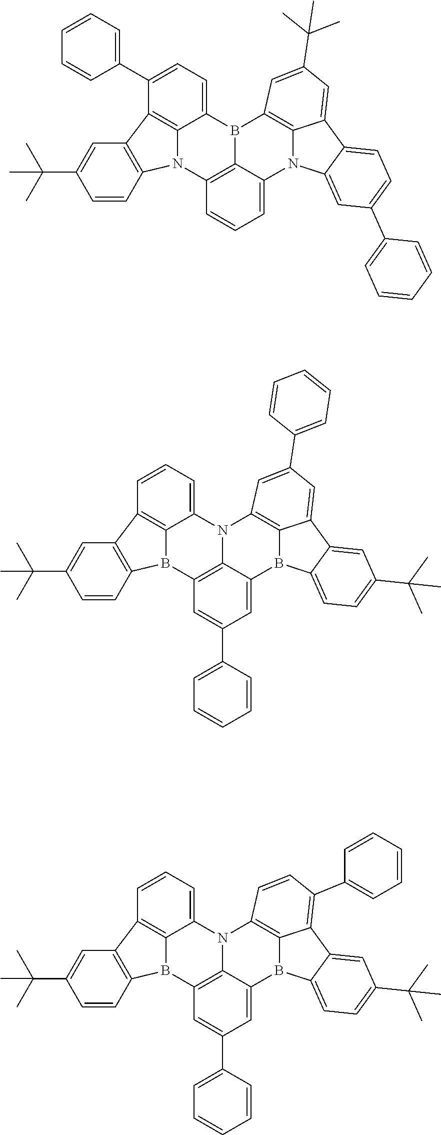

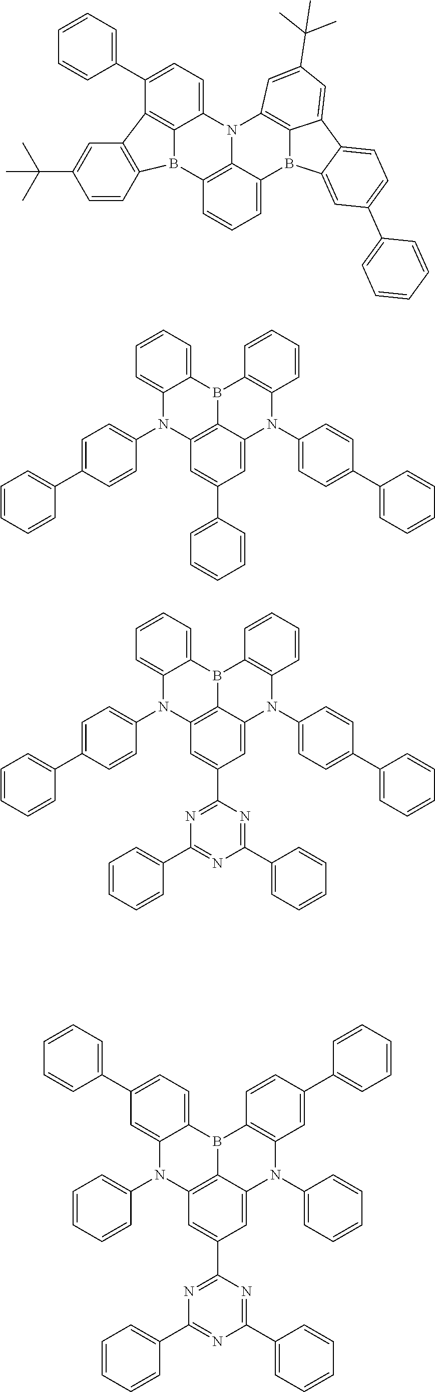 [SMILES: CC(C)(C)c1ccc2c(c1)-c1c(-c3ccccc3)ccc3c1B2c1cccc2c1N3c1cc(C(C)(C)C)cc3c1B2c1cc(-c2ccccc2)ccc1-3 ; c1ccc(-c2ccc(N3c4ccccc4B4c5ccccc5N(c5ccc(-c6ccccc6)cc5)c5cc(-c6ccccc6)cc3c54)cc2)cc1 ; c1ccc(-c2ccc(N3c4ccccc4B4c5ccccc5N(c5ccc(-c6ccccc6)cc5)c5cc(-c6nc(-c7ccccc7)nc(-c7ccccc7)n6)cc3c54)cc2)cc1 ; c1ccc(-c2ccc3c(c2)N(c2ccccc2)c2cc(-c4nc(-c5ccccc5)nc(-c5ccccc5)n4)cc4c2B3c2ccc(-c3ccccc3)cc2N4c2ccccc2)cc1]

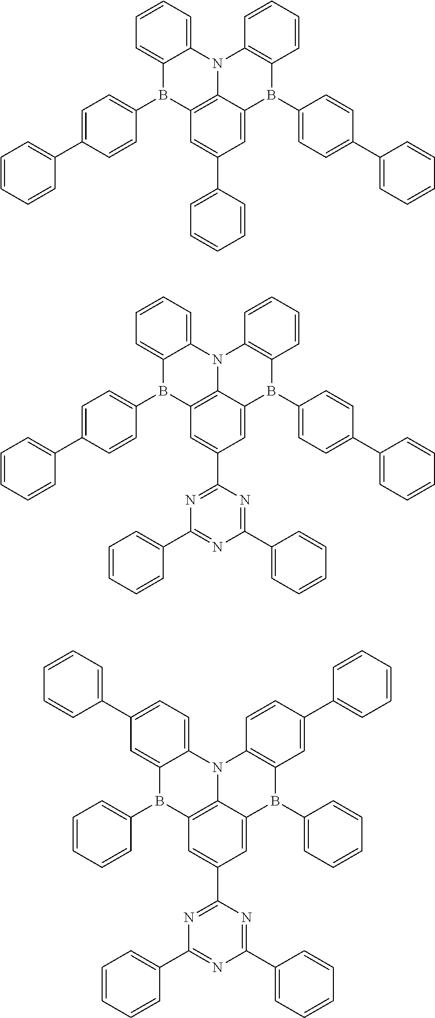 [SMILES: c1ccc(-c2ccc(B3c4ccccc4N4c5ccccc5B(c5ccc(-c6ccccc6)cc5)c5cc(-c6ccccc6)cc3c54)cc2)cc1 ; c1ccc(-c2ccc(B3c4ccccc4N4c5ccccc5B(c5ccc(-c6ccccc6)cc5)c5cc(-c6nc(-c7ccccc7)nc(-c7ccccc7)n6)cc3c54)cc2)cc1 ; c1ccc(B2c3cc(-c4ccccc4)ccc3N3c4ccc(-c5ccccc5)cc4B(c4ccccc4)c4cc(-c5nc(-c6ccccc6)nc(-c6ccccc6)n5)cc2c43)cc1]

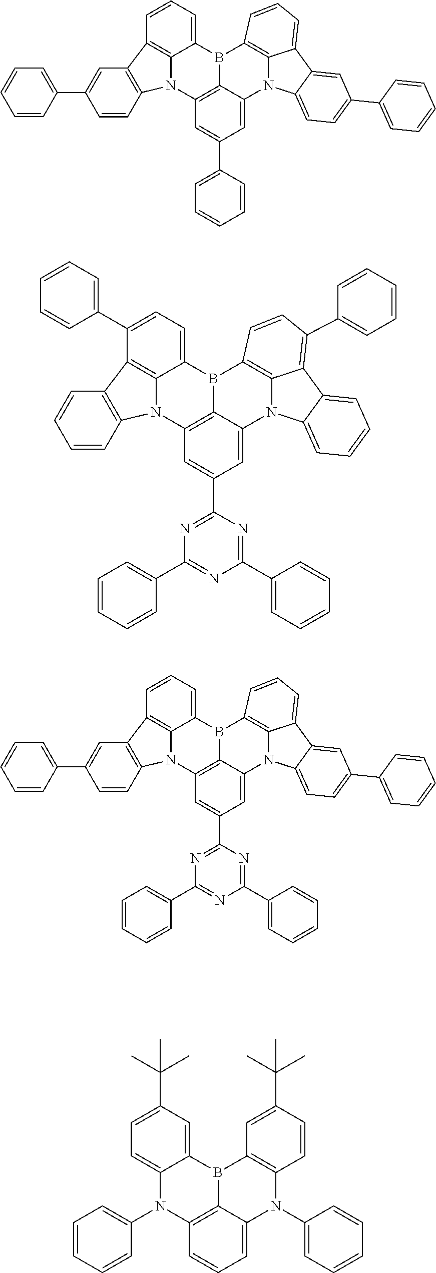 [SMILES: c1ccc(-c2cc3c4c(c2)-n2c5ccc(-c6ccccc6)cc5c5cccc(c52)B4c2cccc4c5cc(-c6ccccc6)ccc5n-3c24)cc1 ; c1ccc(-c2nc(-c3ccccc3)nc(-c3cc4c5c(c3)-n3c6ccccc6c6c(-c7ccccc7)ccc(c63)B5c3ccc(-c5ccccc5)c5c6ccccc6n-4c35)n2)cc1 ; c1ccc(-c2ccc3c(c2)c2cccc4c2n3-c2cc(-c3nc(-c5ccccc5)nc(-c5ccccc5)n3)cc3c2B4c2cccc4c5cc(-c6ccccc6)ccc5n-3c24)cc1 ; CC(C)(C)c1ccc2c(c1)B1c3cc(C(C)(C)C)ccc3N(c3ccccc3)c3cccc(c31)N2c1ccccc1]

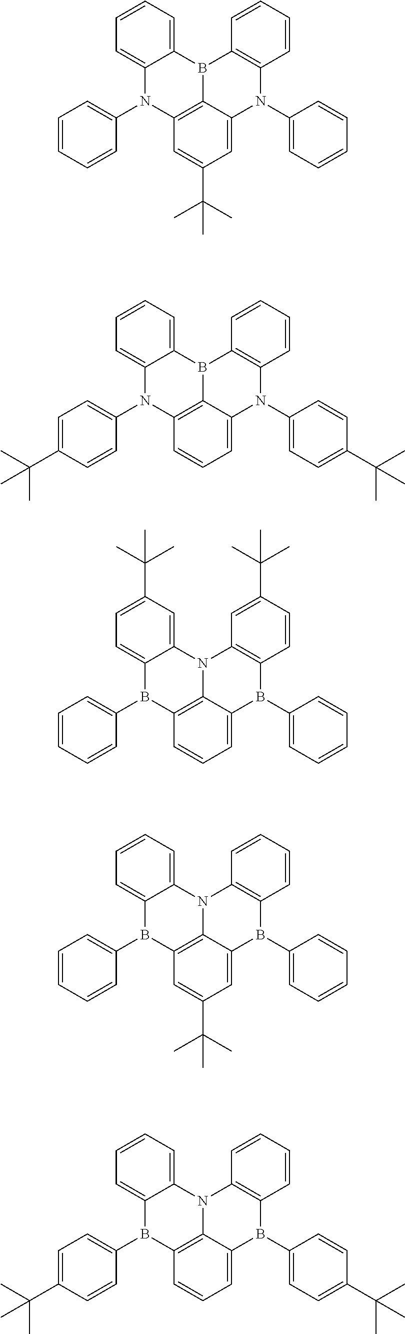

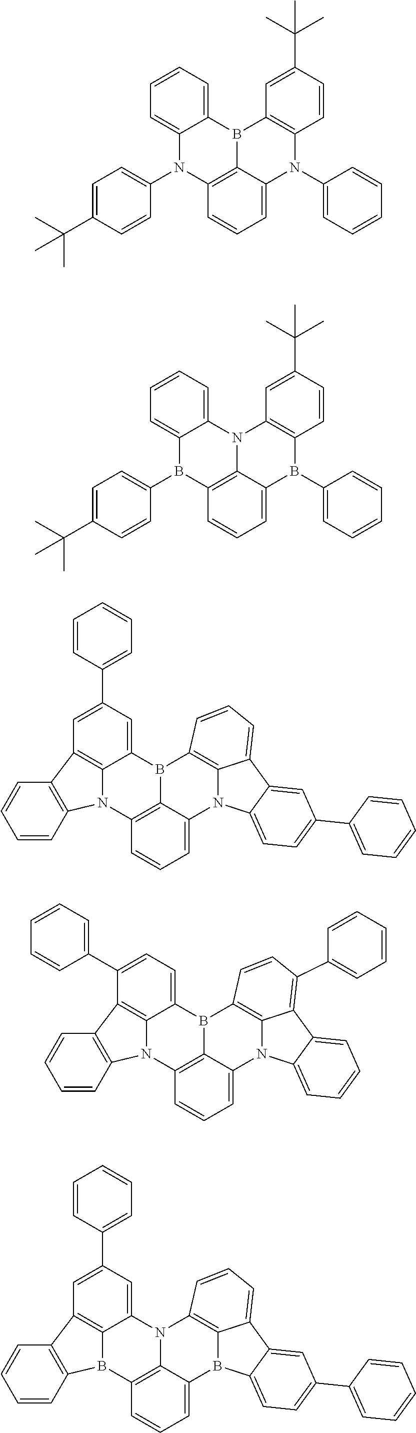 [SMILES: CC(C)(C)c1ccc(N2c3ccccc3B3c4cc(C(C)(C)C)ccc4N(c4ccccc4)c4cccc2c43)cc1 ; CC(C)(C)c1ccc(B2c3ccccc3N3c4cc(C(C)(C)C)ccc4B(c4ccccc4)c4cccc2c43)cc1 ; c1ccc(-c2ccc3c(c2)c2cccc4c2n3-c2cccc3c2B4c2cc(-c4ccccc4)cc4c5ccccc5n-3c24)cc1 ; c1ccc(-c2ccc3c4c2c2ccccc2n4-c2cccc4c2B3c2ccc(-c3ccccc3)c3c5ccccc5n-4c23)cc1 ; c1ccc(-c2ccc3c(c2)-c2cccc4c2B3c2cccc3c2N4c2cc(-c4ccccc4)cc4c2B3c2ccccc2-4)cc1]

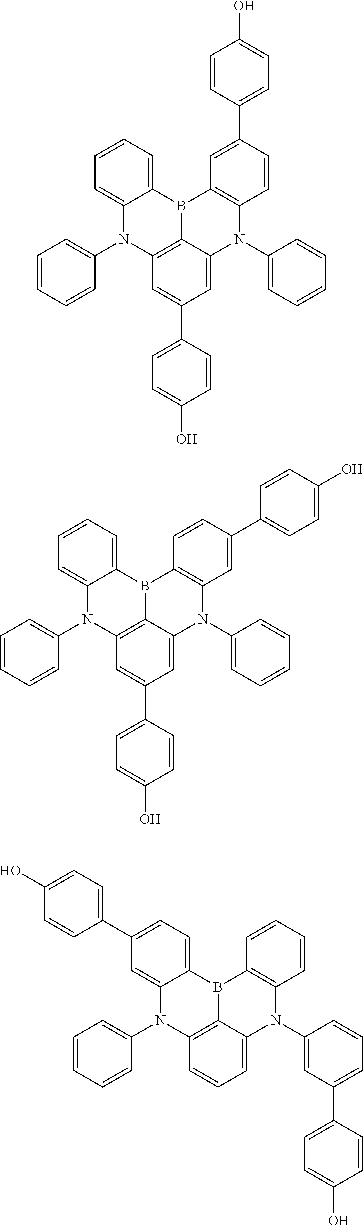 [SMILES: Oc1ccc(-c2ccc3c(c2)B2c4ccccc4N(c4ccccc4)c4cc(-c5ccc(O)cc5)cc(c42)N3c2ccccc2)cc1 ; Oc1ccc(-c2ccc3c(c2)N(c2ccccc2)c2cc(-c4ccc(O)cc4)cc4c2B3c2ccccc2N4c2ccccc2)cc1 ; Oc1ccc(-c2cccc(N3c4ccccc4B4c5ccc(-c6ccc(O)cc6)cc5N(c5ccccc5)c5cccc3c54)c2)cc1]

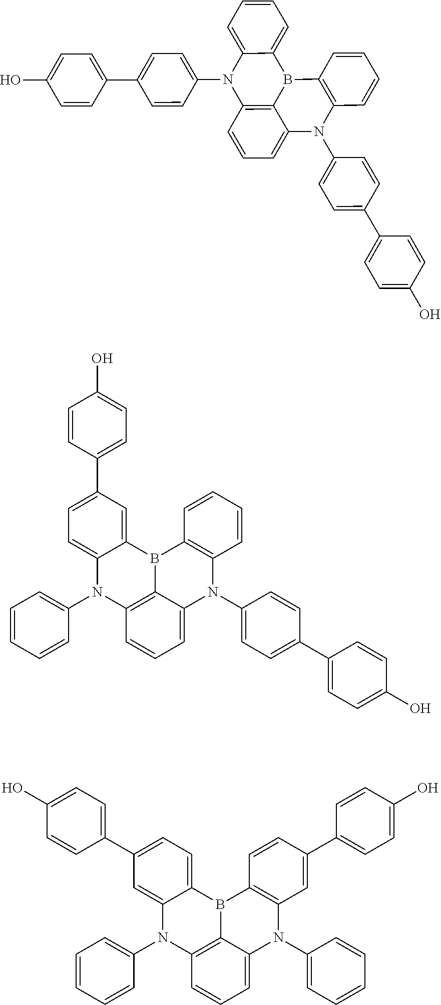 [SMILES: Oc1ccc(-c2ccc(N3c4ccccc4B4c5ccccc5N(c5ccc(-c6ccc(O)cc6)cc5)c5cccc3c54)cc2)cc1 ; Oc1ccc(-c2ccc(N3c4ccccc4B4c5cc(-c6ccc(O)cc6)ccc5N(c5ccccc5)c5cccc3c54)cc2)cc1 ; Oc1ccc(-c2ccc3c(c2)N(c2ccccc2)c2cccc4c2B3c2ccc(-c3ccc(O)cc3)cc2N4c2ccccc2)cc1]

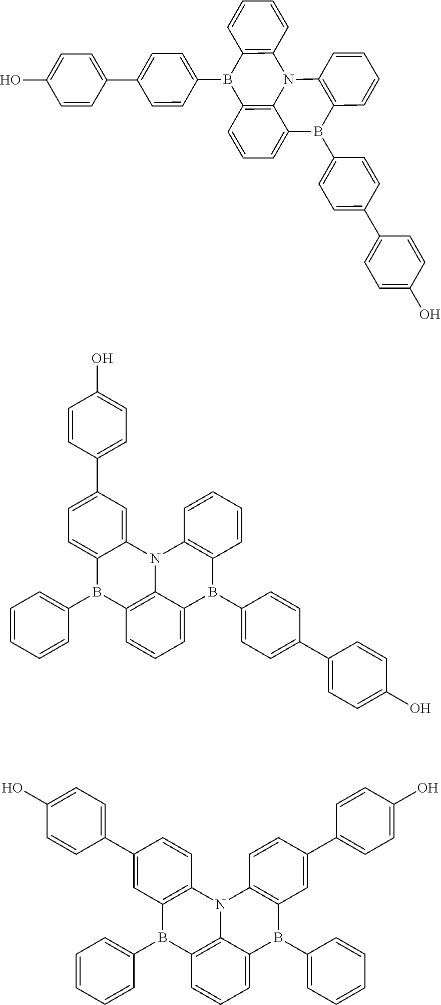 [SMILES: Oc1ccc(-c2ccc(B3c4ccccc4N4c5ccccc5B(c5ccc(-c6ccc(O)cc6)cc5)c5cccc3c54)cc2)cc1 ; Oc1ccc(-c2ccc(B3c4ccccc4N4c5cc(-c6ccc(O)cc6)ccc5B(c5ccccc5)c5cccc3c54)cc2)cc1 ; Oc1ccc(-c2ccc3c(c2)B(c2ccccc2)c2cccc4c2N3c2ccc(-c3ccc(O)cc3)cc2B4c2ccccc2)cc1]

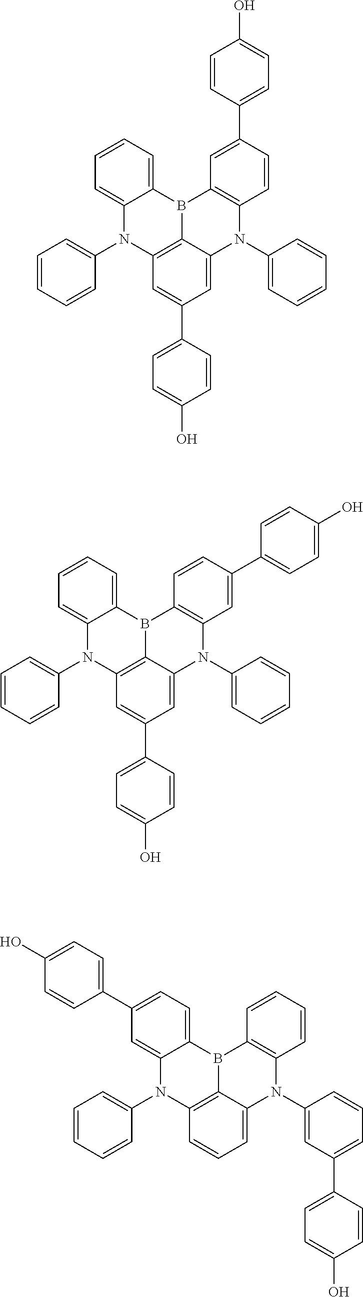 [SMILES: Oc1ccc(-c2ccc3c(c2)B2c4ccccc4N(c4ccccc4)c4cc(-c5ccc(O)cc5)cc(c42)N3c2ccccc2)cc1 ; Oc1ccc(-c2ccc3c(c2)N(c2ccccc2)c2cc(-c4ccc(O)cc4)cc4c2B3c2ccccc2N4c2ccccc2)cc1 ; Oc1ccc(-c2cccc(N3c4ccccc4B4c5ccc(-c6ccc(O)cc6)cc5N(c5ccccc5)c5cccc3c54)c2)cc1]

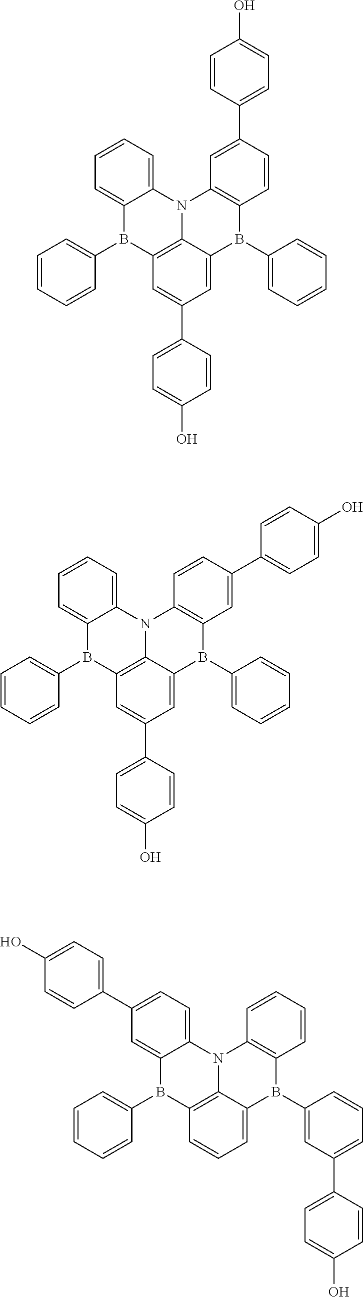 [SMILES: Oc1ccc(-c2cc3c4c(c2)B(c2ccccc2)c2ccc(-c5ccc(O)cc5)cc2N4c2ccccc2B3c2ccccc2)cc1 ; Oc1ccc(-c2ccc3c(c2)B(c2ccccc2)c2cc(-c4ccc(O)cc4)cc4c2N3c2ccccc2B4c2ccccc2)cc1 ; Oc1ccc(-c2cccc(B3c4ccccc4N4c5ccc(-c6ccc(O)cc6)cc5B(c5ccccc5)c5cccc3c54)c2)cc1]

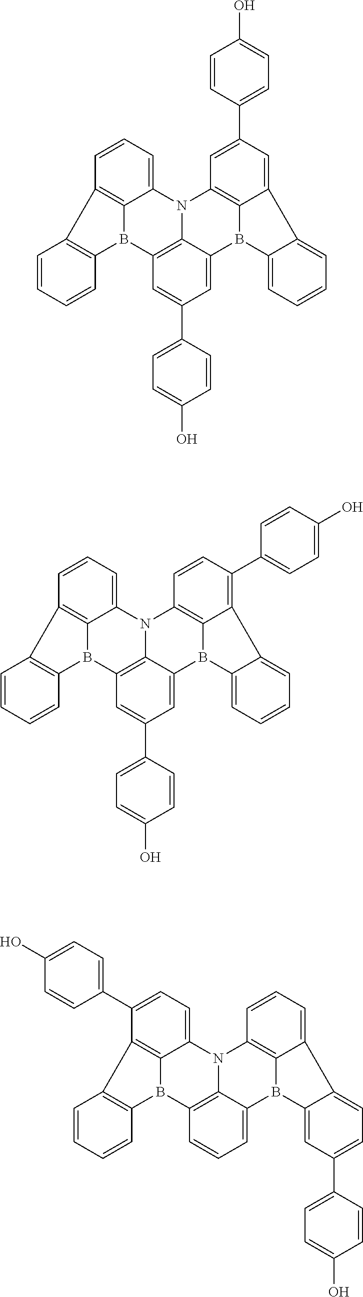 [SMILES: Oc1ccc(-c2cc3c4c(c2)B2c5ccccc5-c5cc(-c6ccc(O)cc6)cc(c52)N4c2cccc4c2B3c2ccccc2-4)cc1 ; Oc1ccc(-c2cc3c4c(c2)B2c5ccccc5-c5c(-c6ccc(O)cc6)ccc(c52)N4c2cccc4c2B3c2ccccc2-4)cc1 ; Oc1ccc(-c2ccc3c(c2)B2c4cccc5c4N(c4cccc-3c42)c2ccc(-c3ccc(O)cc3)c3c2B5c2ccccc2-3)cc1]

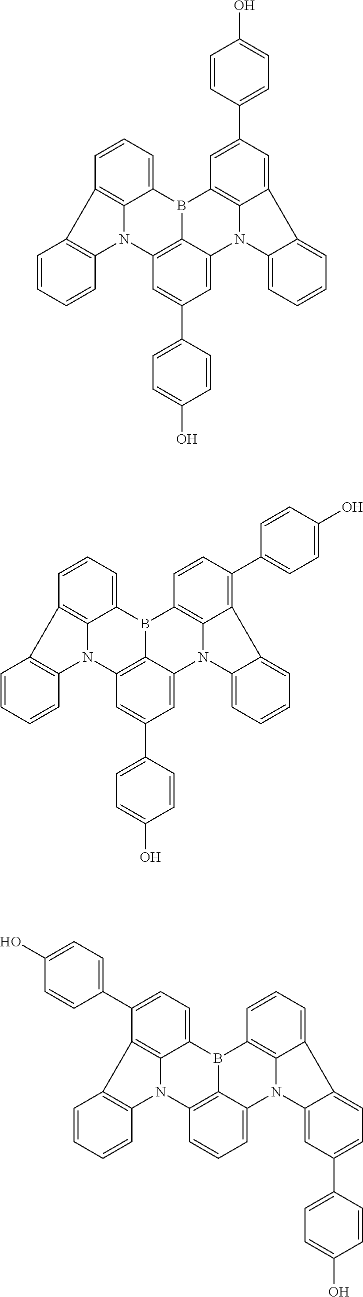 [SMILES: Oc1ccc(-c2cc3c4c(c2)-n2c5ccccc5c5cc(-c6ccc(O)cc6)cc(c52)B4c2cccc4c5ccccc5n-3c24)cc1 ; Oc1ccc(-c2cc3c4c(c2)-n2c5ccccc5c5c(-c6ccc(O)cc6)ccc(c52)B4c2cccc4c5ccccc5n-3c24)cc1 ; Oc1ccc(-c2ccc3c4cccc5c4n(c3c2)-c2cccc3c2B5c2ccc(-c4ccc(O)cc4)c4c5ccccc5n-3c24)cc1]

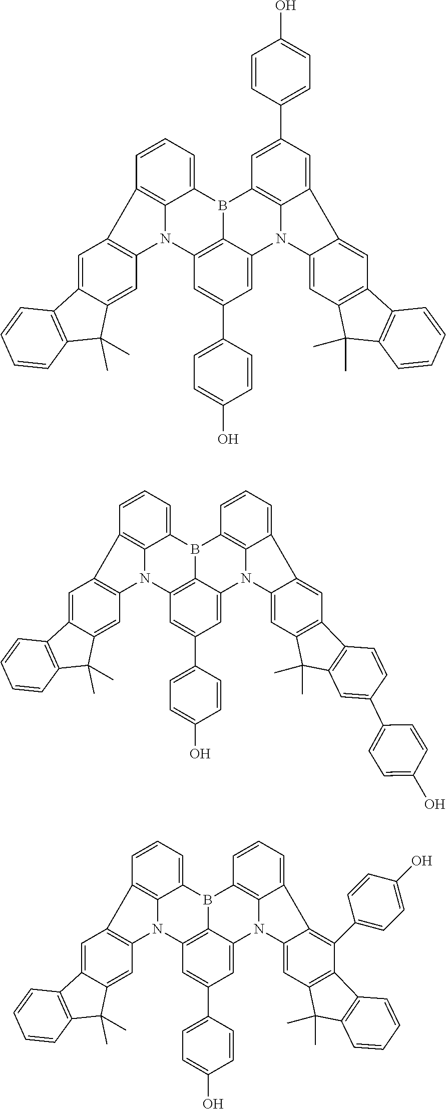 [SMILES: CC1(C)c2ccccc2-c2cc3c4cccc5c4n(c3cc21)-c1cc(-c2ccc(O)cc2)cc2c1B5c1cc(-c3ccc(O)cc3)cc3c4cc5c(cc4n-2c13)C(C)(C)c1ccccc1-5 ; CC1(C)c2ccccc2-c2cc3c4cccc5c4n(c3cc21)-c1cc(-c2ccc(O)cc2)cc2c1B5c1cccc3c4cc5c(cc4n-2c13)C(C)(C)c1cc(-c2ccc(O)cc2)ccc1-5 ; CC1(C)c2ccccc2-c2cc3c4cccc5c4n(c3cc21)-c1cc(-c2ccc(O)cc2)cc2c1B5c1cccc3c4c(-c5ccc(O)cc5)c5c(cc4n-2c13)C(C)(C)c1ccccc1-5]

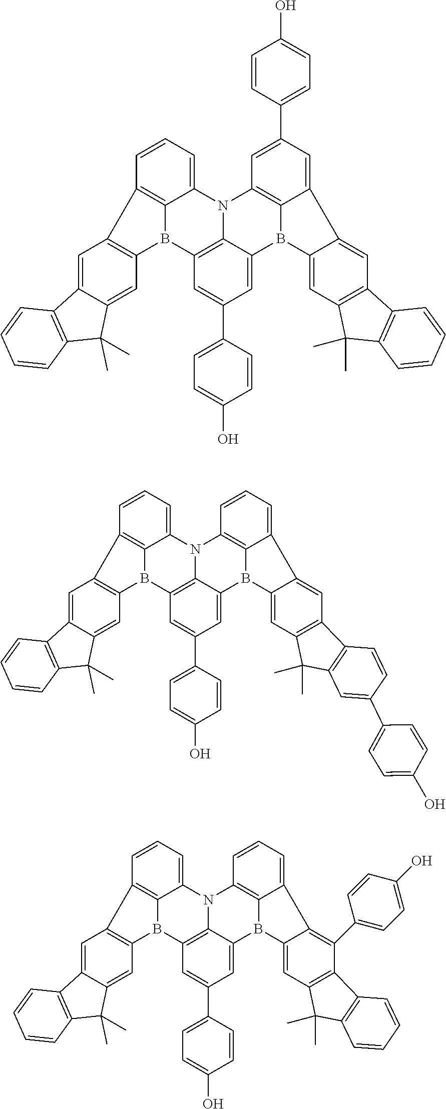 [SMILES: CC1(C)c2ccccc2-c2cc3c(cc21)B1c2cc(-c4ccc(O)cc4)cc4c2N(c2cccc-3c21)c1cc(-c2ccc(O)cc2)cc2c1B4c1cc3c(cc1-2)-c1ccccc1C3(C)C ; CC1(C)c2ccccc2-c2cc3c(cc21)B1c2cc(-c4ccc(O)cc4)cc4c2N(c2cccc-3c21)c1cccc2c1B4c1cc3c(cc1-2)-c1ccc(-c2ccc(O)cc2)cc1C3(C)C ; CC1(C)c2ccccc2-c2cc3c(cc21)B1c2cc(-c4ccc(O)cc4)cc4c2N(c2cccc-3c21)c1cccc2c1B4c1cc3c(c(-c4ccc(O)cc4)c1-2)-c1ccccc1C3(C)C]

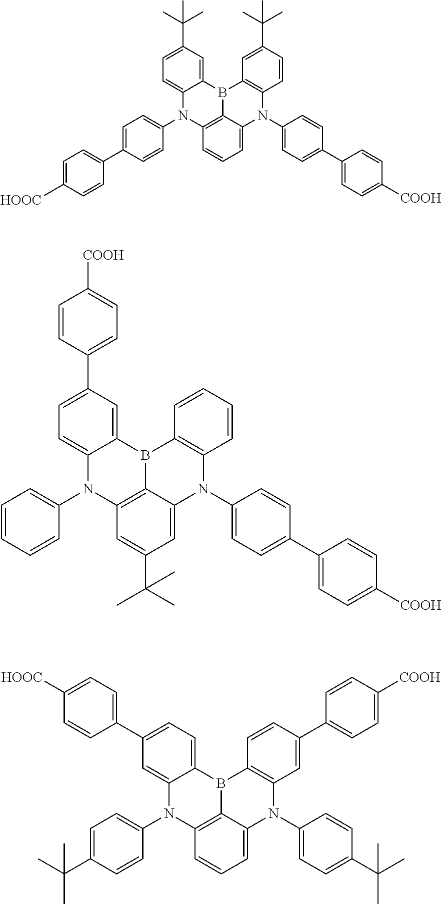 [SMILES: CC(C)(C)c1ccc2c(c1)B1c3cc(C(C)(C)C)ccc3N(c3ccc(-c4ccc(C(=O)O)cc4)cc3)c3cccc(c31)N2c1ccc(-c2ccc(C(=O)O)cc2)cc1 ; CC(C)(C)c1cc2c3c(c1)N(c1ccccc1)c1ccc(-c4ccc(C(=O)O)cc4)cc1B3c1ccccc1N2c1ccc(-c2ccc(C(=O)O)cc2)cc1 ; CC(C)(C)c1ccc(N2c3cc(-c4ccc(C(=O)O)cc4)ccc3B3c4ccc(-c5ccc(C(=O)O)cc5)cc4N(c4ccc(C(C)(C)C)cc4)c4cccc2c43)cc1]

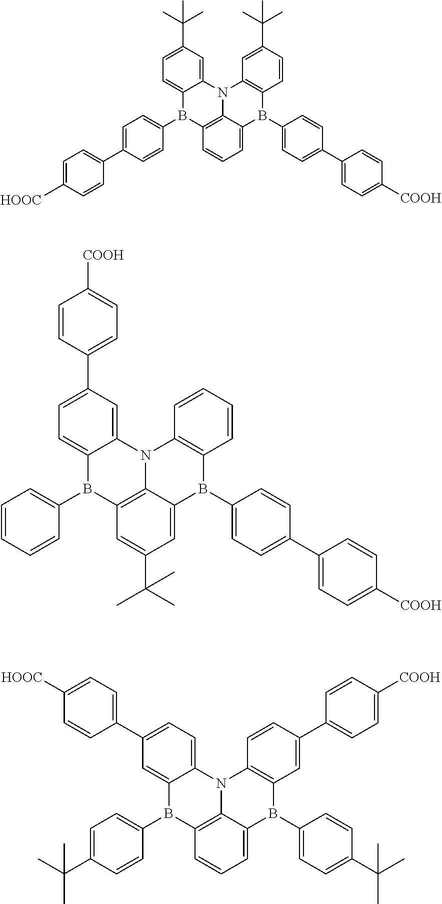 [SMILES: CC(C)(C)c1ccc2c(c1)N1c3cc(C(C)(C)C)ccc3B(c3ccc(-c4ccc(C(=O)O)cc4)cc3)c3cccc(c31)B2c1ccc(-c2ccc(C(=O)O)cc2)cc1 ; CC(C)(C)c1cc2c3c(c1)B(c1ccccc1)c1ccc(-c4ccc(C(=O)O)cc4)cc1N3c1ccccc1B2c1ccc(-c2ccc(C(=O)O)cc2)cc1 ; CC(C)(C)c1ccc(B2c3cc(-c4ccc(C(=O)O)cc4)ccc3N3c4ccc(-c5ccc(C(=O)O)cc5)cc4B(c4ccc(C(C)(C)C)cc4)c4cccc2c43)cc1]

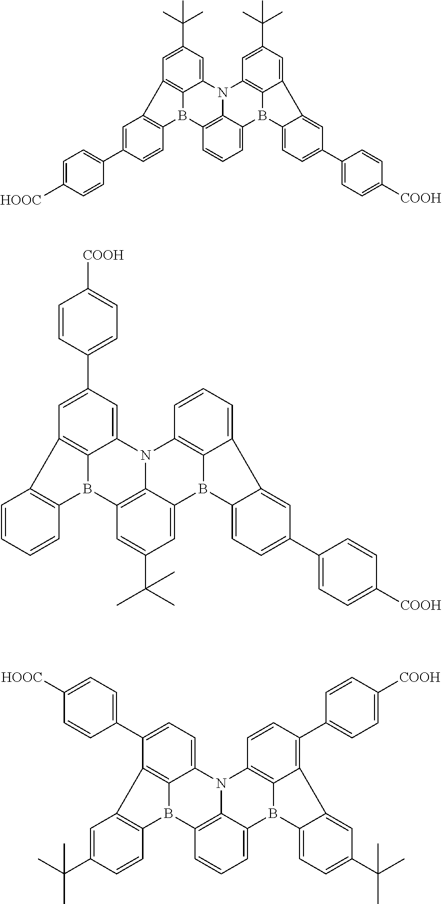 [SMILES: CC(C)(C)c1cc2c3c(c1)N1c4cc(C(C)(C)C)cc5c4B(c4ccc(-c6ccc(C(=O)O)cc6)cc4-5)c4cccc(c41)B3c1ccc(-c3ccc(C(=O)O)cc3)cc1-2 ; CC(C)(C)c1cc2c3c(c1)B1c4ccccc4-c4cc(-c5ccc(C(=O)O)cc5)cc(c41)N3c1cccc3c1B2c1ccc(-c2ccc(C(=O)O)cc2)cc1-3 ; CC(C)(C)c1ccc2c(c1)-c1c(-c3ccc(C(=O)O)cc3)ccc3c1B2c1cccc2c1N3c1ccc(-c3ccc(C(=O)O)cc3)c3c1B2c1ccc(C(C)(C)C)cc1-3]

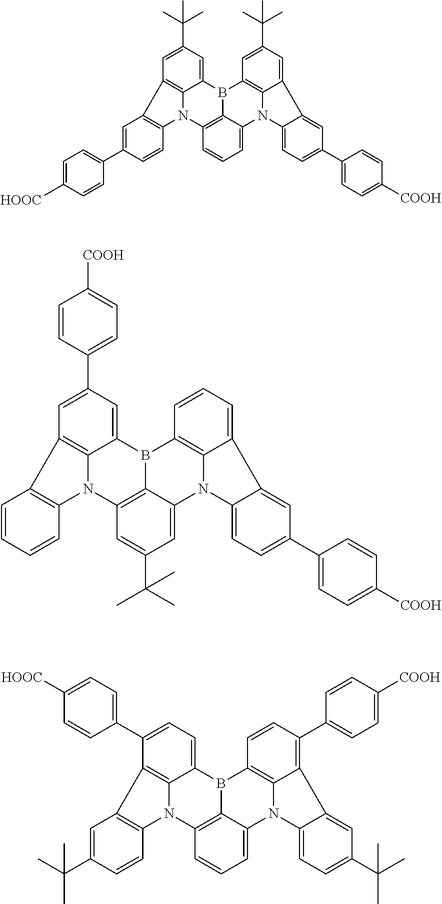 [SMILES: CC(C)(C)c1cc2c3c(c1)c1cc(-c4ccc(C(=O)O)cc4)ccc1n3-c1cccc3c1B2c1cc(C(C)(C)C)cc2c4cc(-c5ccc(C(=O)O)cc5)ccc4n-3c12 ; CC(C)(C)c1cc2c3c(c1)-n1c4ccccc4c4cc(-c5ccc(C(=O)O)cc5)cc(c41)B3c1cccc3c4cc(-c5ccc(C(=O)O)cc5)ccc4n-2c13 ; CC(C)(C)c1ccc2c(c1)c1c(-c3ccc(C(=O)O)cc3)ccc3c1n2-c1cccc2c1B3c1ccc(-c3ccc(C(=O)O)cc3)c3c4cc(C(C)(C)C)ccc4n-2c13]

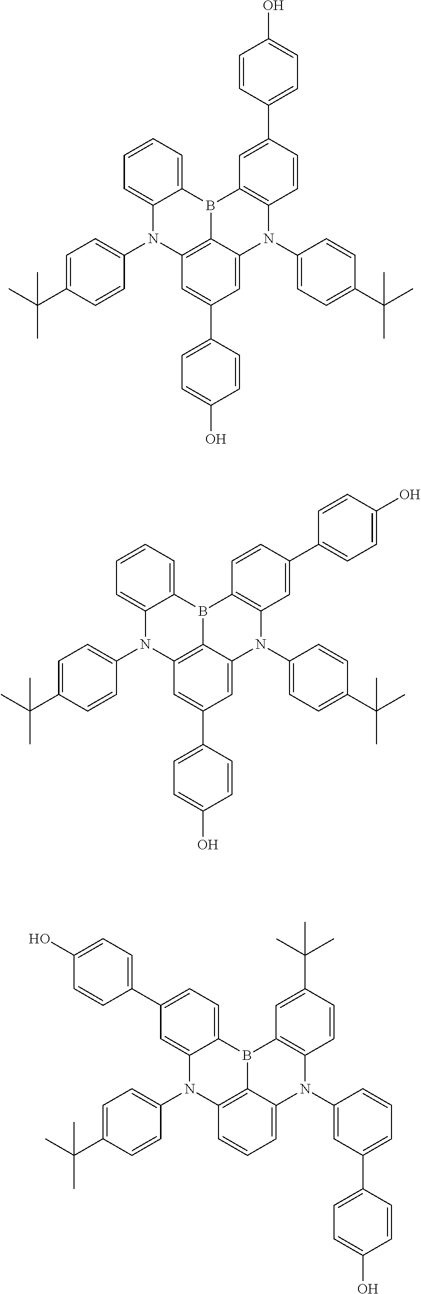

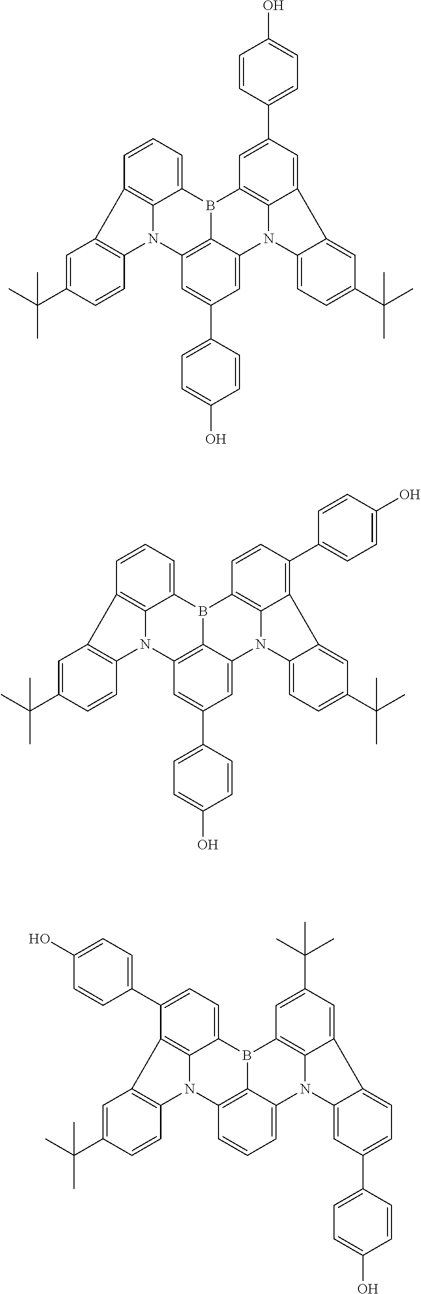

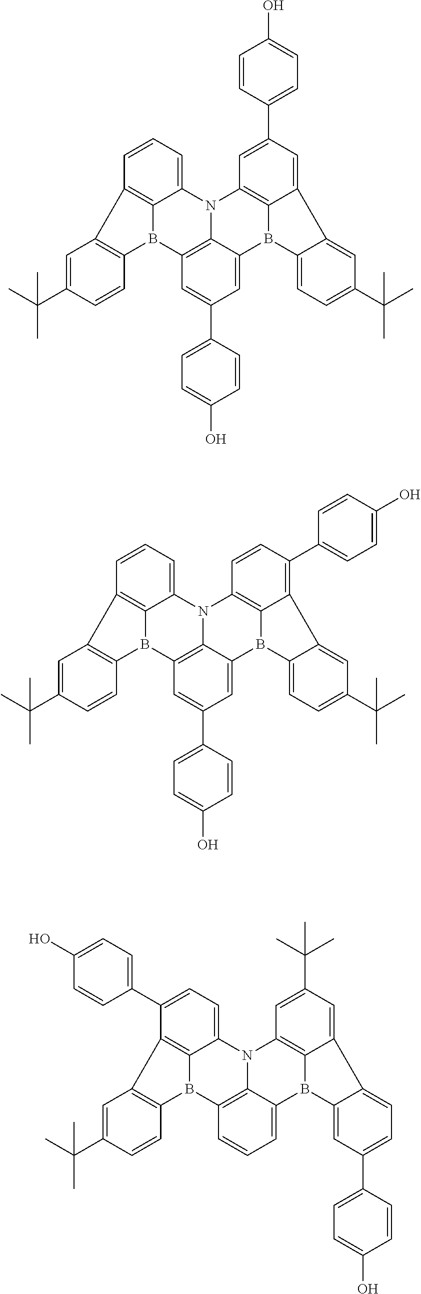

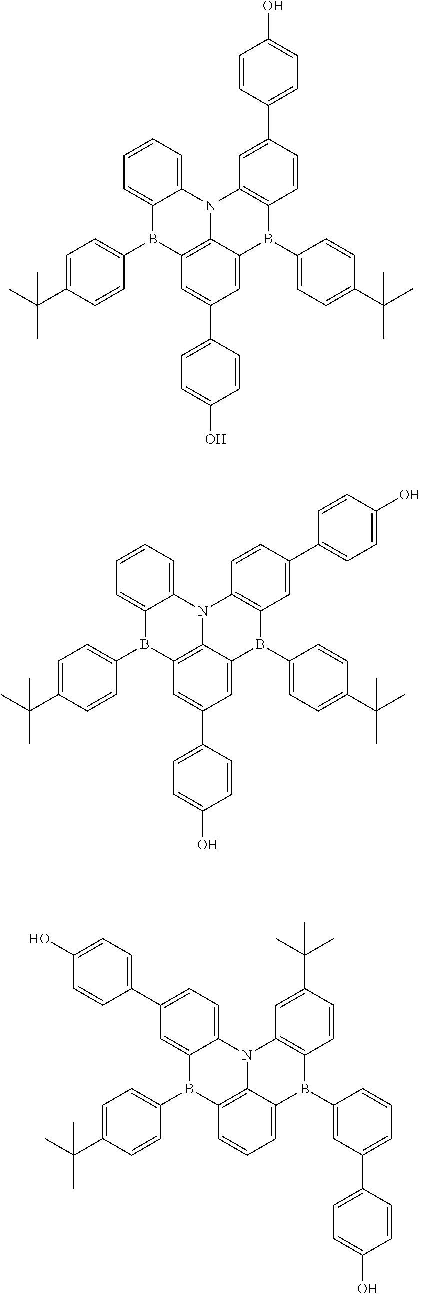

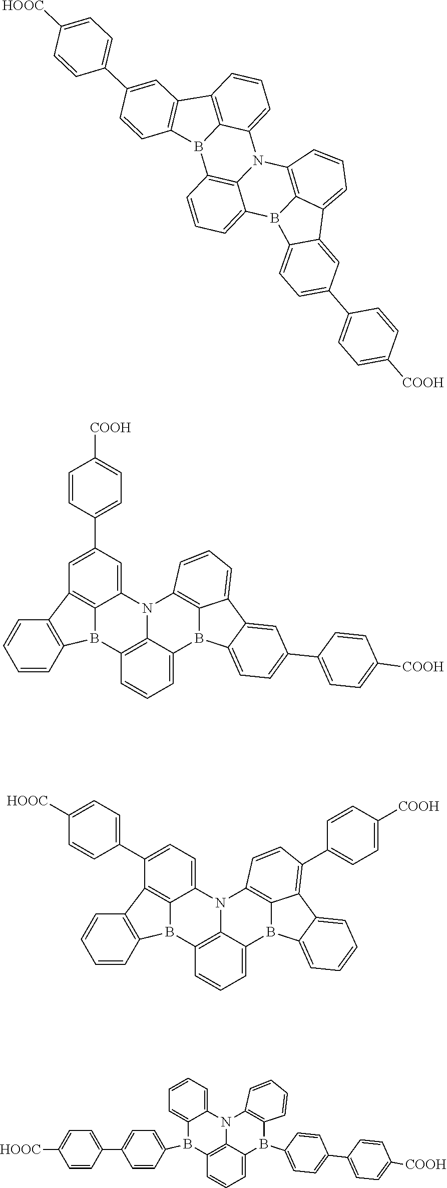 [SMILES: O=C(O)c1ccc(-c2ccc3c(c2)-c2cccc4c2B3c2cccc3c2N4c2cccc4c2B3c2ccc(-c3ccc(C(=O)O)cc3)cc2-4)cc1 ; O=C(O)c1ccc(-c2ccc3c(c2)-c2cccc4c2B3c2cccc3c2N4c2cc(-c4ccc(C(=O)O)cc4)cc4c2B3c2ccccc2-4)cc1 ; O=C(O)c1ccc(-c2ccc3c4c2-c2ccccc2B4c2cccc4c2N3c2ccc(-c3ccc(C(=O)O)cc3)c3c2B4c2ccccc2-3)cc1 ; O=C(O)c1ccc(-c2ccc(B3c4ccccc4N4c5ccccc5B(c5ccc(-c6ccc(C(=O)O)cc6)cc5)c5cccc3c54)cc2)cc1]

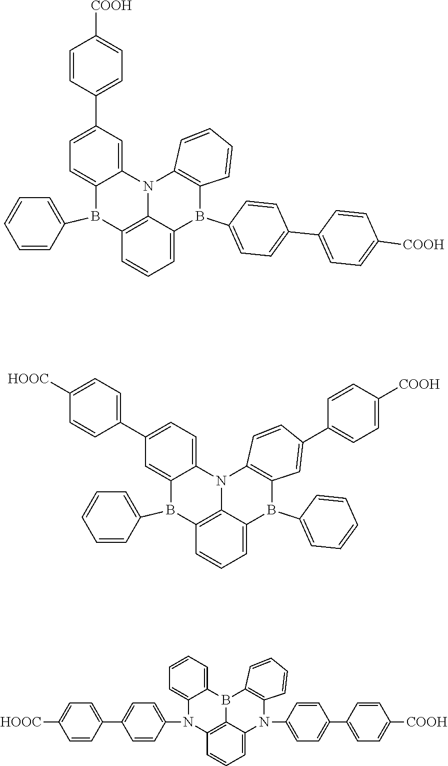 [SMILES: O=C(O)c1ccc(-c2ccc(B3c4ccccc4N4c5cc(-c6ccc(C(=O)O)cc6)ccc5B(c5ccccc5)c5cccc3c54)cc2)cc1 ; O=C(O)c1ccc(-c2ccc3c(c2)B(c2ccccc2)c2cccc4c2N3c2ccc(-c3ccc(C(=O)O)cc3)cc2B4c2ccccc2)cc1 ; O=C(O)c1ccc(-c2ccc(N3c4ccccc4B4c5ccccc5N(c5ccc(-c6ccc(C(=O)O)cc6)cc5)c5cccc3c54)cc2)cc1]

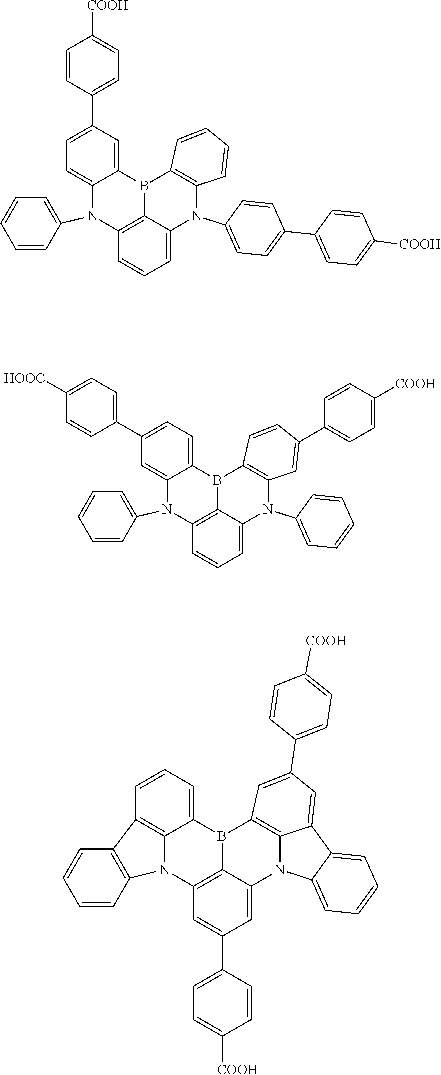 [SMILES: O=C(O)c1ccc(-c2ccc(N3c4ccccc4B4c5cc(-c6ccc(C(=O)O)cc6)ccc5N(c5ccccc5)c5cccc3c54)cc2)cc1 ; O=C(O)c1ccc(-c2ccc3c(c2)N(c2ccccc2)c2cccc4c2B3c2ccc(-c3ccc(C(=O)O)cc3)cc2N4c2ccccc2)cc1 ; O=C(O)c1ccc(-c2cc3c4c(c2)-n2c5ccccc5c5cc(-c6ccc(C(=O)O)cc6)cc(c52)B4c2cccc4c5ccccc5n-3c24)cc1]

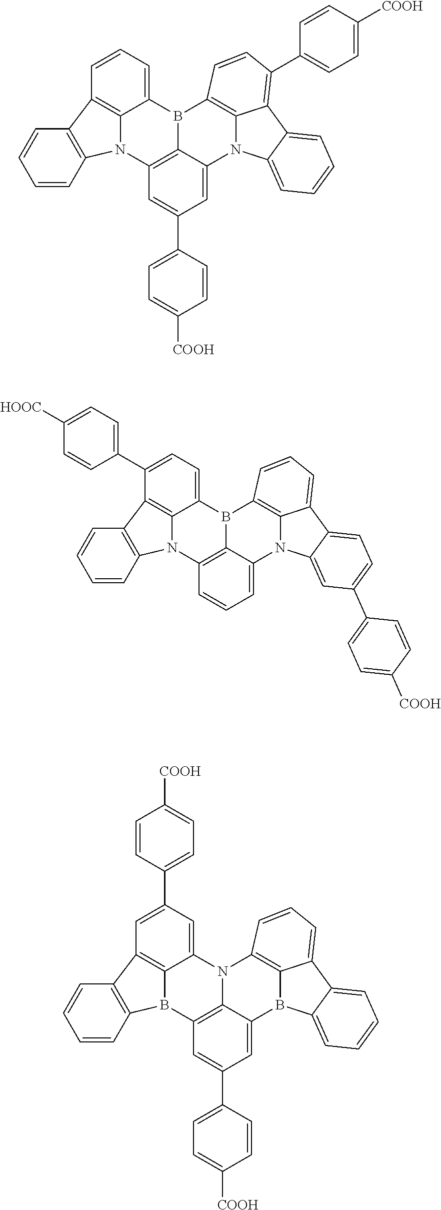 [SMILES: O=C(O)c1ccc(-c2cc3c4c(c2)-n2c5ccccc5c5c(-c6ccc(C(=O)O)cc6)ccc(c52)B4c2cccc4c5ccccc5n-3c24)cc1 ; O=C(O)c1ccc(-c2ccc3c4cccc5c4n(c3c2)-c2cccc3c2B5c2ccc(-c4ccc(C(=O)O)cc4)c4c5ccccc5n-3c24)cc1 ; O=C(O)c1ccc(-c2cc3c4c(c2)B2c5ccccc5-c5cc(-c6ccc(C(=O)O)cc6)cc(c52)N4c2cccc4c2B3c2ccccc2-4)cc1]

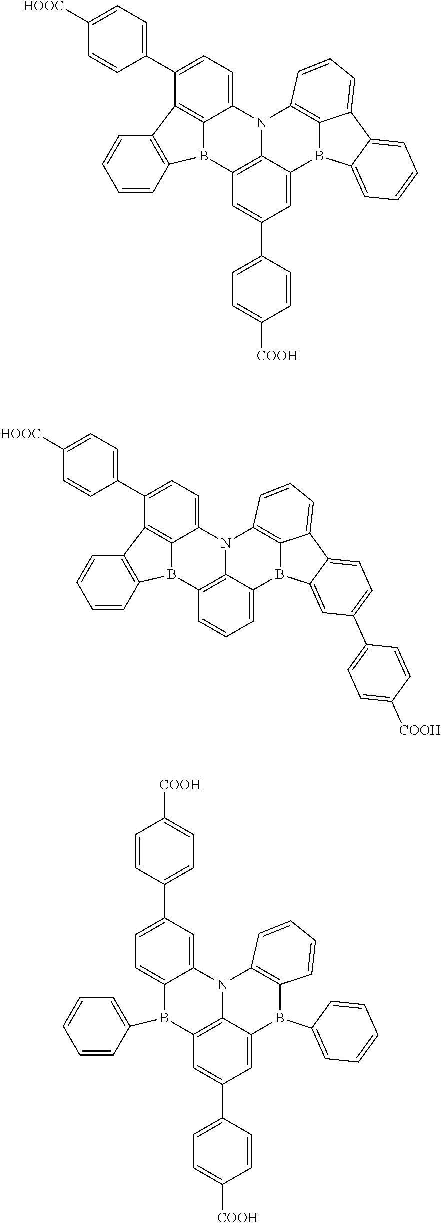 [SMILES: O=C(O)c1ccc(-c2cc3c4c(c2)B2c5ccccc5-c5c(-c6ccc(C(=O)O)cc6)ccc(c52)N4c2cccc4c2B3c2ccccc2-4)cc1 ; O=C(O)c1ccc(-c2ccc3c(c2)B2c4cccc5c4N(c4cccc-3c42)c2ccc(-c3ccc(C(=O)O)cc3)c3c2B5c2ccccc2-3)cc1 ; O=C(O)c1ccc(-c2cc3c4c(c2)B(c2ccccc2)c2ccc(-c5ccc(C(=O)O)cc5)cc2N4c2ccccc2B3c2ccccc2)cc1]

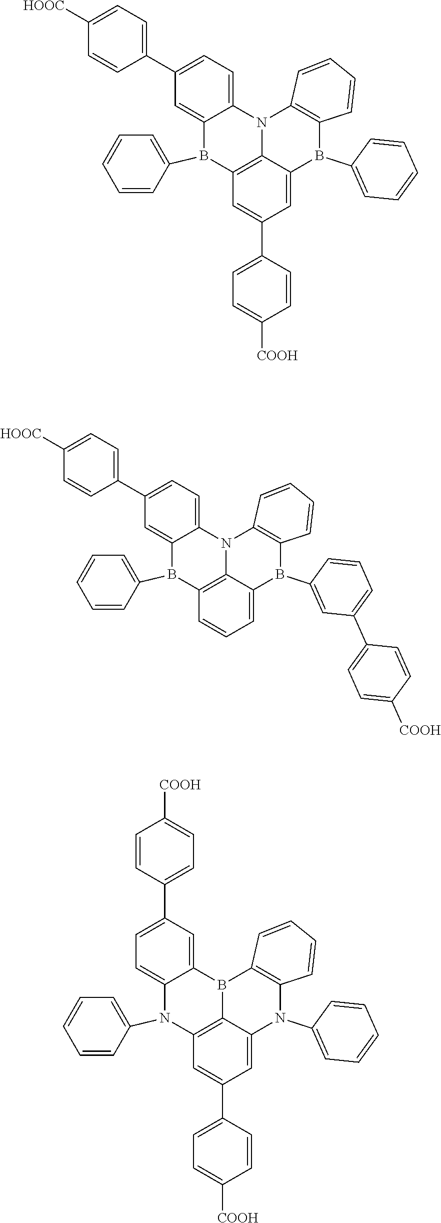 [SMILES: O=C(O)c1ccc(-c2ccc3c(c2)B(c2ccccc2)c2cc(-c4ccc(C(=O)O)cc4)cc4c2N3c2ccccc2B4c2ccccc2)cc1 ; O=C(O)c1ccc(-c2cccc(B3c4ccccc4N4c5ccc(-c6ccc(C(=O)O)cc6)cc5B(c5ccccc5)c5cccc3c54)c2)cc1 ; O=C(O)c1ccc(-c2ccc3c(c2)B2c4ccccc4N(c4ccccc4)c4cc(-c5ccc(C(=O)O)cc5)cc(c42)N3c2ccccc2)cc1]

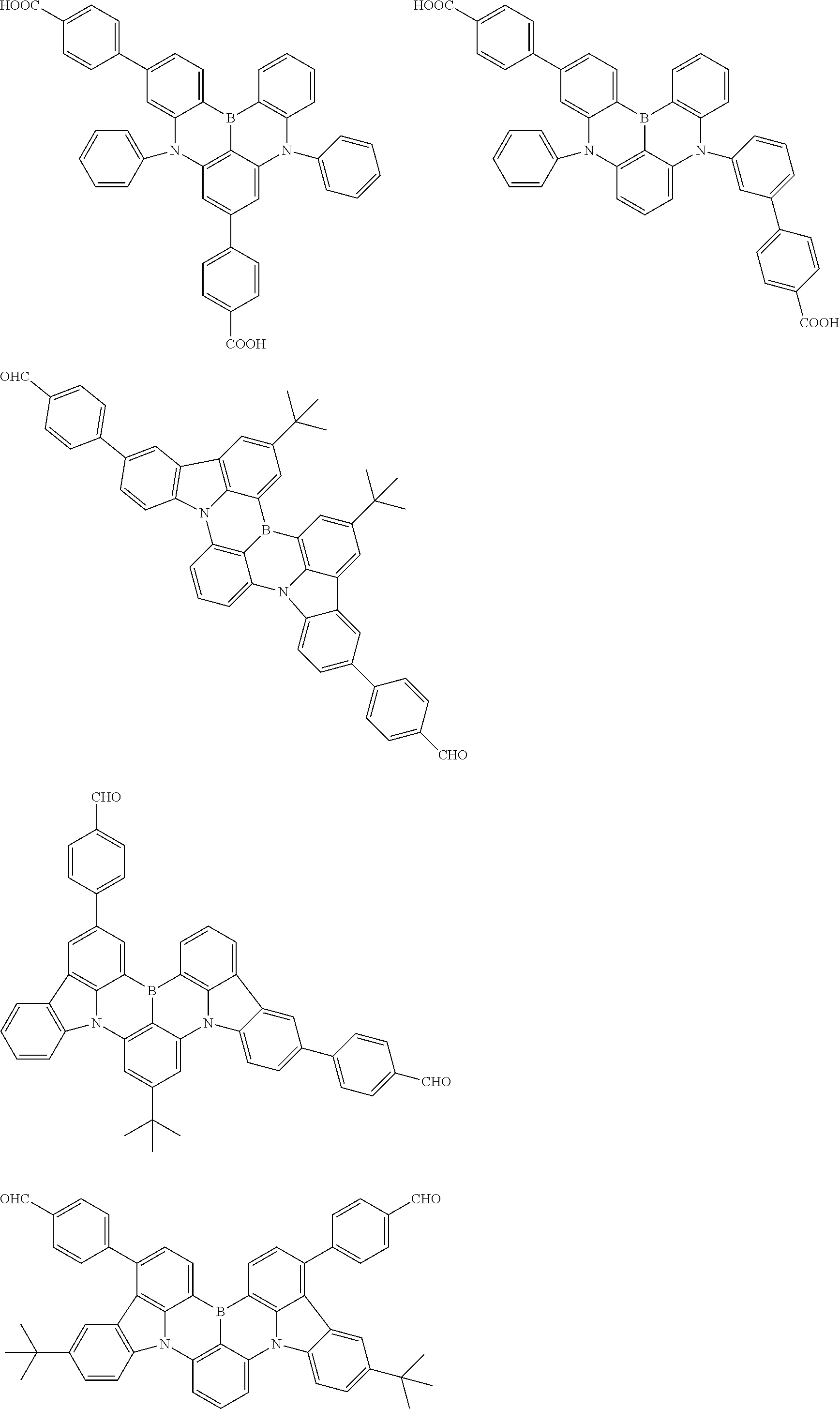 [SMILES: O=C(O)c1ccc(-c2ccc3c(c2)N(c2ccccc2)c2cc(-c4ccc(C(=O)O)cc4)cc4c2B3c2ccccc2N4c2ccccc2)cc1 ; O=C(O)c1ccc(-c2cccc(N3c4ccccc4B4c5ccc(-c6ccc(C(=O)O)cc6)cc5N(c5ccccc5)c5cccc3c54)c2)cc1 ; CC(C)(C)c1cc2c3c(c1)c1cc(-c4ccc(C=O)cc4)ccc1n3-c1cccc3c1B2c1cc(C(C)(C)C)cc2c4cc(-c5ccc(C=O)cc5)ccc4n-3c12 ; CC(C)(C)c1cc2c3c(c1)-n1c4ccccc4c4cc(-c5ccc(C=O)cc5)cc(c41)B3c1cccc3c4cc(-c5ccc(C=O)cc5)ccc4n-2c13 ; CC(C)(C)c1ccc2c(c1)c1c(-c3ccc(C=O)cc3)ccc3c1n2-c1cccc2c1B3c1ccc(-c3ccc(C=O)cc3)c3c4cc(C(C)(C)C)ccc4n-2c13]

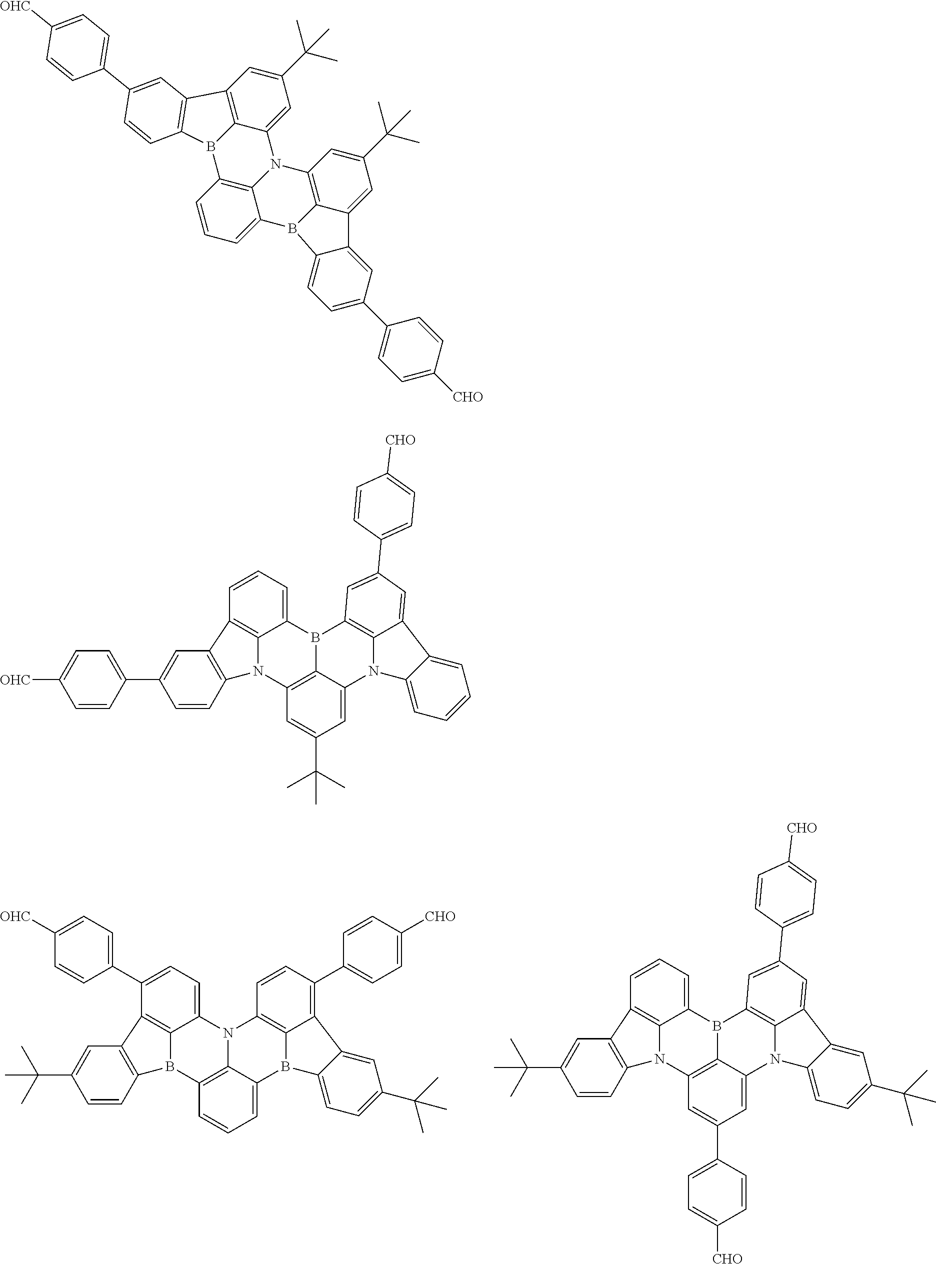 [SMILES: CC(C)(C)c1cc2c3c(c1)N1c4cc(C(C)(C)C)cc5c4B(c4ccc(-c6ccc(C=O)cc6)cc4-5)c4cccc(c41)B3c1ccc(-c3ccc(C=O)cc3)cc1-2 ; CC(C)(C)c1cc2c3c(c1)-n1c4ccccc4c4cc(-c5ccc(C=O)cc5)cc(c41)B3c1cccc3c4cc(-c5ccc(C=O)cc5)ccc4n-2c13 ; CC(C)(C)c1ccc2c(c1)-c1c(-c3ccc(C=O)cc3)ccc3c1B2c1cccc2c1N3c1ccc(-c3ccc(C=O)cc3)c3c1B2c1ccc(C(C)(C)C)cc1-3 ; CC(C)(C)c1ccc2c(c1)c1cccc3c1n2-c1cc(-c2ccc(C=O)cc2)cc2c1B3c1cc(-c3ccc(C=O)cc3)cc3c4cc(C(C)(C)C)ccc4n-2c13]

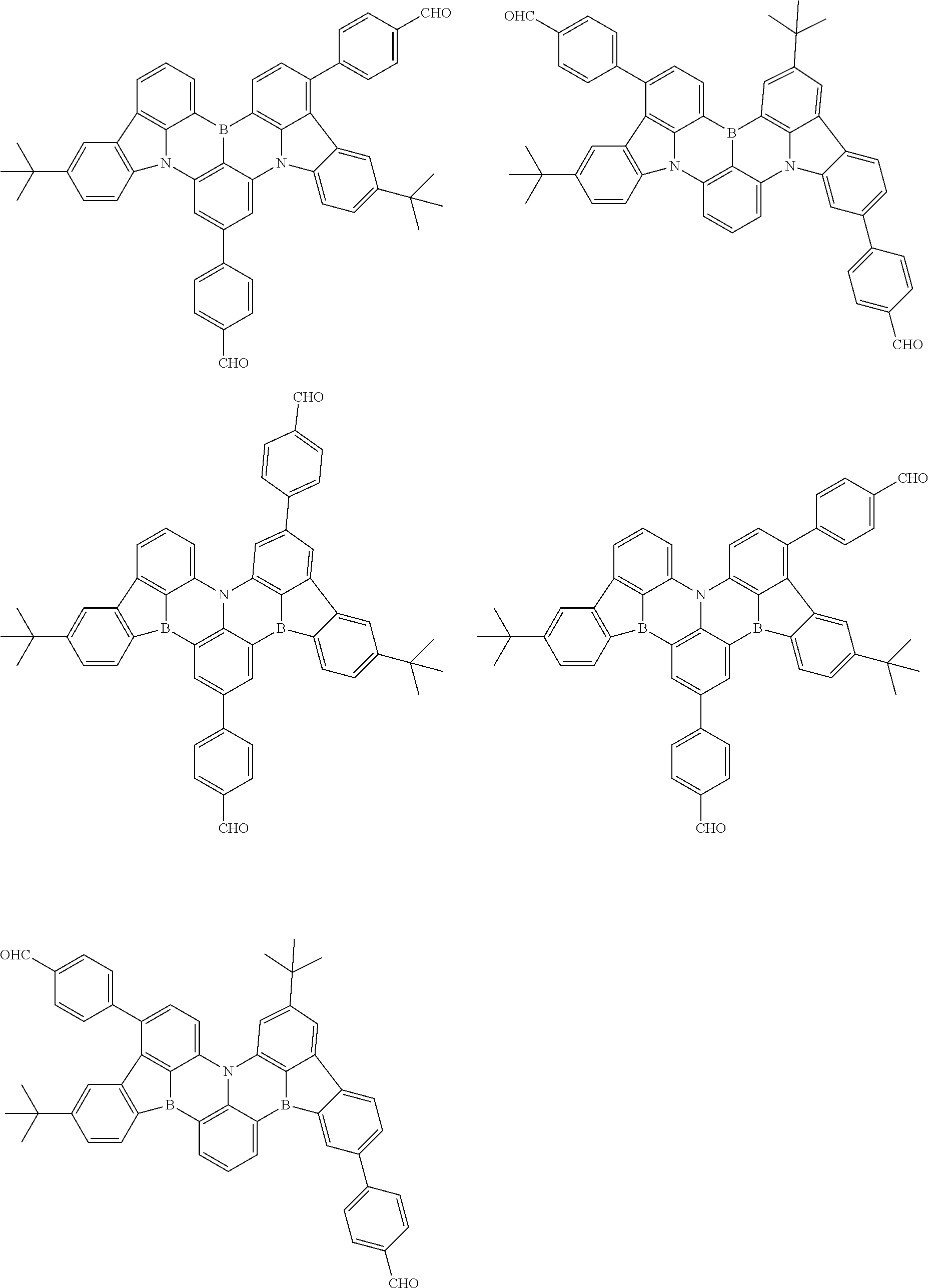

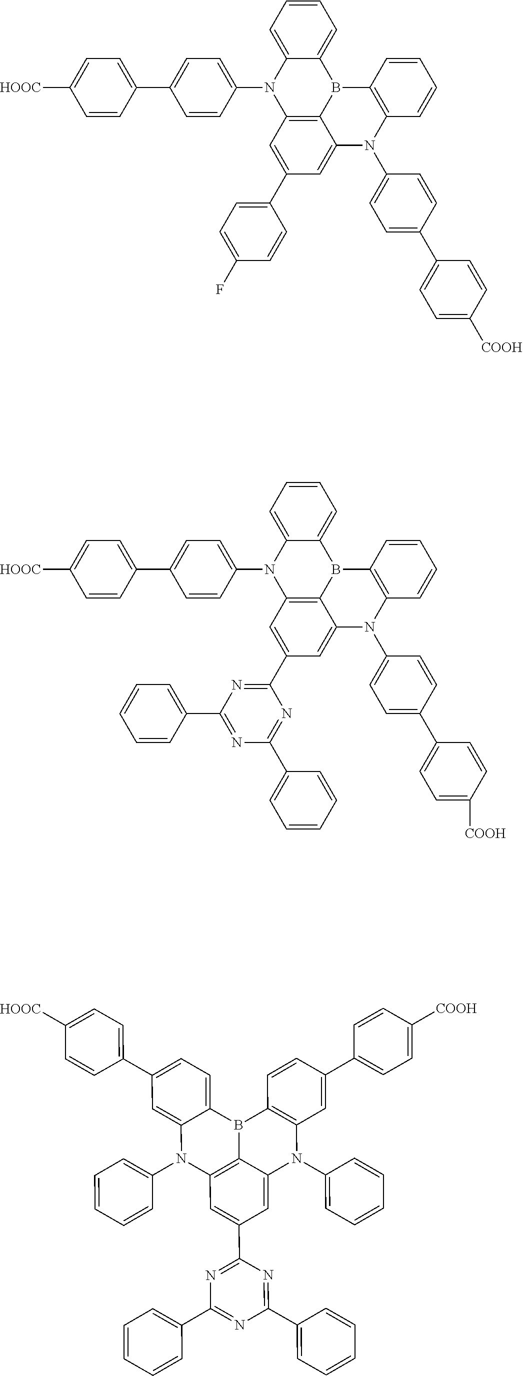 [SMILES: O=C(O)c1ccc(-c2ccc(N3c4ccccc4B4c5ccccc5N(c5ccc(-c6ccc(C(=O)O)cc6)cc5)c5cc(-c6ccc(F)cc6)cc3c54)cc2)cc1 ; O=C(O)c1ccc(-c2ccc(N3c4ccccc4B4c5ccccc5N(c5ccc(-c6ccc(C(=O)O)cc6)cc5)c5cc(-c6nc(-c7ccccc7)nc(-c7ccccc7)n6)cc3c54)cc2)cc1 ; O=C(O)c1ccc(-c2ccc3c(c2)N(c2ccccc2)c2cc(-c4nc(-c5ccccc5)nc(-c5ccccc5)n4)cc4c2B3c2ccc(-c3ccc(C(=O)O)cc3)cc2N4c2ccccc2)cc1]

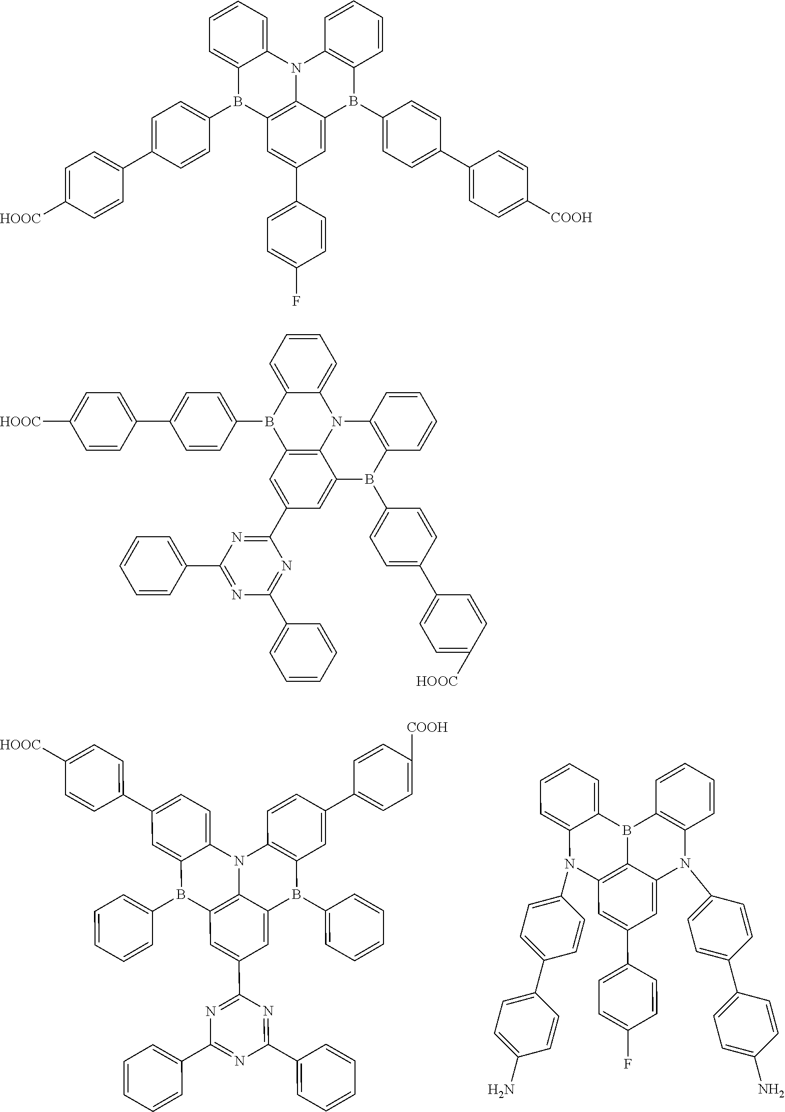

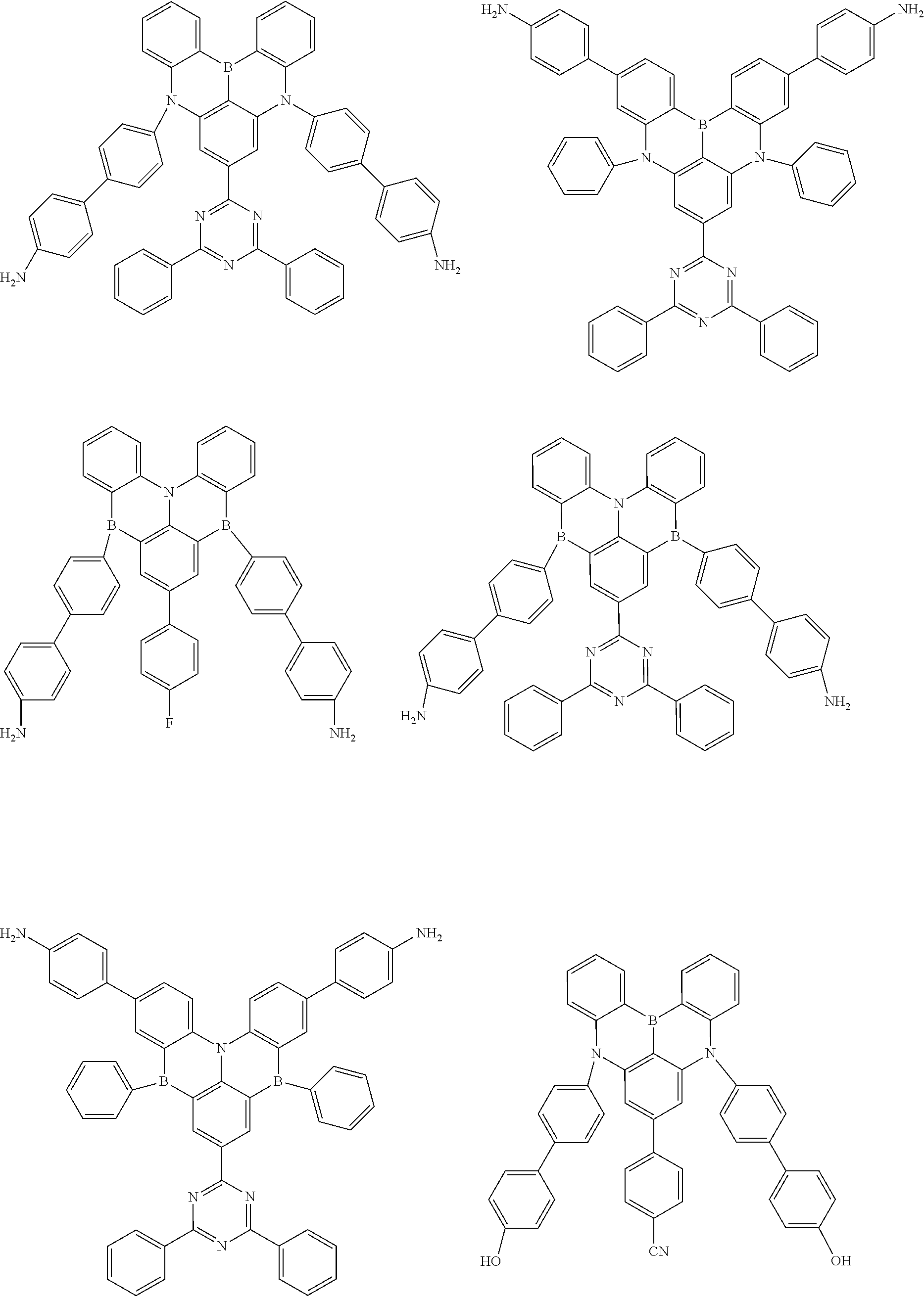

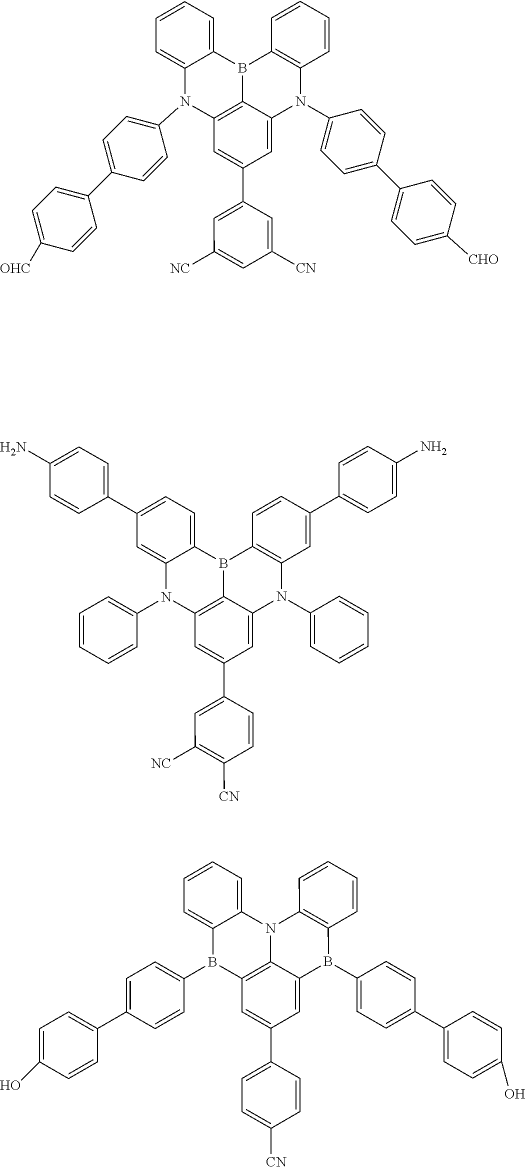 [SMILES: N#Cc1cc(C#N)cc(-c2cc3c4c(c2)N(c2ccc(-c5ccc(C=O)cc5)cc2)c2ccccc2B4c2ccccc2N3c2ccc(-c3ccc(C=O)cc3)cc2)c1 ; N#Cc1ccc(-c2cc3c4c(c2)N(c2ccccc2)c2cc(-c5ccc(N)cc5)ccc2B4c2ccc(-c4ccc(N)cc4)cc2N3c2ccccc2)cc1C#N ; N#Cc1ccc(-c2cc3c4c(c2)B(c2ccc(-c5ccc(O)cc5)cc2)c2ccccc2N4c2ccccc2B3c2ccc(-c3ccc(O)cc3)cc2)cc1]

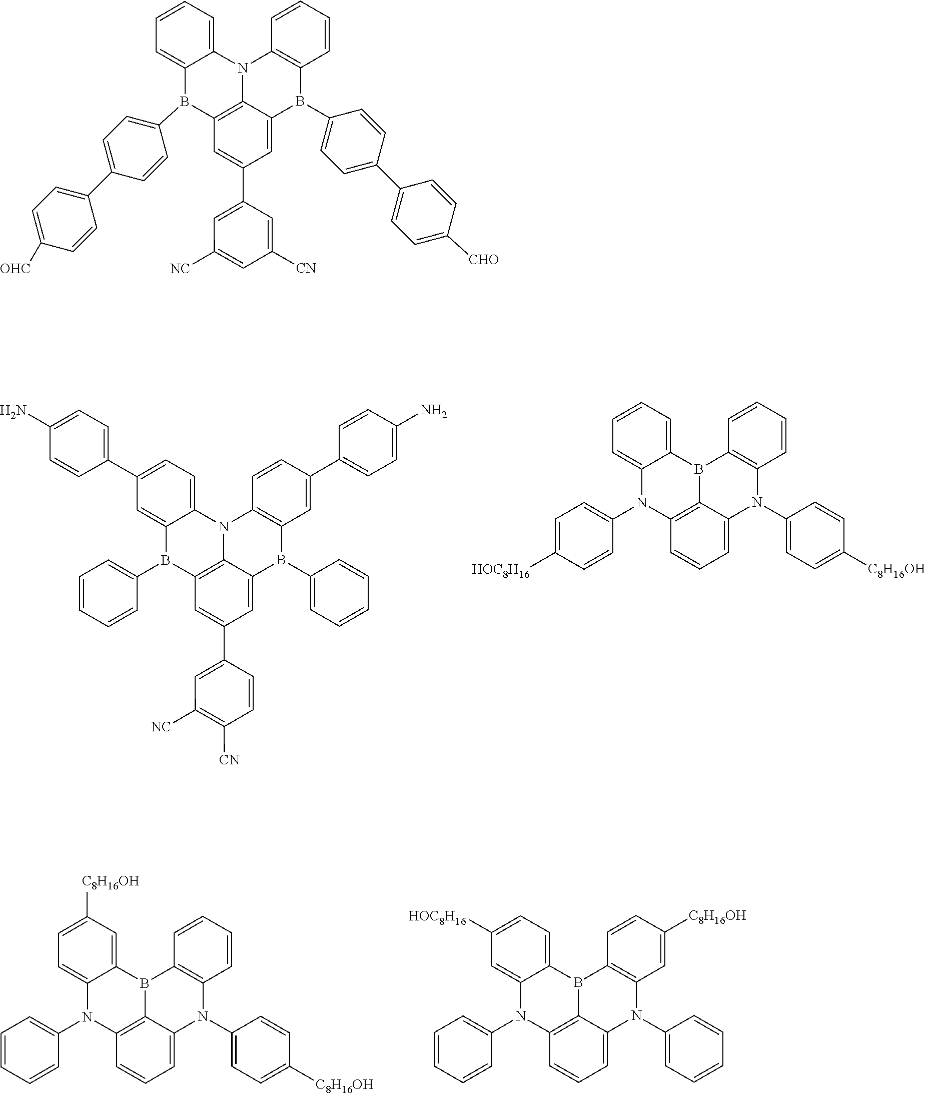

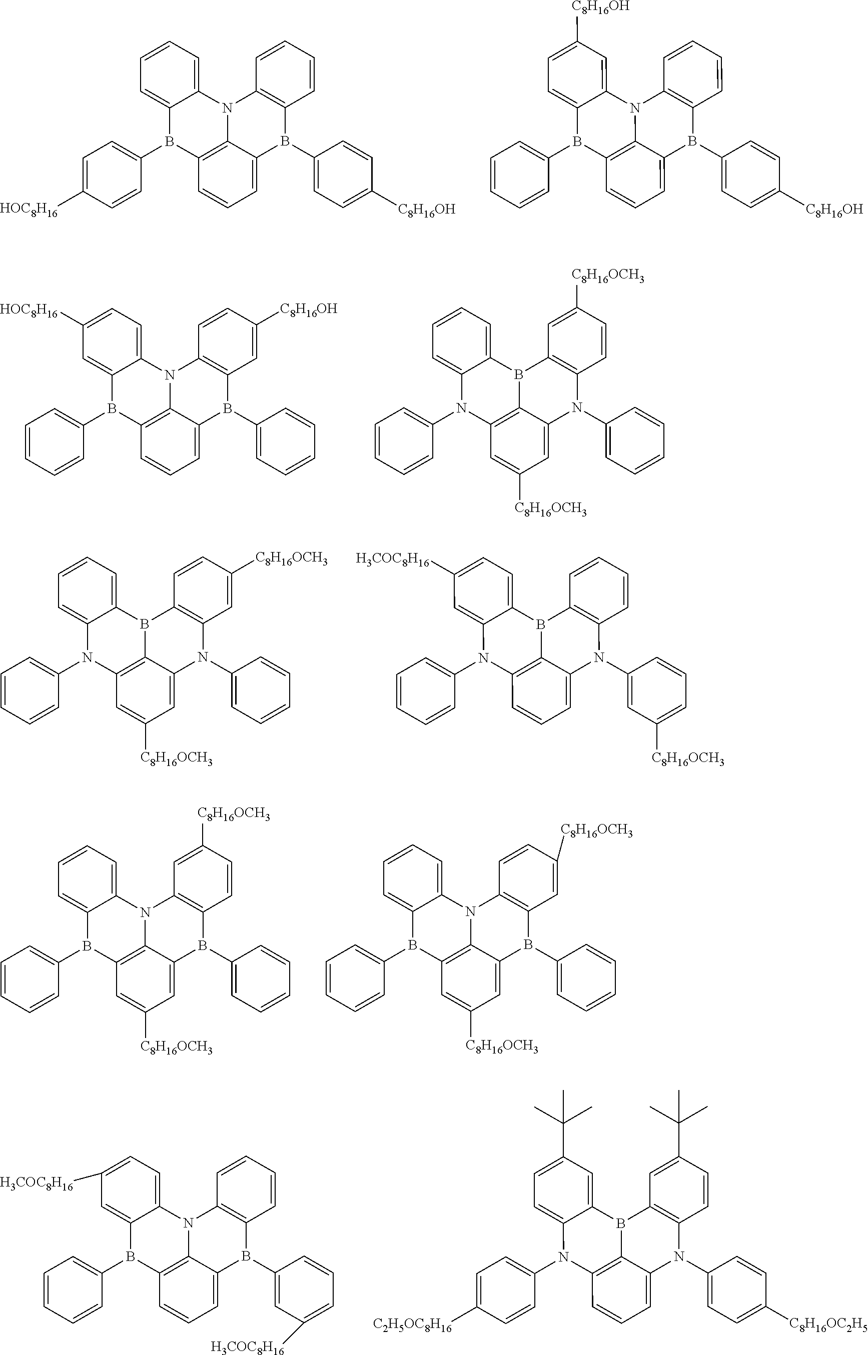

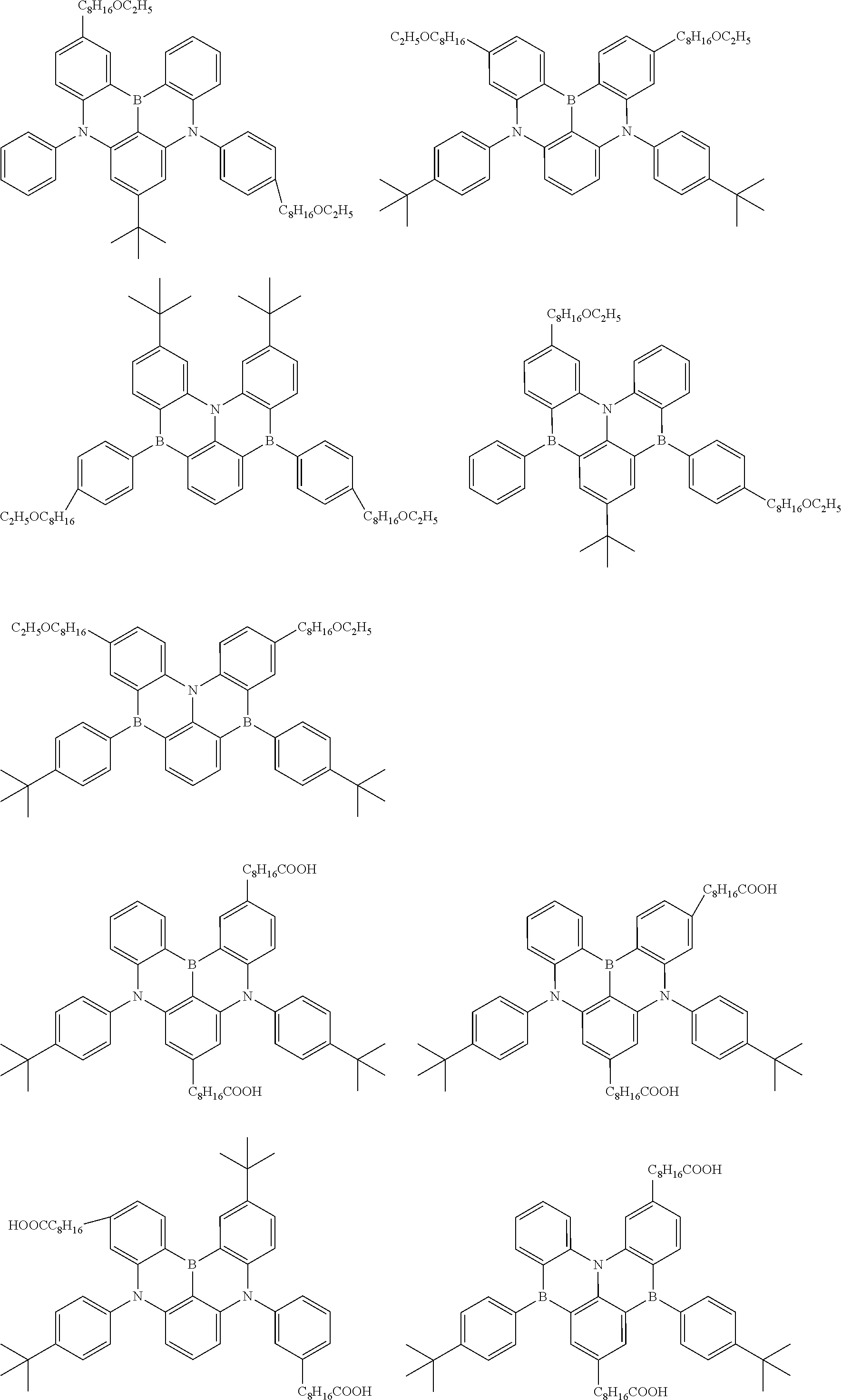

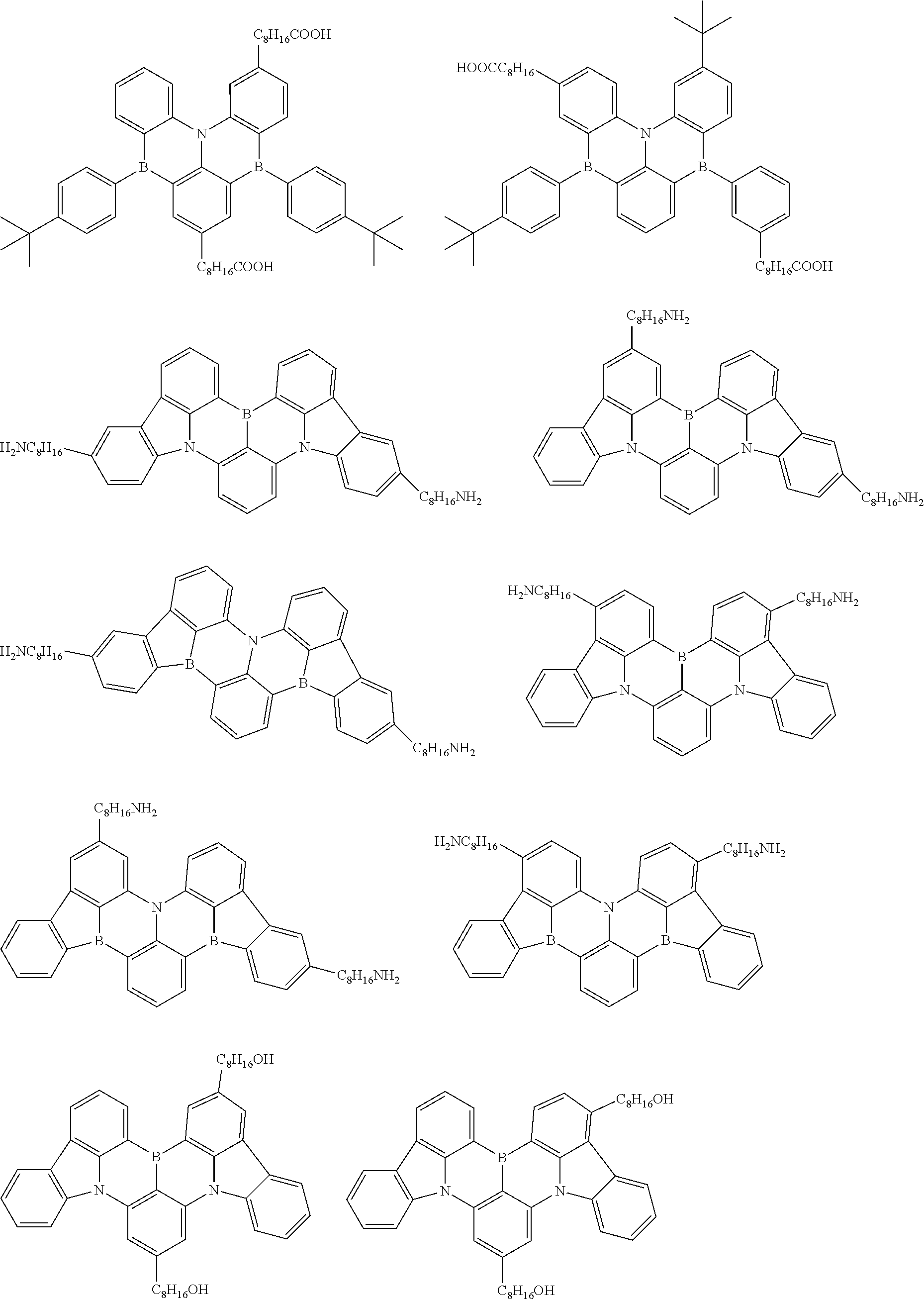

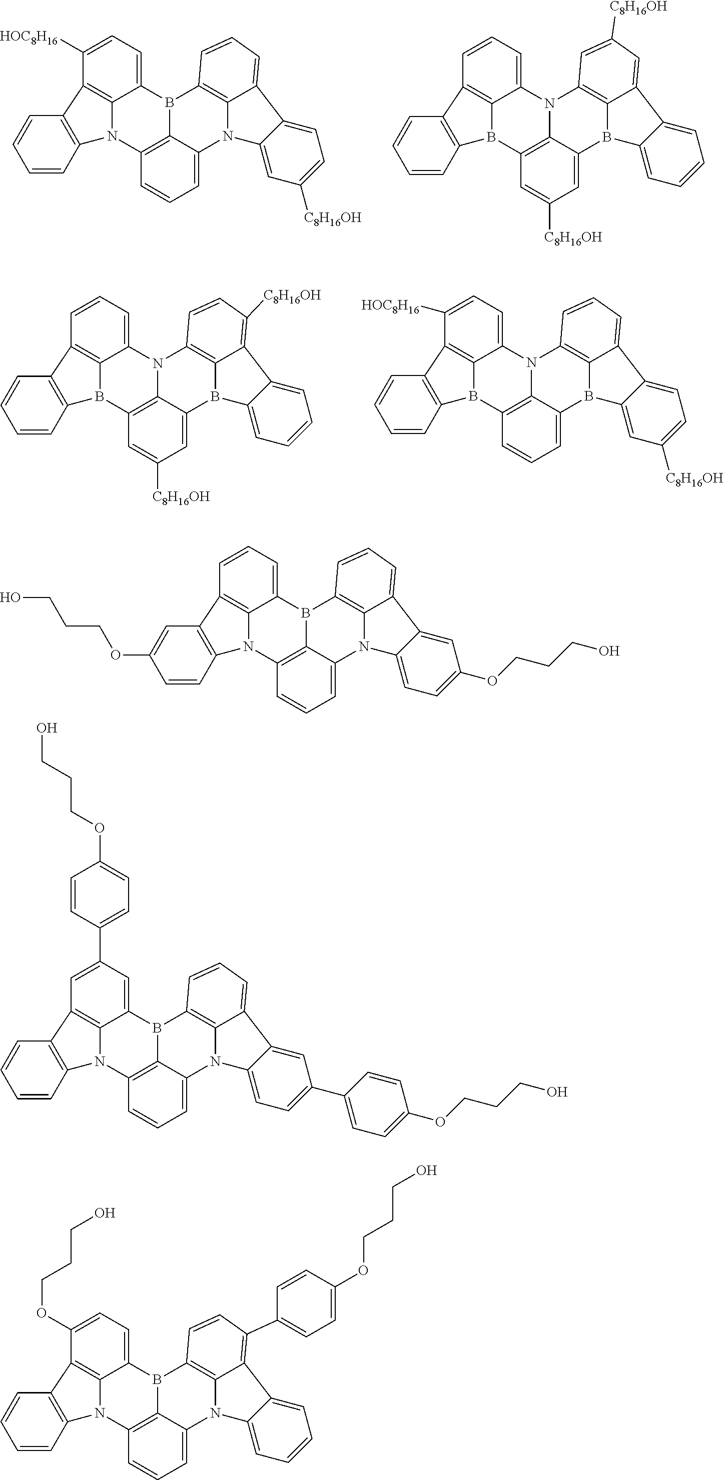

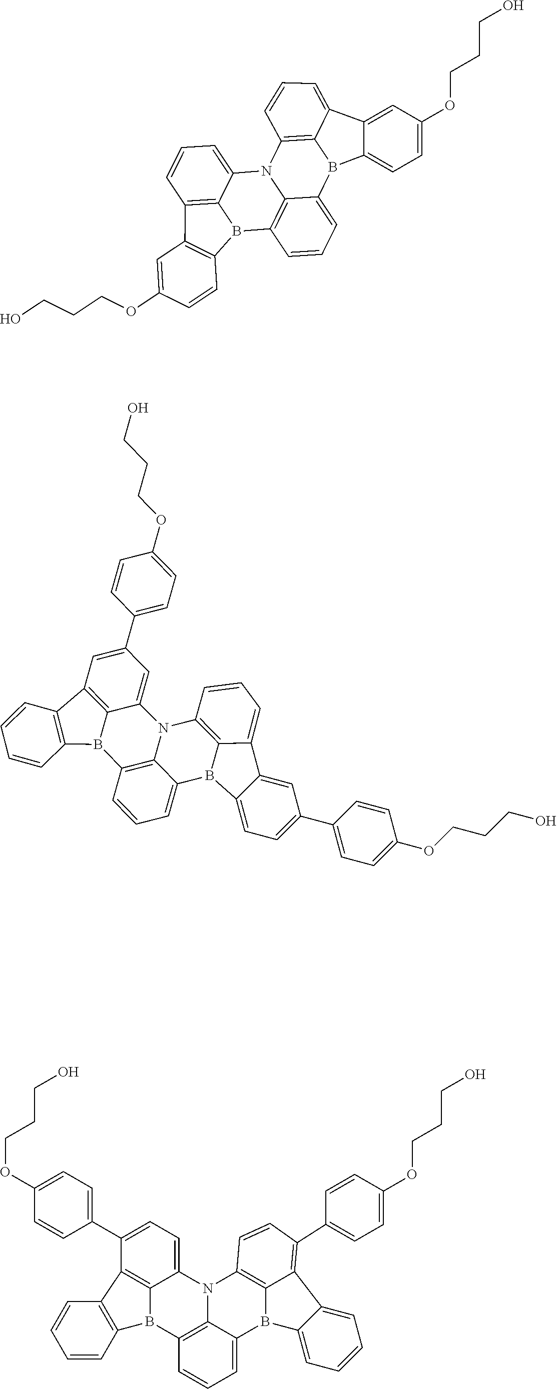 [SMILES: OCCCOc1ccc2c(c1)-c1cccc3c1B2c1cccc2c1N3c1cccc3c1B2c1ccc(OCCCO)cc1-3 ; OCCCOc1ccc(-c2ccc3c(c2)-c2cccc4c2B3c2cccc3c2N4c2cc(-c4ccc(OCCCO)cc4)cc4c2B3c2ccccc2-4)cc1 ; OCCCOc1ccc(-c2ccc3c4c2-c2ccccc2B4c2cccc4c2N3c2ccc(-c3ccc(OCCCO)cc3)c3c2B4c2ccccc2-3)cc1]

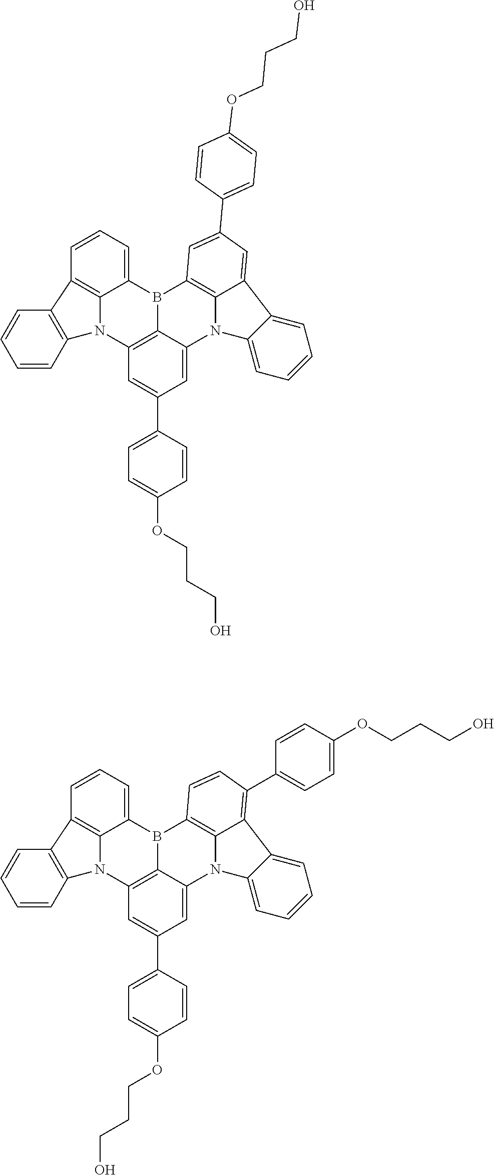 [SMILES: OCCCOc1ccc(-c2cc3c4c(c2)-n2c5ccccc5c5cc(-c6ccc(OCCCO)cc6)cc(c52)B4c2cccc4c5ccccc5n-3c24)cc1 ; OCCCOc1ccc(-c2cc3c4c(c2)-n2c5ccccc5c5c(-c6ccc(OCCCO)cc6)ccc(c52)B4c2cccc4c5ccccc5n-3c24)cc1]

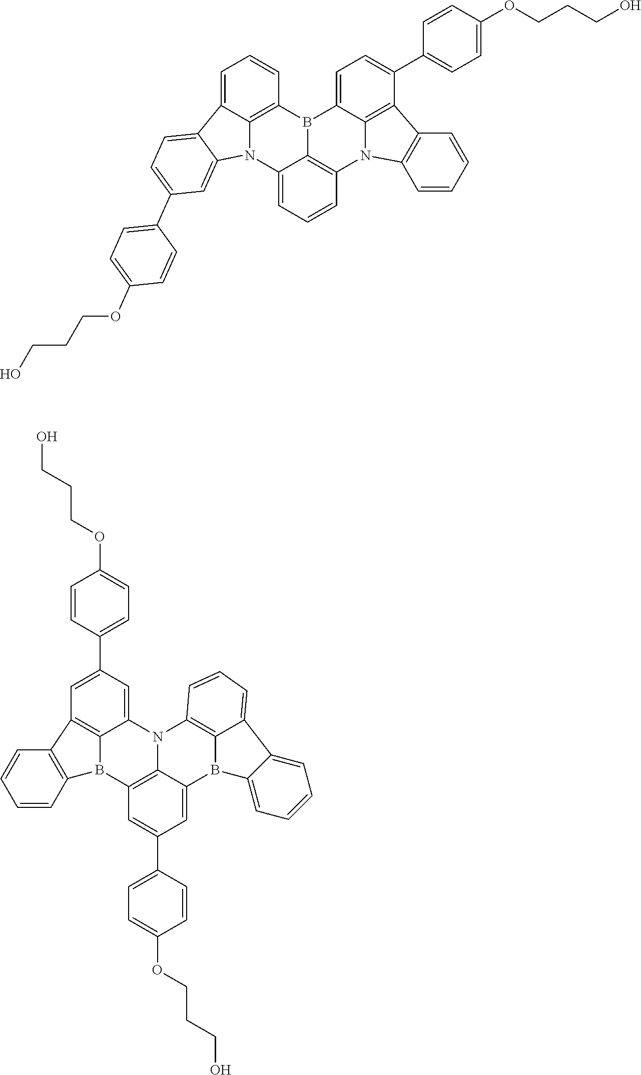 [SMILES: OCCCOc1ccc(-c2ccc3c4cccc5c4n(c3c2)-c2cccc3c2B5c2ccc(-c4ccc(OCCCO)cc4)c4c5ccccc5n-3c24)cc1 ; OCCCOc1ccc(-c2cc3c4c(c2)B2c5ccccc5-c5cc(-c6ccc(OCCCO)cc6)cc(c52)N4c2cccc4c2B3c2ccccc2-4)cc1]

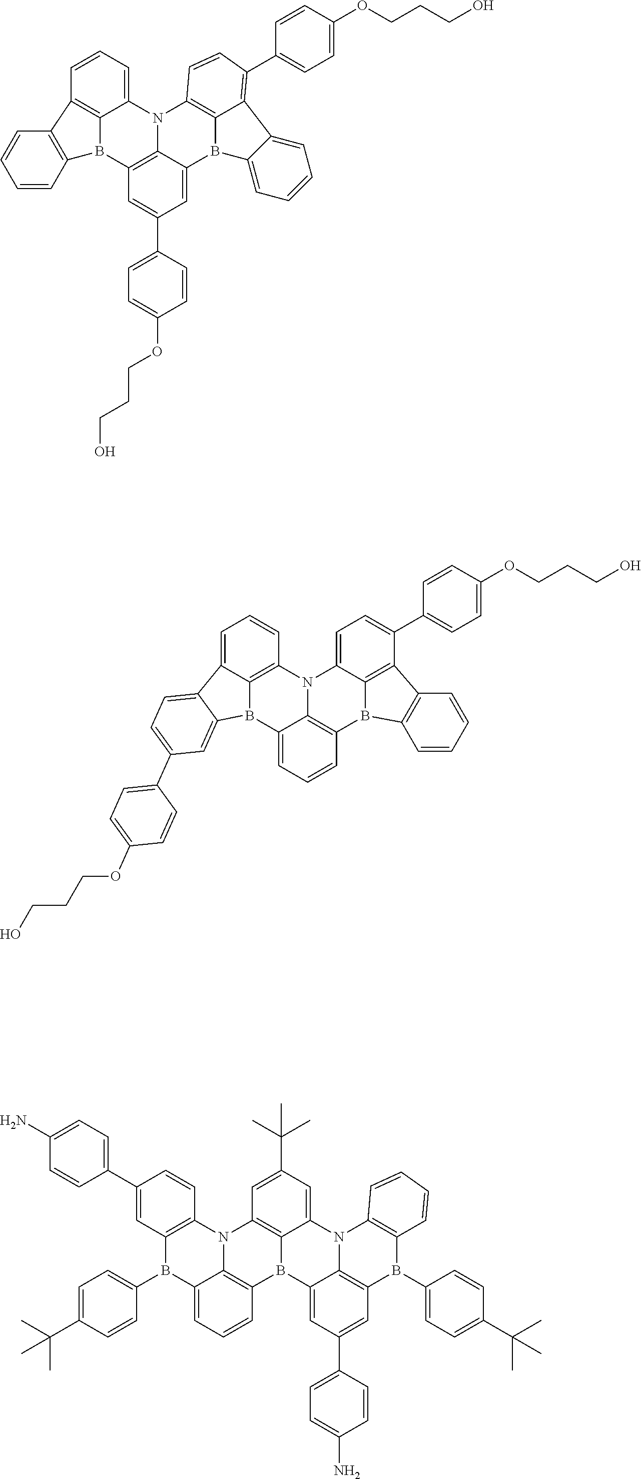 [SMILES: OCCCOc1ccc(-c2cc3c4c(c2)B2c5ccccc5-c5c(-c6ccc(OCCCO)cc6)ccc(c52)N4c2cccc4c2B3c2ccccc2-4)cc1 ; OCCCOc1ccc(-c2ccc3c(c2)B2c4cccc5c4N(c4cccc-3c42)c2ccc(-c3ccc(OCCCO)cc3)c3c2B5c2ccccc2-3)cc1 ; CC(C)(C)c1ccc(B2c3cc(-c4ccc(N)cc4)ccc3N3c4cc(C(C)(C)C)cc5c4B(c4cccc2c43)c2cc(-c3ccc(N)cc3)cc3c2N5c2ccccc2B3c2ccc(C(C)(C)C)cc2)cc1]

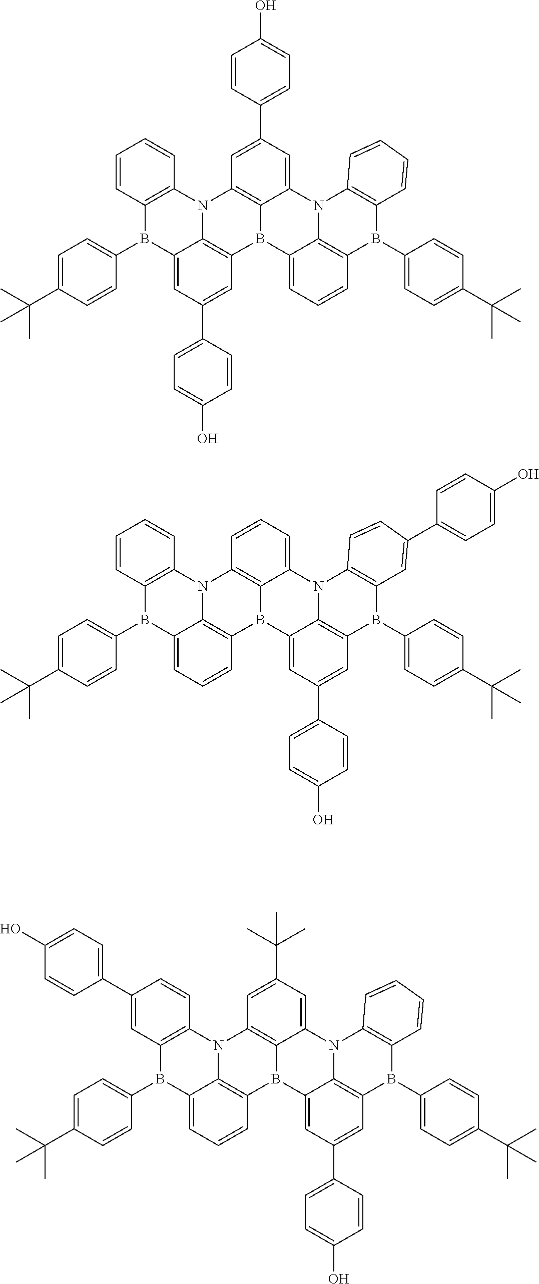

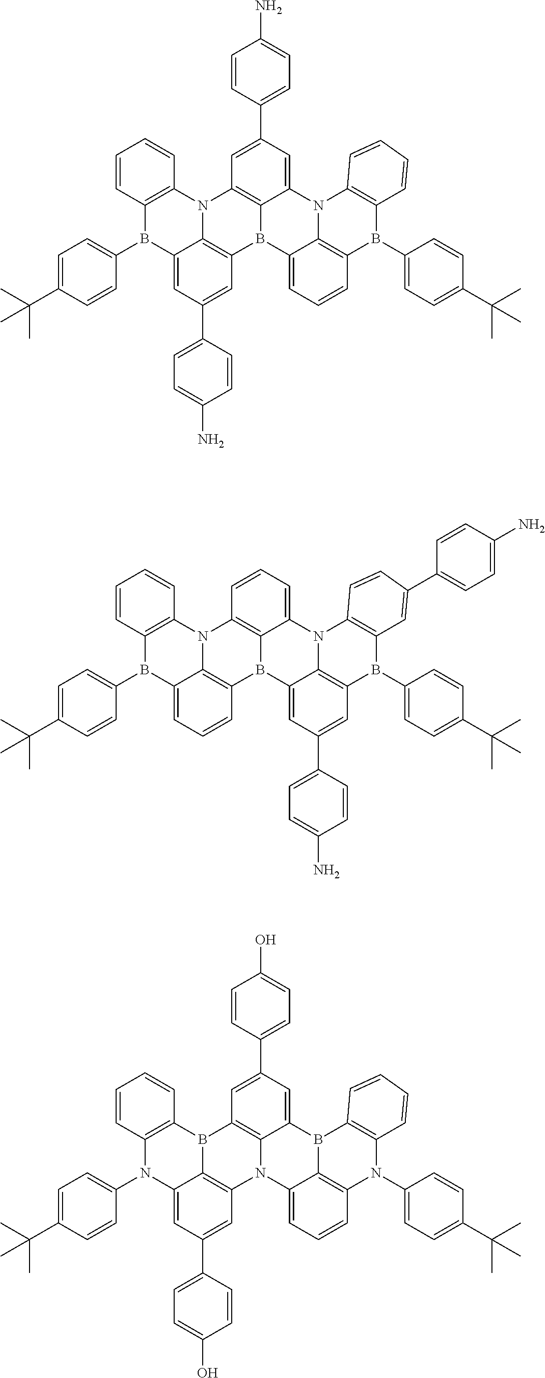

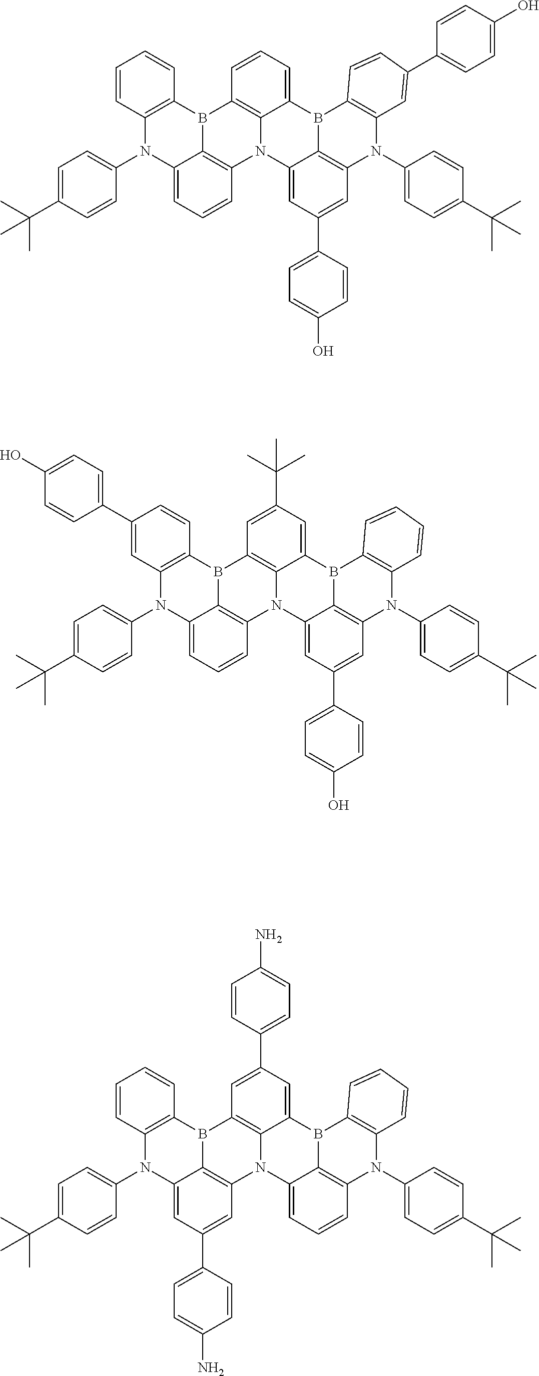

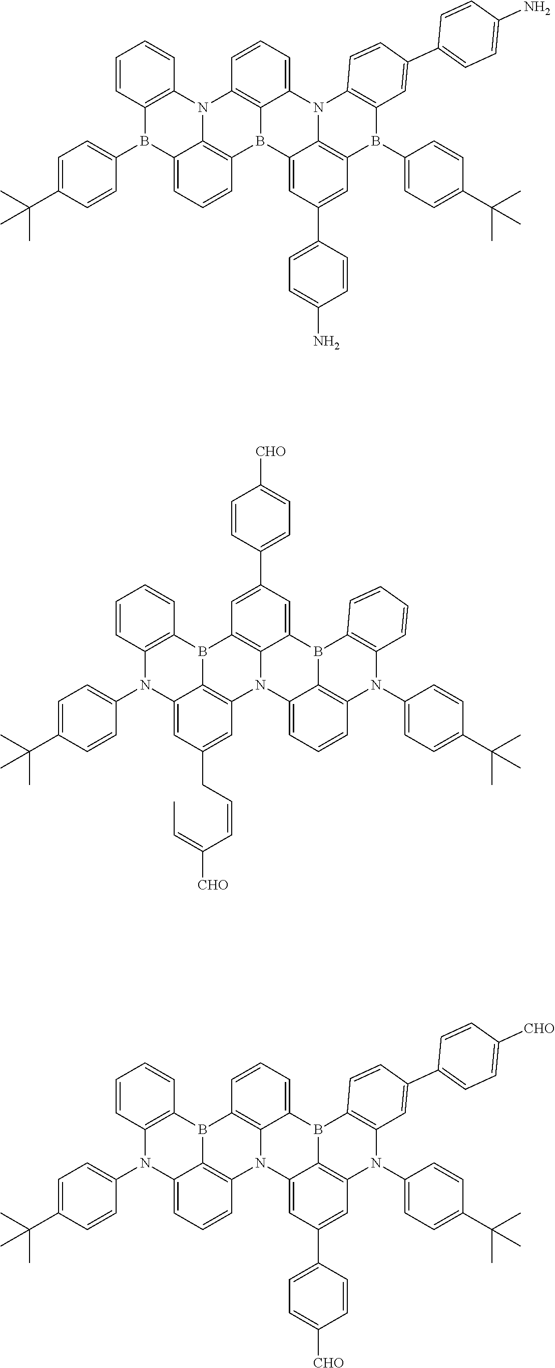

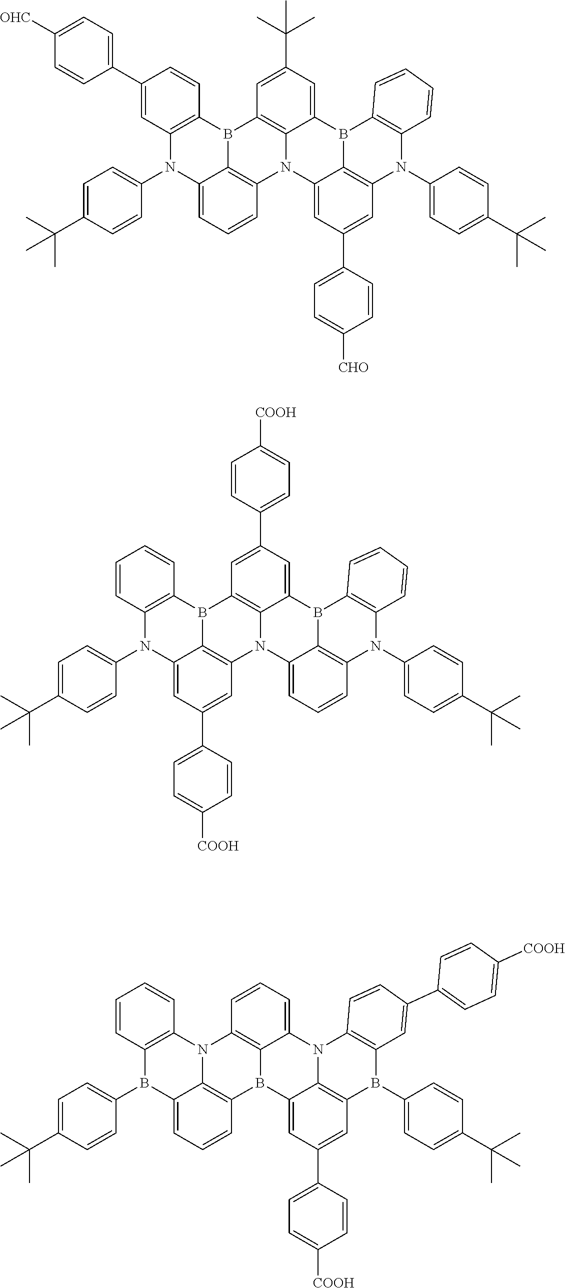

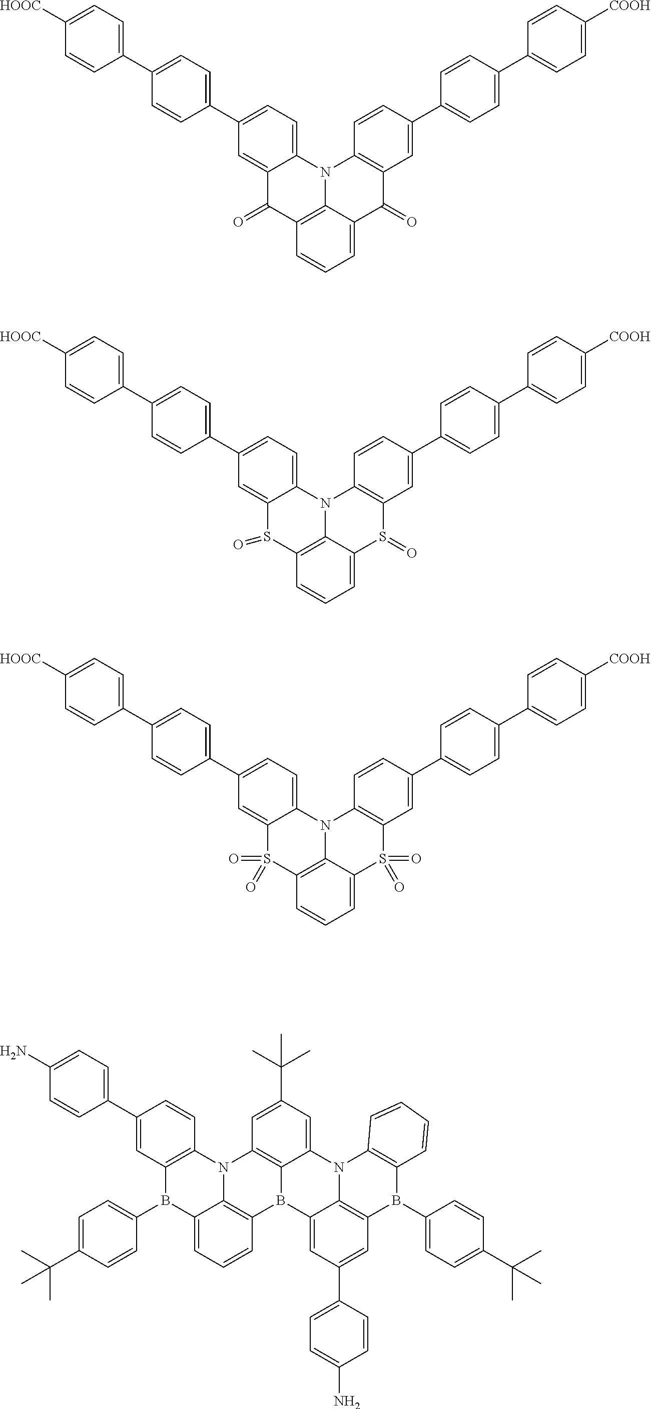 [SMILES: O=C(O)c1ccc(-c2ccc(-c3ccc4c(c3)c(=O)c3cccc5c(=O)c6cc(-c7ccc(-c8ccc(C(=O)O)cc8)cc7)ccc6n4c35)cc2)cc1 ; O=C(O)c1ccc(-c2ccc(-c3ccc4c(c3)S(=O)c3cccc5c3N4c3ccc(-c4ccc(-c6ccc(C(=O)O)cc6)cc4)cc3S5=O)cc2)cc1 ; O=C(O)c1ccc(-c2ccc(-c3ccc4c(c3)S(=O)(=O)c3cccc5c3N4c3ccc(-c4ccc(-c6ccc(C(=O)O)cc6)cc4)cc3S5(=O)=O)cc2)cc1 ; CC(C)(C)c1ccc(B2c3cc(-c4ccc(N)cc4)ccc3N3c4cc(C(C)(C)C)cc5c4B(c4cccc2c43)c2cc(-c3ccc(N)cc3)cc3c2N5c2ccccc2B3c2ccc(C(C)(C)C)cc2)cc1]

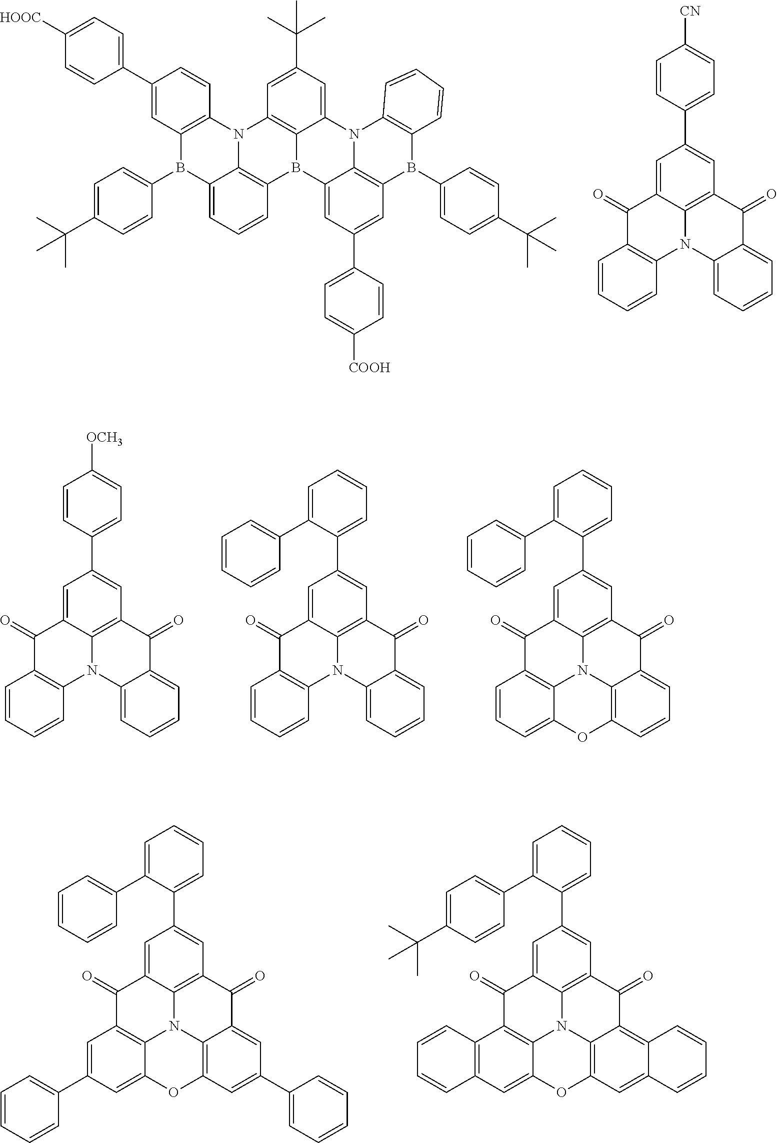

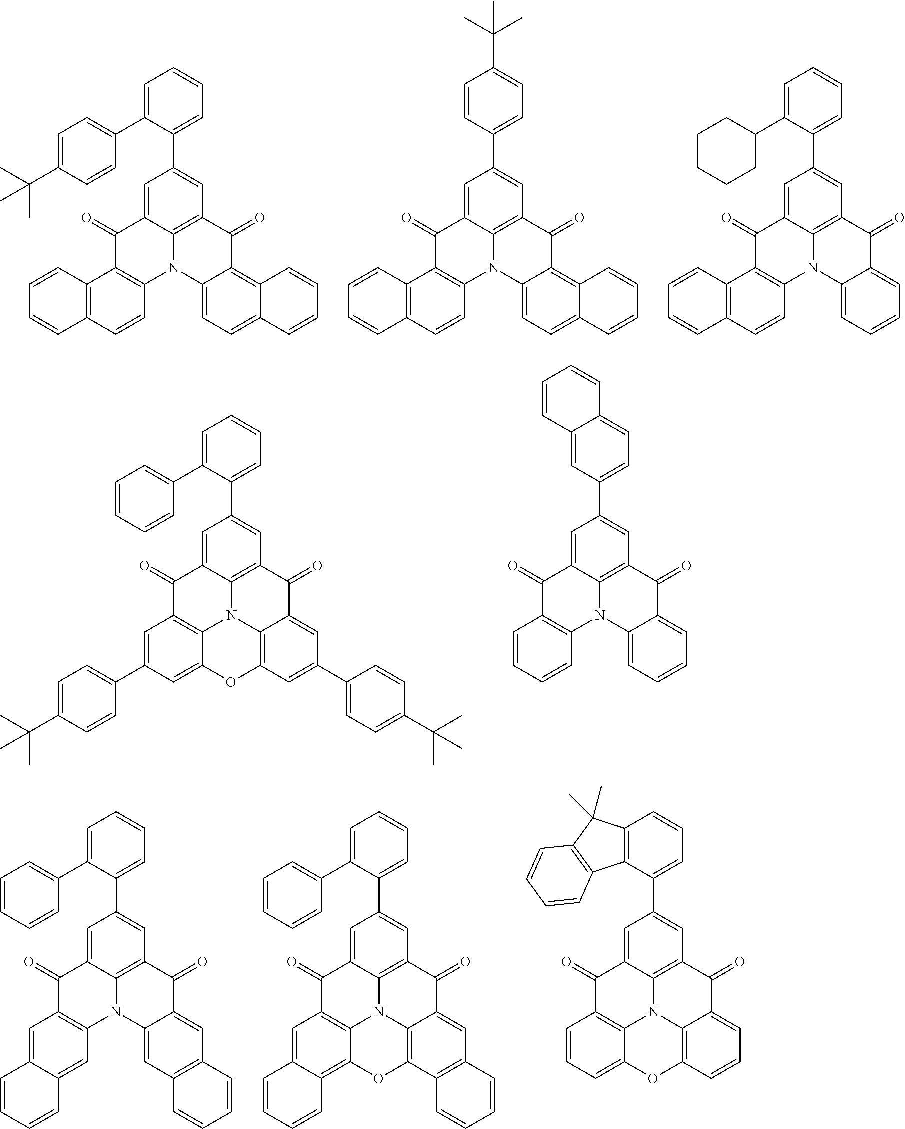

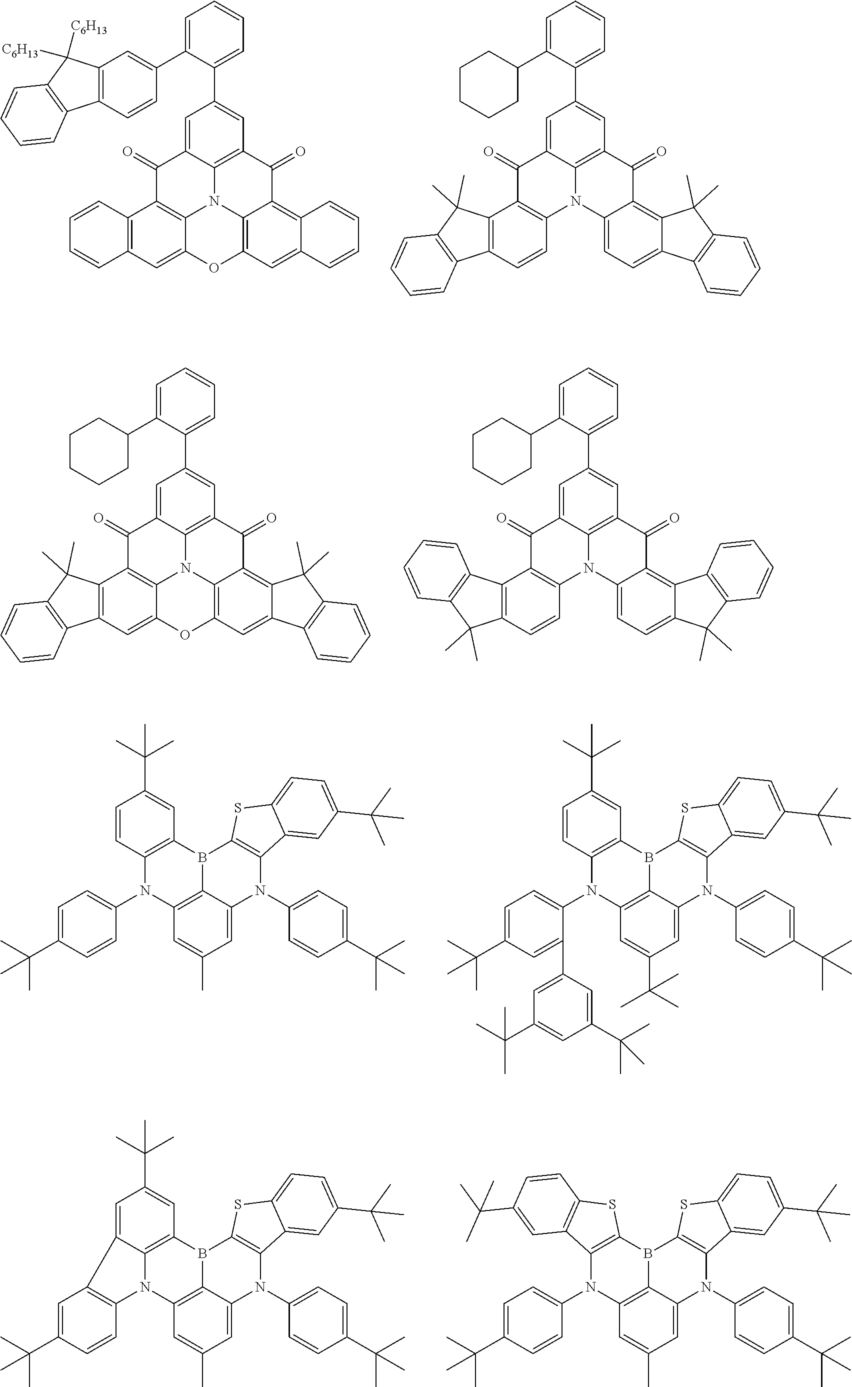

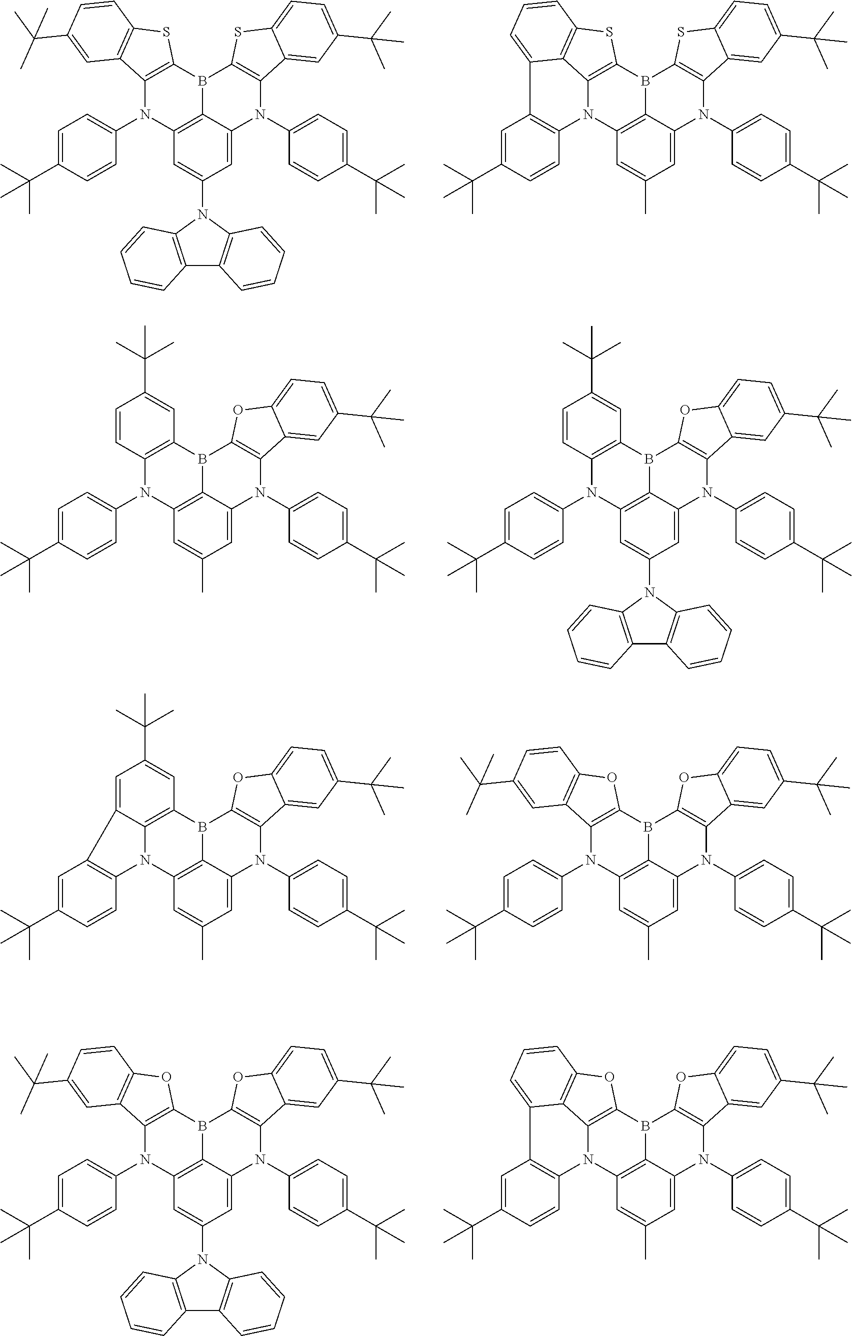

In some embodiments, the emitter E comprises at least one cross-linkable group, as disclosed in a contemporaneous patent with the application No. CN 202110370910.9, the patent documents above are specially incorporated herein by reference in their entirety. The advantage is that when the resin prepolymer is copolymerized or homopolymerized, the emitter E can at least partially or completely participate in the polymerisation.

In some embodiments, the emitter E comprises at least two cross-linkable groups.

In some embodiments, the emitter E comprises at least three cross-linkable groups.

In some embodiments, the emitter E is a polymer, comprising at least one repeating structural unit of formula (1) or (2). Preferably, the polymer is a side chain polymer as disclosed in the contemporaneous patent with the application No. CN202110370854.9. The patent document above are specially incorporated herein by reference in their entirety.

In the formulation as described herein, the organic compound H has relatively high extinction coefficient. The extinction coefficient is also known as the molar extinction coefficient, which refers to the absorption coefficient at a concentration of 1 mol/L, and is represented by the symbol ε, in unit of Lmol⁻¹cm⁻¹. The extinction coefficient (ε) preferably ≥1*10³; more preferably ≥1*10⁴; particularly preferably ≥5*10⁴; and most preferably ≥1*10⁵. Preferably, the extinction coefficient refers to the extinction coefficient at the wavelength corresponding to the absorption peak.

In some embodiments, the absorption spectrum of the organic compound H is between 380 nm and 500 nm.

In some embodiments, the emission spectrum of the organic compound H is between 440 nm to 500 nm.

In some embodiments, the wavelength of the emission peak of the organic compound H corresponds to a wavelength <500 nm.

In some embodiments, the emission spectrum of the organic compound H is between 500 nm to 580 nm.

For the purposes of the present disclosure, the proportion of the organic compound H in the formulation is large, so the synthesis of the organic compound H is required to be low-cost and environmentally friendly.

In some embodiments, the organic compound H does not comprise a cyano group.

In some embodiments, the organic compound H is not a Bodipy derivative.

The energy structure of the organic compound H has an important influence on its optoelectronic properties and stability.

Preferably, the organic compound H as described herein has a large S1-T1, where the S1-T1 generally ≥0.70 eV, preferably ≥0.80 eV, more preferably ≥0.90 eV, further preferably ≥1.00 eV, and most preferably ≥1.10 eV.

In some embodiments, the organic compound H has a large ΔHOMO and/or ΔLUMO, generally ≥0.50 eV, preferably ≥0.60 eV, more preferably ≥0.70 eV, further preferably ≥0.80 eV, and most preferably ≥0.90 eV; where ΔHOMO=HOMO−(HOMO−1), ΔLUMO=(LUMO+1)−LUMO.

For the purposes of the present disclosure, (HOMO−1) is defined as the energy level of the second highest occupied molecular orbital, (HOMO−2) is defined as the energy level of the third highest occupied molecular orbital, and so on. (LUMO+1) is defined as the energy level of the second lowest unoccupied molecular orbital, (LUMO+2) is defined as the energy level of the third lowest occupied molecular orbital, and so on; these energy levels can be determined by the following simulation method.

In some embodiments, the organic compound H has relatively large oscillator strength f(Sn) (n≥1); f(S1) generally ≥0.20 eV, preferably ≥0.30 eV, more preferably ≥0.40 eV, further preferably ≥0.50 eV, and most preferably ≥0.60 eV.

In some embodiments, the organic compound H has relatively low HOMO, generally ≤−5.0 eV, preferably ≤−5.1 eV, more preferably ≤−5.2 eV, further preferably ≤−5.3 eV, and most preferably ≤−5.4 eV.

More preferably, the HOMO of the organic compound H≤−5.5 eV, preferably ≤−5.6 eV, more preferably ≤−5.7 eV, further preferably ≤−5.8 eV, and most preferably ≤−5.9 eV.

In some embodiments, the organic compound H has relatively high LUMO, generally ≥−3.5 eV, preferably ≥−3.3 eV, more preferably ≥−3.1 eV, further preferably ≥−2.9 eV, and most preferably ≥−2.7 eV.

The suitable organic compounds H may be selected from organic small molecules, polymers, or metal complexes.

In some embodiments, the organic compound H may be selected from cyclic aromatic hydrocarbon compound, such as benzene, biphenyl, triphenylbenzene, benzophenanthrene, triphenylene, anthracene, phenalene, phenanthrene, fluorene, pyrene, chrysene, perylene, azulene. The organic compound used as the singlet host material may be also selected from aromatic heterocyclic compound, such as dibenzothiophene, dibenzofuran, dibenzothiophene, furan, thiophene, benzofuran, benzothiophene, benzoselenophene, carbazole, indocarbazole, pyridindole, pyrroledipyridine, pyrazole, imidazole, triazole, isoxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indolizine, benzoxazole, Benzoisoxazole benzothiazole, quinoline, isoquinoline, cinnoline, quinazoline, quinoxaline, naphthalene, phthalein pteridine, xanthene, acridine, phenazine, phenothiazine, phenoxazine, benzofuranopyridine, furandipyridine, benzothienopyridine, thiophenedipyridine, benzoselenophenopyridine, or selenophenodipyridine. The organic compound used as the singlet host material may be selected from groups containing 2 to 10 ring structures, which may be the same or different types of cyclic aryl or heterocyclic aryl, and are linked to each other directly or by at least one of the following groups, such as oxygen atom, nitrogen atom, sulfur atom, silicon atom, phosphorus atom, boron atom, chain structure unit, or aliphatic ring group.

In some embodiments, the organic compound H may be selected from compounds comprising at least one of the following groups:

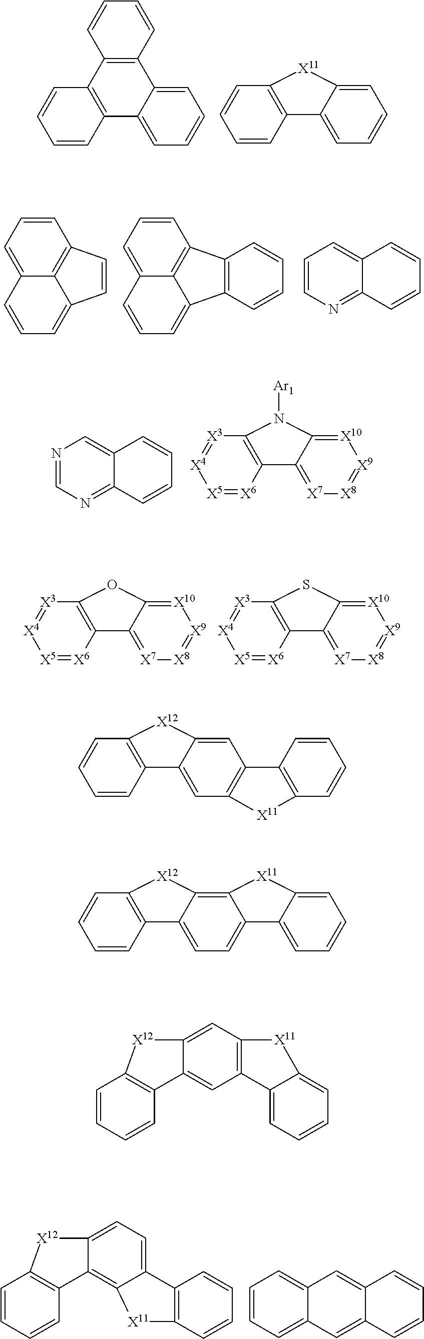

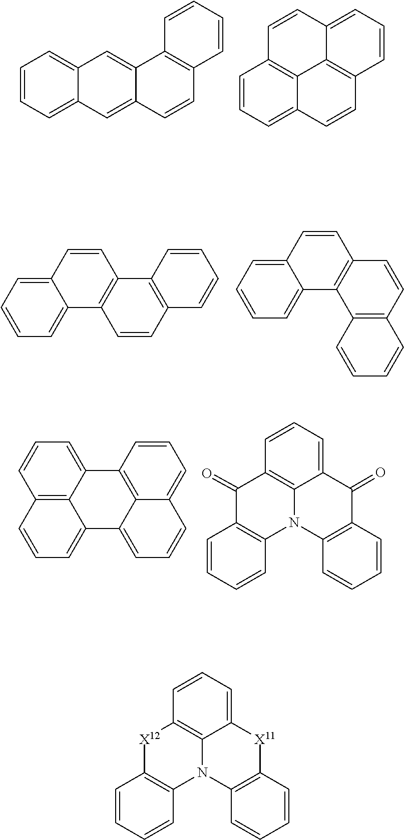

Where Ar₁ is aryl or heteroaryl group; each of X³ to X¹⁰ at each occurrence is CR¹ or N; each of X¹¹ and X¹² at each occurrence is independently selected from CR¹R², NR¹, or O; each of R¹ and R² at each occurrence is independently selected from the group consisting of —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, and a disubstituted unit in any position of the above substituents or a combination thereof, wherein one or more R¹-R² may form a monocyclic or polycyclic aliphatic or aromatic ring system with each other and/or with the rings bonded thereto.

In some embodiments, the organic compound H has a long conjugated π-electron system. Hitherto, there have been many examples of styryl amines and derivatives thereof as disclosed in JP2913116B and WO2001021729A1, and indenofluorenes and derivatives thereof as disclosed in WO2008006449 and WO2007140847.

In some embodiments, the organic compound H can be selected from the group consisting of monostyrylamines, distyrylamines, tristyrylamines, tetrastyrylamines, styrenphosphines, styrenethers, and arylamines.

A monostyrylamine refers to a compound which comprises one unsubstituted or substituted styryl group and at least one amine, most preferably an aryl amine. Distyrylamine refers to a compound comprising two unsubstituted or substituted styryl groups and at least one amine, most preferably an aryl amine. Ternarystyrylamine refers to a compound which comprises three unsubstituted or substituted styryl groups and at least one amine, most preferably an aryl amine. Quaternarystyrylamine refers to a compound comprising four unsubstituted or substituted styryl groups and at least one amine, most preferably an aryl amine. Preferred styrene is stilbene, which may be further substituted. The corresponding phosphines and ethers are defined similarly as amines. Aryl amine or aromatic amine refers to a compound comprising three unsubstituted or substituted cyclic or heterocyclic aryl systems directly attached to nitrogen. At least one of these cyclic or heterocyclic aryl systems is preferably selected from fused ring systems and most preferably has at least 14 aryl ring atoms. Among the preferred examples are aryl anthramine, aryl anthradiamine, aryl pyrene amines, aryl pyrene diamines, aryl chrysene amines and aryl chrysene diamine. Aryl anthramine refers to a compound in which one diarylamino group is directly attached to anthracene, most preferably at position 9. Aryl anthradiamine refers to a compound in which two diarylamino groups are directly attached to anthracene, most preferably at positions 9,10. Aryl pyrene amines, aryl pyrene diamines, aryl chrysene amines and aryl chrysene diamine are similarly defined, where the diarylarylamino group is most preferably attached to position 1 or 1,6 of pyrene.

Examples of organic compounds H based on vinylamines and arylamines may be found in the following patent documents: WO2006000388, WO2006058737, WO2006000389, WO2007065549, WO2007115610, U.S. Pat. No. 7,250,532B2, DE102005058557A1, CN1583691A, JP08053397A, U.S. Pat. No. 6,251,531B1, US2006210830A, EP1957606A1, and US20080113101A1. The patent documents listed above are specially incorporated herein by reference in their entirety.

Examples of organic compounds H based on stilbene and its derivatives may be found in U.S. Pat. No. 5,121,029.

Further preferred organic compounds H can be selected from the group consisting of indenofluorene-amine and indenofluorene-diamine, as disclosed in WO2006122630, benzoindenofluorene-amine and benzoindenofluorene-diamine, as disclosed in WO2008006449, dibenzoindenofluorene-amine and dibenzoindenofluorene-diamine, as disclosed in WO2007140847.

Other materials that can be used as organic compound H include polycyclic aromatic hydrocarbon compounds, in particular selected from the derivatives of the following compounds: anthracene such as 9,10-di(2-naphthyl)anthracene, naphthalene, tetraphenyl, phenanthrene, perylene such as 2,5,8,11-tetra-t-butylatedylene, indenoperylene, phenylene (benzo fused ring such as 4,4′-(bis (9-ethyl-3-carbazovinylene)-1,1′-biphenyl)), periflanthene, decacyclene, coronene, fluorene, spirobifluorene, arylpyren (e.g., US20060222886), arylenevinylene (e.g. U.S. Pat. Nos. 5,121,029, 5,130,603), cyclopentadiene such as tetraphenylcyclopentadiene, rubrene, coumarine, rhodamine, quinacridone, pyrane such as 4 (dicyanoethylene)-6-(4-dimethylaminostyryl-2-methyl)-4H-pyrane (DCM), thiapyran, bis (azinyl) imine-boron compounds (e.g. US20070092753A1), bis (azinyl) methene compounds, carbostyryl compounds, oxazone, benzoxazole, benzothiazole, benzimidazole, or diketopyrrolopyrrole. Some singlet emitter materials may be found in the following patent documents: US20070252517A1, U.S. Pat. Nos. 4,769,292, 6,020,078. The patent documents listed above are specially incorporated herein by reference in their entirety.

The publications of organic functional material presented above are incorporated herein by reference for the purpose of disclosure.

In some embodiments, the organic compound H comprises at least one alcohol-soluble or water-soluble group as described above; preferably comprises at least two alcohol-soluble or water-soluble groups; and most preferably comprises at least three alcohol-soluble or water-soluble groups.

In some embodiments, the organic compound H comprises at least one cross-linkable group as described above; preferably comprises at least two cross-linkable groups; and most preferably comprises at least three cross-linkable groups.

Examples of some suitable organic compounds H, are listed below (but not limited to), which may be further substituted arbitrarily:

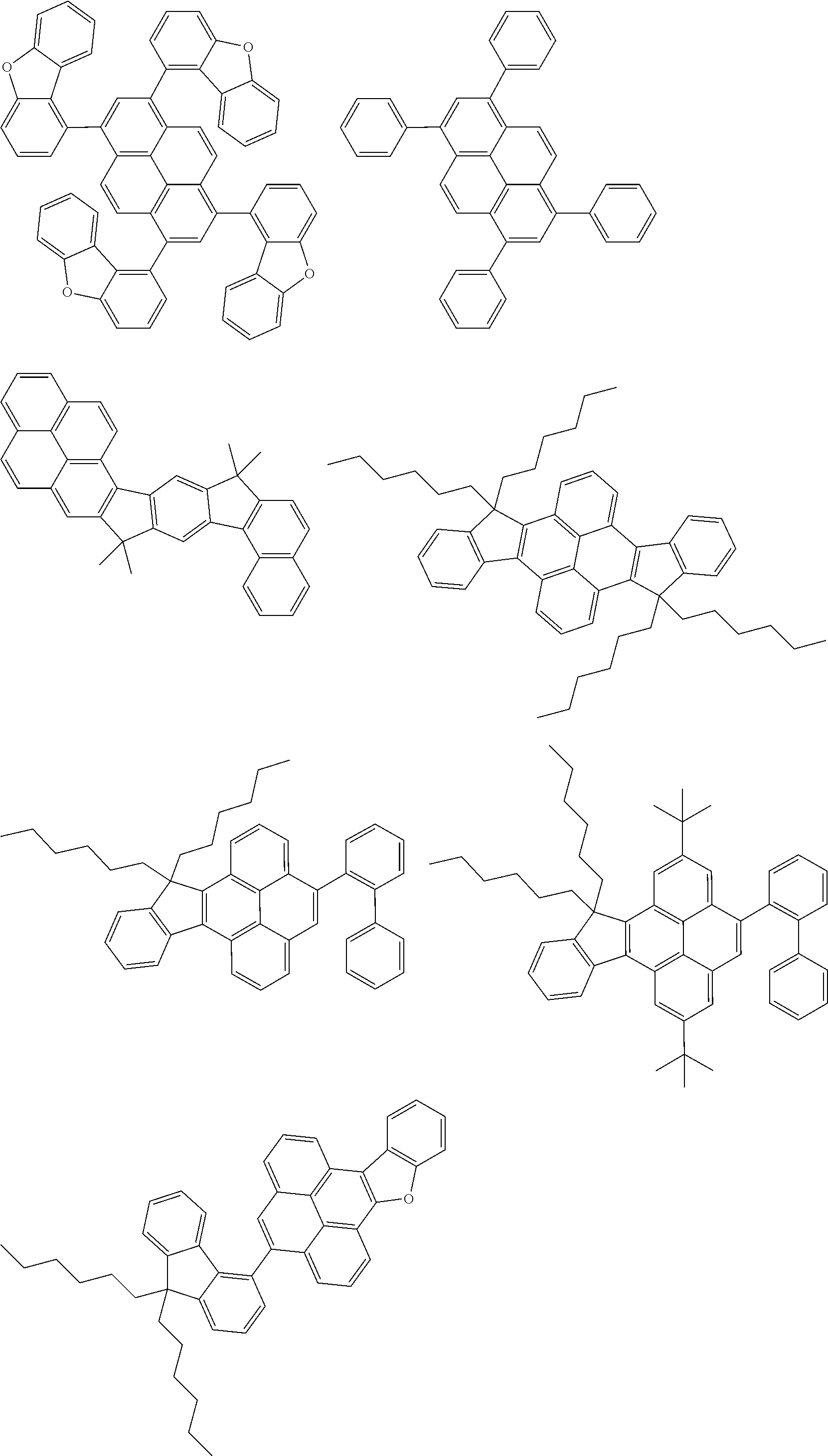

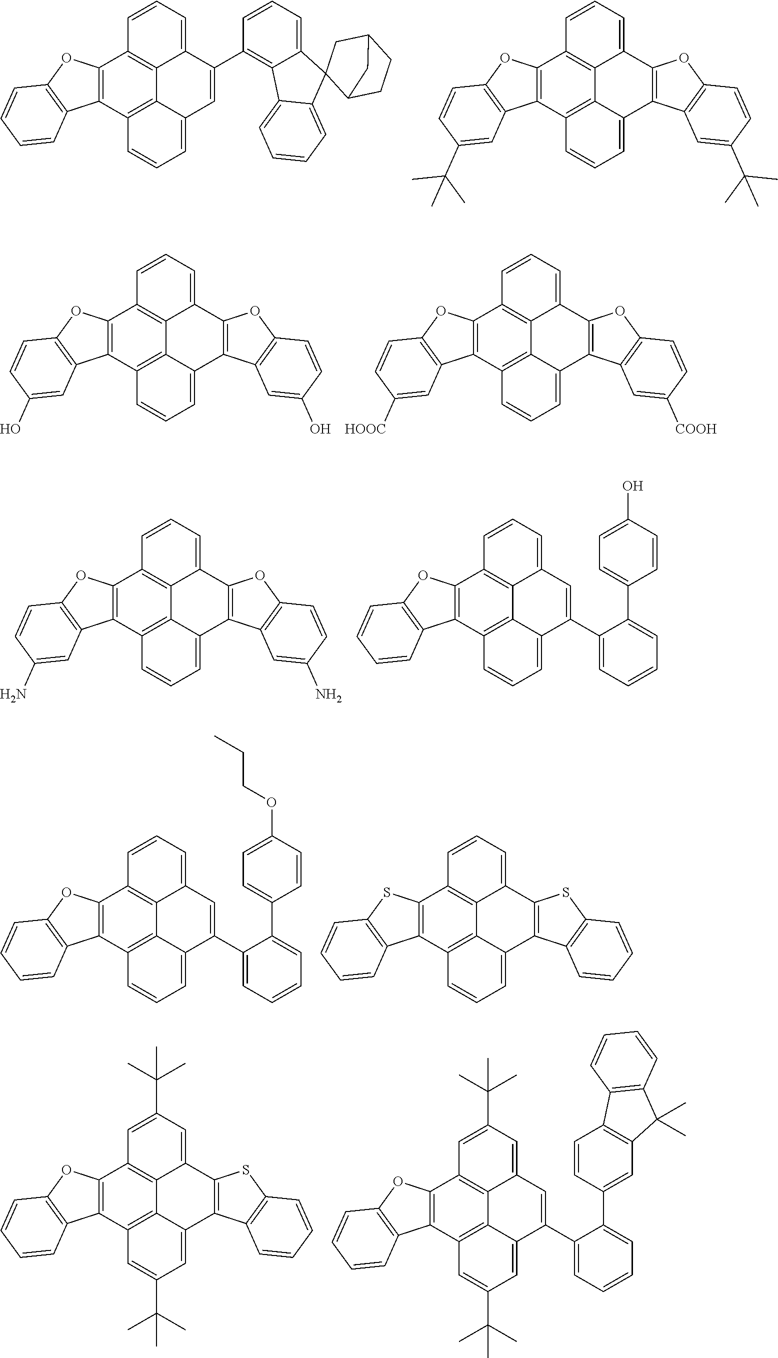

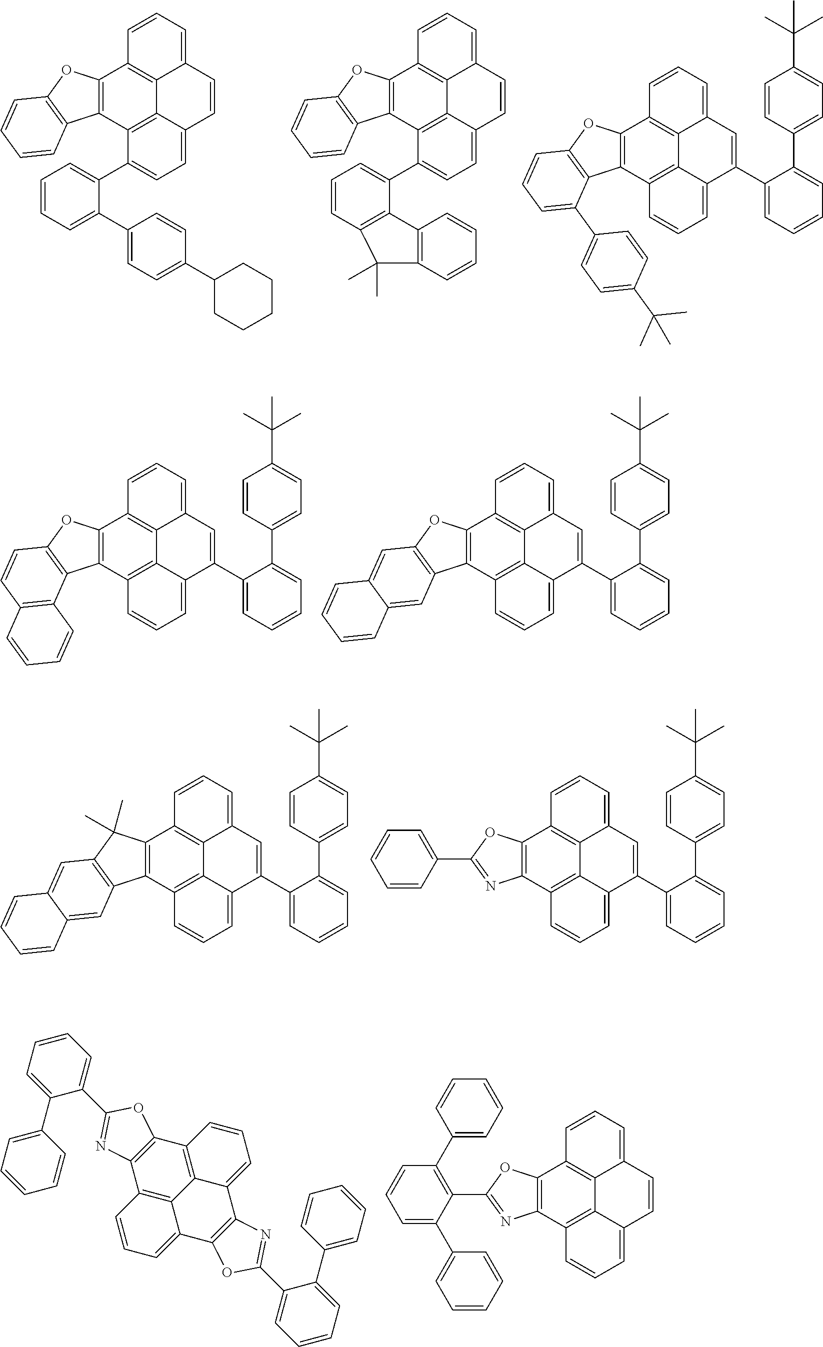

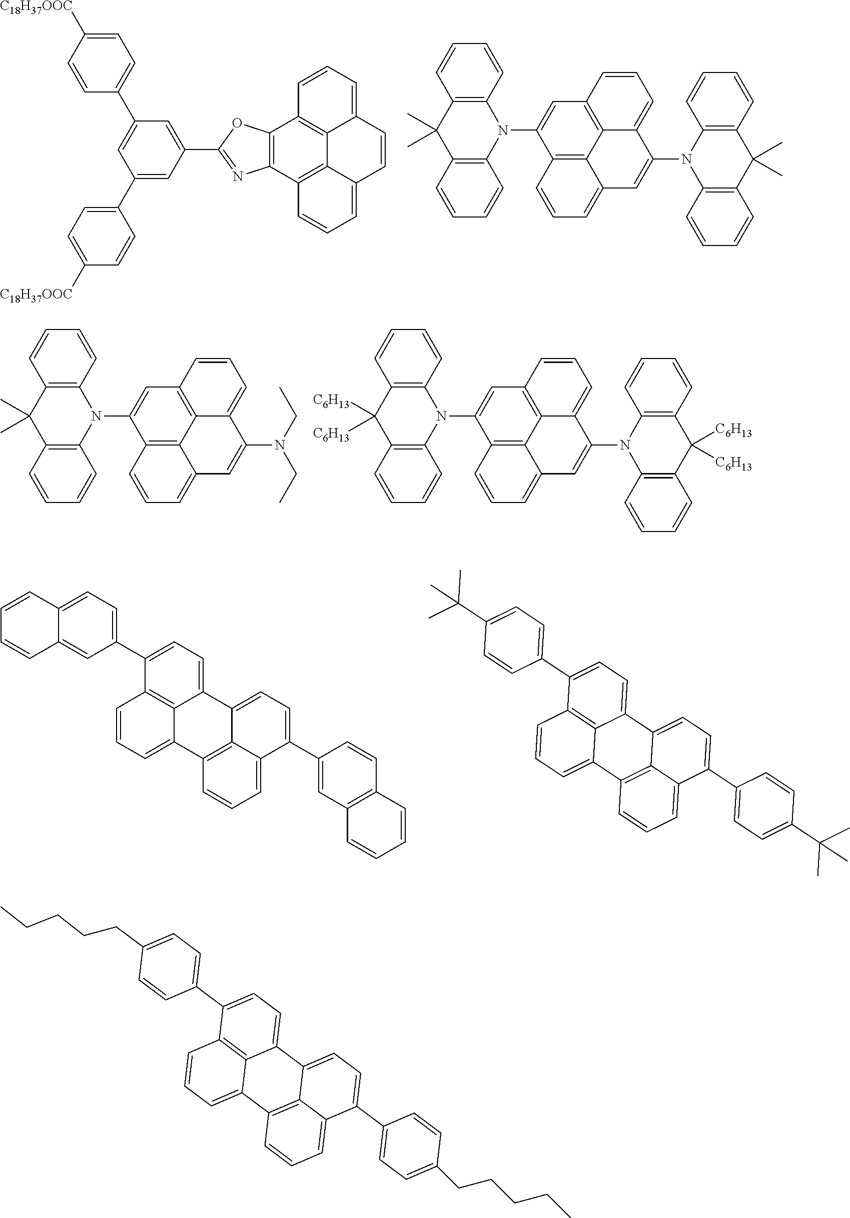

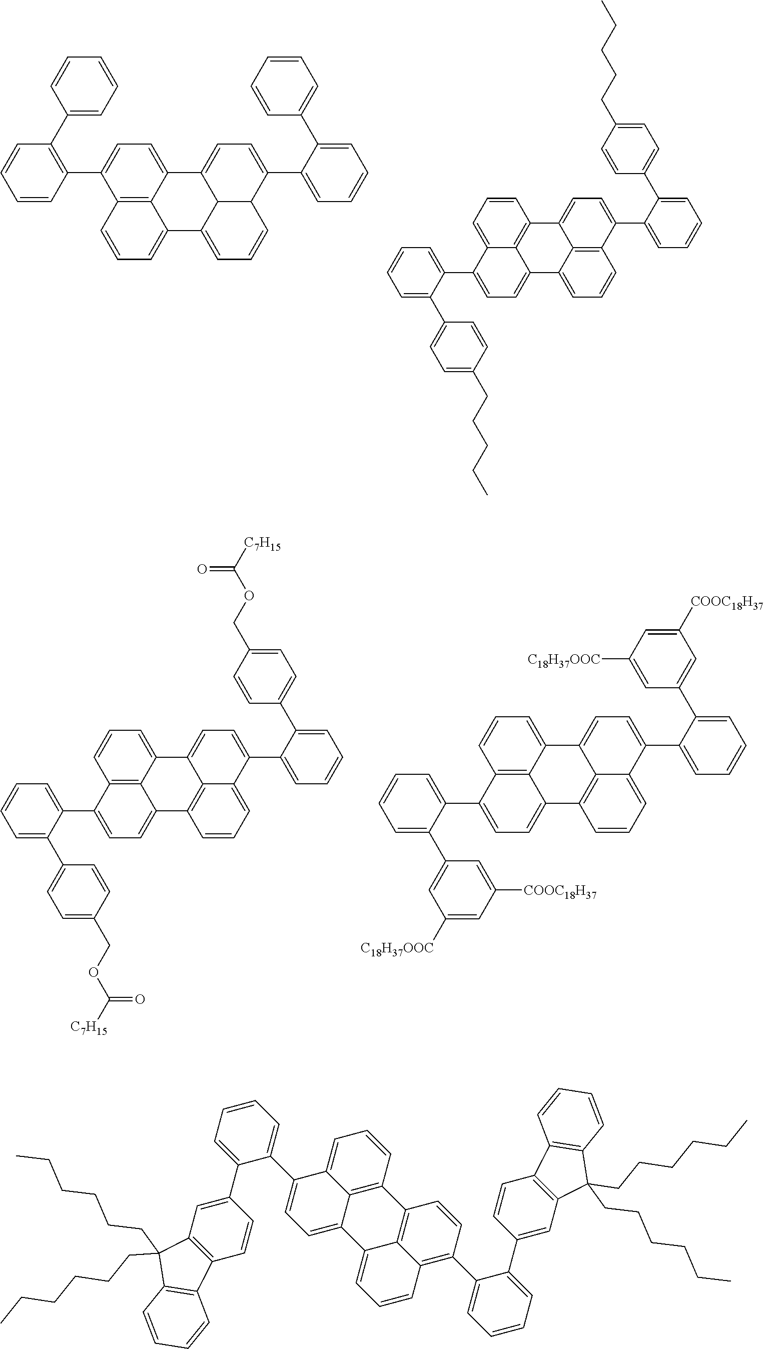

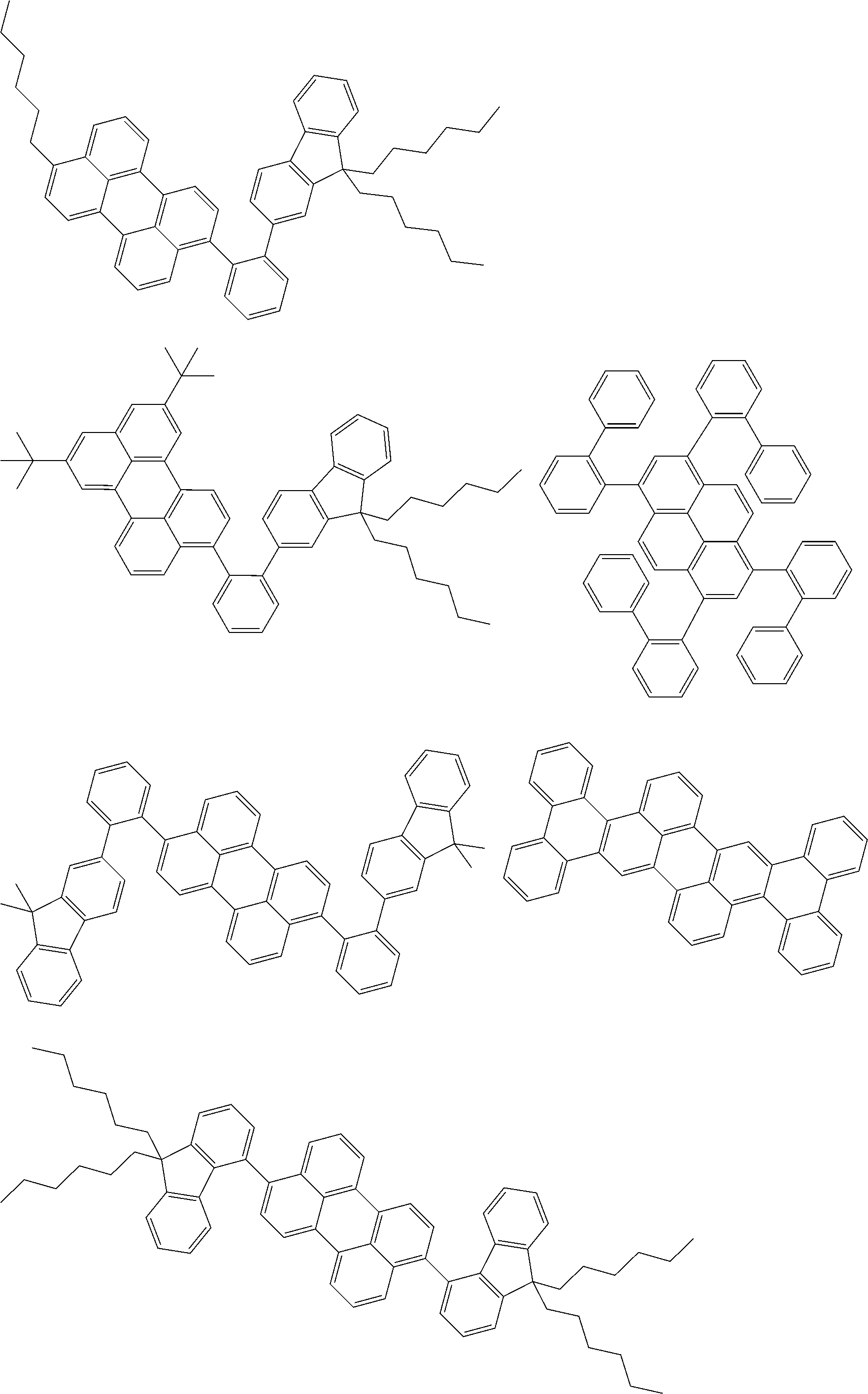

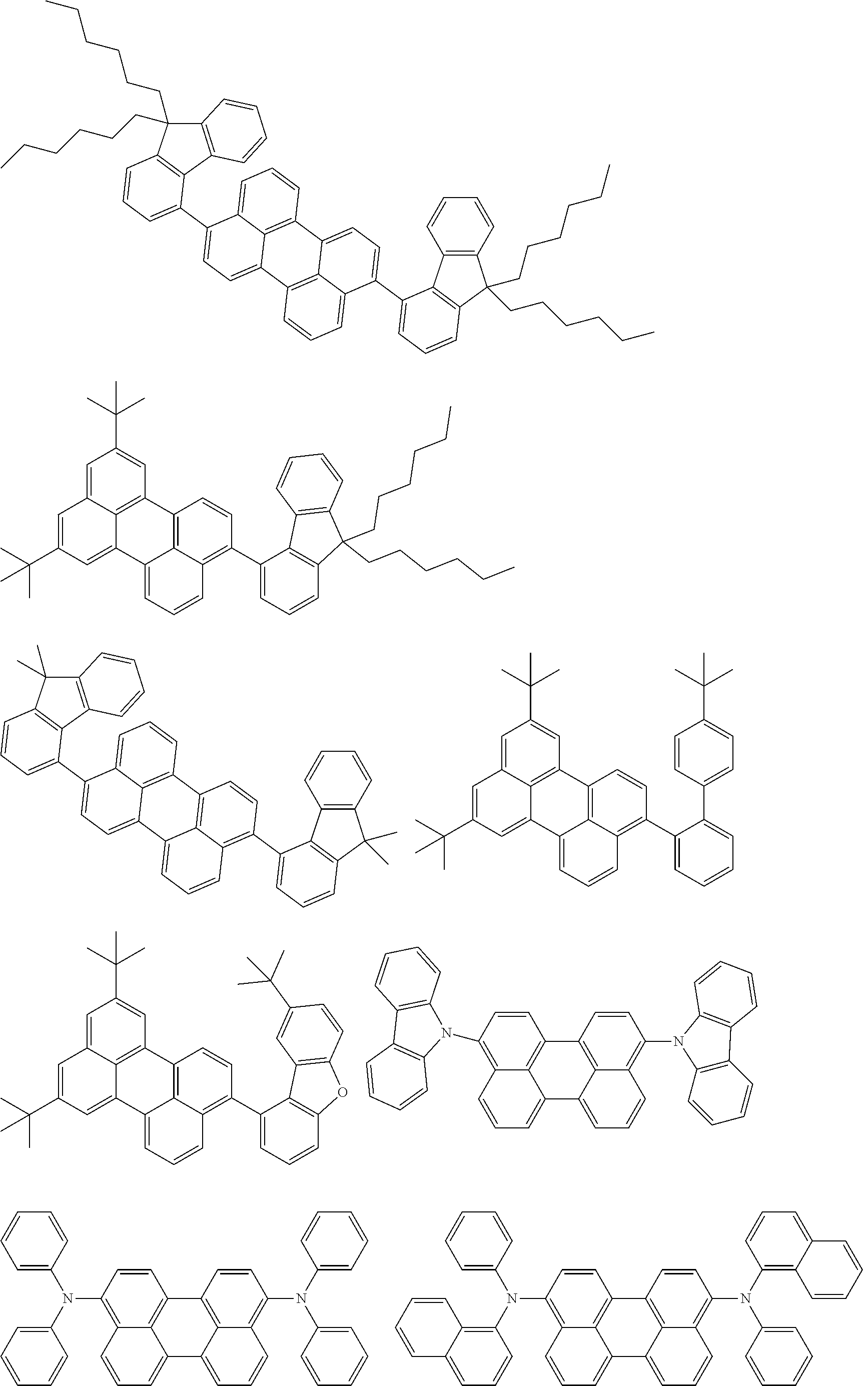

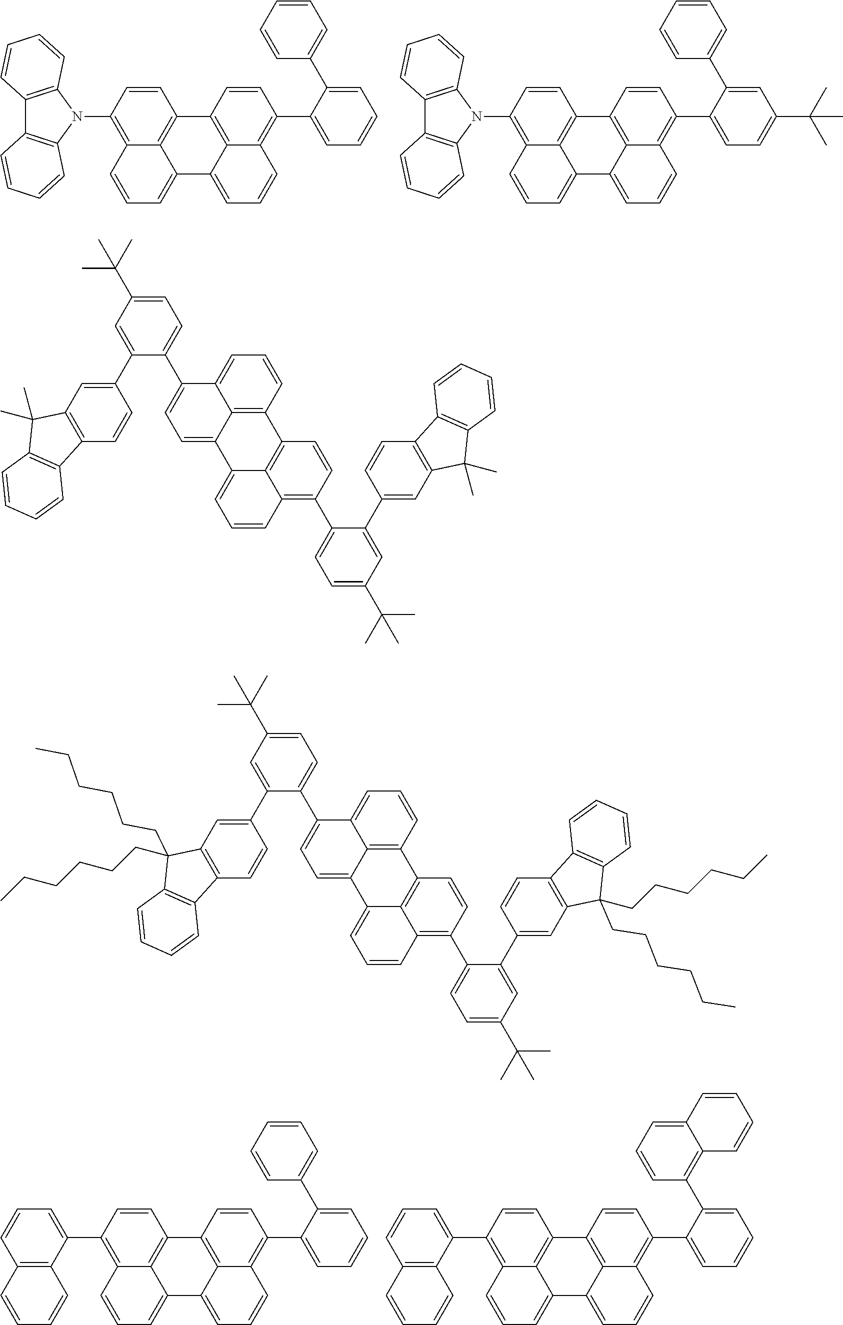

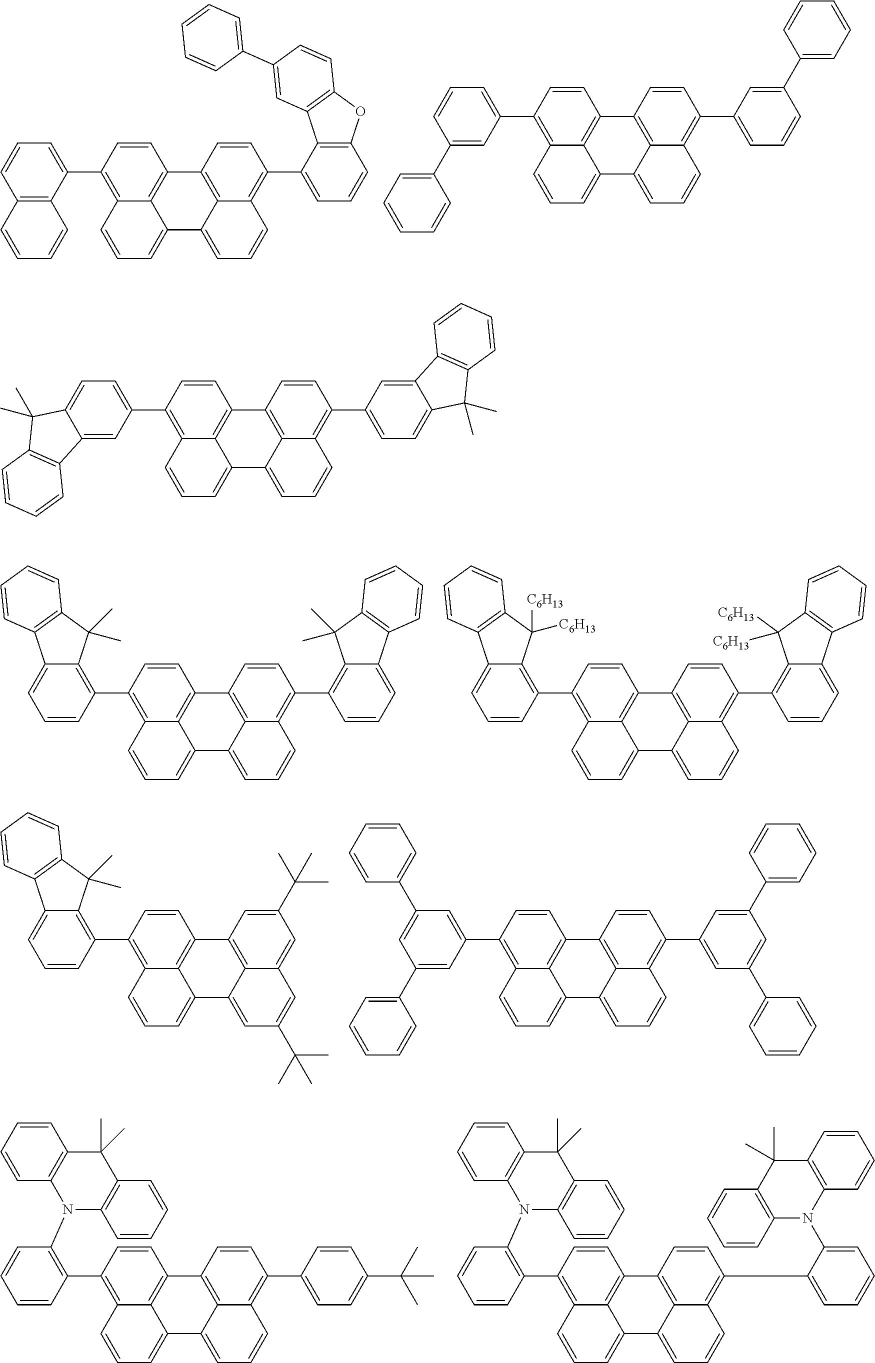

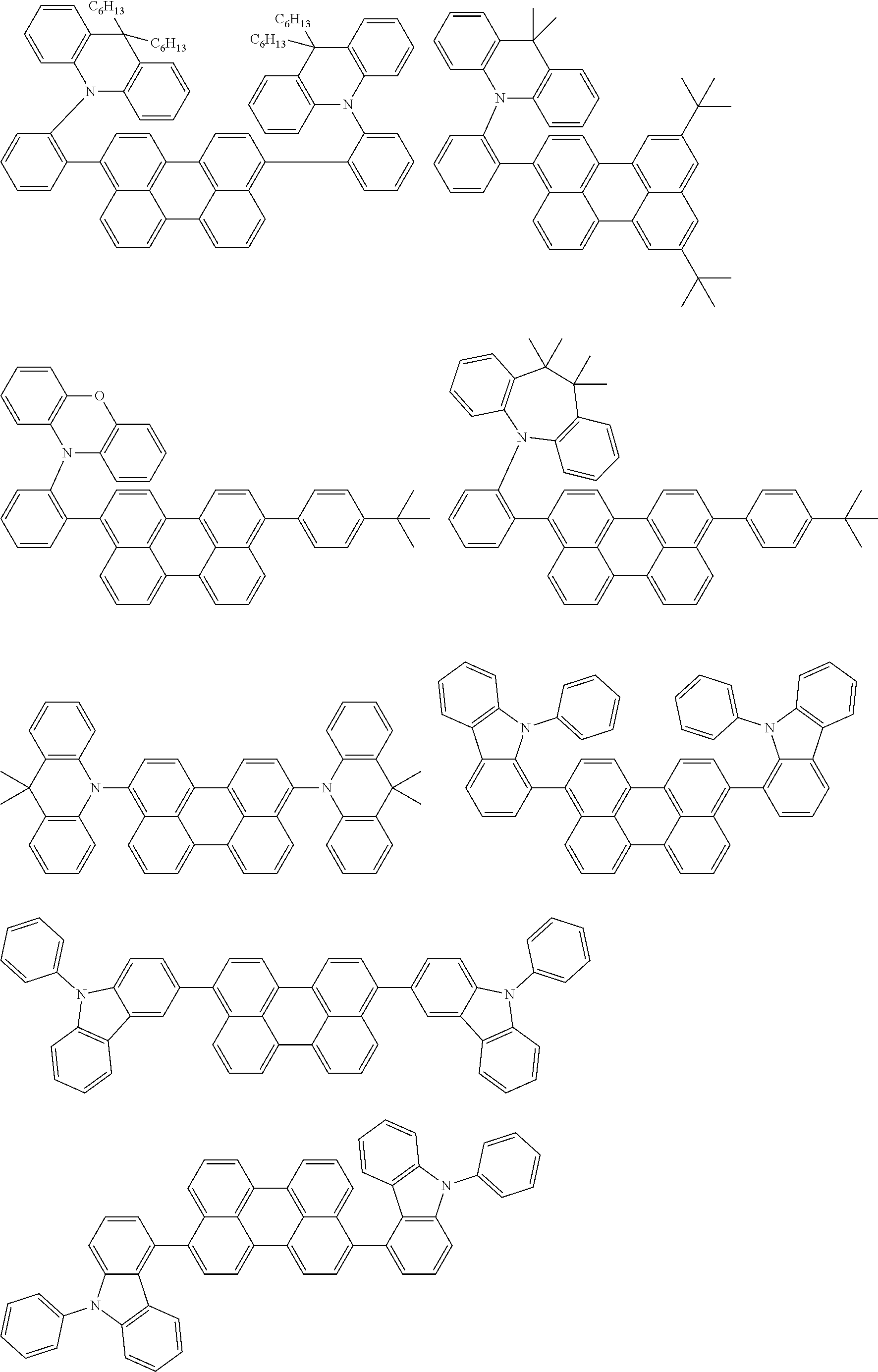

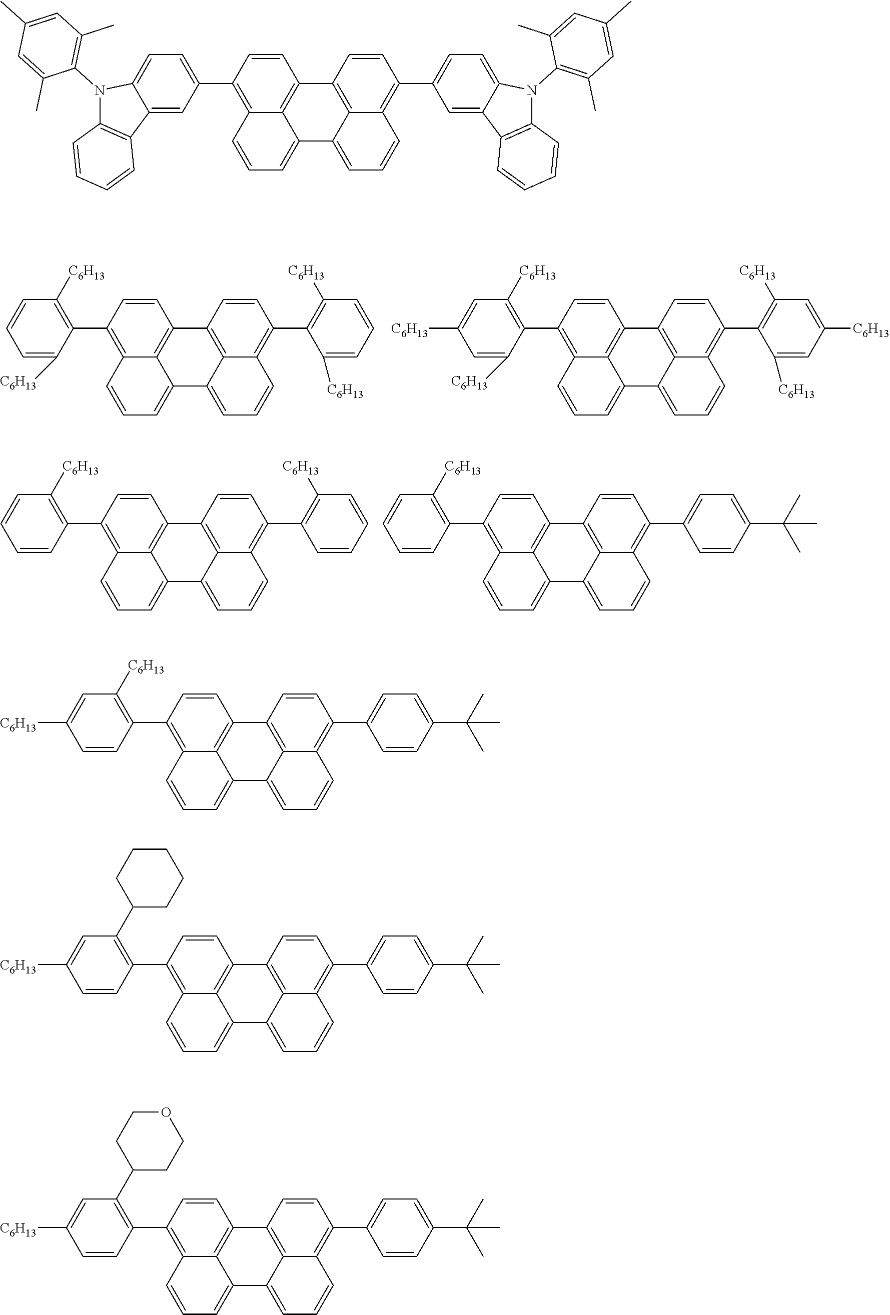

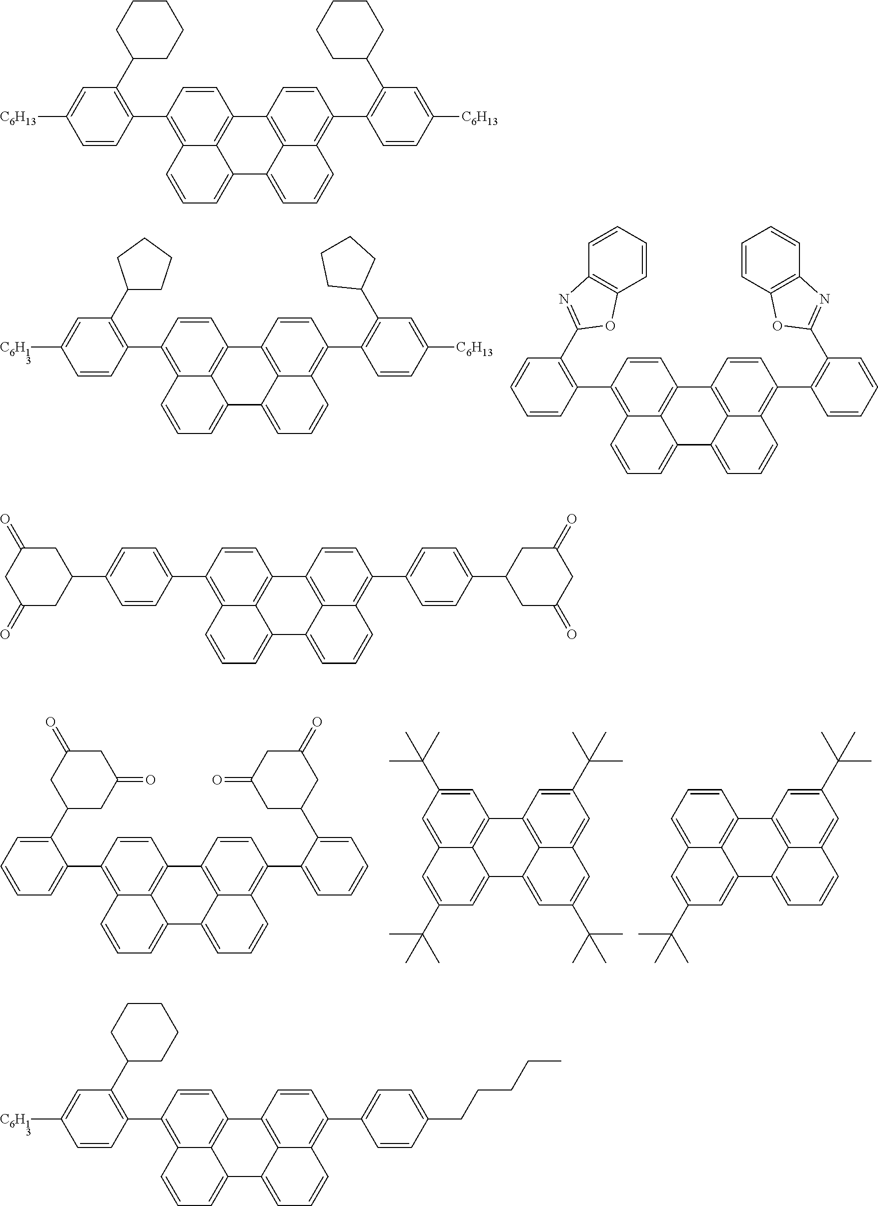

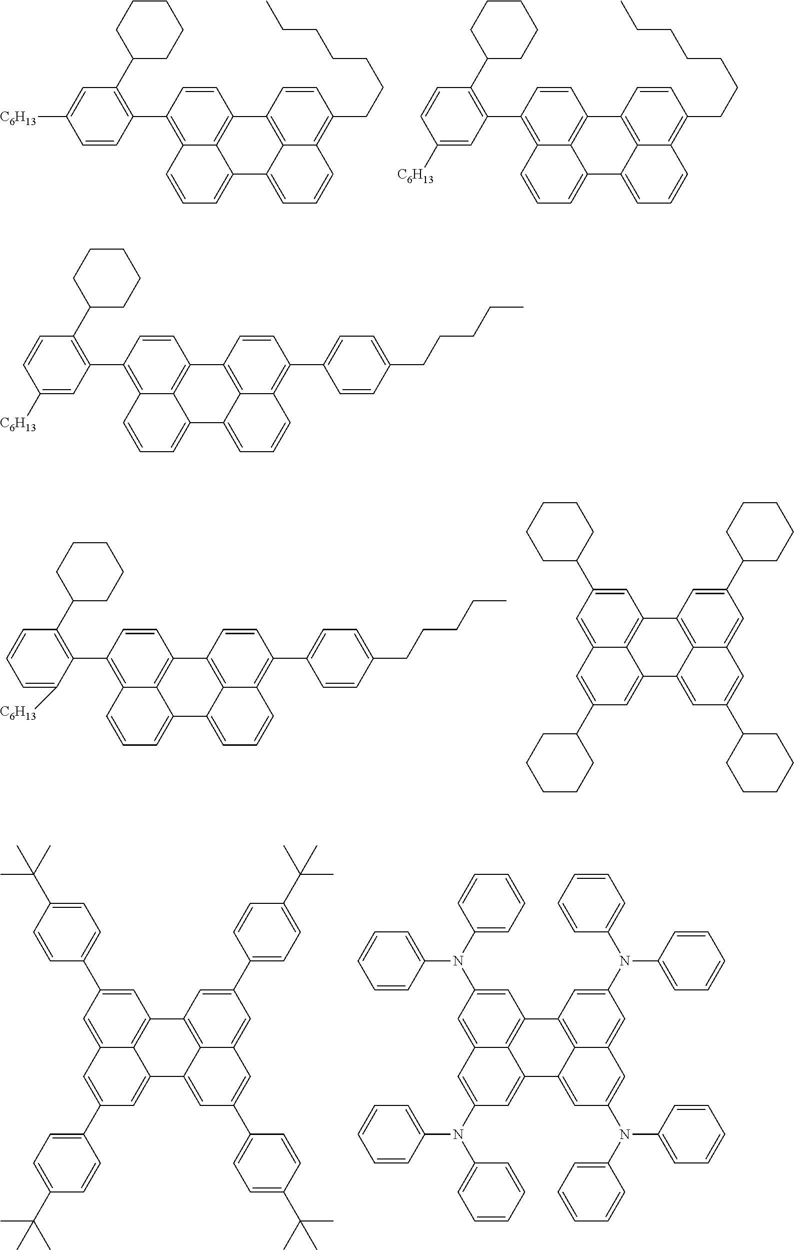

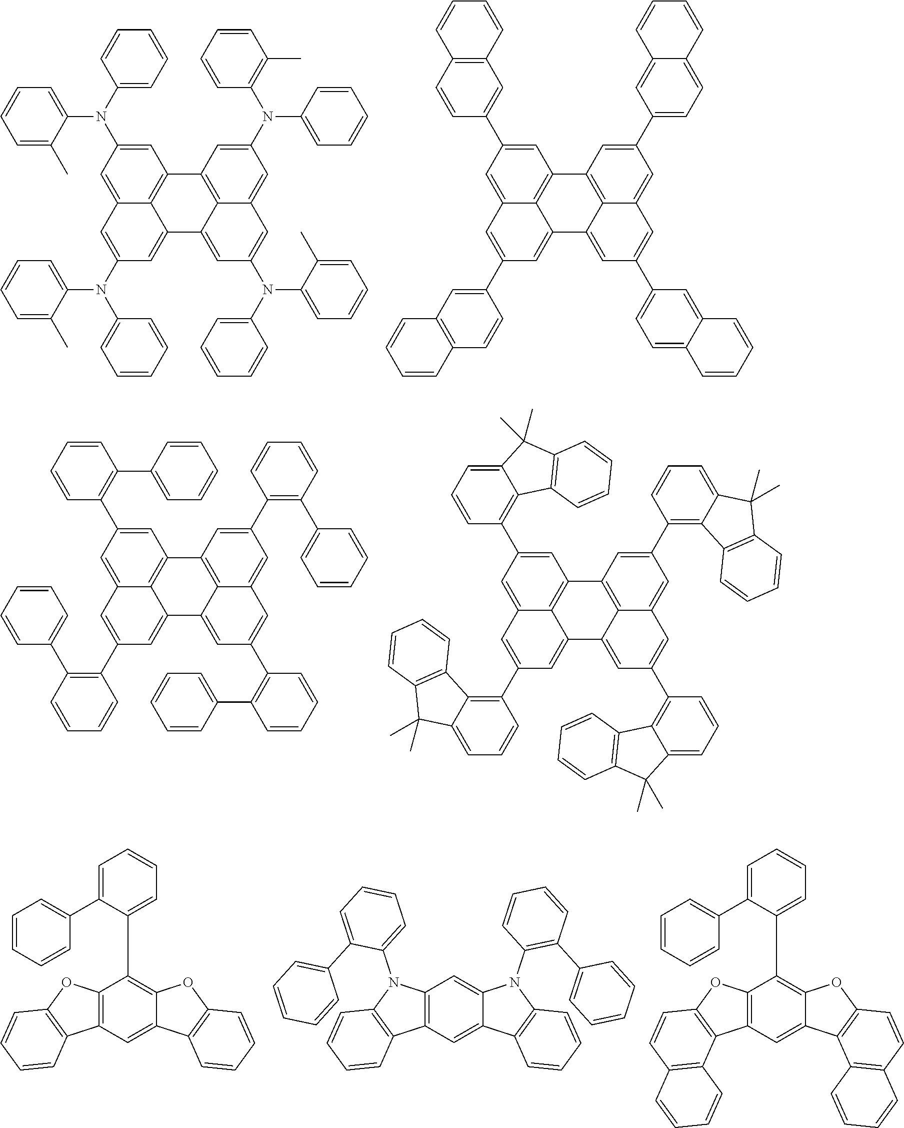

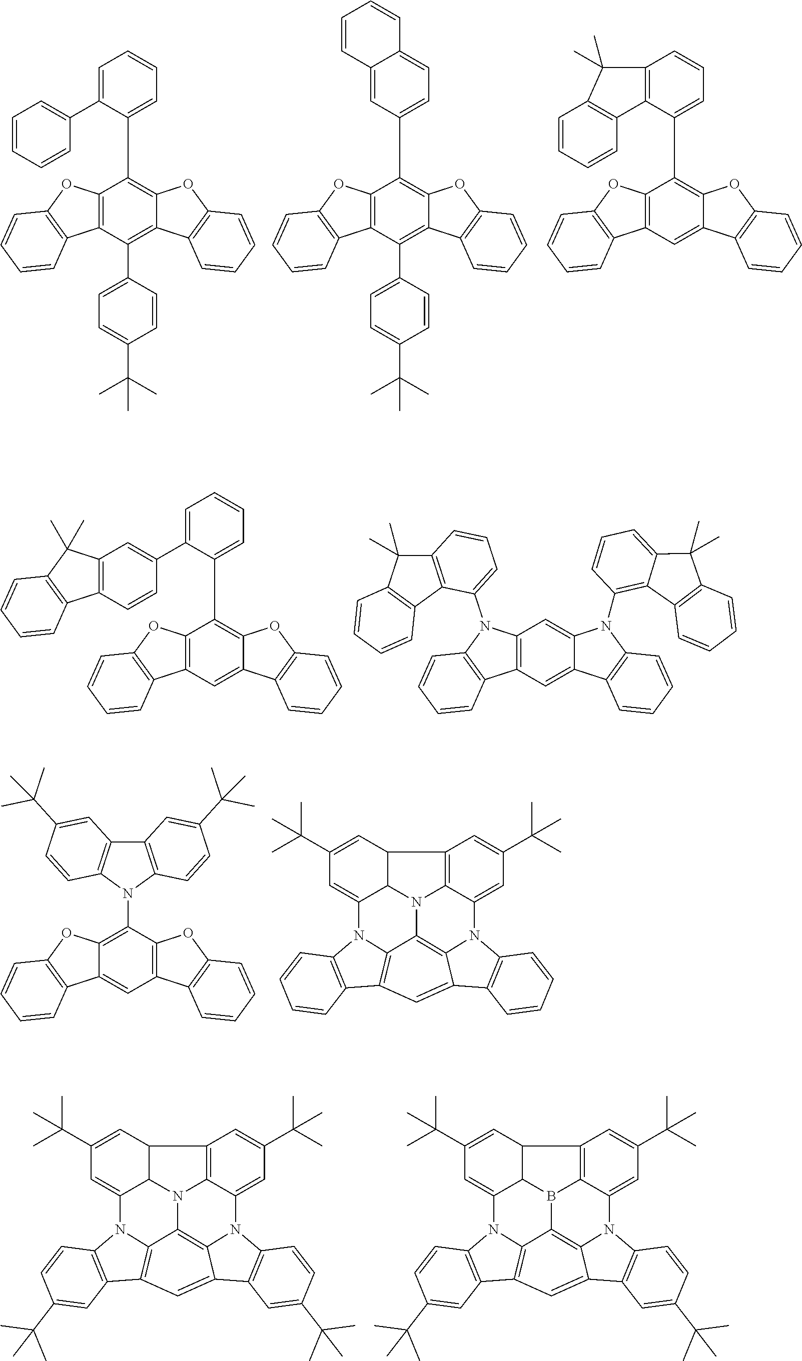

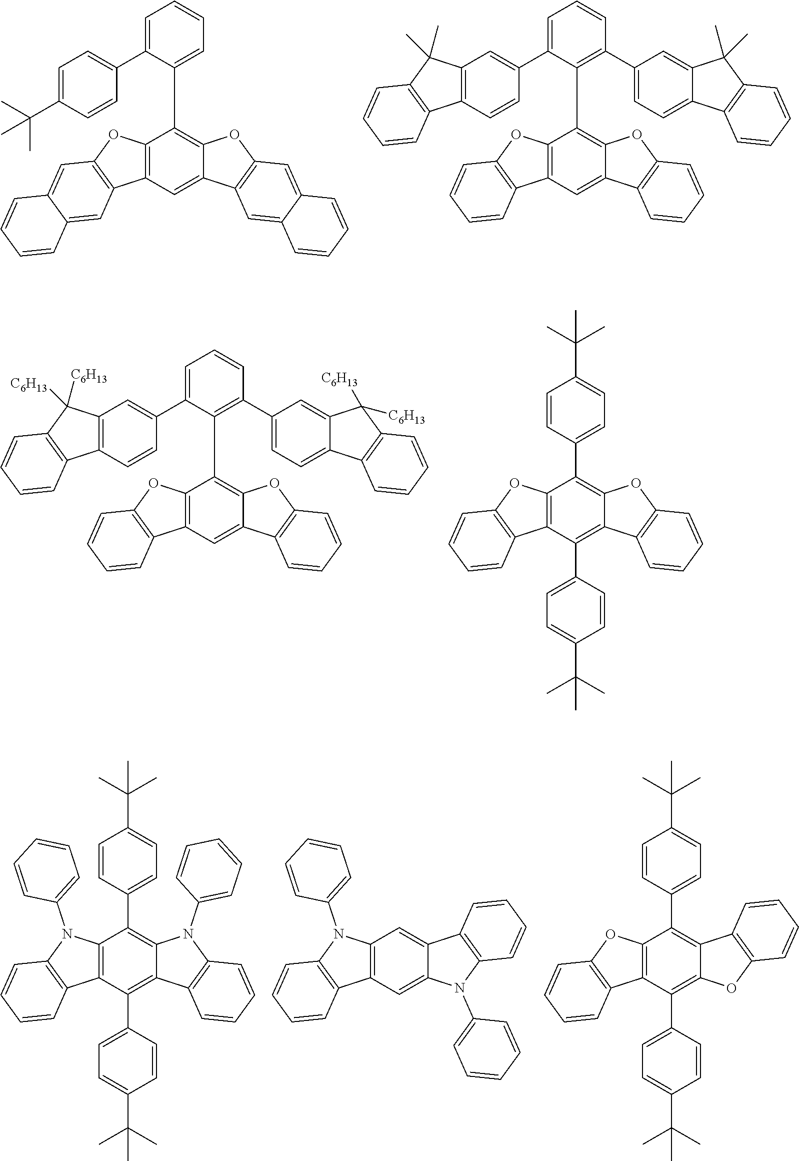

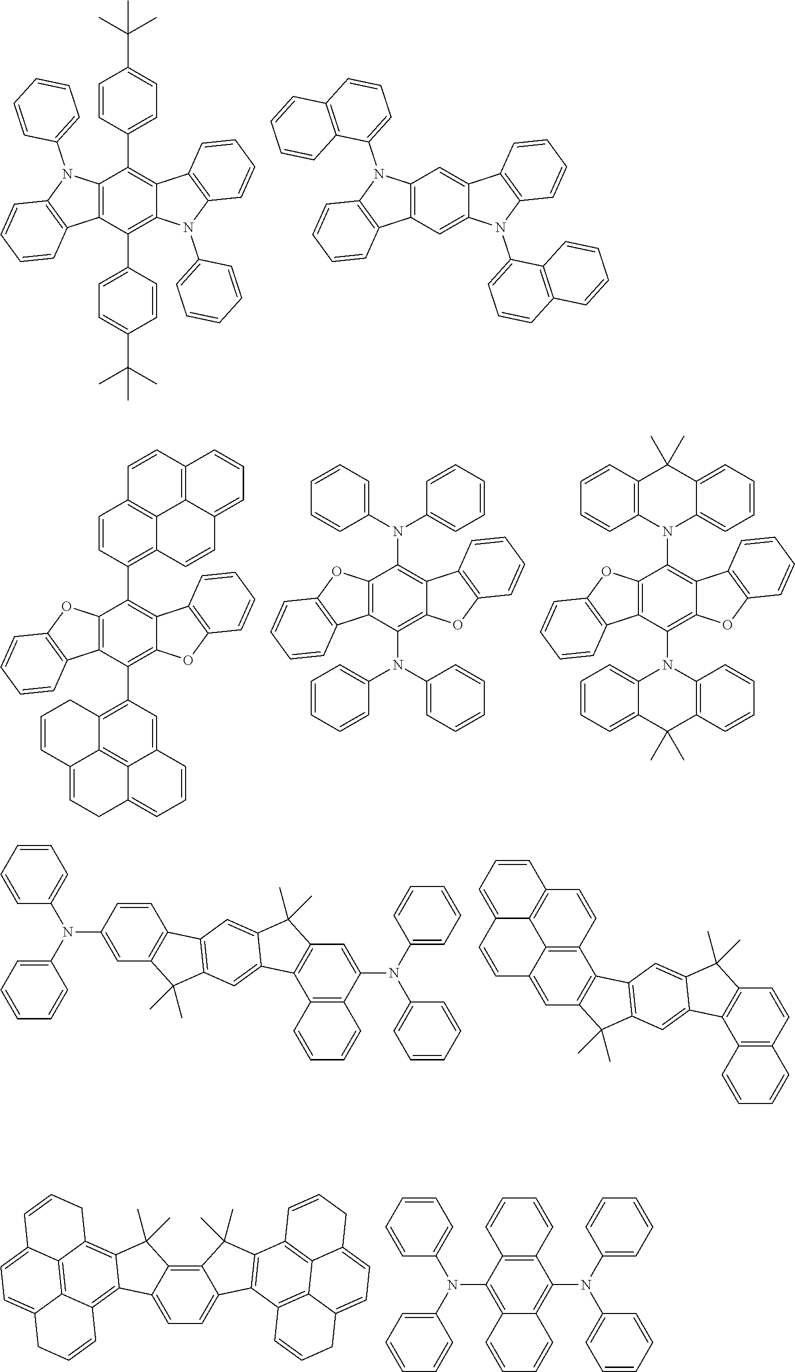

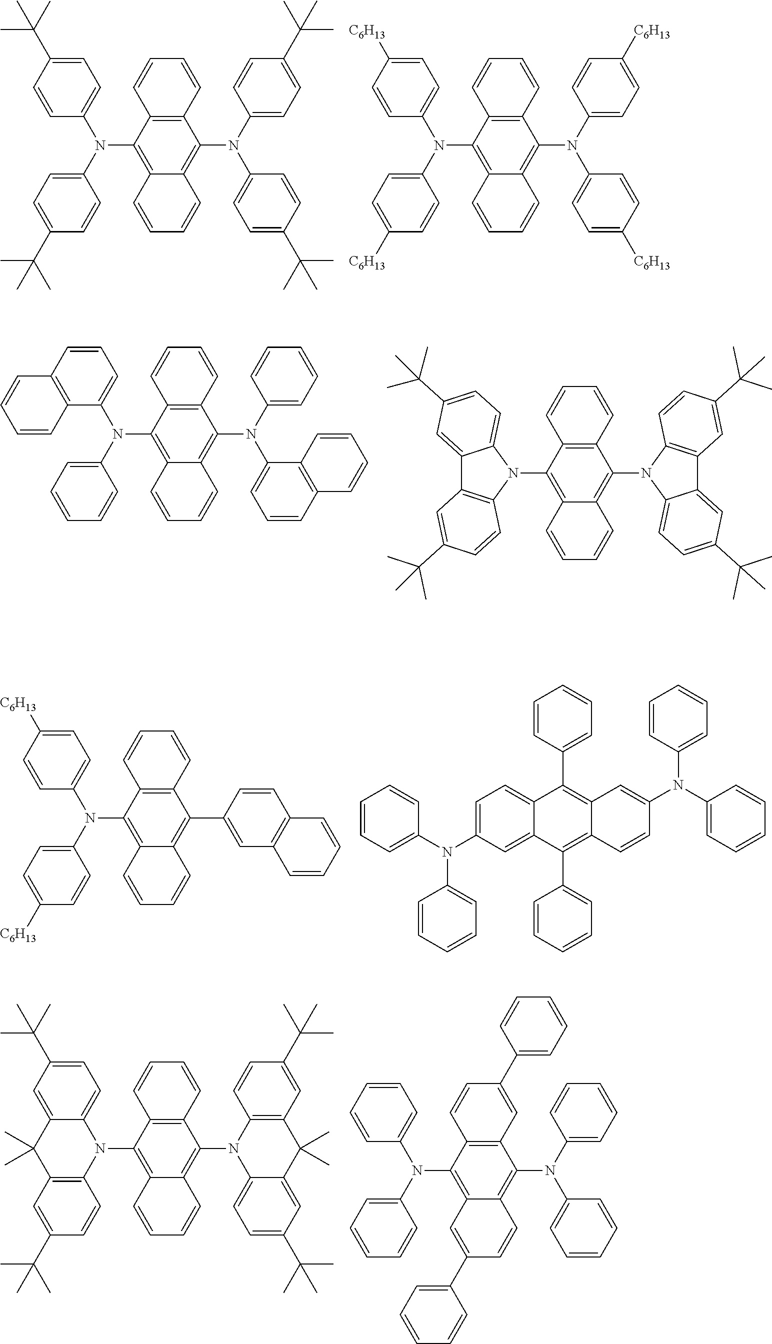

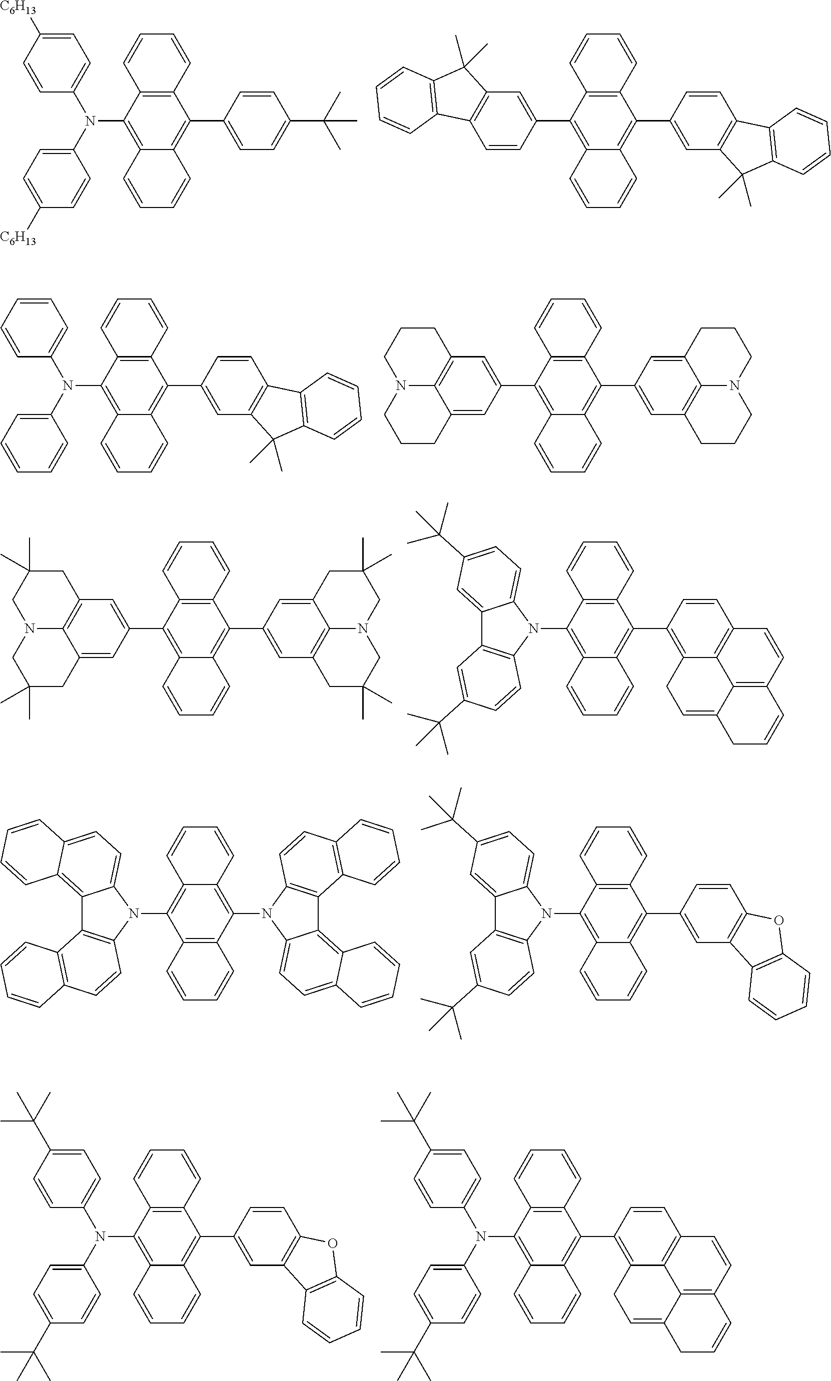

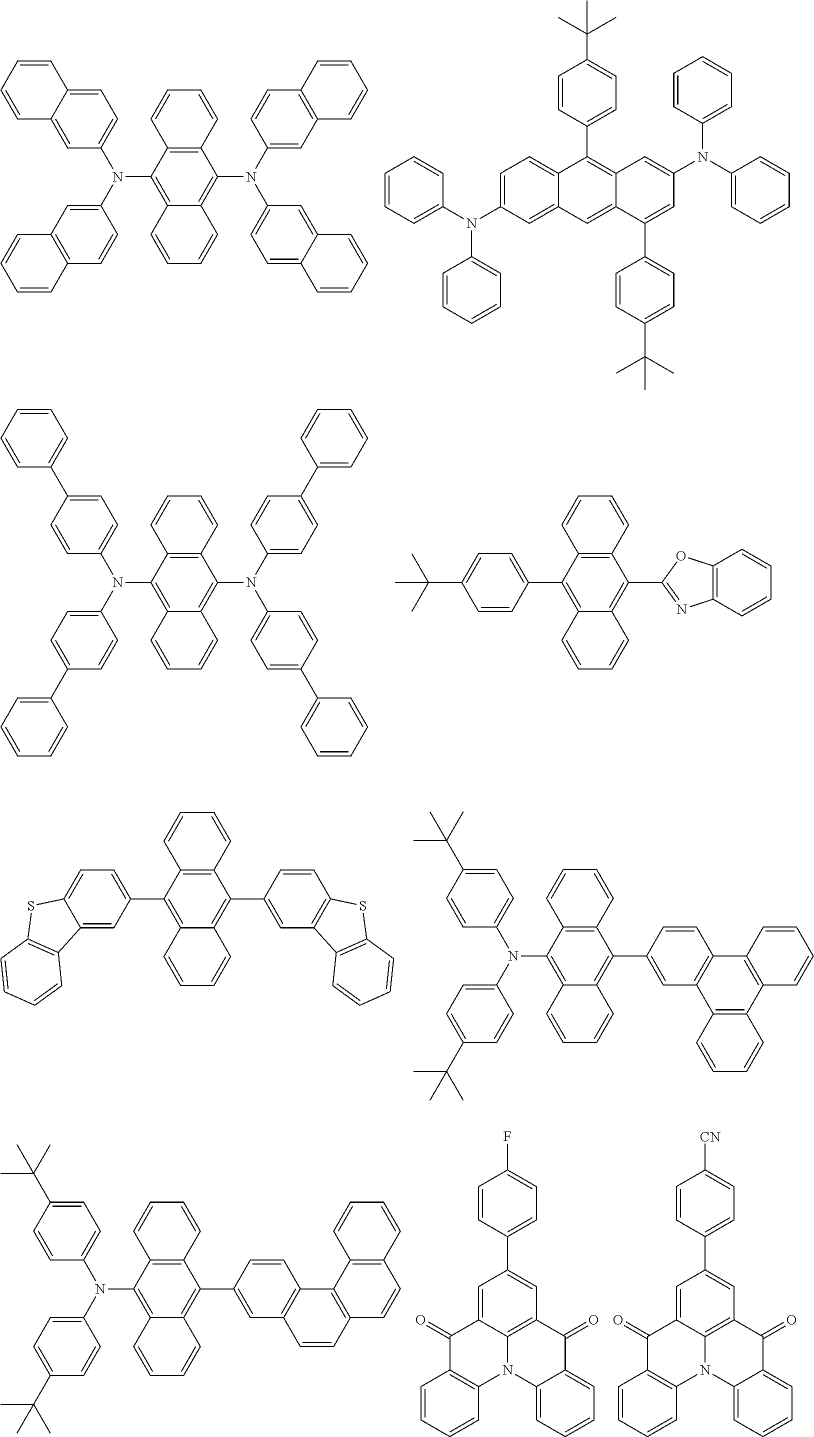

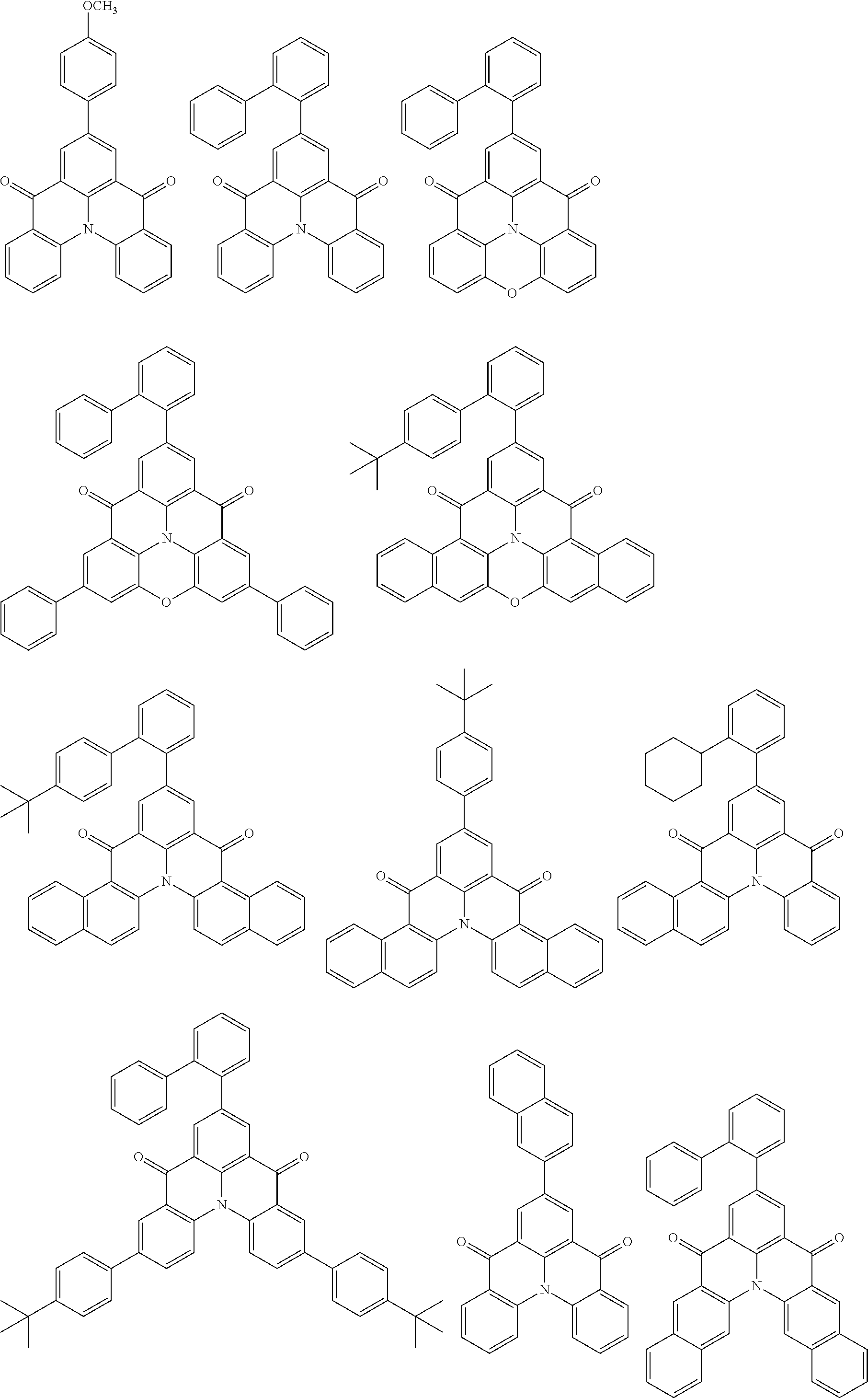

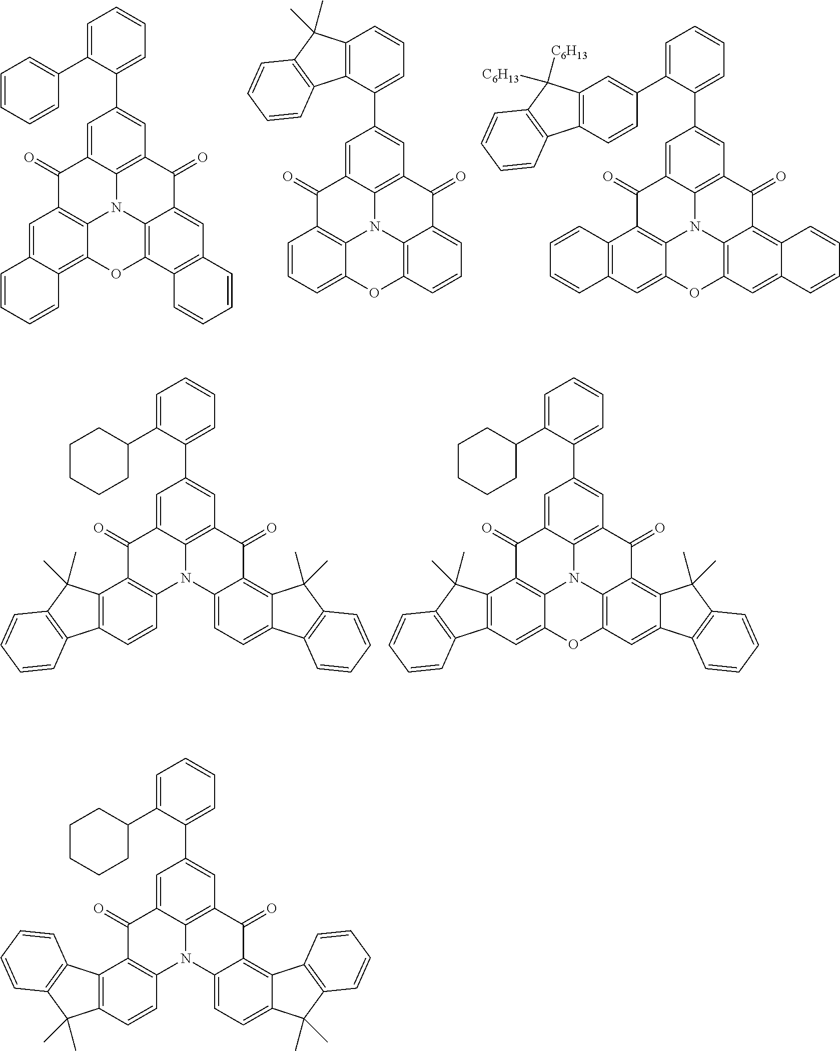

In the formulations as described herein, the absorption spectrum of the emitter E and the emission spectrum of the organic compound H have a large overlap, so that the efficient energy transfer (i.e., Förster resonance energy transfer (FRET)) can be realized therebetween.

In some embodiments, the emission spectrum of the formulation is derived exclusively from the emitter E, i.e. complete energy transfer is realized between the emitter E and the organic compound H.

In some embodiments, the formulation comprises more than two organic compounds H.

In some embodiments, the organic compound H is selected from one of formulas (1)-(1e) or (2)-(2e).

In some embodiments, in the formulation as described herein, the weight ratio of the organic compound H and the emitter E ranges from 50:50 to 99:1, preferably from 60:40 to 98:2, more preferably from 70:30 to 97:3, and most preferably from 80:20 to 95:5.

In some embodiments, the formulation as described herein comprises an organic resin. For the purposes of the present disclosure, the organic resin refers to a resin prepolymer or a resin formed after the resin prepolymer is crosslinked or cured.

In some embodiments, the formulation comprises two or more organic resins.

The organic resins suitable for the present disclosure include, but not limited to: polystyrene, polyacrylate, polymethacrylate, polycarbonate, polyurethane, polyvinylpyrrolidone, polyvinyl acetate, polyvinyl chloride, polybutylene, polyethylene glycol, polysiloxane, polyacrylate, epoxy resin, polyvinyl alcohol, polyacrylonitrile, polyvinylidene chloride (PVDC), polystyrene-acrylonitrile (SAN), polybutylene terephthalate (PBT), polyethylene terephthalate (PET), polyvinyl butyrate (PVB), polyvinyl chloride (PVC), polyamide, polyoxymethylene, polyimide, polyetherimide, and mixtures thereof.

Further, the organic resins suitable for the present disclosure include, but not limited to, those formed by homopolymerization or copolymerization from the following monomers (resin prepolymers): styrene derivatives, acrylate derivatives, acrylonitrile derivatives, acrylamide derivatives, vinyl ester derivatives, vinyl ether derivatives, maleimide derivatives, conjugated diene derivatives.

Examples of styrene derivatives include, but not limited to alkylstyrenes, such as α-methylstyrene, o-, m-, p-methylstyrene, p-butylstyrene; especially 4-tert-butylstyrene, alkoxystyrene, such as p-methoxystyrene, p-butoxystyrene, p-tert-butoxystyrene.

Examples of acrylate derivatives include, but not limited to methyl acrylate, methyl methacrylate, ethyl acrylate, ethyl methacrylate, n-propyl acrylate, n-propyl methacrylate, isopropyl acrylate, isopropyl methacrylate, n-butyl acrylate, n-butyl methacrylate, isobutyl acrylate, isobutyl methacrylate, sec-butyl acrylate, sec-butyl methacrylate, tert-butyl acrylate, tert-butyl methacrylate, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, 2-hydroxybutyl acrylate, 2-hydroxybutyl methacrylate, 3-hydroxybutyl acrylate, 3-hydroxybutyl methacrylate, 4-hydroxybutyl acrylate, 4-hydroxybutyl methacrylate, allyl acrylate, allyl methacrylate, benzyl acrylate, benzyl methacrylate, cyclohexyl acrylate, cyclohexyl methacrylate, phenyl acrylate, phenyl methacrylate, 2-methoxyethyl acrylate, 2-methoxyethyl methacrylate, 2-phenoxyethyl acrylate, 2-phenoxyethyl methacrylate, methoxydiethylene glycol acrylate, methoxydiethylene glycol methacrylate, methoxytriethylene glycol acrylate, methoxytriethylene glycol methacrylate, methoxypropylene glycol acrylate, methoxypropylene glycol methacrylate, methoxy dipropylene glycol acrylate, methoxydipropylene glycol methacrylate, isobornyl acrylate, isobornyl methacrylate, dicyclopentadiene acrylate, dicyclopentadiene methacrylate, adamantane (meth) acrylate, norbornene (meth) acrylate, 2-hydroxy-3-phenoxypropyl acrylate, 2-hydroxy-3-phenoxypropyl methacrylate, glyceryl monoacrylate, and glyceryl monostearate; 2-aminoethyl acrylate, 2-aminoethyl methacrylate, 2-dimethylaminoethyl acrylate, 2-dimethylaminoethyl methacrylate, N,N-dimethylaminoethyl (meth) acrylic acid, N,N-diethylaminoethyl (meth) acrylate, 2-dimethylaminopropyl methacrylate, 3-aminopropyl acrylate, 3-aminopropyl methacrylate, N,N-dimethyl-1,3-propane diamine (meth) acrylate, 3-dimethylaminopropyl acrylate, 3-dimethylaminopropyl methacrylate, glycidyl acrylate, and glycidyl methacrylate.

Examples of the acrylonitrile derivatives include, but not limited to acrylonitrile, methacrylonitrile, α-chloroacrylonitrile, and vinylidene cyanide.

Examples of acrylamide derivatives include, but are not limited to acrylamide, methacrylamide, α-chloroacrylamide, N-2-hydroxyethyl acrylamide, and N-2-hydroxyethyl methacrylamide.

Examples of vinyl ester derivatives include, but not limited to vinyl acetate, vinyl propionate, vinyl butyrate, and vinyl benzoate.

Examples of vinyl ether derivatives include, but not limited to vinyl methyl ether, vinyl ethyl ether, and allyl glycidyl ether.

Examples of maleimide derivatives include, but not limited to maleimide, benzylmaleimide, N-phenylmaleimide, and N-cyclohexylmaleimide.

Examples of conjugated diene derivatives include, but not limited to 1,3-butadiene, isoprene, and chloroprene.

The homopolymers or copolymers can be prepared by free-radical polymerization, cationic polymerization, anionic polymerization, or organometallic catalysis polymerization (for example Ziegler-Natta catalysis). The polymerization process may be suspension polymerization, emulsion polymerization, solution polymerization, or bulk polymerization.

The number average molecualr weight Mn (as determined by GPC) of the organic resins is generally in the range of 10 000 g/mol to 1 000 000 g/mol, preferably in the range of 20 000 g/mol to 750 000 g/mol, more preferably in the range of 30 000 g/mol to 500 000 g/mol.

In some embodiments, the organic resin is a thermosetting resin or an UV curable resin. In some embodiments, the organic resin is cured by a method that will enable roll-to-roll processing.

Thermosetting resins require curing in which they undergo an irreversible process of molecular cross-linking, which makes the resin non-fusible. In some embodiments, the thermosetting resin is an epoxy resin, a phenolic resin, a vinyl resin, a melamine resin, a urea-formaldehyde resin, an unsaturated polyester resin, a polyurethane resin, an allyl resin, an acrylic resin, a polyamide resin, a polyamide-imide resin, a phenol-amide polycondensation resin, an urea-melamine polycondensation resin, or combinations thereof.

In some embodiments, the thermosetting resin is an epoxy resin. The epoxy resins are easy to cure and do not give off volatiles or generate by-products from a wide range of chemicals. The epoxy resins can also be compatible with most substrates and tend to readily wet surfaces. See also Boyle, M. A. et al., “Epoxy Resins”, Composites, Vol.21, ASM Handbook, pages 78-89 (2001).

In some embodiments, the organic resin is a silicone thermosetting resin. In some embodiments, the silicone thermosetting resin is OE6630A or OE6630B (Dow Corning Corporation (Auburn, Michigan.)).

In some embodiments, a thermal initiator is used. In some embodiments, the thermal initiator is AIBN[2,2′-azobis(2-methylpropionitrile)] or benzoyl peroxide.

The UV curable resin is a polymer that will cure and rapidly harden upon exposure to light of a specific wavelength. In some embodiments, the UV curable resin is a resin having a free radical polymerization group, and a cationic polymerizable group as functional groups; the radical polymerizable group is such as (meth)acryloyloxy group, vinyloxy group, styryl group, or vinyl group. The cationically polymerizable group is such as epoxy group, thioepoxy group, vinyloxy group, or oxetanyl group. In some embodiments, the UV curable resin is a polyester resin, a polyether resin, a (meth)acrylic resin, an epoxy resin, a polyurethane resin, an alkyd resin, a spiroacetal resin, a polybutadiene resin, or a thiolene resin.

In some embodiments, the UV curable resin is selected from polyurethane acrylate, allyloxy diacrylate, bis(acryloyloxyethyl) hydroxyisocyanurate, bis(acryloyloxyneopentyl glycol) adipate, bisphenol A diacrylate, bisphenol A dimethacrylate, 1,4-butanediol diacrylate, 1,4-butanediol dimethacrylate, 1,3-butanediol diacrylate, 1,3-butanediol dimethacrylate, dicyclopentyl diacrylate, diethylene glycol diacrylate, diethylene glycol dimethacrylate, dipentaerythritol hexaacrylate, dipentaerythritol monohydroxy pentacrylate, bis(trimethylolpropane) tetraacrylate, triethylene glycol dimethacrylate, glyceryl methacrylate, 1,6-hexanediol diacrylate, neopentyl glycol dimethacrylate, neopentyl glycol hydroxypivalonate, pentaerythritol triacrylate, pentaerythritol tetraacrylate, dimethacrylate, polyethylene glycol diacrylate, polypropylene glycol diacrylate, tetraethylene glycol diacrylate, tetrabromobisphenol A diacrylate, triethylene glycol divinyl ether, glycerol diacrylate, trimethylolpropane triacrylate, tripropylene glycol diacrylate, tris(acryloyloxyethyl) isocyanurate, triacrylate, diacrylate, propyl acrylate, vinyl-terminated polydimethylsiloxane, vinyl-terminated diphenyl siloxane-dimethyl siloxane copolymer, vinyl-terminated polyphenyl methyl siloxane, vinyl-terminated difluoromethyl siloxane-dimethyl siloxane copolymer, vinyl-terminated diethyl siloxane-dimethyl siloxane copolymer, vinyl methyl siloxane, monomethacryloxypropyl-terminated polydimethylsiloxane, monovinyl-terminated polydimethylsiloxane, monoallyl-mono-trimethylsilyloxy-terminated polyethylene oxide, or any combination thereof.

In some embodiments, the UV curable resin is a mercapto functional compound that can be cross-linked under UV curing conditions with an isocyanate, an epoxy resin, or an unsaturated compound. In some embodiments, the mercapto functional compound is a polythiol. In some embodiments, the polythiol is selected from: pentaerythritol tetrakis(3-mercaptopropionate) (PETMP), trimethylolpropane tris(3-mercaptopropionate) (TMPMP), ethylene glycol bis(3-mercaptopropionate) (GDMP); tris [25-(3-mercapto-propionyloxy)ethyl]isocyanurate (TEMPIC), dipentaerythritol hexa(3-mercaptopropionate) (Di-PETMP), ethoxylated trimethylolpropane tri(3-mercaptopropionate) (ETMP1300 and ETTMP700), polycaprolactone tetra(3-mercaptopropionate) (PCL4MP1350), pentaerythritol tetramercaptoacetate (PETMA), trimethylolpropane trimercaptoacetate (TMPMA), or ethylene glycol dimercaptoacetate (GDMA). These compounds are sold under the trade name THIOCURE® by Bruno Bock (Malsacht, Germany).

In some embodiments, the UV curable resin further comprises photoinitiator. The photoinitiator will initiate crosslinking and/or curing reactions of the photosensitive material during exposure to light. In some embodiments, the photoinitiator is a compound, such as acetophenone-based, benzoin-based, or thidrone-based, that initiates the polymerization, crosslinking and curing of monomers.

In some embodiments, the UV curable resin comprises mercapto-functional compounds, methacrylates, acrylates, isocyanates, or any combination thereof. In some embodiments, the UV curable resin comprises polythiols, methacrylates, acrylates, isocyanates, or any combination thereof.

In some embodiments, the photoinitiator is MINS-311RM (Minuta Technology Co., Ltd (Korea)).

In some embodiments, the photoinitiator is Irgacure® 127, Irgacure® 184, Irgacure® 184D, Irgacure® 2022, Irgacure® 2100, Irgacure® 250, Irgacure® 270, Irgacure® 2959, Irgacure® 369, Irgacure® 369EG, Irgacure® 379, Irgacure® 500, Irgacure® 651, Irgacure® 754, Irgacure® 784, Irgacure® 819, Irgacure® 819DW, Irgacure® 907, Irgacure® 907FF, Irgacure® OxeOl, Irgacure® TPO-L, Irgacure® 1173, Irgacure® 1173D, Irgacure® 4265, Irgacure® BP, or Irgacure® MBF (BASF Corporation (Wyandotte, Michigan)). In some embodiments, the photoinitiator is TPO (2,4,6-trimethylbenzoyl-diphenyl-oxide) or MBF (methyl benzoyl formate).

In some embodiments, the weight percentage of organic resin in the formulation is about 20% to about 99%, about 20% to about 95%, about 20% to about 90%, about 20% to about 85%, about 20% to about 80%, about 20% to about 70%, about 20% to about 60%, about 40% to about 99%, about 40% to about 95%, about 40% to about 90%, about 40% to about 85%, about 40% to about 80%, about 40% to about 70%, about 70% to about 99%, about 70% to about 95%, about 70% to about 90%, about 70% to about 85%, about 70% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%.

The formulation as described herein further comprises at least one solvent. In some embodiments, the formulation as described herein is a solution.

In some embodiments, the formulation as described herein is a dispersion.

The formulation in the embodiments as described herein may comprise the emitter E of 0.01 wt % to 20 wt %, preferably 0.1 wt % to 20 wt %, more preferably 0.2 wt % to 20 wt %, and most preferably 2 wt % to 15 wt %.

Using the formulation as described herein, the color conversion layer may be fabricated by ink-jet printing, transfer printing, photolithography, etc. In this case, the compound (i.e., the color conversion material) needs to be dissolved alone or together with other materials in a resin (prepolymer) and/or an organic solvent, to form the ink. The mass concentration of the compound (i.e. the color conversion material) in the ink is not less than 0.1 wt %. The color conversion ability of the color conversion layer can be tuned by adjusting the concentration of the color conversion material in the ink and the thickness of the color conversion layer. In general, the higher the concentration of the color conversion material or the thickness of the layer, the higher the color conversion efficiency of the color conversion layer would be.

In some embodiments, the at least one solvent is selected from water, alcohols, esters, aromatic ketones, aromatic ethers, aliphatic ketones, aliphatic ethers, borates, phosphorates, or a combination of two or more of them.

In some embodiments, the suitable and preferred solvents include aliphatics, alicyclics, aromatics, amines, thiols, amides, nitriles, esters, ethers, polyethers, alcohols, diols, or polyols.

In some embodiments, the alcohol represents a solvent of the suitable class. The preferred alcohols include alkylcyclohexanols, particularly methylated aliphatic alcohols, naphthols, etc.

Other examples of the suitable alcohol solvents include dodecanol, phenyltridecanol, benzyl alcohol, ethylene glycol, ethylene glycol methyl ether, glycerol, propylene glycol, 1-ethoxy-2-propanol, etc.

The solvent may be used alone or as a combination of two or more organic solvents.

In some embodiments, the formulation as described herein comprises an organic functional compound as described herein and at least one organic solvent, and further comprising another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4 dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or any combination thereof.

In some embodiments, the organic solvent of the formulation is selected from aromatic, heteroaromatic, ester, aromatic ketone, aromatic ether, aliphatic ketone, aliphatic ether, cycloaliphatic, olefinic compound, alicyclic or olefin compounds, borate, phosphorate, or a combination of two or more of them.

Examples of aromatic or heteroaromatic solvents as described herein include, but not limited to: 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylcumene, dipentylbenzene, o-diethylbenzene, m-diethylbenzene, p-diethylbenzene, 1,2,3,4-tetramethyl benzene, 1,2,3,5-tetramethyl benzene, 1,2,4,5-tetramethyl benzene, butylbenzene, dodecyl benzene, 1-methylnaphthalene, 1,2,4-trichlorobenzene, 1,3-dipropoxybenzene, 4,4-difluorodiphenylmethane, diphenyl ether, 1,2-dimethoxy-4-(1-propenyl) benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-Isopropylbiphenyl, α,α-dichlorodiphenylmethane, 4-(3-phenylpropyl) pyridine, benzyl benzoate, 1,1-bis (3,4-dimethylphenyl) ethane, 2-isopropylnaphthalene, dibenzyl ether, etc.

In some embodiments, the suitable and preferred solvents are aliphatics, alicyclics, aromatics, amines, thiols, amides, nitriles, esters, ethers, or polyethers.

The solvent may be a cycloalkane, such as decahydronaphthalene.

In some embodiments, the formulation as described herein comprises at least 50 wt % of an alcoholic solvent, preferably at least 80 wt %, particularly preferably at least 90 wt %.

In some embodiments, the solvent particularly suitable for the present disclosure is a solvent having Hansen solubility parameters in the following ranges:

δ_{d} (dispersion force) is in the range of 17.0 MPa^{1/2} to 23.2 MPa^{1/2}, especially in the range of 18.5 MPa^{1/2} to 21.0 MPa^{1/2};

δₚ (polarity force) is in the range of 0.2 MPa^{1/2} to 12.5 MPa^{1/2}, especially in the range of 2.0 MPa^{1/2} to 6.0 MPa^{1/2};

δₕ (hydrogen bonding force) is in the range of 0.9 MPa^{1/2} to 14.2 MPa^{1/2}, especially in the range of 2.0 MPa^{1/2} to 6.0 MPa^{1/2}.

In the formulation as described herein, the boiling point parameter should be taken into account when selecting the organic solvents. In the present disclosure, the boiling points of the organic solvents ≥150° C.; preferably ≥180° C.; more preferably ≥200° C.; further preferably ≥250° C.; and most preferably ≥275° C. or ≥300° C. The boiling points in these ranges are beneficial in terms for preventing nozzle clogging of the inkjet printhead. The organic solvent can be evaporated from solution system to form a functional film.

In some embodiments, in the formulation as described herein,
1) the viscosity is in the range of 1 cps to 100 cps at 25° C.; and/or
2) the surface tension is in the range of 19 dyne/cm to 50 dyne/cm at 25° C.

In the formulation as described herein, the surface tension parameter should be taken into account when selecting the resins (prepolymers) or the organic solvents. The suitable surface tension parameters of the inks are suitable for the particular substrate and particular printing method. For example, for the ink-jet printing, in some embodiments, the surface tension of the resin (prepolymer) or the organic solvent at 25° C. is in the range of 19 dyne/cm to 50 dyne/cm, more preferably in the range of 22 dyne/cm to 35 dyne/cm, and most preferably in the range of 25 dyne/cm to 33 dyne/cm.

In some embodiments, the surface tension of the formulation as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In the formulation as described herein, the viscosity parameters of the ink should be taken into account when selecting the resins (prepolymers) or the organic solvents. The viscosity can be adjusted by election different methods, such as by the suitable resin (prepolymer) or organic solvent and the concentration of functional materials in the ink. In some embodiments, the viscosity of the resin (prepolymer) or the organic solvent is less than 100 cps, more preferably less than 50 cps, and most preferably from 1.5 cps to 20 cps. The viscosity herein refers to the viscosity during printing at the ambient temperature that is generally at 15-30° C., preferably at 18-28° C., more preferably at 20-25° C., and most preferably at 23-25° C. The resulting formulation will be particularly suitable for ink-jet printing.

In some embodiments, the viscosity of the formulation as described herein at 25° C. is in the range of about 1 cps to 100 cps; more preferably in the range of 1 cps to 50 cps; and most preferably in the range of 1.5 cps to 20 cps.

The ink obtained from the resin (prepolymer) or the organic solvent satisfying the above-mentioned boiling point parameter, surface tension parameter and viscosity parameter can form a functional film with uniform thickness and formulation property.

The present disclosure also provides an organic functional film comprising a formulation as described herein.

The present disclosure further provides a method for preparing the organic functional film, as shown in the following steps:
1) prepare a formulation as described herein;
2) the formulation is coated on a substrate by printing or coating to form a film, where the method of printing or coating is selected from the group consisting of ink-jet printing, nozzle printing, typographic printing, screen printing, dip coating, spin coating, blade coating, roller printing, torsional roll printing, planographic printing, flexographic printing, rotary printing, spray coating, brush or pad printing, and slit die coating;
3) the obtained film is heated at 50° C. and above, optionally in combination with ultraviolet irradiation, to allow the film to undergo a crosslinking reaction and be cured.

The thickness of the organic functional film is generally 50 nm to 200 m, preferably 100 nm to 150 m, more preferably 500 nm to 100 m, further preferably 1 m to 50 m, and most preferably 1 m to 20 m.

In another aspect, the present disclosure further provides the applications of the formulation and the organic functional film in optoelectronic devices.

In some embodiments, the optoelectronic device may be selected from an organic light emitting diode (OLED), an organic photovoltaic cell (OPV), an organic light emitting electrochemical cell (OLEEC), an organic light emitting field effect transistor, or an organic laser.

In yet another aspect, the present disclosure further provides an optoelectronic device comprising a formulation or an organic functional film as described herein.

Preferably, the optoelectronic device is an electroluminescent device, such as an organic light emitting diode (OLED), an organic light emitting electrochemical cell (OLEEC), an organic light emitting field effect transistor, a perovskite light emitting diode (PeLED), and a quantum dot light emitting diode (QD-LED), where one of the functional layers comprises an organic functional film as described above. The functional layer may be selected from a hole-injection layer, a hole-transport layer, an electron-injection layer, an electron-transport layer, a light-emitting layer, or a cathodic passivation layer (CPL).

In some embodiments, the optoelectronic device is an electroluminescent device comprising two electrodes, where the functional layer is located on the same side of the two electrodes.

In some embodiments, the optoelectronic device comprises a light emitting unit and a color conversion layer (functional layer), where the color conversion layer comprises a formulation or an organic functional film as described herein.

In some embodiments, the color conversion layer absorbs 95% or more of the light from the light emitting unit, preferably 97% or more, more preferably 99% or more, and most preferably 99.9% or more.

In some embodiments, the light emitting unit is selected from a solid-state light emitting device. The solid-state light emitting device is preferably selected from a LED, an organic light emitting diode (OLED), an organic light emitting electrochemical cell (OLEEC), an organic light emitting field effect transistor, a perovskite light emitting diode (PeLED), a quantum dot light emitting diode (QD-LED), or a nanorod LED (see DOI: 10.1038/srep28312).

In some embodiments, the light emitting unit emits blue light, which is converted into green light or red light by the color conversion layer.

In some embodiments, the light emitting unit emits green light, which is converted into yellow light or red light by the color conversion layer.

The present disclosure further relates to a display comprising at least three pixels of red, green and blue. As shown in the attached FIG. 1, the blue pixel comprises a blue emitting unit, and the pixel of red or green comprises a blue emitting unit and a corresponding red or green color conversion layer.

In yet another aspect, the present disclosure further provides an organic light-emitting device comprising a substrate, a first electrode, an organic light-emitting layer, a second electrode, a color conversion layer, and an encapsulation layer (e.g., an outermost encapsulation layer) in sequence from bottom to top, and the second electrode is at least partially transparent, where 1) the color conversion layer comprises a formulation as described herein; 2) the color conversion layer absorbs 90% or more of the light emitted by the organic light-emitting layer through the second electrode; 3) the emission spectrum of the organic compound H is on the short wavelength side of the absorption spectrum of the emitter E, and at least partially overlaps with the absorption spectrum of the emitter E. Preferably, the FWHM of the emission spectrum of the emitter E≤55 nm.

The organic compound H, the emitter E, and the some embodiments thereof are as described above.

In addition, for the purposes of the organic light-emitting device as described herein, the emitter E may be further selected from a compound (i.e., Bodipy derivatives) having the following structural formula:

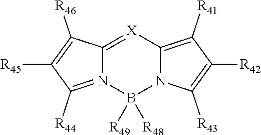

Where X is CR₄₇ or N; R₄₁ to R₄₉ are independently selected from a hydrogen, an alkyl group, a cycloalkyl group, a heterocyclic group, an alkenyl group, a cycloalkenyl group, an alkynyl group, a hydroxyl group, a mercapto group, an alkoxy group, an alkylthio group, an aryl ether group, an aryl thioether group, an aryl group, a heteroaryl group, a halogen, a cyano group, an aldehyde group, a carbonyl group, a carboxy group, an oxycarboxyl group, a carbamoyl group, an amino group, a nitro group, a silyl group, a siloxanyl group, a boronalkyl group, or a phosphorus oxide group; R₄₁-R₄₉ may form a fused ring and an aliphatic ring with the adjacent substituents.

In some embodiments, each of R₄₈ and R₄₉ is independently selected from a collection of electron-withdrawing groups. Suitable electron-withdrawing groups include, but not limited to, F, Cl, a cyano group, a partial/perfluorinated alkyl chain, or one of the following groups:

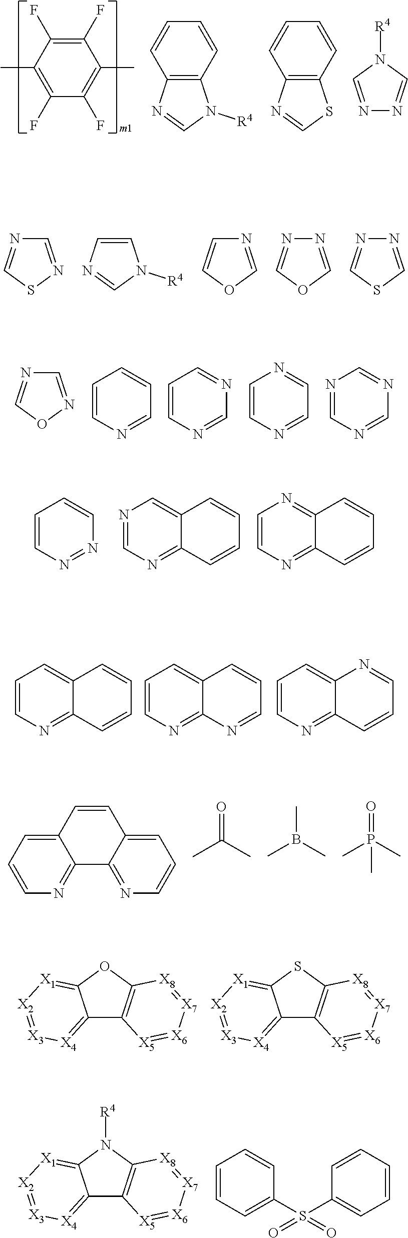

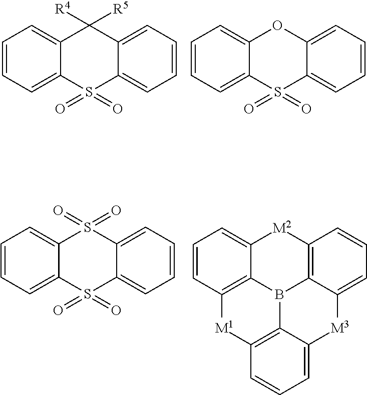

Where m1 is 1, or 2, or 3; each of X₁ to X₈ is CR⁴⁰ or N, and at least one of them is N; M¹, M², M³ independently represent N(R⁴⁰), C(R⁴⁰R⁵⁰), si(R⁴⁰R⁵⁰), O, C═N(R⁴⁰), C═C(R⁴⁰R⁵⁰), P(R⁴⁰), p(═O)R⁴⁰, S, S═O, SO2, or null; R⁴ and R⁵ are identically defined as described above, and R⁴⁰ and R⁵⁰ are identically defined as the above-mentioned R⁴.

Examples of suitable Bodipy derivatives include, but not limited to:

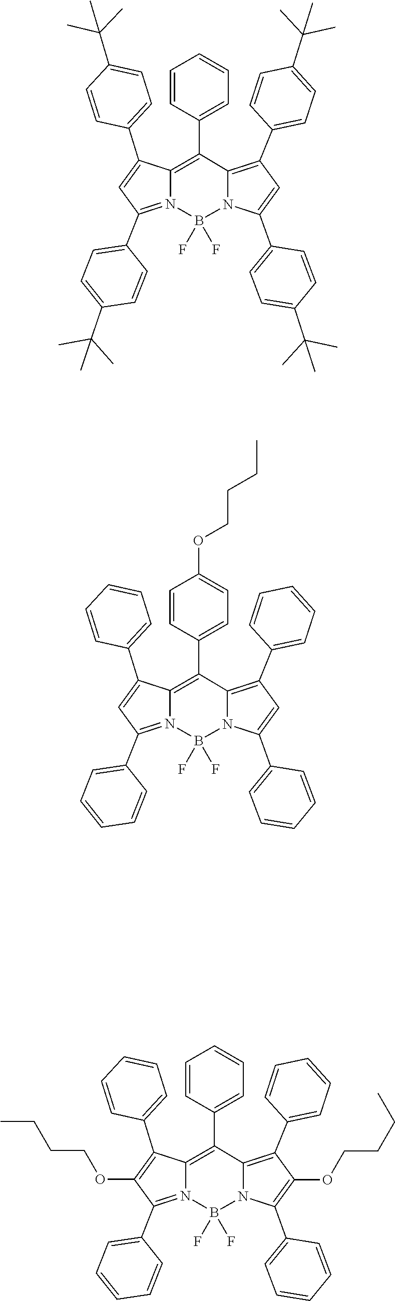

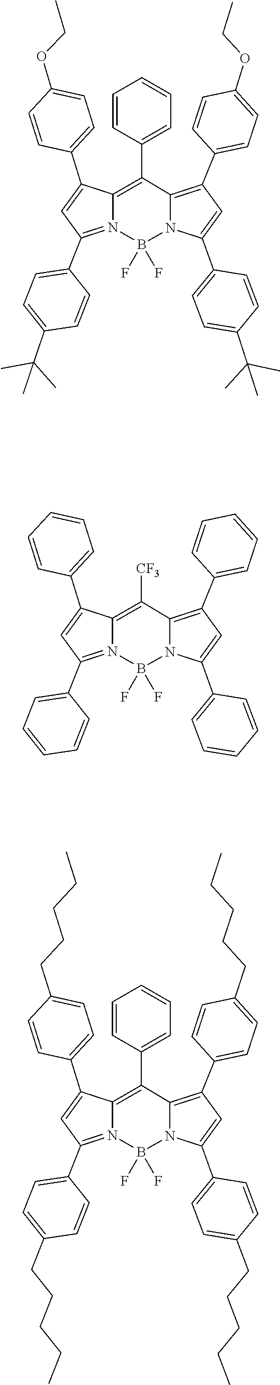

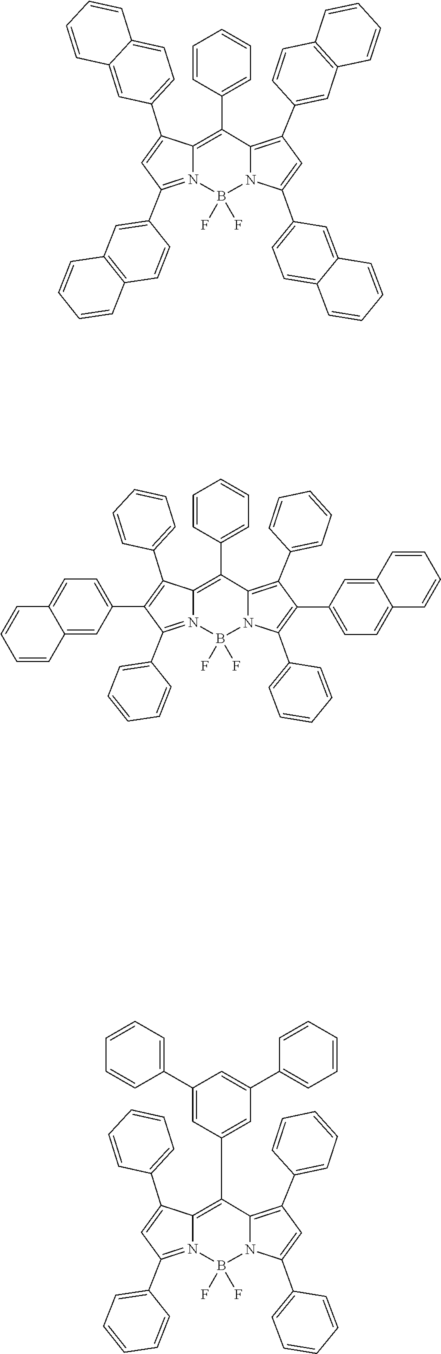

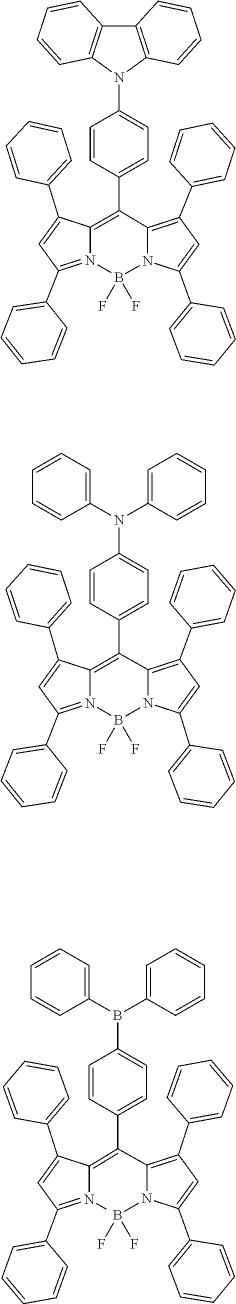

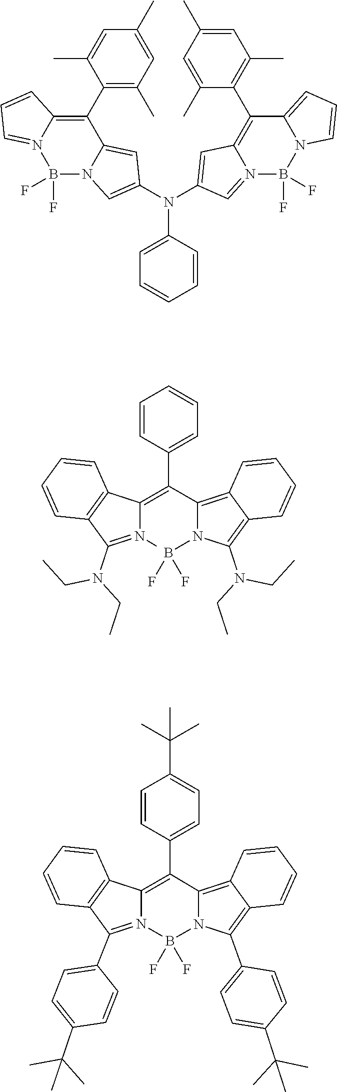

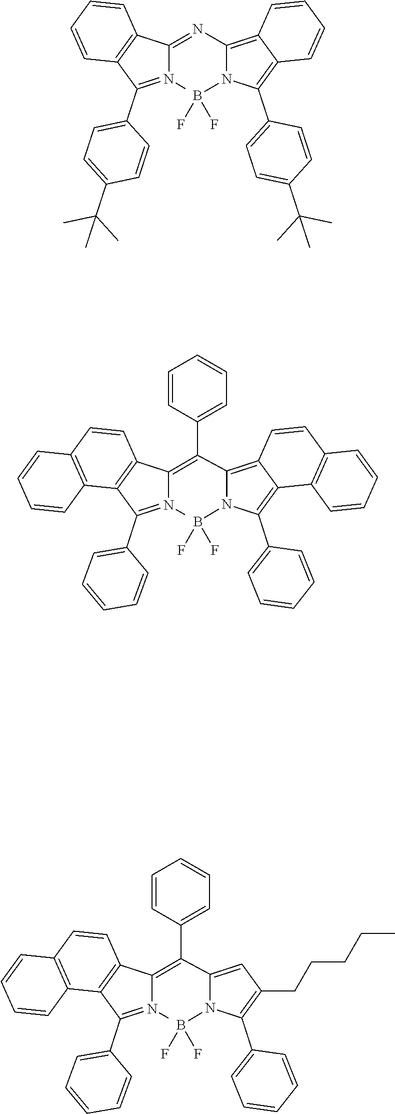

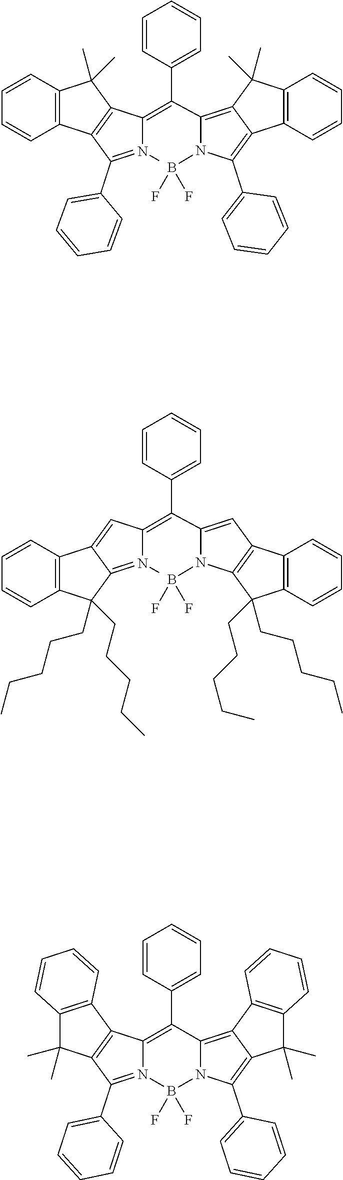

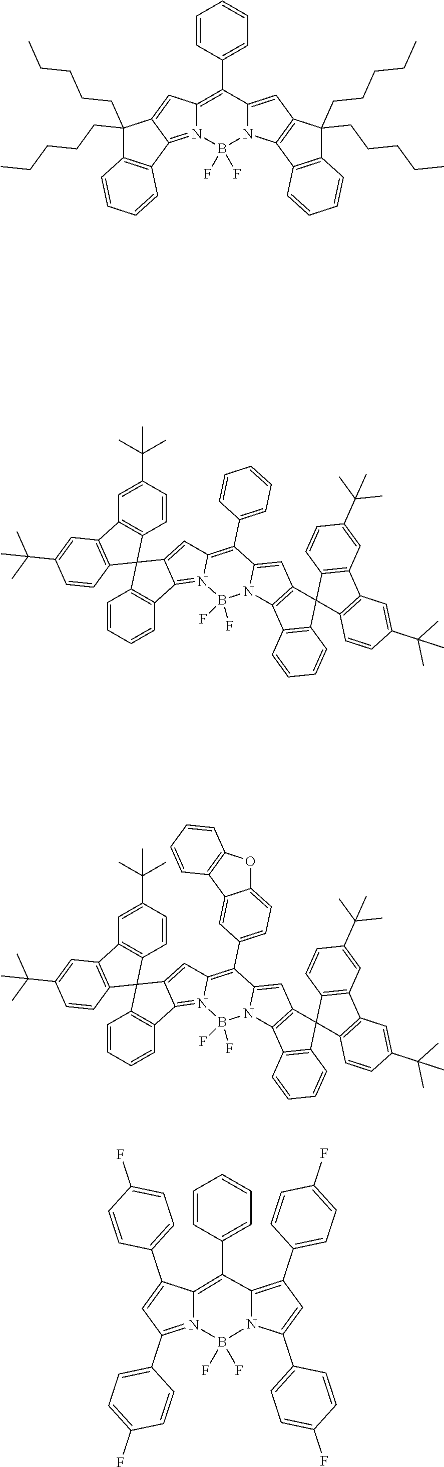

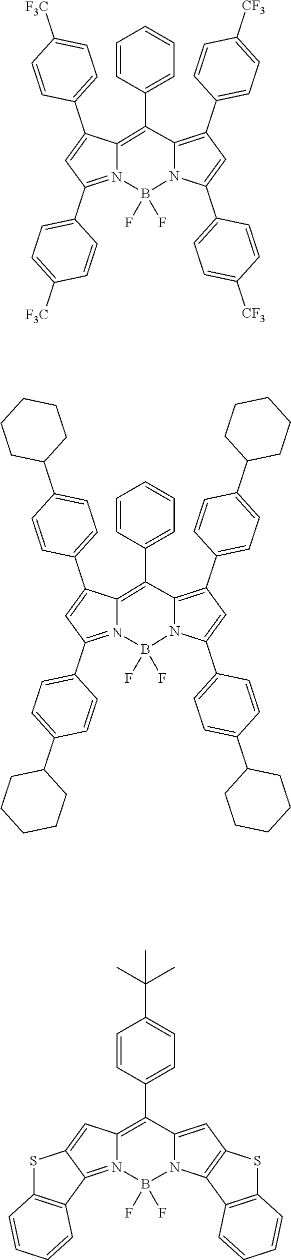

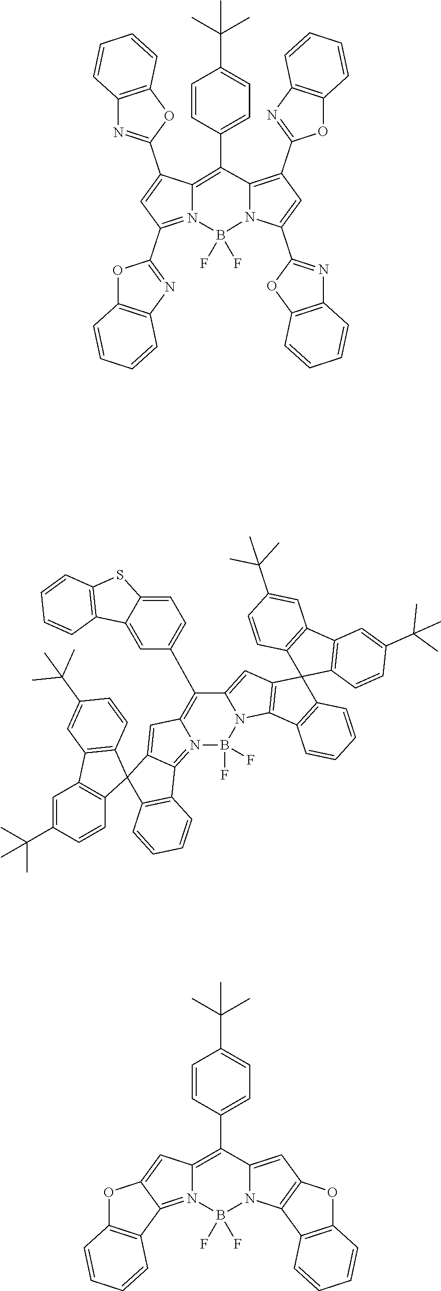

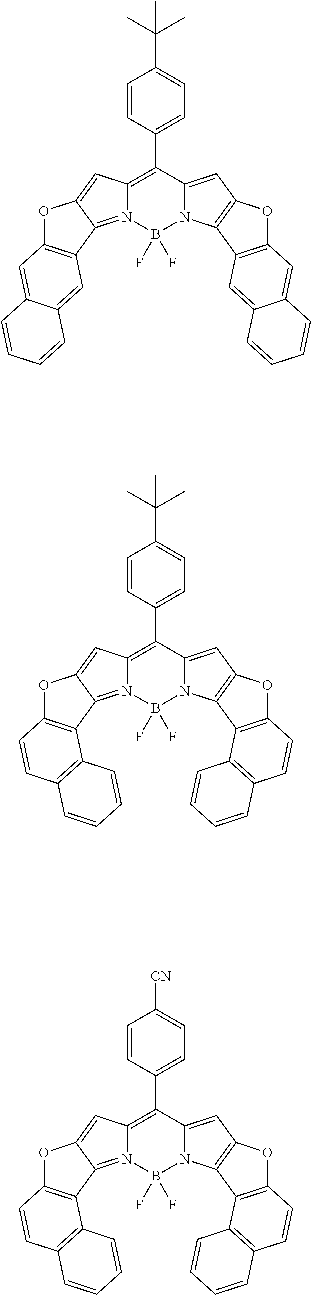

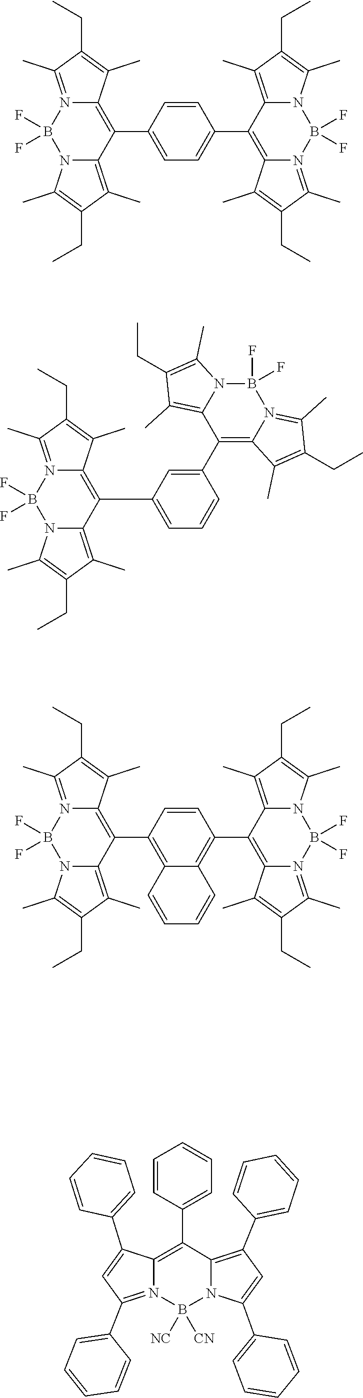

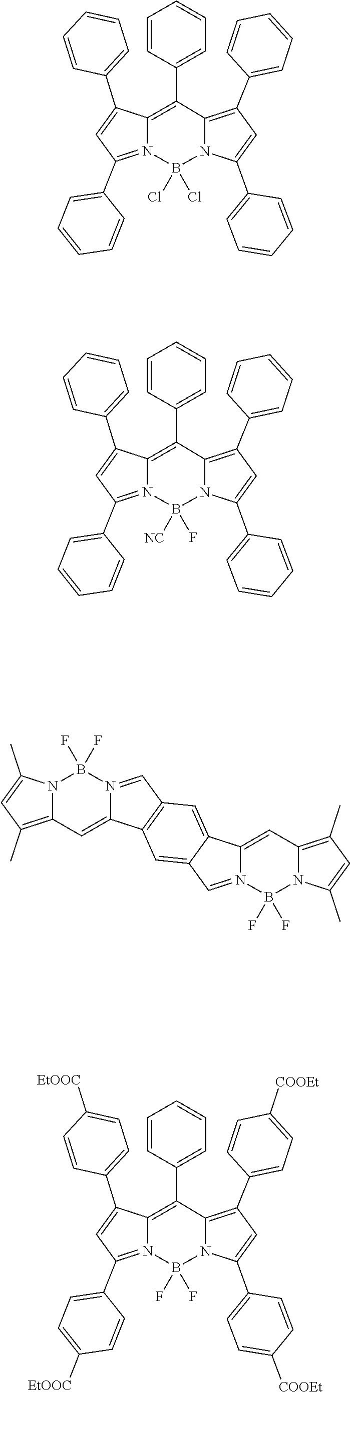

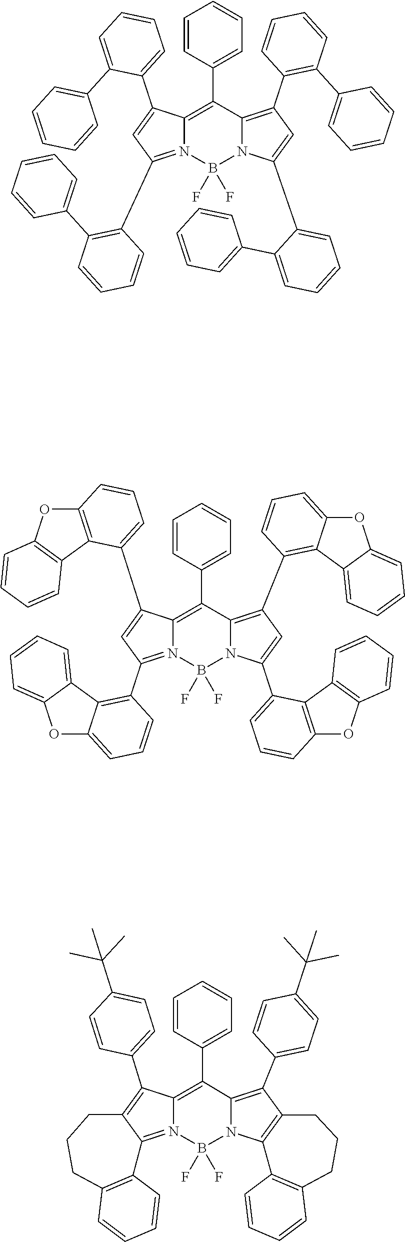

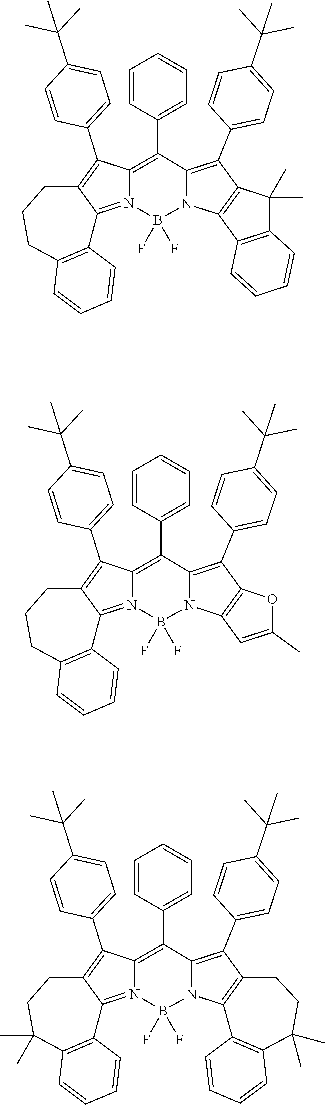

In some embodiments, the color conversion layer absorbs 95% or more of the light from the light emitting unit, preferably 97% or more, more preferably 99% or more, and most preferably 99.9% or more.

In some embodiments, the thickness of the color conversion layer is between 100 nm and 5 m, preferably between 150 nm and 4 m, more preferably between 200 nm and 3 m, and most preferably between 200 nm and 2 m.

In some embodiments, the organic light-emitting device is an OLED. More preferably, the first electrode is an anode, the second electrode is a cathode. Particularly preferably, the organic light-emitting device is a top emission OLED.

The substrate should be opaque or transparent. A transparent substrate could be used to produce a transparent light-emitting device (for example: Bulovic et al. , Nature 1996, 380, p29, and Gu et al., Appl. Phys. Lett. 1996, 68, p2606). The substrate can be rigid/flexible, e.g. it can be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature (Tg)>150° C., preferably >200° C., more preferably >250° C., and most preferably >300° C. Examples of the suitable flexible substrate includes poly ethylene terephthalate(PET) and polyethylene glycol (2,6-naphthalene) (PEN).

The choice of anodes may include a conductive metal, a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level of the emitter of the light-emitting layer, or the HOMO energy level/valence band energy level of the p-type semiconductor materials of the hole-injection layer (HIL)/hole-transport layer (HTL)/electron-blocking layer (EBL)<0.5 eV, preferably <0.3 eV, more preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode material can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as described herein.

The choice of cathode may include a conductive metal or a metal oxide. The cathode should be able to easily inject electrons into the EIL, the ETL, or the directly into the light emitting layer. In some embodiments, the absolute value of the difference between the work function of the cathode and the LUMO energy level of the emitter of the light-emitting layer, or the LUMO energy level/conduction band energy level of the n-type semiconductor materials of the electron-injection layer (EIL)/electron-transport layer (ETL)/hole-blocking layer (HBL)<0.5 eV, preferably <0.3 eV, and most preferably <0.2 eV. In principle, all materials that can be used as cathodes for OLEDs may be applied as cathode materials for the devices as described herein. Examples of cathode materials include, but not limited to: Al, Au, Ag, Ca, Ba, Mg, LiF/Al, MgAg alloys, BaF₂/Al, Cu, Fe, Co, Ni, Mn, Pd, Pt, ITO, etc. The cathode material can be deposited using any suitable technique, such as the suitable physical vapor deposition method, including RF magnetron sputtering, vacuum thermal evaporation, e-beam, etc. In some embodiments, the transmittance of the cathode in the range of 400-680 nm≥40%, preferably ≥45%, more preferably ≥50%, and most preferably ≥60%. Typically, 10-20 nm of Mg:Ag alloys can be used as transparent cathodes, and the ratio of the Mg:Ag can range from 2:8 to 0.5:9.5.

The light emitting layer of the organic light-emitting device preferably comprises a blue fluorescent host and a blue fluorescent dopant. In some embodiments, the light emitting layer comprises a blue phosphorescent host and a blue phosphorescent dopant. The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1, the entire contents of these three documents are hereby incorporated herein for reference.

Further, the organic light-emitting device further comprises a cathode capping layer (CPL).

In some embodiments, the CPL is disposed between the second electrode and the color conversion layer.

In some embodiments, the CPL is disposed on the top of the color conversion layer.

The CPL material generally requires a high refractive index (n), such as n≥1.95@460 nm, n ≥1.90@520 nm, n≥1.85@620 nm. Examples of the CPL materials include:

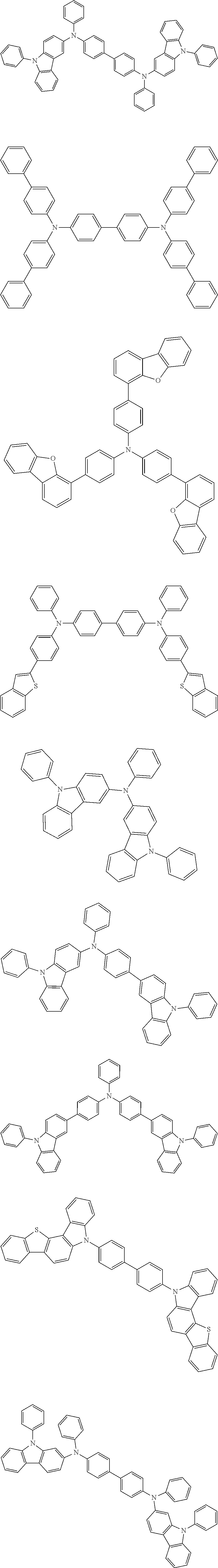

More further examples of the CPL materials can be found in the following patent literature: KR20140128653A, KR20140137231A, KR20140142021A, KR20140142923A, KR20140143618A, KR20140145370A, KR20150004099A, KR20150012835A, U.S. Pat. No. 9,496,520B2, US2015069350A1, CN103828485B , CN104380842B , CN105576143A, TW201506128A, CN103996794A, CN103996795A, CN104744450A, CN104752619A, CN101944570A, US2016308162A1, U.S. Pat. No. 9,095,033B2, US2014034942A1, WO2017014357A1. The above patent documents are incorporated herein by reference in their entirety.

In some embodiments, the color conversion layer comprises a CPL material as described herein. In some embodiments, the color conversion layer is co-evaporated by one above-mentioned CPL material, the organic compound H, and the emitter E. In some embodiments, the mass ratio of the organic compound H is 50-20%, and the mass ratio of the emitter E is 10-15%.

Preferably, the encapsulation layer of the organic light-emitting device is thin-film encapsulated (TFE).

The present disclosure also relates to a display panel, where at least one pixel comprises an organic light-emitting device as described herein.

The present disclosure will be described below in conjunction with the preferred embodiments, but the present disclosure is not limited to the following embodiments. It should be understood that the appended claims summarize the scope of the present disclosure, and those skilled in the art, guided by the conception of the disclosure should realize that certain changes to the various embodiments of the disclosure will be covered by the scope of the claims of the present disclosure.

### SPECIFIC EMBODIMENT

### Example 1

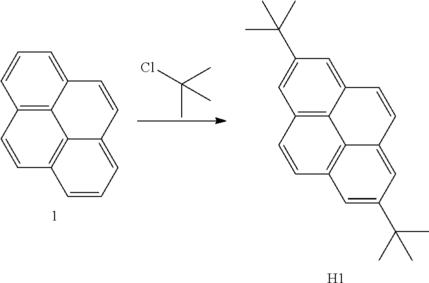

### Synthesis of Compound H1

Compound 1 (1.0 g, 5 mmol) was dissolved in 20 g of BmimPF6, and 0.4 g of aluminum trichloride (AlCl₃) and 20 mL of tert-butyl chloride were added into the above mixture in sequence. The resulting mixture was then heated to 70° C., and reacted for 24 h. After the reaction was completed, the product was diluted with DCM and the resulting organic phase was washed with water. After removing the solvent, the resulting sample was purified by column chromatography to yield 1.5 g (96.2% yield) of compound H1.

### Example 2

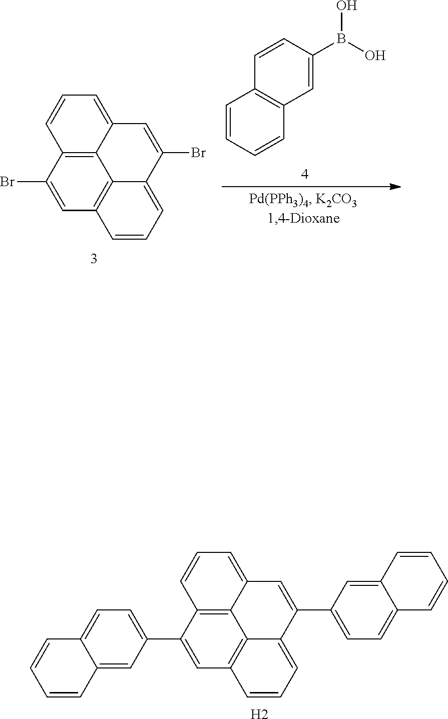

### Synthesis of Compound H2

Compound 3 (3.6 g, 10 mmol) and compound 4 (3.8 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.9 g (86.7% yield) of compound H2.

### Example 3

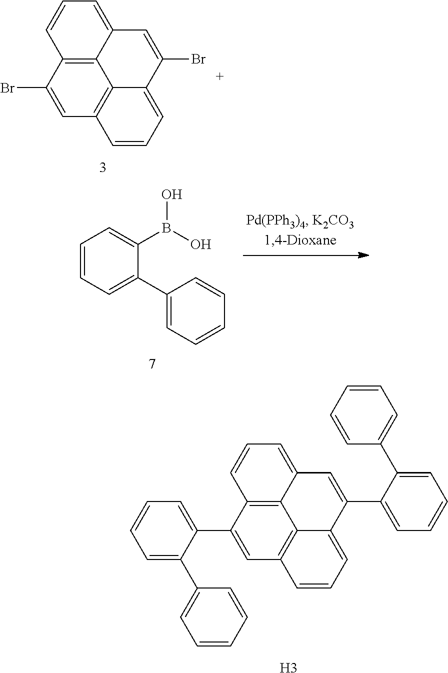

### Synthesis of Compound H3

Compound 3 (3.6 g, 10 mmol) and compound 7 (4.4 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 4.5 g (88.2% yield) of compound H3.

### Example 4

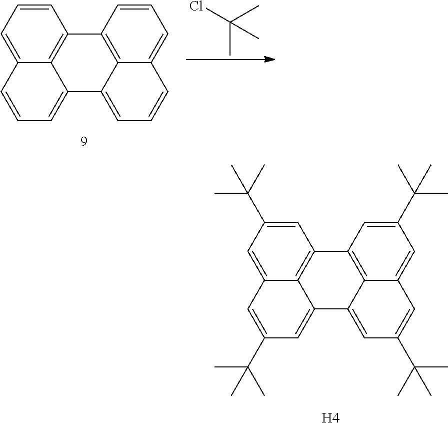

### Synthesis of Compound H4

Compound 9 (1.3 g, 5 mmol) was dissolved in 20 g of BmimPF6 and 0.4 g of aluminum trichloride (AlCl₃) and 20 mL of tert-butyl chloride were added into the above mixture in sequence. The resulting mixture was then heated to 70° C., and reacted for 24 h. After the reaction was completed, the product was diluted with DCM and the resulting organic phase was washed with water. After removing the solvent, the resulting sample was purified by column chromatography to yield 2.2 g (92.7% yield) of compound H4.

### Example 5

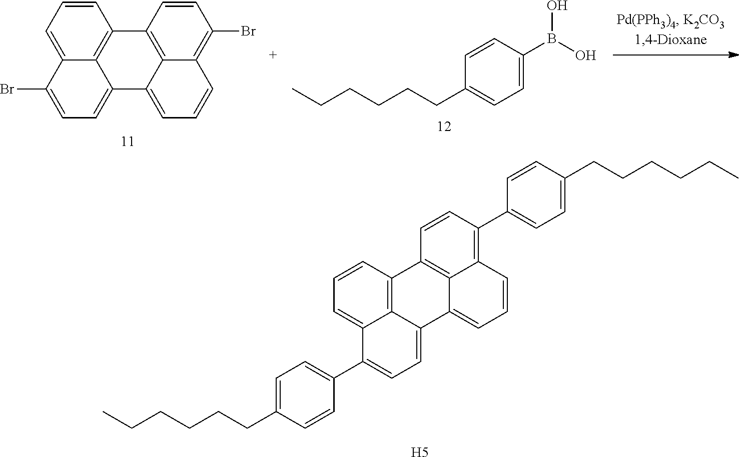

### Synthesis of compound H5

Compound 11 (4.1 g, 10 mmol) and compound 12 (4.5 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the reaction was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 4.8 g (84.2% yield) of compound H5.

### Example 6

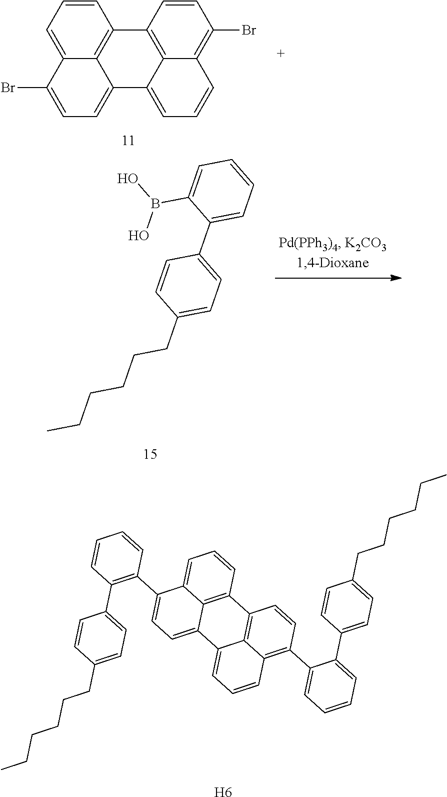

### Synthesis of Compound H6

Compound 11 (4.1 g, 10 mmol) and compound 15 (6.2 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the reaction was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 6.4 g (88.2% yield) of compound H6.

### Example 7

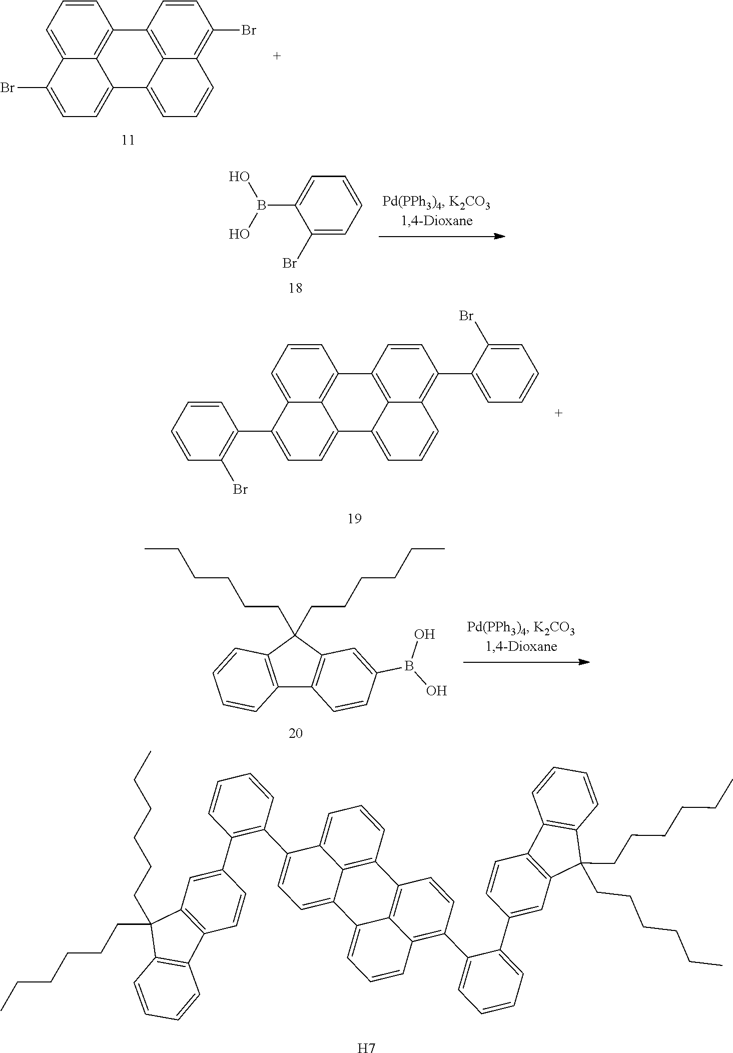

### Synthesis of Intermediate 19

Compound 11 (4.1 g, 10 mmol) and compound 18 (4.4 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.6 g (64.2% yield) of intermediate 19.

### Synthesis of Compound H7

Intermediate 19 (2.8 g, 5 mmol) and compound 20 (4.2 g, 11 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 4.5 g (84.2% yield) of compound H7.

### Example 8

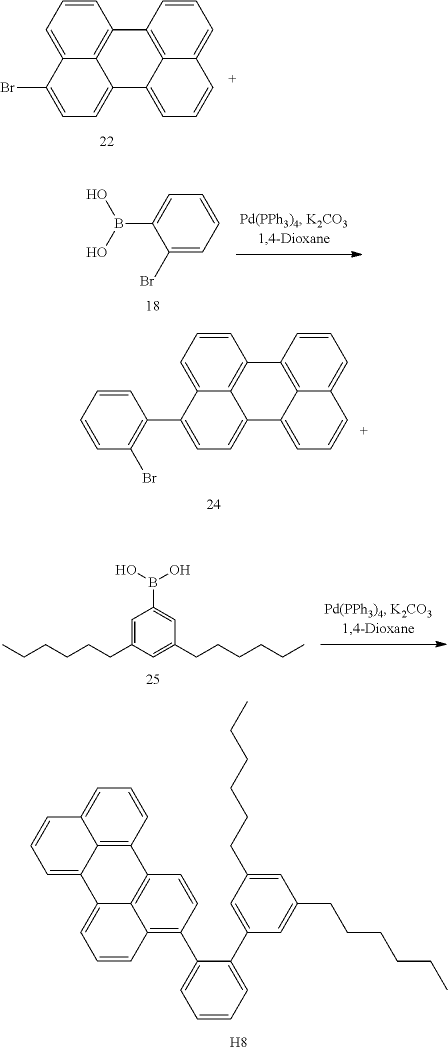

### Synthesis of Intermediate 24

Compound 22 (3.3 g, 10 mmol) and compound 18 (2.2 g, 11 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.4 g (84.2% yield) of intermediate 24.

### Synthesis of Compound H8

Intermediate 24 (2.0 g, 5 mmol) and compound 25 (1.5 g, 5.2 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 2.6 g (89.2% yield) of compound H8.

### Example 9

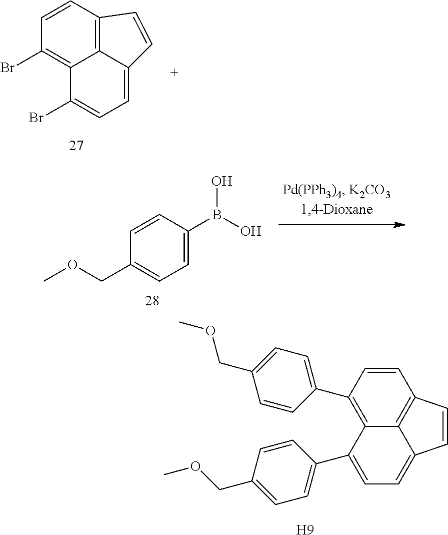

### Synthesis of Compound H9

Compound 27 (3.1 g, 10 mmol) and compound 28 (3.7 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.4 g (87.6% yield) of compound H9.

### Example 10

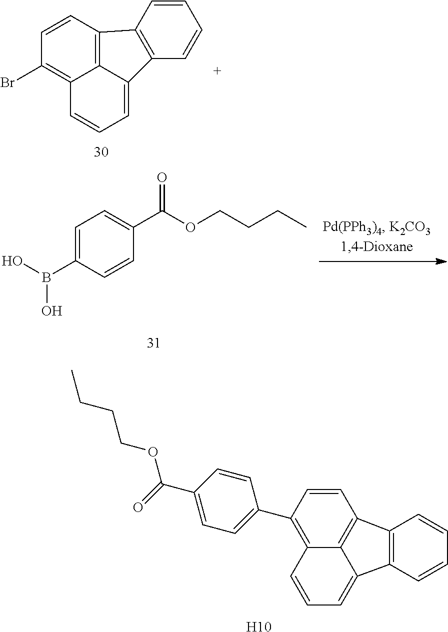

### Synthesis of Compound H10

Compound 30 (2.8 g, 10 mmol) and compound 31 (2.5 g, 11 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.5 g (92.6% yield) of compound H10.

### Example 11

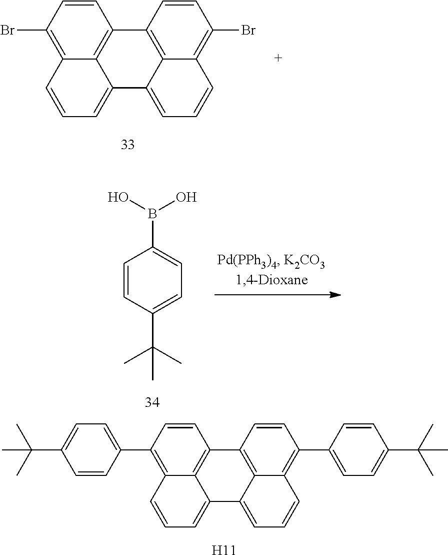

Synthesis of Compound H11

Compound 33 (4.1 g, 10 mmol) and compound 34 (3.9 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 4.4 g (84.2% yield) of compound H11.

### Example 12

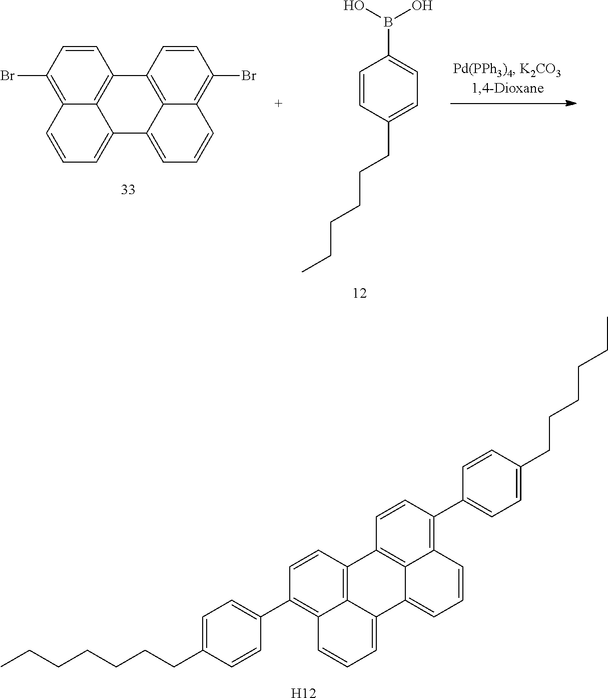

### Synthesis of Compound H12

Compound 33 (4.1 g, 10 mmol) and compound 12 (4.5 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 4.9 g (83.2% yield) of compound H12.

### Example 13

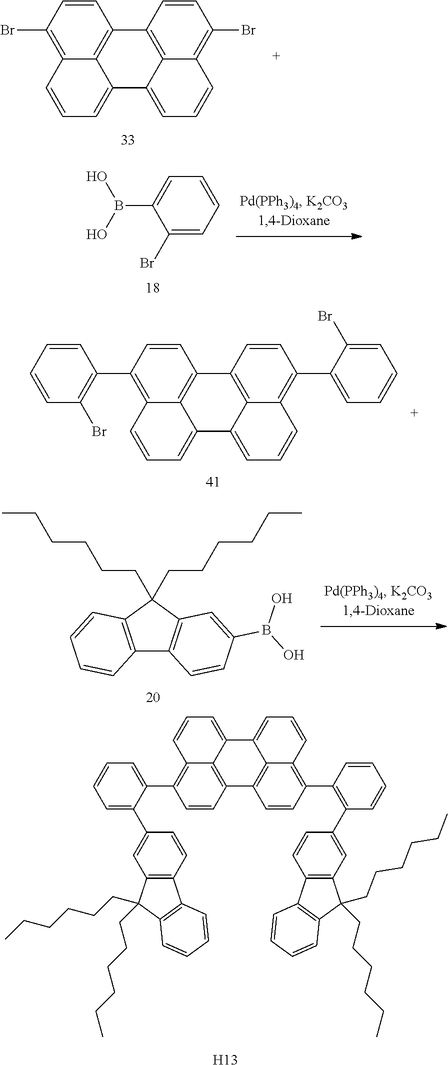

### Synthesis of Intermediate 41

Compound 33 (4.1 g, 10 mmol) and compound 18 (4.4 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.6 g (64.2% yield) of intermediate 41.

### Synthesis of Compound H13

Intermediate 41 (2.8 g, 5 mmol) and compound 20 (4.2 g, 11 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 4.5 g (84.2% yield) of compound H13.

### Example 14

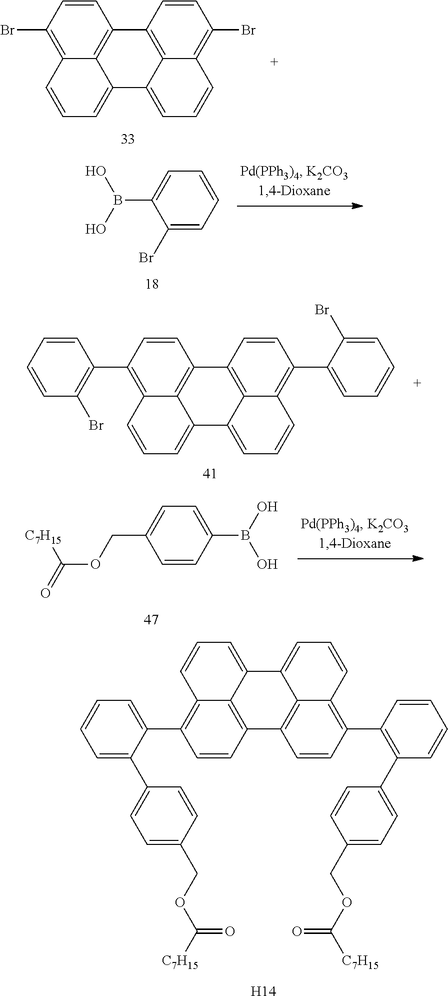

### Synthesis of intermediate 41

Compound 33 (4.1 g, 10 mmol) and compound 18 (4.4 g, 22 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.6 g (64.2% yield) of compound intermediate 41.

### Synthesis of Compound H14

Intermediate 41 (2.8 g, 5 mmol) and compound 47 (3.1 g, 11 mmol) were dissolved in potassium carbonate aqueous solution (2 M, 10 mL) and 1,4-dioxane (40 mL). After bubbling for 30 minutes, catalyst Pd(PPh₃)₄ (0.3 g) was added to the above mixture in N₂ atmosphere, and the mixture was refluxed for 6 h. After the reaction was completed and cooled down to room temperature, 1,4-dioxane was removed, the obtained crude product was dissolved in dichloromethane and the resulting organic phase was washed with water for three times. After the separation, the organic phase was dried and further purified by silica gel column chromatography to yield a solid. Then the solid was washed with ethanol absolute in reflux for 24 h. After that, the resulting sample was dried to obtained 3.5 g (81.2% yield) of compound H14.

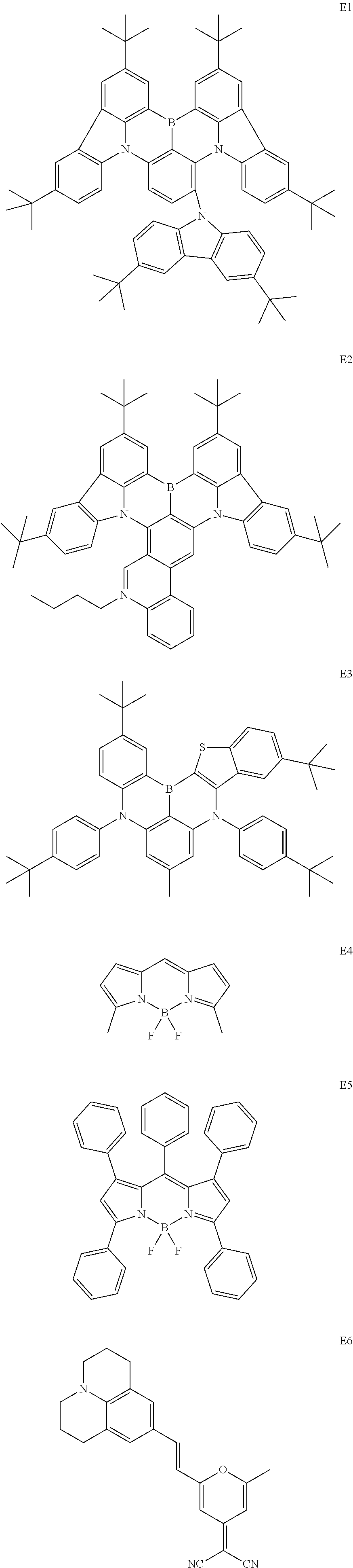

E1 is a green light emitter and was synthesized with reference to Angew. Chem. Int. Ed. 10.1002/anie.202007210; E2 is a green light emitter and was synthesized with reference to Angew. Chem. Int. Ed. 10.1002/anie.202008264; E3 is a blue light emitter and was synthesized with reference to US2020395553A1. E4 is a green light emitter, both E5 and E6 are red light emitters.

### Quantum Chemical Simulation of Compounds

The energy level of the organic compounds (H1-H14) can be calculated by quantum computation, for example, using TD-DFT (time-dependent density functional theory) by Gaussian03W (Gaussian Inc.). The specific simulation method is as follows: firstly, the molecular geometry is optimized by semi-empirical method “Ground State/DFT/Default Spin/B3PW91” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, Where S1 and T1 are used directly.

HOMO(eV)=((HOMO(Gaussian)×27.212)−0.9899)/1.1206

LUMO(eV)=((LUMO(Gaussian)×27.212)−2.0041)/1.385

Where HOMO(G) and LUMO(G) are the direct calculation results of Gaussian 03W, in units of Hartree. The results are shown in Table 1 below:

TABLE 1

| | Homo Corr. | Lumo Corr. | Singlet S1 | Triplet T1 | | S1 − T1 | ΔHomo Corr. | ΔLumo Corr. |
|---|---|---|---|---|---|---|---|---|
| Compound | [eV] | [eV] | [eV] | [eV] | f(S1) | [eV] | [eV] | [eV] |
| H14 | −5.39 | −2.98 | 2.72 | 1.43 | 0.67 | 1.28 | 1.09 | 1.15 |
| H13 | −5.32 | −2.94 | 2.66 | 1.43 | 0.57 | 1.23 | 0.59 | 1.00 |
| H12 | −5.27 | −2.90 | 2.71 | 1.43 | 0.80 | 1.28 | 1.25 | 1.46 |
| H11 | −5.26 | −2.89 | 2.71 | 1.43 | 0.81 | 1.27 | 1.25 | 1.45 |
| H10 | −6.13 | −2.97 | 3.28 | 2.22 | 0.06 | 1.06 | 0.20 | 0.85 |
| H9 | −5.99 | −2.99 | 2.88 | 1.99 | 0.03 | 0.89 | 0.23 | 1.71 |
| H8 | −5.34 | −2.88 | 2.83 | 1.47 | 0.48 | 1.36 | 0.97 | 1.27 |
| H7 | −5.32 | −2.94 | 2.66 | 1.43 | 0.57 | 1.23 | 0.59 | 1.00 |
| H6 | −5.27 | −2.86 | 2.75 | 1.45 | 0.61 | 1.30 | 0.95 | 1.19 |
| H5 | −5.26 | −2.89 | 2.71 | 1.43 | 0.81 | 1.28 | 1.16 | 1.36 |
| H4 | −5.24 | −2.74 | 2.89 | 1.54 | 0.39 | 1.36 | 1.24 | 1.41 |
| H3 | −5.63 | −2.60 | 3.56 | 2.02 | 0.26 | 1.54 | 0.73 | 0.74 |
| H2 | −5.68 | −2.68 | 3.47 | 2.02 | 0.40 | 1.45 | 0.49 | 0.49 |
| H1 | −5.64 | −2.52 | 3.71 | 2.06 | 0.32 | 1.64 | 0.61 | 0.89 |

### Optical Performance Testing

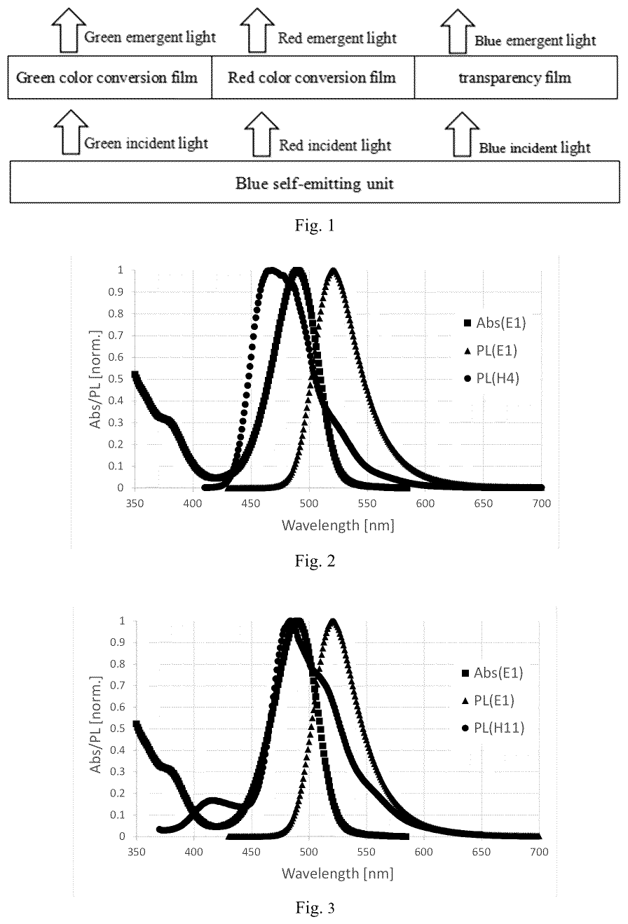

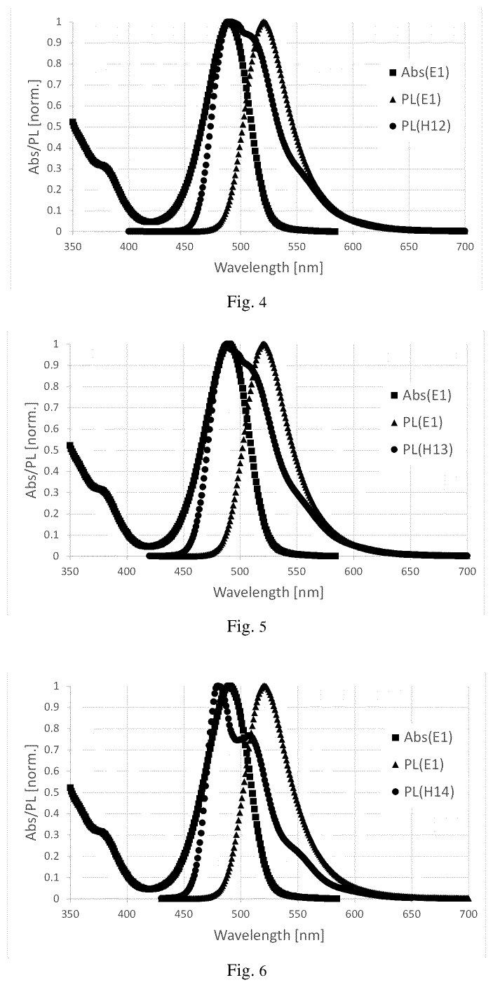

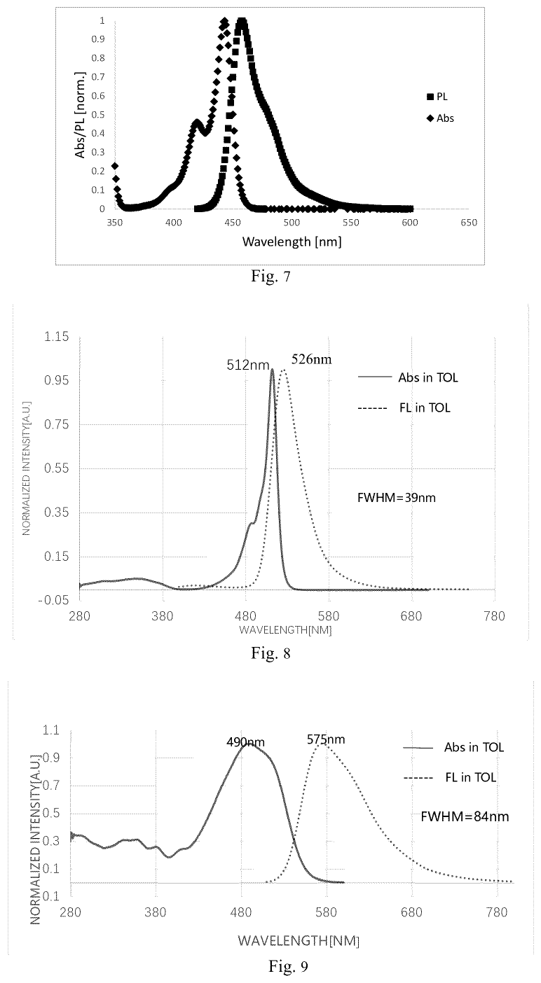

The absorption and emission spectrum of the compounds E1, E3, E4, and E6 in toluene are respectively shown in FIGS. 1, 7, 8, and 9; and the emission spectrum of compounds H4, H11-H14 in toluene are respectively shown in FIGS. 2-6. The emission spectrum of the compounds H4, H11-H14 have a large overlap with the absorption spectrum of the compound E1, allowing for an efficient energy transfer (i.e., Förster resonance energy transfer (FRET)) therebetween. In addition, the compound E1 exhibits a narrow-band green emission with a FWHM of less than 50 nm, while the compound E3 shows a narrow-band blue emission with a FWHM of about 30 nm.

### Preparation of Polymers-Containing Formulations and Organic Functional Films

100 mg of polymethyl methacrylate (PMMA), 50 mg of the host material Hx (i.e., H1-H14) for color conversion, and 5 mg of the emitter (i.e., the dopant material (E1) for green color conversion) were dissolved in 1 mL of n-butyl acetate to obtain a clear solution (i.e., a formulation or a printing ink). Using a KW-4a spin coater, the above clear solution was spun-coated on the surface of the quartz glass to form an uniform thin film, which is an organic functional film (i.e., a color conversion film). When thinner than 3 μm, most of the obtained color conversion films have an optical density (OD) larger than 3.

### Preparation of Resin Prepolymers-Containing Formulations and Organic Functional Films

The resin prepolymers-containing formulations and organic functional films could be obtained that the above-mentioned host and dopant materials for color conversion were premixed with a resin prepolymer such as methyl methacrylate, styrene, or methylstyrene. Initiated by 1-5 wt % of a photoinitiator (such as TPO (diphenyl (2,4,6-trimethylbenzoyl) phosphine oxide, 97%, CAS: 75980-60-8), the obtained formulation could form a thin film by spin coating, coating method, etc., and the obtained film was further cured by irradiation with a 365 nm or 390 nm UV LED lamp to form a color conversion film.

The green color conversion film can be disposed in a blue self-emitting unit that exhibits blue emission in the range of 400-490 nm. Through the green color converter, the blue light could change to a green light ranging from 490 nm to 550 nm.

### Preparation of Top-Emission OLED Devices

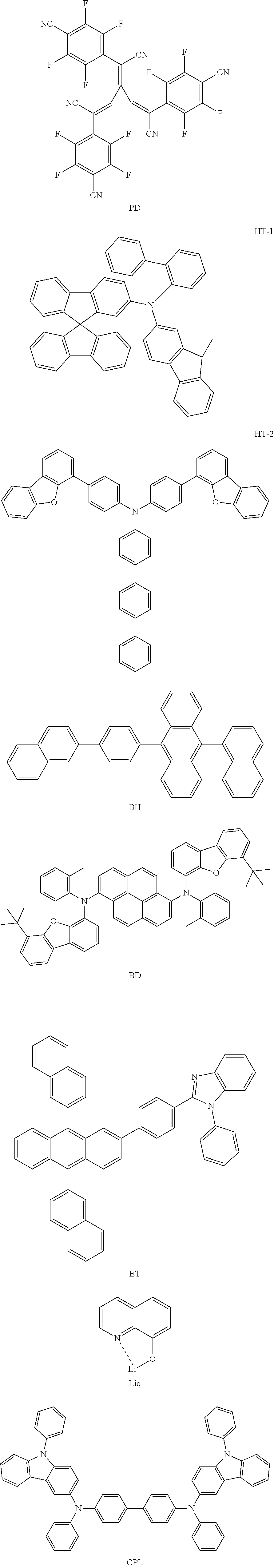 [0260] 1. Green light-emitting device 1:
a. Cleaning of Ag-containing ITO (indium tin oxide) top substrate: the substrate was ultrasonically cleaned with strip liquid, pure water, and isopropyl alcohol in sequence, then treated with ozone in argon after drying.
b. Evaporation: the resultant substrate was mounted on a vacuum deposition apparatus in high vacuum (1×10⁻⁶ mbar), the weight ratio of PD and HT-1 was controlled to be 3:100 to form a hole-injection layer (HIL) having a thickness of 10 nm, followed by evaporation of compound HT-1 on the hole-injection layer to form a hole-transportation layer (HTL) having a thickness of 120 nm, and then immediately followed by evaporation of compound HT-2 on the hole-transport layer to form a hole-buffer layer having a thickness of 10 nm. Then BH and BD at a weight ratio of 100:3 formed a light-emitting layer film having a thickness of 25 nm. Subsequently, ET and Liq were respectively placed in two different evaporation sources, and co-deposited on the light emitting layer at a weight ratio of 50:50 to form an electron-transport layer having a thickness of 35 nm. Yb was then deposited on the electron-transport layer to form an electron-injection layer having a thickness of 1.5 nm, and Mg:Ag (1:9) alloy was deposited on the electron-injection layer to form a cathode having a thickness of 16 nm.
c. CPL with a thickness of 70 nm was evaporated on the cathode and used as an optical capping layer.
d. H7:E2 (9:1) was evaporated on the CPL to form a color conversion layer with a thickness of 800 nm.
e. Encapsulation: encapsulating the device in a nitrogen-regulated glove box with UV curable resin.
2. Green light-emitting device 2: Except the steps c and d, other steps are the same as described in the procedures for preparing the above-mentioned green light-emitting device 1.
c. H7:E2 (9:1) was evaporated on the cathode to form a color conversion layer with a thickness of 800 nm.
d. CPL with a thickness of 70 nm was evaporated on the color conversion layer and used as an optical capping layer.
3. Green light-emitting device 3: Except the steps c and d, other steps are the same as described in the procedures for preparing the above-mentioned green light-emitting device 1.
c. CPL:H7:E2 (5.5:5.5:1) was evaporated on the cathode to form a color conversion layer with a thickness of 800 nm.
d. Encapsulation: encapsulating the device in a nitrogen-regulated glove box with UV curable resin.
4. Green light-emitting device 4: Except the following, other steps are the same as described in the procedures for preparing the above-mentioned green light-emitting device 1.
b. Evaporation: other procedures are consistent with those in the preparation of green light emitting device 1, except that pure BH was used to form the light-emitting layer with a thickness of 25 nm.
d. BD:H7:E2 (5.5:5.5:1) was evaporated on the CPL to form a color conversion layer with a thickness of 800 nm.
5. Blue light-emitting device 1: Except the following, other steps are the same as described in the procedures for preparing the above-mentioned green light-emitting device 1.
b. Evaporation: other procedures are consistent with those in the preparation of green light emitting device 1, except that pure BH was used to form the light-emitting layer with a thickness of 25 nm.
d. BH:E3 (8:2) was evaporated on the CPL to form a color conversion layer with a thickness of 800 nm.
6. Green light-emitting device 5: Except the steps c and d, other steps are the same as described in the procedures for preparing the above-mentioned green light-emitting device 1.
c. H7:E4 (9:1) was evaporated on the cathode to form a color conversion layer with a thickness of 800 nm.
d. CPL with a thickness of 70 nm was evaporated on the color conversion layer and used as an optical capping layer.
7. Red light-emitting device 1: Except the steps c and d, other steps are the same as described in the procedures for preparing the above-mentioned green light-emitting device 1.
c. H7:E5 (9:1) was evaporated on the cathode to form a color conversion layer with a thickness of 800 nm.
d. CPL with a thickness of 70 nm was evaporated on the color conversion layer and used as an optical capping layer.
8. Red light-emitting device 2: Except the steps c and d, other steps are the same as described in the procedures for preparing the above-mentioned green light-emitting device 1.
c. H7:E6 (9:1) was evaporated as on the cathode to form the color conversion layer with a thickness of 800 nm.
d. CPL with a thickness of 70 nm was evaporated on the color conversion layer and used as an optical capping layer.

All of the above light-emitting devices 1-8 have high color purity, where the FWHMs of emission spectrum of the light-emitting devices 1-7 are within 55 nm; the FWHMs of emission spectrum of the light-emitting devices 1-5 are below 30 nm.

Similar results can be obtained by replacing the evaporation-based OLEDs with QD-LEDs or printed OLEDs.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. A formulation, comprising an organic compound H, an emitter E, and an organic resin, wherein an emission spectrum of the organic compound H is on a short wavelength side of an absorption spectrum of the emitter E, and at least partially overlaps with the absorption spectrum of the emitter E; and a full width at half maximum (FWHM) of an emission spectrum of the emitter E≤55 nm.

2. The formulation according to claim 1, wherein the emitter E comprises a structural unit of formula (1) or (2): 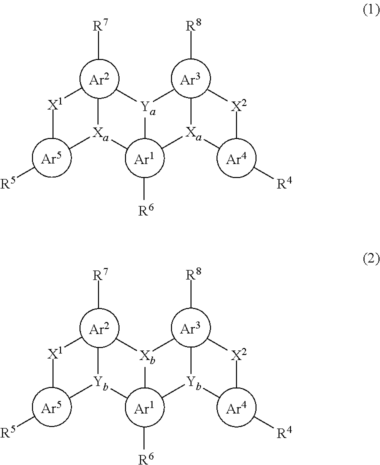
wherein:
each of Ar¹ to Ar³ is independently selected from an aromatic or a heteroaromatic group containing 5 to 24 ring atoms;
each of Ar⁴ and Ar⁵ is independently selected from null, an aromatic or a heteroaromatic group containing 5 to 24 ring atoms;
when neither Ar⁴ nor Ar⁵ is null, each of Xₐ and X_{b} at each occurrence is independently selected from N, C(R⁹), or Si(R⁹); each of Yₐ and Y_{b} at each occurrence is independently selected from B, P═O, C(R⁹), or Si(R⁹);
when Ar⁴ and/or Ar⁵ is null, each X_{b} is selected from N, C(R⁹), or Si(R⁹); each Yₐ is selected from B, P═O, C(R⁹), or Si(R⁹); each of Xₐ and Y_{b} at each occurrence is independently selected from N(R⁹), C(R⁹R¹⁰), Si(R⁹R¹⁰), C═O, O, C═N(R⁹), C═C(R⁹R¹⁰), P(R⁹), P(═O)R⁹, S, S═O, or SO₂;
each of X¹ and X² is independently selected from null or a bridging group;
each of R⁴ to R¹⁰ at each occurrence is independently selected from the group consisting of —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, and a disubstituted unit in any position of the above substituents or a combination thereof, wherein one or more R⁴-R¹⁰ may form a monocyclic or polycyclic aliphatic or aromatic ring system with each other and/or with the rings bonded thereto.

3. The formulation according to claim 2, wherein the emitter E comprises a structural unit of one of the following formulas (1a)-(1e) or (2a)-(2e):

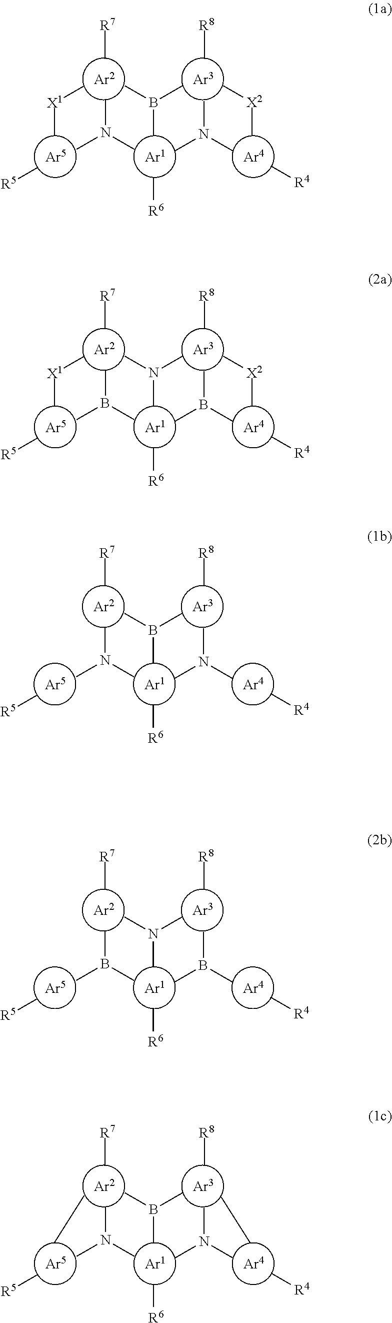

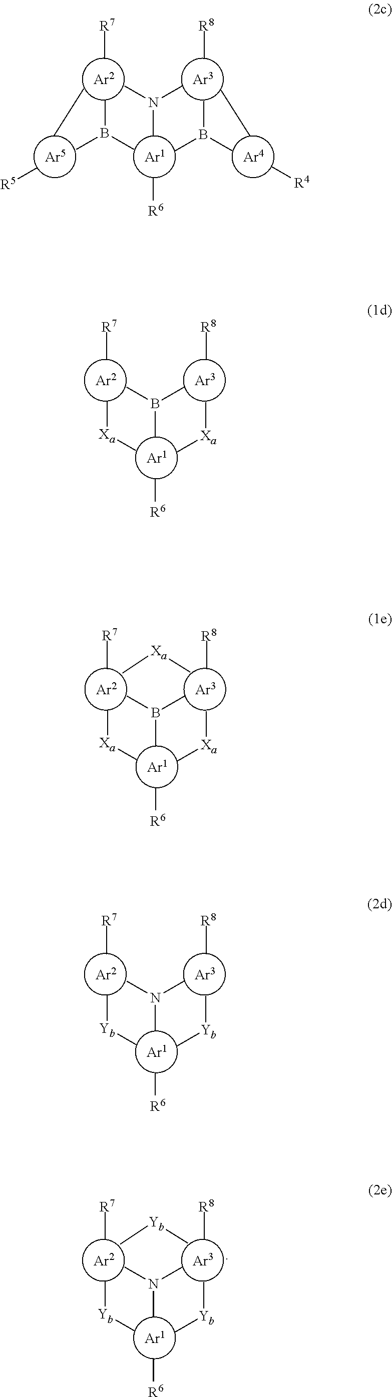

4. The formulation according to claim 2, wherein each of Ar¹, Ar², Ar³, Ar⁴, and Ar⁵ is independently selected from one of the following structural formulas or any combination thereof:

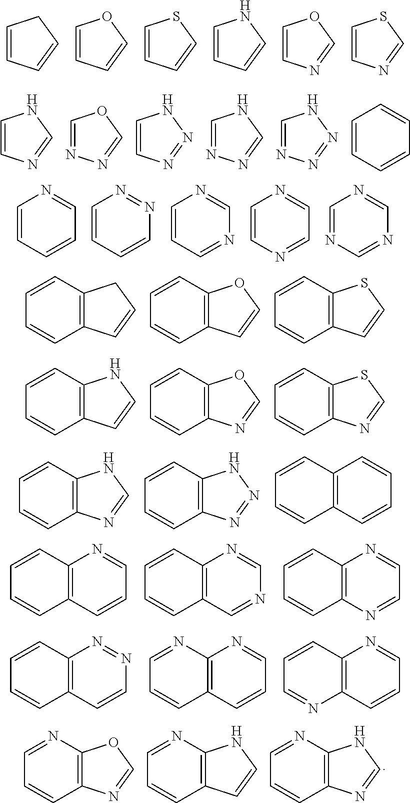

5. The formulation according to claim 3, wherein each of Ar¹, Ar², Ar³, Ar⁴, and Ar⁵ is independently selected from one of the following structural formulas or any combination thereof:

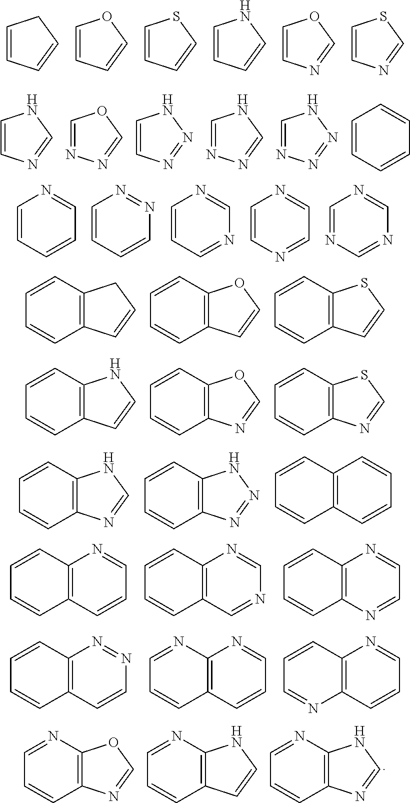

6. The formulation according to claim 1, wherein the organic compound H comprises at least one of the following groups: 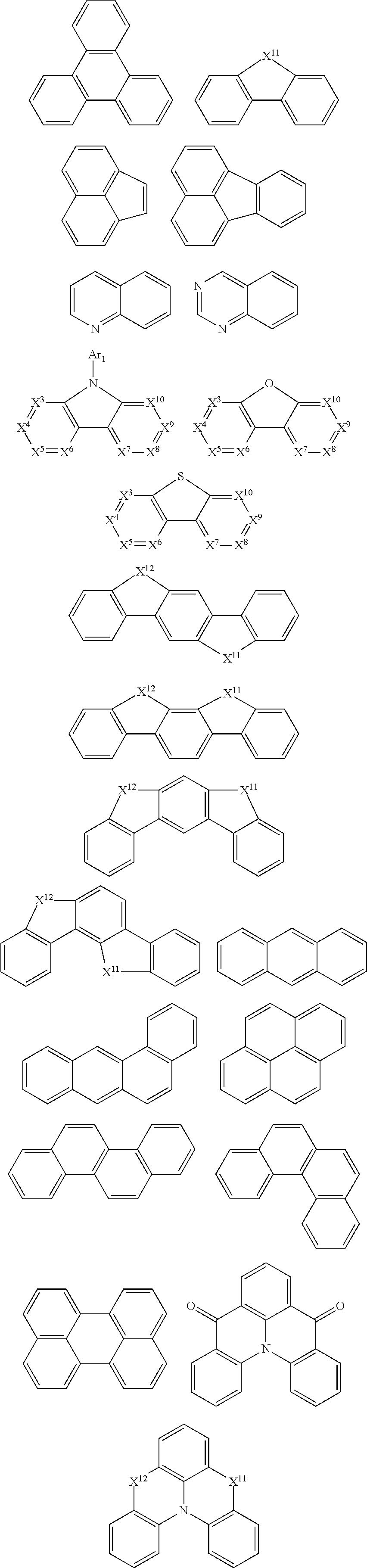
wherein: Ar₁ is aryl or heteroaryl group; each of X³ to X¹⁰ at each occurrence is CR¹ or N; each of X¹¹ and X¹² at each occurrence is independently selected from CR¹R², NR¹, or O; each of R¹ and R² at each occurrence is independently selected from the group consisting of —H, -D, a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, and a disubstituted unit in any position of the above substituents or a combination thereof, wherein one or more R¹-R² may form a monocyclic or polycyclic aliphatic or aromatic ring system with each other and/or with the rings bonded thereto.

7. The formulation according to claim 1, wherein the organic resin is a thermosetting resin or a UV curable resin.

8. The formulation according to claim 1, wherein the weight ratio of the organic resin is between 20 wt % and 99 wt %.

9. The formulation according to claim 1, wherein the formulation further comprises at least one solvent.

10. The formulation according to claim 9, wherein the at least one solvent is selected from water, alcohols, esters, aromatic ketones, aromatic ethers, aliphatic ketones, aliphatic ethers, borates, phosphorates, or a combination of two or more of them.

11. An organic functional film, comprising the formulation according to claim 1.

12. An organic light-emitting device, comprising a substrate, a first electrode, an organic light-emitting layer, a second electrode, a color conversion layer, and an encapsulation layer in sequence from bottom to top, wherein the second electrode is at least partially transparent, the color conversion layer comprises the formulation according to claim 1; the color conversion layer absorbs 90% or more of the light emitted by the organic light-emitting layer through the second electrode; the emission spectrum of the organic compound H is on the short wavelength side of the absorption spectrum of the emitter E, and at least partially overlaps with the absorption spectrum of the emitter E; and the FWHM of the emission spectrum of the emitter E≤55 nm.
